(19) United States
(12) Patent Application Publication
(10) Pub. No.: US 2022/0006018 A1
(43) Pub. Date: Jan. 6, 2022

(54) COMPOUNDS THAT CAN BE USED FOR PRODUCING AN ORGANIC ELECTRONIC DEVICE

(71) Applicant: Merck Patent GmbH, Darmstadt (DE)

(72) Inventor: Philipp STOESSEL, Frankfurt am Main (DE)

(21) Appl. No.: 17/293,515

(22) PCT Filed: Nov. 12, 2019
(86) PCT No.: PCT/EP2019/080916

§ 371 Date: May 13, 2021

(30) Foreign Application Priority Data
Nov. 14, 2018 (EP) 18206254.7

(51) Int. Cl. H01L 51/00; C07D 213/16; C07F 15/00; C07D 209/94; C07D 215/06; C07D 405/14; C07D 213/74; C07D 213/06; C07D 403/04; C07C 211/54; C07D 251/24

(52) U.S. Cl. CPC H01L 51/0054; H01L 51/5016; C07F 15/0033; H01L 51/0067; C07D 209/94; H01L 51/0072; C07D 215/06; C07D 405/14; H01L 51/0073; C07D 213/74; C07D 213/06; C07D 403/04; C07C 211/54; C07D 251/24; H01L 51/0085; C07F 15/0086; H01L 51/0087; C07C 2603/76; C07D 213/16

(57) ABSTRACT

The invention relates to compounds that can be used for producing functional layers of electronic devices, in particular for use in electronic devices. The invention further relates to a process for preparing the compounds according to the invention, and to electronic devices comprising same.

The present invention describes compounds, especially for use in electronic devices. The invention further relates to a process for preparing the compounds of the invention and to electronic devices comprising these compounds.

The structure of organic electroluminescent devices (OLEDs) in which organic semiconductors are used as functional materials is described, for example, in U.S. Pat. Nos. 4,539,507, 5,151,629, EP 0676461 and WO 98/27136. Emitting materials used are frequently organometallic complexes which exhibit phosphorescence. For quantum-mechanical reasons, up to four times the energy efficiency and power efficiency is possible using organometallic compounds as phosphorescent emitters. In general terms, there is still a need for improvement in OLEDs, especially also in OLEDs which exhibit phosphorescence, for example with regard to efficiency, operating voltage and lifetime. Also known are organic electroluminescent devices comprising phosphorescent emitters, fluorescent emitters or emitters that exhibit TADF (thermally activated delayed fluorescence).

The properties of organic electroluminescent devices are not only determined by the emitters used. Also of particular significance here are especially the other materials used, such as host/matrix materials, hole blocker materials, electron transport materials, hole transport materials and electron or exciton blocker materials. Improvements to these materials can lead to distinct improvements to electroluminescent devices.

In general terms, in the case of these materials, for example for use as matrix materials, hole conductor materials or electron transport materials, there is still a need for improvement, particularly in relation to the lifetime, but also in relation to the efficiency and operating voltage of the device. Moreover, the compounds should have high color purity.

It is a further object of the present invention to provide compounds which are suitable for use in an organic electronic device, especially in an organic electroluminescent device, as emitters, preferably as phosphorescent emitters, fluorescent emitters or emitters that exhibit TADF (thermally activated delayed fluorescence), and which lead to good device properties when used in this device, and to provide the corresponding electronic device.

It is therefore an object of the present invention to provide compounds which are suitable for use in an organic electronic device, especially in an organic electroluminescent device, and which lead to good device properties when used in this device, and to provide the corresponding electronic device.

It is a particular object of the present invention to provide compounds which lead to a high lifetime, good efficiency and low operating voltage. Particularly the properties of the matrix materials, the hole conductor materials or the electron transport materials too have an essential influence on the lifetime and efficiency of the organic electroluminescent device.

A further problem addressed by the present invention can be considered that of providing compounds suitable for use in a phosphorescent or fluorescent OLED, especially as a matrix material. More particularly, a problem addressed by the present invention is that of providing matrix materials suitable for red-, yellow- and green-phosphorescing OLEDs.

In addition, the compounds, especially when they are used as matrix materials, as hole conductor materials or as electron transport materials in organic electroluminescent devices, should lead to devices having excellent color purity.

Moreover, the compounds should be processible in a very simple manner, and especially exhibit good solubility and film formation. For example, the compounds should exhibit elevated oxidation stability and an improved glass transition temperature.

A further problem addressed can be considered that of providing electronic devices having excellent performance very inexpensively and in constant quality.

Furthermore, it should be possible to use or adapt the electronic devices for many purposes. More particularly, the performance of the electronic devices should be maintained over a broad temperature range.

It has been found that, surprisingly, particular compounds that are described in detail hereinafter solve these problems and eliminate the disadvantage from the prior art. The use of the compounds leads to very good properties of organic electronic devices, especially of organic electroluminescent devices, especially with regard to lifetime, efficiency and operating voltage. The present invention therefore provides electronic devices, especially organic electroluminescent devices, comprising compounds of this kind, and the corresponding preferred embodiments.

The present invention therefore provides an organofunctional compound usable for production of functional layers of electronic devices, which is characterized in that the compound comprises at least one structural element of the formula (I) and/or (Ia), the compound preferably having the formulae mentioned,

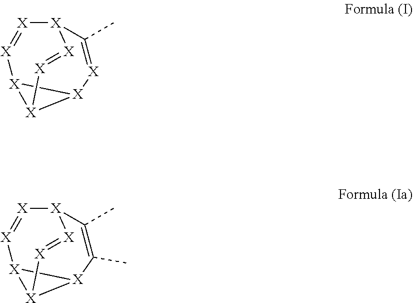

where the dotted bond represents the linkage of this group to another part of the organofunctional compound and in addition:
X is the same or different at each instance and is CR or N, with the proviso that not more than three, preferably not more than two, symbols X are N;
R is the same or different at each instance and is H, D, OH, F, Cl, Br, I, CN, NO₂, N(Ar)₂, N(R¹)₂, C(═O)N(Ar)₂, C(═O)N(R¹)₂, Si(Ar)₃, Si(R¹)₃, Ge(Ar)₃, Ge(R¹)₃, B(Ar)₂, B(R¹)₂, C(═O)Ar, C(═O)R¹, P(═O)(Ar)₂, P(═O)(R¹)₂, P(Ar)₂, P(R¹)₂, S(═O)Ar, S(═O)R¹, S(═O)₂Ar, S(═O)₂R¹, OSO₂Ar, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C≡C, Si(R¹)₂, Ge(R¹)₂, Sn(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals together may also form a ring system;
Ar is the same or different at each instance and is an aromatic or heteroaromatic ring system which has 5 to 60 aromatic ring atoms and may be substituted by one or more R¹ radicals; at the same time, it is possible for two Ar radicals bonded to the same silicon atom, nitrogen atom, phosphorus atom or boron atom also to be joined together via a bridge by a single bond or a bridge selected from B(R¹), C(R¹)₂, Si(R¹)₂, Ge(R¹)₂, C═O, C═NR¹, C═C(R¹)₂, O, S, S═O, SO₂, N(R¹), P(R¹) and P(═O)R¹;
R¹ is the same or different at each instance and is H, D, F, Cl, Br, I, CN, NO₂, N(Ar¹)₂, N(R²)₂, C(═O)Ar¹, C(═O)R², P(═O)(Ar¹)₂, P(Ar¹)₂, B(Ar¹)₂, B(R²)₂, Si(Ar¹)₃, Si(R²)₃, Ge(Ar¹)₃, Ge(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, Ge(R²)₂, Sn(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more, preferably adjacent R¹ radicals together may form a ring system; at the same time, one or more R¹ radicals with a further part of the compound may form a ring system;
Ar¹ is the same or different at each instance and is an aromatic or heteroaromatic ring system which has 5 to 30 aromatic ring atoms and may be substituted by one or more, preferably nonaromatic R² radicals; at the same time, it is possible for two Ar¹ radicals bonded to the same silicon atom, nitrogen atom, phosphorus atom or boron atom also to be joined to one another via a bridge by a single bond or a bridge selected from B(R²), C(R²)₂, Si(R²)₂, C═O, C═NR², C═C(R²)₂, O, S, S═O, SO₂, N(R²), P(R²) and P(═O)R²;
R² is the same or different at each instance and is H, D, F, Cl, Br, I, CN, B(OR³)₂, NO₂, C(═O)R³, CR³═C(R³)₂, C(═O)OR³, C(═O)N(R³)₂, Si(R³)₃, Ge(R³)₃, P(R³)₂, B(R³)₂, N(R³)₂, NO₂, P(═O)(R³)₂, OSO₂R³, OR³, S(═O)R³, S(═O)₂R³, a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R³ radicals, where one or more nonadjacent CH₂ groups may be replaced by —R³C═CR³—, —C≡C—, Si(R³)₂, Ge(R³)₂, Sn(R³)₂, C═O, C═S, C═NR³, —C(═O)O—, —C(═O)NR³—, NR³, P(═O)(R³), —O—, —S—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R³ radicals, or an aryloxy or heteroaryloxy group which has 5 to 40 aromatic ring atoms and may be substituted by one or more R³ radicals, or a combination of these systems; at the same time, two or more, preferably adjacent substituents R² together may also form a ring system;
R³ is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, Cl, Br, I or CN and which may be substituted by one or more alkyl groups each having 1 to 4 carbon atoms; at the same time, two or more, preferably adjacent substituents R³ together may form a ring system.

Compounds usable for production of functional layers of electronic devices are generally the organic or inorganic materials introduced between anode and cathode, for example in an organic electronic device, especially in an organic electroluminescent device, for example charge injection, charge transport or charge blocker materials, but especially emission materials and matrix materials. Preference is given here to organic materials.

In a preferred embodiment, the compound usable for production of functional layers of electronic devices is a purely organic compound. A purely organic compound is a compound not associated with a metal atom, i.e. not forming a coordination compound with a metal atom nor forming a covalent bond with a metal atom. A purely organic compound here preferably does not comprise any metal atom which is used in phosphorescent emitters. These metals, such as copper, molybdenum, etc., especially rhenium, ruthenium, osmium, rhodium, iridium, palladium, will be discussed in detail later on.

The compound usable for production of functional layers of electronic devices is preferably selected from the group consisting of fluorescent emitters, phosphorescent emitters, emitters that exhibit TADF (thermally activated delayed fluorescence), host materials, electron transport materials, exciton blocker materials, electron injection materials, hole conductor materials, hole injection materials, n-dopants, p-dopants, wide bandgap materials, electron blocker materials and/or hole blocker materials.

In a preferred configuration, the compounds of the invention may comprise at least one structural element of the formula (II) and/or (IIa), the compound preferably having the formula specified:

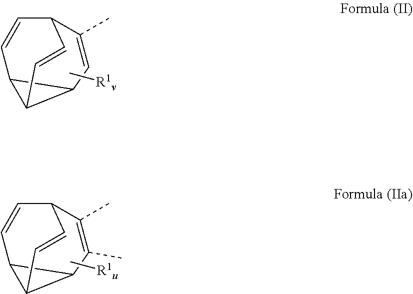

where the dotted bond represents the linkage of this group to another part of the organofunctional compound, the R¹ radicals have the definition given above, especially for formula (I) and/or (IIa), the index v is 0, 1, 2, 3, 4, 5, 6, 7, 8 or 9, preferably 0, 1, 2, 3, 4, 5 or 6, more preferably 0, 1, 2, 3 or 4 and especially preferably 0 or 1, and the index u is 0, 1, 2, 3, 4, 5, 6, 7 or 8, preferably 0, 1, 2, 3, 4, 5 or 6, more preferably 0, 1, 2, 3 or 4 and especially preferably 0 or 1.

Adjacent carbon atoms in the context of the present invention are carbon atoms bonded directly to one another. In addition, “adjacent radicals” in the definition of the radicals means that these radicals are bonded to the same carbon atom or to adjacent carbon atoms. These definitions apply correspondingly, inter alia, to the terms “adjacent groups” and “adjacent substituents”.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

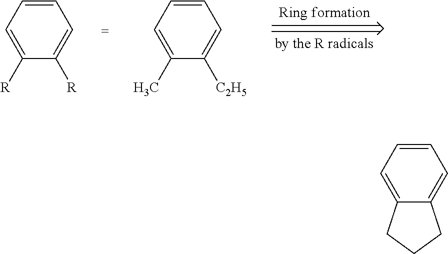

In addition, however, the abovementioned wording shall also be understood to mean that, if one of the two radicals is hydrogen, the second radical binds to the position to which the hydrogen atom was bonded, forming a ring. This shall be illustrated by the following scheme:

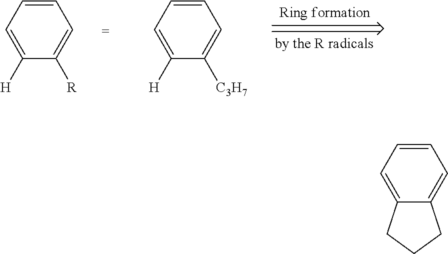

A fused aryl group, a fused aromatic ring system or a fused heteroaromatic ring system in the context of the present invention is a group in which two or more aromatic groups are fused, i.e. annelated, to one another along a common edge, such that, for example, two carbon atoms belong to the at least two aromatic or heteroaromatic rings, as, for example, in naphthalene. By contrast, for example, fluorene is not a fused aryl group in the context of the present invention, since the two aromatic groups in fluorene do not have a common edge. Corresponding definitions apply to heteroaryl groups and to fused ring systems which may but need not also contain heteroatoms.

If two or more, preferably adjacent R, R¹, R² and/or R³ radicals together form a ring system, the result may be a monocyclic or polycyclic, aliphatic, aromatic or heteroaromatic ring system.

An aryl group in the context of this invention contains 6 to 60 carbon atoms, preferably 6 to 40 carbon atoms, more preferably 6 to 30 carbon atoms; a heteroaryl group in the context of this invention contains 2 to 60 carbon atoms, preferably 2 to 40 carbon atoms, more preferably 2 to 30 carbon atoms, and at least one heteroatom, with the proviso that the sum total of carbon atoms and heteroatoms is at least 5. The heteroatoms are preferably selected from N, O and/or S. Here, an aryl group or heteroaryl group is understood to mean either a simple aromatic cycle, i.e. benzene, or a simple heteroaromatic cycle, for example pyridine, pyrimidine, thiophene, etc., or a fused aryl or heteroaryl group, for example naphthalene, anthracene, phenanthrene, quinoline, isoquinoline, etc.

An aromatic ring system in the context of this invention contains 6 to 60 carbon atoms, preferably 6 to 40 carbon atoms, more preferably 6 to 30 carbon atoms, in the ring system. A heteroaromatic ring system in the context of this invention contains 1 to 60 carbon atoms, preferably 1 to 40 carbon atoms, more preferably 1 to 30 carbon atoms, and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for a plurality of aryl or heteroaryl groups to be interrupted by a nonaromatic unit (preferably less than 10% of the atoms other than H), for example a carbon, nitrogen or oxygen atom or a carbonyl group. For example, systems such as 9,9′-spirobifluorene, 9,9-diarylfluorene, triarylamine, diaryl ethers, stilbene, etc. shall thus also be regarded as aromatic ring systems in the context of this invention, and likewise systems in which two or more aryl groups are interrupted, for example, by a linear or cyclic alkyl group or by a silyl group. In addition, systems in which two or more aryl or heteroaryl groups are bonded directly to one another, for example biphenyl, terphenyl, quaterphenyl or bipyridine, shall likewise be regarded as an aromatic or heteroaromatic ring system.

A cyclic alkyl, alkoxy or thioalkoxy group in the context of this invention is understood to mean a monocyclic, bicyclic or polycyclic group.

In the context of the present invention, a C₁- to C₂₀-alkyl group in which individual hydrogen atoms or CH₂ groups may also be substituted by the abovementioned groups is understood to mean, for example, the methyl, ethyl, n-propyl, i-propyl, cyclopropyl, n-butyl, i-butyl, s-butyl, t-butyl, cyclobutyl, 2-methylbutyl, n-pentyl, s-pentyl, t-pentyl, 2-pentyl, neopentyl, cyclopentyl, n-hexyl, s-hexyl, t-hexyl, 2-hexyl, 3-hexyl, neohexyl, cyclohexyl, 1-methylcyclopentyl, 2-methyl pentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2.2.2]octyl, 2-bicyclo[2.2.2]octyl, 2-(2,6-dimethyl)octyl, 3-(3,7-dimethyl)octyl, adamantyl, trifluoromethyl, pentafluoroethyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)cyclohex-1-yl, 1-(n-butyl)cyclohex-1-yl, 1-(n-hexyl)cyclohex-1-yl, 1-(n-octyl)cyclohex-1-yl and 1-(n-decyl)cyclohex-1-yl radicals. An alkenyl group is understood to mean, for example, ethenyl, propenyl, butenyl, pentenyl, cyclopentenyl, hexenyl, cyclohexenyl, heptenyl, cycloheptenyl, octenyl, cyclooctenyl or cyclooctadienyl. An alkynyl group is understood to mean, for example, ethynyl, propynyl, butynyl, pentynyl, hexynyl, heptynyl or octynyl. A C₁- to C₄₀-alkoxy group is understood to mean, for example, methoxy, trifluoromethoxy, ethoxy, n-propoxy, i-propoxy, n-butoxy, i-butoxy, s-butoxy, t-butoxy or 2-methylbutoxy.

An aromatic or heteroaromatic ring system which has 5 to 60, preferably 5-40, aromatic ring atoms, more preferably 5 to 30 aromatic ring atoms, and may also be substituted in each case by the abovementioned radicals and which may be joined to the aromatic or heteroaromatic system via any desired positions is understood to mean, for example, groups derived from benzene, naphthalene, anthracene, benzanthracene, phenanthrene, benzophenanthrene, pyrene, chrysene, perylene, fluoranthene, benzofluoranthene, naphthacene, pentacene, benzopyrene, biphenyl, biphenylene, terphenyl, terphenylene, triphenylene, fluorene, spirobifluorene, dihydrophenanthrene, dihydropyrene, tetrahydropyrene, cis- or trans-indenofluorene, cis- or trans-monobenzoindenofluorene, cis-ortrans-dibenzoindenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

In a further configuration, preference is given to compounds in which the structural element of the formula (I), (Ia), (II) and/or (IIa) of the organofunctional compound has high symmetry, and is preferably symmetrically substituted by a further part of the organofunctional compound based on the linkage site(s) of this group.

It may further be the case that the organofunctional compound is selected from the group of the fluorenes, indenofluorenes, spirobifluorenes, carbazoles, indenocarbazoles, indolocarbazoles, spirocarbazoles, pyrimidines, triazines, lactams, triarylamines, dibenzofurans, dibenzothiophenes, dibenzothienes, imidazoles, benzimidazoles, benzoxazoles, benzothiazoles, 5-arylphenanthridin-6-ones, 9,10-dehydrophenanthrenes, fluoranthenes, anthracenes, benzanthracenes, fluoradenes.

It may further be the case that the organofunctional compound comprises a group selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 9,9′-diarylfluorenyl 1-, 2-, 3- or 4-spirobifluorenyl, pyridyl, pyrimidinyl, 1-, 2-, 3- or 4-dibenzofuranyl, 1-, 2-, 3- or 4-dibenzothienyl, pyrenyl, triazinyl, imidazolyl, benzimidazolyl, benzoxazolyl, benzothiazolyl, 1-, 2-, 3- or 4-carbazolyl, 1- or 2-naphthyl, anthracenyl, preferably 9-anthracenyl, trans- and cis-indenofluorenyl, indenocarbazolyl, indolocarbazolyl, spirocarbazolyl, 5-aryl-phenanthridin-6-on-yl, 9,10-dehydrophenanthrenyl, fluoranthenyl, tolyl, mesityl, phenoxytolyl, anisolyl, triarylaminyl, bis(triarylaminyl), tris(triarylaminyl), hexamethylindanyl, tetralinyl, monocycloalkyl, biscycloalkyl, tricycloalkyl, alkyl, for example tert-butyl, methyl, propyl, alkoxyl, alkylsulfanyl, alkylaryl, triarylsilyl, trialkylsilyl, xanthenyl, 10-arylphenoxazinyl, phenanthrenyl and/or triphenylenyl, each of which may be substituted by one or more radicals, but are preferably unsubstituted, particular preference being given to phenyl, spirobifluorene, fluorene, dibenzofuran, dibenzothiophene, anthracene, phenanthrene, triphenylene groups, where a functional structural element A^{a} preferably comprises a corresponding group or can be represented by a corresponding group.

When the bullvalene structure of the formulae (I), (Ia), (II) and/or (IIa) is substituted by substituents R and/or R¹, these substituents R and/or R¹ are preferably selected from the group consisting of H, D, F, CN, N(Ar)₂, N(Ar¹)₂, C(═O)Ar, C(═O)Ar¹, P(═O)(Ar)₂, P(═O)(Ar¹)₂, a straight-chain alkyl or alkoxy group having 1 to 10 carbon atoms or a branched or cyclic alkyl or alkoxy group having 3 to 10 carbon atoms or an alkenyl group having 2 to 10 carbon atoms, each of which may be substituted by one or more R¹ or R² radicals, where one or more nonadjacent CH₂ groups may be replaced by O and where one or more hydrogen atoms may be replaced by D or F, an aromatic or heteroaromatic ring system which has 5 to 24 aromatic ring atoms and may be substituted in each case by one or more R¹ or R² radicals, but is preferably unsubstituted, or an aralkyl or heteroaralkyl group which has 5 to 25 aromatic ring atoms and may be substituted by one or more R¹ or R² radicals; at the same time, it is optionally possible for two substituents R and/or R¹ preferably bonded to adjacent carbon atoms to form a monocyclic or polycyclic, aliphatic, aromatic or heteroaromatic ring system which may be substituted by one or more R¹ or R² radicals; where the Ar or Ar¹ group has the definition given above, especially for formula (I) or (Ia).

More preferably, these substituents R and/or R¹ are selected from the group consisting of H, D, F, CN, N(Ar)₂, N(Ar¹)₂, a straight-chain alkyl group having 1 to 8 carbon atoms, preferably having 1, 2, 3 or 4 carbon atoms, or a branched or cyclic alkyl group having 3 to 8 carbon atoms, preferably having 3 or 4 carbon atoms, or an alkenyl group having 2 to 8 carbon atoms, preferably having 2, 3 or 4 carbon atoms, each of which may be substituted by one or more R¹ radicals, but is preferably unsubstituted, or an aromatic or heteroaromatic ring system which has 5 to 24 aromatic ring atoms, preferably 6 to 18 aromatic ring atoms, more preferably 6 to 13 aromatic ring atoms, and may be substituted in each case by one or more nonaromatic R¹ or R² radicals, but is preferably unsubstituted; at the same time, two substituents R¹ or R² preferably bonded to adjacent carbon atoms may optionally form a monocyclic or polycyclic aliphatic ring system which may be substituted by one or more R² or R³ radicals, but is preferably unsubstituted, where Ar or Ar¹ may have the definition set out above.

More preferably, the substituents R are selected from the group consisting of H or an aromatic or heteroaromatic ring system having 6 to 18 aromatic ring atoms, preferably having 6 to 13 aromatic ring atoms, each of which may be substituted by one or more nonaromatic R¹ radicals, but is preferably unsubstituted. Examples of suitable substituents R are selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl, 1-, 2-, 3- or 4-carbazolyl and indenocarbazolyl, each of which may be substituted by one or more R¹ radicals, but are preferably unsubstituted.

Most preferably, the substituents R¹ are selected from the group consisting of an aromatic or heteroaromatic ring system which has 6 to 18 aromatic ring atoms, preferably 6 to 13 aromatic ring atoms, and may be substituted in each case by one or more nonaromatic R² radicals, but is preferably unsubstituted. Examples of suitable substituents R¹ are selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl, 1-, 2-, 3- or 4-carbazolyl and indenocarbazolyl, each of which may be substituted by one or more R² radicals, but are preferably unsubstituted.

It may additionally be the case that the substituents R and/or R¹ of the bullvalene structure of the formulae (I), (Ia), (II) and/or (IIa) do not form a fused aromatic or heteroaromatic ring system, and preferably do not form any fused ring system. This includes the formation of a fused ring system with possible substituents R¹, R², R³ which may be bonded to the R¹ or R² radicals.

It may further be the case that the organofunctional compound comprises at least one group conforming to at least one of the formulae (IIIa), (IIIb), (IIIc), (IIId), (IIIe), (IIIf), (IIIg) and/or (IIIh)

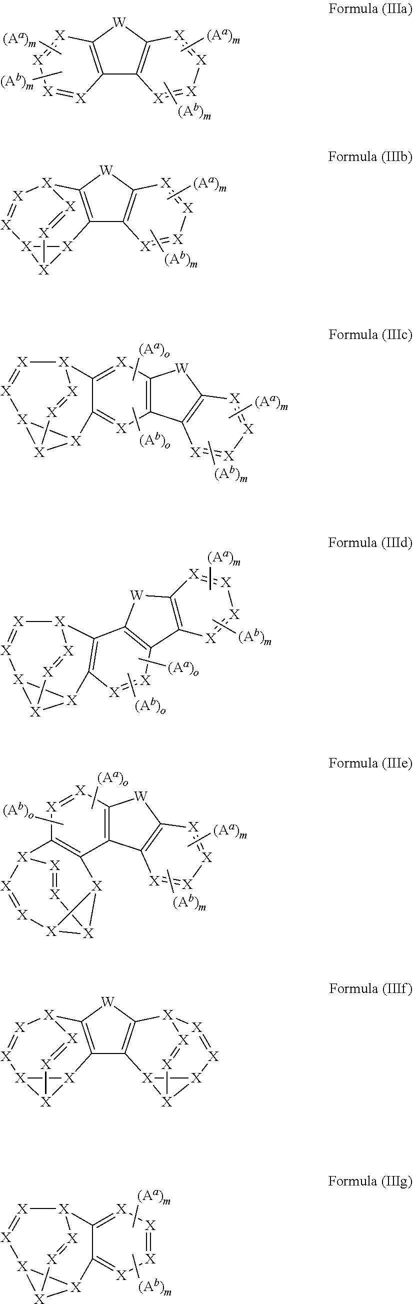

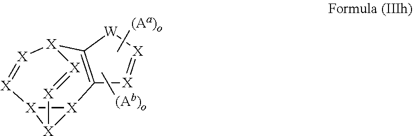

where the symbols used are as follows:
X is the same or different at each instance and is N or CR, preferably CR, or C if an A^{a} or A^{b} group is bonded to this atom, with the proviso that not more than two of the X groups in one cycle are N;
W is O, S, NR, NA^{a}, NA^{b}, BR, BA^{a}, BA^{b}, C(R)₂, CRA^{a}, C(A^{a})₂, CRA^{b}, C(A^{b})₂, CA^{a}A^{b}, —RC═CR—, —RC═CA^{a}-, -A^{a}C═CA^{a}-, —RC═CA^{b}-,
A^{b}C═CA^{b}-, -A^{b}C═CA^{a}-, SO, SO₂, Ge(R)₂, Ge(A^{a})₂, Ge(A^{b})₂, GeA^{a}A^{b}, Si(R)₂, Si(A^{a})₂, Si(A^{b})₂, SiA^{a}A^{b} or C═O;
m at each instance is independently 0, 1, 2, 3 or 4, preferably 0, 1 or 2, with the proviso that the sum total of the indices m per ring is not more than 4, preferably not more than 2;
o at each instance is independently 0, 1 or 2, preferably 0 or 1, with the proviso that the sum total of the indices o per ring is not more than 2, preferably not more than 1;
A^{a} is a functional structural element, preferably an aromatic or heteroaromatic ring system which has 5 to 40 ring atoms in each case and may be substituted by one or more substituents R;
A^{b} comprises, preferably is, a structure of formula (I) or (Ia) and/or a structure of formula (II) or (IIa),
   where the symbol R has the definition given above, especially for formula (I) or (Ia), with the proviso that the structure of formula (IIIa) has at least one A^{b} group. N—N bonds are preferably ruled out here.

In a further configuration, it may be the case that the organofunctional compound comprises at least one group conforming to at least one of the formulae (IVa), (IVb), (IVc), (IVd), (IVe), (IVf), (IVg) and/or (IVh)

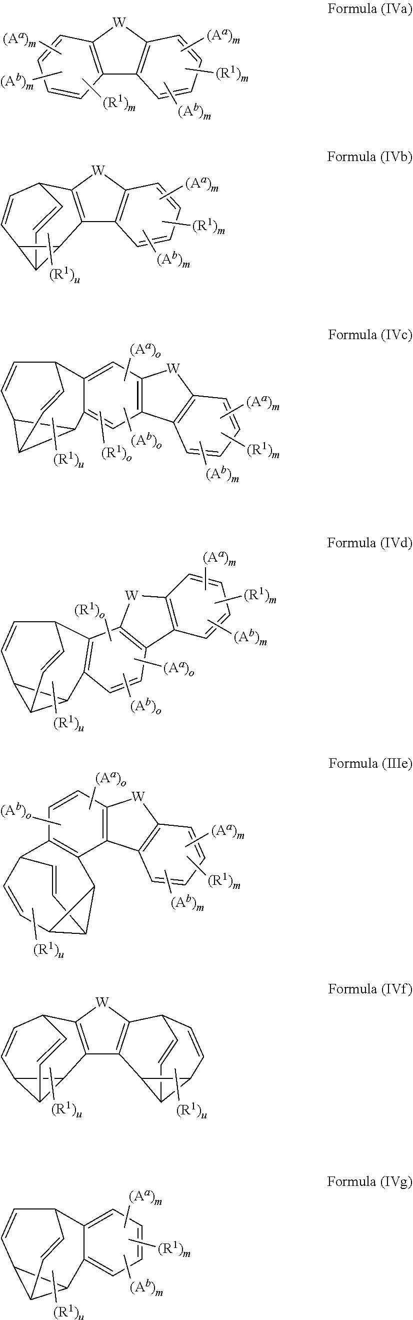

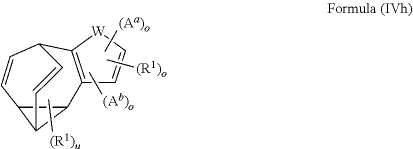

where the symbols A^{a}, A^{b}, W, m, o and R¹ have the definitions given above, especially for formula (I) or (Ia) or (IIIa) to (IIIh), and the index u is 0, 1, 2, 3, 4, 5, 6, 7 or 8, preferably 0, 1, 2, 3, 4, 5 or 6, more preferably 0, 1, 2, 3 or 4 and especially preferably 0 or 1, with the proviso that the structure of formula (IVa) comprises at least one A^{b} group.

The sum total of the A^{a} and/or A^{b} groups is preferably 1 to 10, particularly preferably 1 to 5, and is especially preferably 1, 2, 3 or 4.

Preferably, the sum total of the indices m, o and u in each of the structures of the formulae (IIIa) to (IIIh) and (IVa) to (IVh) is not more than 6, preferably not more than 4 and more preferably not more than 2.

It may preferably be the case that the functional structural element A^{a} in the structures of the formulae (IIIa) to (IIIh) and (IVa) to (IVh) has at least one aromatic or heteroaromatic ring system having 5 to 40 ring atoms in each case, which may be substituted by one or more substituents R¹.

Preferably, the functional structural element A^{a} in the structures of the formulae (IIIa) to (IIIh) and (IVa) to (IVh) is selected from the group of the fluorenes, indenofluorenes, spirobifluorenes, carbazoles, indenocarbazoles, indolocarbazoles, spirocarbazoles, pyrimidines, triazines, lactams, triarylamines, dibenzofurans, dibenzothiophenes, dibenzothienes, imidazoles, benzimidazoles, benzoxazoles, benzothiazoles, 5-arylphenanthridin-6-ones, 9,10-dehydrophenanthrenes, fluoranthenes, anthracenes, benzanthracenes, fluoradenes.

It may further be the case that the functional structural element A^{a} is selected from hole transport groups, electron transport groups, host material groups and wide bandgap groups. These groups are known as such and are described hereinafter.

In a further embodiment, it may be the case that the compound usable for production of functional layers of electronic devices comprises a hole transport group, the latter preferably being triarylamine or carbazole groups.

In a preferred embodiment, it may be the case that the hole transport group is joined to at least one bullvalene structure via one or two linkages represented as a dotted bond in formula (I) or (Ia).

In addition, at least one of the R and/or R¹ groups in a structure of the formulae (I), (Ia), (II), (IIa) (IIIa) to (IIIh) and/or (IVa) to (IVh), (IVc) (V) comprises, preferably represents, a hole transport group.

Hole transport groups are known in the technical field, and they preferably include triarylamine or carbazole groups.

It may preferably be the case that the hole transport group comprises a group and preferably is a group selected from the formulae (H-1) to (H-3)

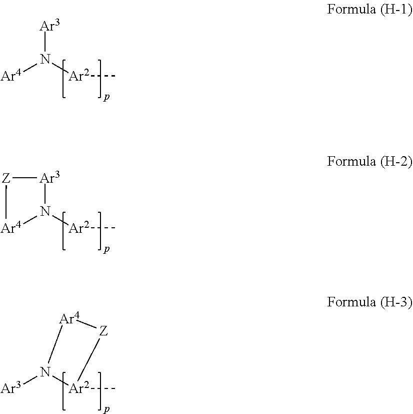

where the dotted bond marks the position of attachment and the symbols are defined as follows:
Ar², Ar³, Ar⁴ is in each case independently an aromatic ring system having 6 to 40 carbon atoms or a heteroaromatic ring system having 3 to 40 carbon atoms, each of which may be substituted by one or more R¹ radicals;
p is 0 or 1;
Z is a bond or C(R¹)₂, Si(R¹)₂, Ge(R¹)₂, C═O, NR¹, N—Ar¹, BR¹, PR¹, PO(R¹), SO, SO₂, Se, O or S, preferably a bond or C(R¹)₂, N—Ar¹, O or S;
   where the symbols Ar¹ and R¹ have the definition given above, especially for formula (I) and (Ia). At the same time, the presence of an N—N bond is preferably ruled out.

It may additionally be the case that the hole transport group comprises a group and preferably is a group selected from the formulae (H-4) to (H-26)

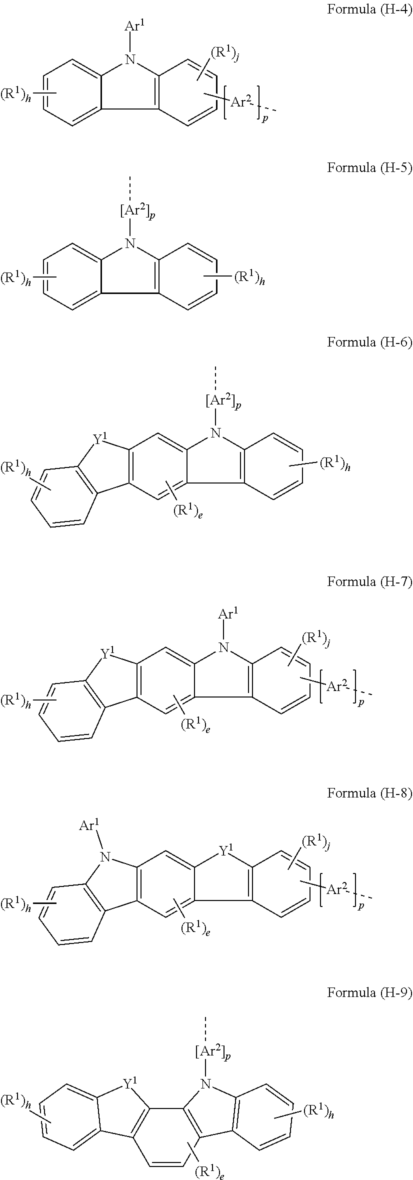

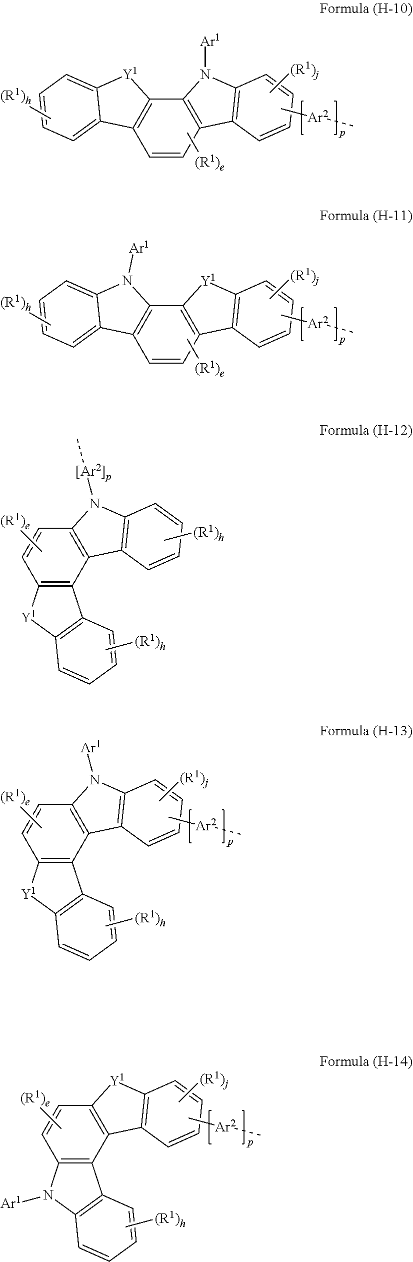

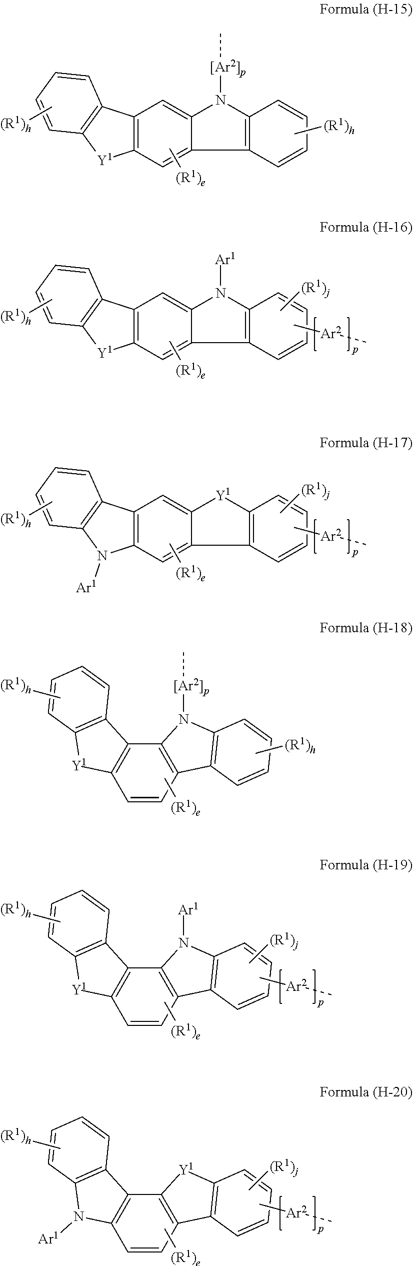

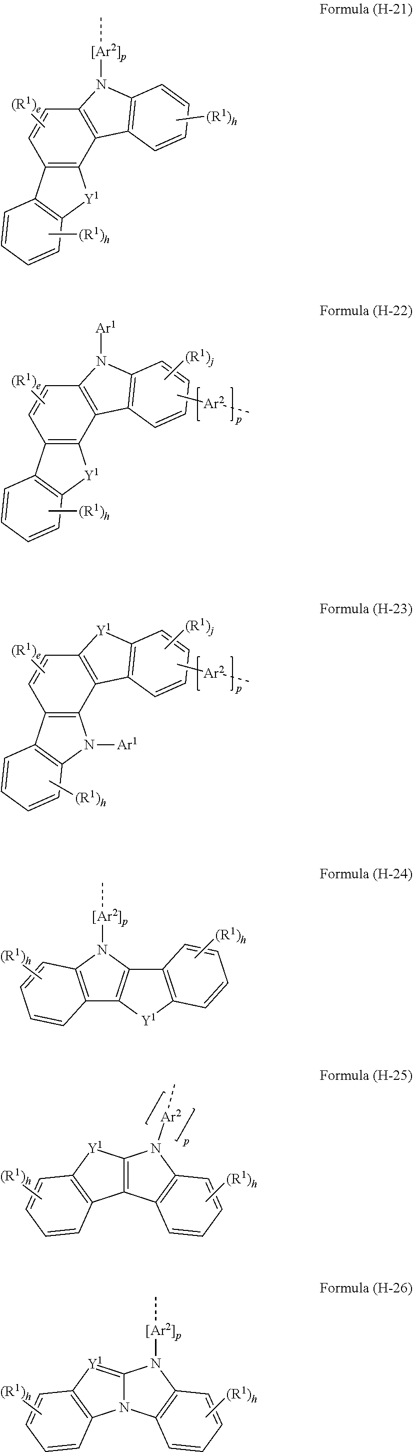

where Y¹ is O, S, C(R¹)₂, NR¹ or NAr¹, the dotted bond marks the position of attachment, e is 0, 1 or 2, j is 0, 1, 2 or 3, h is the same or different at each instance and is 0, 1, 2, 3 or 4, p is 0 or 1, Ar¹ and R¹ have the definitions given above, especially for formula (I) or (Ia), and Ar² has the definitions given above, especially for formula (H-1), (H-2) or (H-3). At the same time, the presence of an N—N bond is preferably ruled out.

The hole transport groups of the formulae (H-1) to (H-26) detailed above constitute preferred R¹ radicals of formulae (II), (IIa) and (IVa) to (IVh) or preferred embodiments of these formulae, where in this case the R¹ groups detailed in the formulae (H-1) to (H-26) should be replaced by R² radicals.

It is clear from the above wording that, if the index is p=0, the corresponding Ar² group is absent and a bond is formed.

Preferably, the Ar² group may form through-conjugation with the aromatic or heteroaromatic radical or the nitrogen atom to which the Ar² group of the formulae (H-1) to (H-26) may be bonded.

In a further preferred embodiment of the invention, Ar² is an aromatic or heteroaromatic ring system which has 5 to 14 aromatic or heteroaromatic ring atoms, preferably an aromatic ring system which has 6 to 12 carbon atoms, and which may be substituted by one or more R¹ radicals, but is preferably unsubstituted, where R¹ may have the definition given above, especially for formula (I). More preferably, Ar² is an aromatic ring system having 6 to 10 aromatic ring atoms or a heteroaromatic ring system having 6 to 13 heteroaromatic ring atoms, each of which may be substituted by one or more R¹ radicals, but is preferably unsubstituted, where R¹ may have the definition given above, especially for formula (I).

Further preferably, the symbol Ar² shown in formulae (H-1) to (H-26) inter alia is an aryl or heteroaryl radical having 5 to 24 ring atoms, preferably 6 to 13 ring atoms, more preferably 6 to 10 ring atoms, such that an aromatic or heteroaromatic group of an aromatic or heteroaromatic ring system is bonded to the respective atom of the further group directly, i.e. via an atom of the aromatic or heteroaromatic group.

It may further be the case that the Ar² group shown in formulae (H-1) to (H-26) comprises an aromatic ring system having not more than two fused aromatic and/or heteroaromatic 6-membered rings; preferably it does not comprise any fused aromatic or heteroaromatic ring system with fused 6-membered rings. Accordingly, naphthyl structures are preferred over anthracene structures. In addition, fluorenyl, spirobifluorenyl, dibenzofuranyl and/or dibenzothienyl structures are preferred over naphthyl structures. Particular preference is given to structures having no fusion, for example phenyl, biphenyl, terphenyl and/or quaterphenyl structures.

It may further be the case that the Ar² group shown in formulae (H-1) to (H-26) inter alia has not more than 1 nitrogen atom, preferably not more than 2 heteroatoms, particularly preferably not more than one heteroatom and especially preferably no heteroatom.

In a further preferred embodiment of the invention, Ar³ and/or Ar⁴ are the same or different at each instance and are an aromatic or heteroaromatic ring system having 6 to 24 aromatic ring atoms, preferably 6 to 18 aromatic ring atoms, and are more preferably an aromatic ring system having 6 to 12 aromatic ring atoms or a heteroaromatic ring system having 6 to 13 aromatic ring atoms, each of which may be substituted by one or more R¹ radicals, but is preferably unsubstituted, where R¹ may have the definition given above, especially in formula (I) or (Ia).

In a further embodiment, it may be the case that the compound usable for production of functional layers of electronic devices comprises an electron transport group-comprising radical.

In a preferred embodiment, it may be the case that the electron transport group-comprising radical is joined to at least one bullvalene structure via one or two linkages represented as a dotted bond in formula (I) or (Ia).

In addition, at least one of the R and/or R¹ groups in a structure of the formulae (I), (Ia), (II), (IIa) (IIIa) to (IIIh) and/or (IVa) to (IVh) comprises, preferably represents, an electron transport group-comprising radical.

Electron transport groups are widely known in the technical field and promote the ability of compounds to transport and/or to conduct electrons.

In addition, surprising advantages are shown by compounds usable for production of functional layers of electronic devices that comprise at least one structure selected from the group of the pyridines, pyrimidines, pyrazines, pyridazines, triazines, quinazolines, quinoxalines, quinolines, isoquinolines, imidazoles and/or benzimidazoles, particular preference being given to pyrimidines, triazines and quinazolines. These structures generally promote the ability of compounds to transport and/or to conduct electrons.

In a preferred configuration of the present invention, it may be the case that the electron transport group-comprising radical is a group that can be represented by the formula (QL)

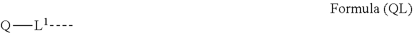

in which L¹ represents a bond or an aromatic or heteroaromatic ring system which has 5 to 40, preferably 5 to 30, aromatic ring atoms and may be substituted by one or more R¹ radicals, Q is an electron transport group, where R¹ has the definition given above, especially for formula (I), and the dotted bond marks the position of attachment.

The substituents R¹ here in the structure of the formula (QL) should be replaced in structures of formulae (II), (IIa) and (IVa) to (IVh) by substituents R².

Preferably, the L¹ group may form through-conjugation with the Q group and the atom, preferably the carbon or nitrogen atom, to which the L¹ group of formula (QL) is bonded. Through-conjugation of the aromatic or heteroaromatic systems is formed as soon as direct bonds are formed between adjacent aromatic or heteroaromatic rings. A further bond between the aforementioned conjugated groups, for example via a sulfur, nitrogen or oxygen atom or a carbonyl group, is not detrimental to conjugation. In the case of a fluorene system, the two aromatic rings are bonded directly, where the sp³-hybridized carbon atom in position 9 does prevent fusion of these rings, but conjugation is possible since this sp³-hybridized carbon atom in position 9 does not necessarily lie between the electron-transporting Q group and the atom via which the group of formula (QL) is bonded to further structural elements of a compound of the invention. In contrast, in the case of a second spirobifluorene structure, through-conjugation can be formed if the bond between the Q group and the aromatic or heteroaromatic radical to which the L¹ group of formula (QL) is bonded is via the same phenyl group in the spirobifluorene structure or via phenyl groups in the spirobifluorene structure that are bonded directly to one another and are in one plane. If the bond between the Q group and the aromatic or heteroaromatic radical to which the L¹ group of formula (QL) is bonded is via different phenyl groups in the second spirobifluorene structure bonded via the sp³-hybridized carbon atom in position 9, the conjugation is interrupted.

In a further preferred embodiment of the invention, L¹ is a bond or an aromatic or heteroaromatic ring system which has 5 to 14 aromatic or heteroaromatic ring atoms, preferably an aromatic ring system which has 6 to 12 carbon atoms, and which may be substituted by one or more R¹ radicals, but is preferably unsubstituted, where R¹ may have the definition given above, especially for formula (I). More preferably, L¹ is an aromatic ring system having 6 to 10 aromatic ring atoms or a heteroaromatic ring system having 6 to 13 heteroaromatic ring atoms, each of which may be substituted by one or more R² radicals, but is preferably unsubstituted, where R² may have the definition given above, especially for formula (I).

Further preferably, the symbol L¹ shown in formula (QL) inter alia is the same or different at each instance and is a bond or an aryl or heteroaryl radical having 5 to 24 ring atoms, preferably 6 to 13 ring atoms, more preferably 6 to 10 ring atoms, such that an aromatic or heteroaromatic group of an aromatic or heteroaromatic ring system is bonded to the respective atom of the further group directly, i.e. via an atom of the aromatic or heteroaromatic group.

It may additionally be the case that the L¹ group shown in formula (QL) comprises an aromatic ring system having not more than two fused aromatic and/or heteroaromatic 6-membered rings, preferably does not comprise any fused aromatic or heteroaromatic ring system. Accordingly, naphthyl structures are preferred over anthracene structures. In addition, fluorenyl, spirobifluorenyl, dibenzofuranyl and/or dibenzothienyl structures are preferred over naphthyl structures.

Particular preference is given to structures having no fusion, for example phenyl, biphenyl, terphenyl and/or quaterphenyl structures.

Examples of suitable aromatic or heteroaromatic ring systems L¹ are selected from the group consisting of ortho-, meta- or para-phenylene, ortho-, meta- or para-biphenylene, terphenylene, especially branched terphenylene, quaterphenylene, especially branched quaterphenylene, fluorenylene, spirobifluorenylene, dibenzofuranylene, dibenzothienylene and carbazolylene, each of which may be substituted by one or more R¹ radicals, but are preferably unsubstituted.

It may further be the case that the L¹ group shown in formula (QL) inter alia has not more than 1 nitrogen atom, preferably not more than 2 heteroatoms, especially preferably not more than one heteroatom and more preferably no heteroatom.

Preferably, the Q group shown in the formula (QL) inter alia, or the electron transport group, may be selected from structures of the formulae (Q-1), (Q-2), (Q-4), (Q-4), (Q-5), (Q-6), (Q-7), (Q-8), (Q-9) and/or (Q-10)

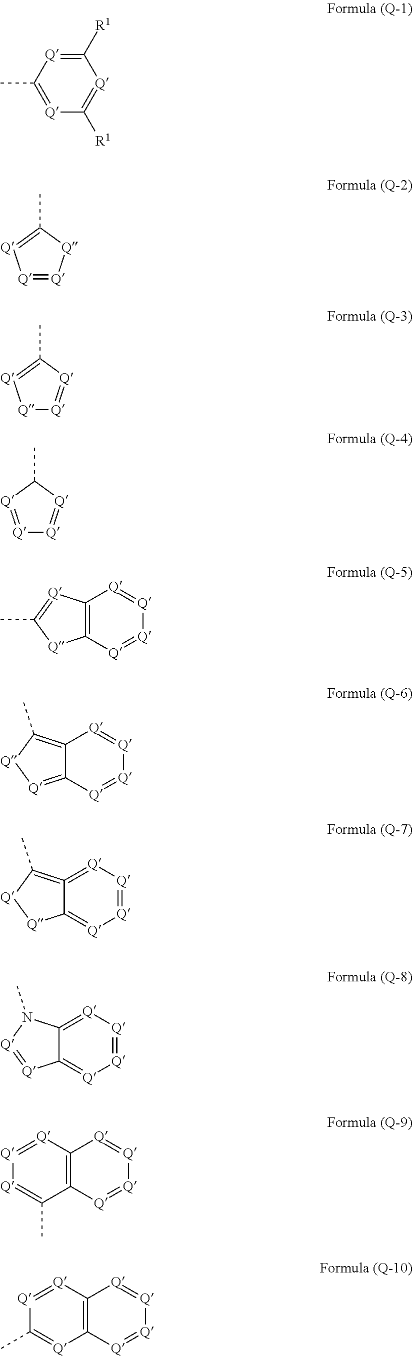

where the dotted bond marks the position of attachment,
Q′ is the same or different at each instance and is CR¹ or N, and

### Q″ is NR¹, O or S;

where at least one Q′ is N and
R¹ is as defined above, especially in formula (I) or (Ia).

The substituents R¹ in the structures of the formula (Q-1) to (Q-10) should be replaced in structures of formulae (II), (IIa) and (IVa) to (IVh) by substituents R².

In addition, the Q group shown in the formula (QL) inter alia, or the electron transport group, may preferably be selected from a structure of the formulae (Q-11), (Q-12), (Q-13), (Q-14) and/or (Q-15)

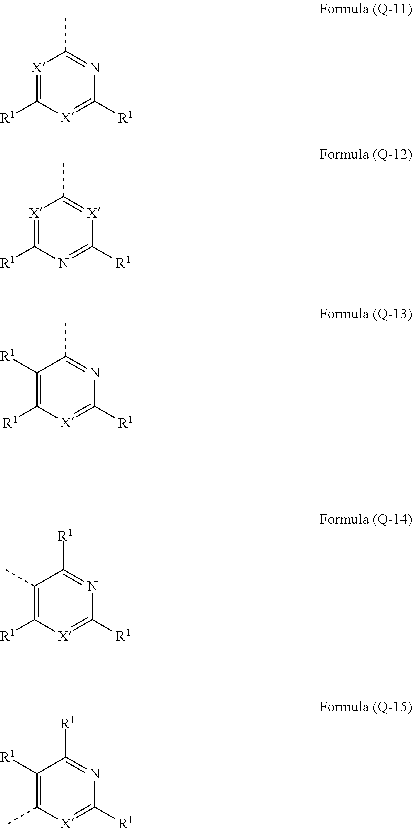

where the symbol R¹ has the definition given above for formula (I) inter alia, X¹ is N or CR¹ and the dotted bond marks the position of attachment, where X¹ is preferably a nitrogen atom.

In a further embodiment, the Q group shown in the formula (QL) inter alia, or the electron transport group, may be selected from structures of the formulae (Q-16), (Q-17), (Q-18), (Q-19), (Q-20), (Q-21) and/or (Q-22)

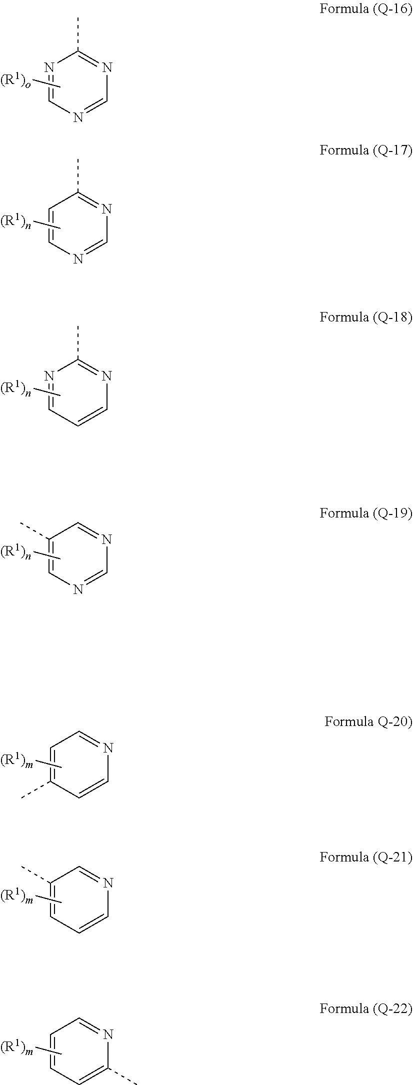

in which the symbol R¹ has the definition detailed above for formula (I) or (Ia) inter alia, the dotted bond marks the position of attachment and m is 0, 1, 2, 3 or 4, preferably 0, 1 or 2, n is 0, 1, 2 or 3, preferably 0, 1 or 2, and o is 0, 1 or 2, preferably 1 or 2. Preference is given here to the structures of the formulae (Q-16), (Q-17), (Q-18) and (Q-19).

In a further embodiment, the Q group shown in the formula (QL) inter alia, or the electron transport group, may be selected from structures of the formulae (Q-23), (Q-24) and/or (Q-25)

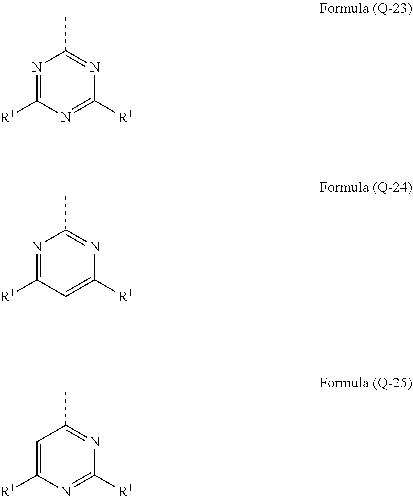

in which the symbol R¹ has the definition set out above for formula (I) or (Ia) inter alia, and the dotted bond marks the position of attachment.

In a further embodiment, the Q group shown in the formula (QL) inter alia, or the electron transport group, may be selected from structures of the formulae (Q-26), (Q-27), (Q-28), (Q-29) and/or (Q-30)

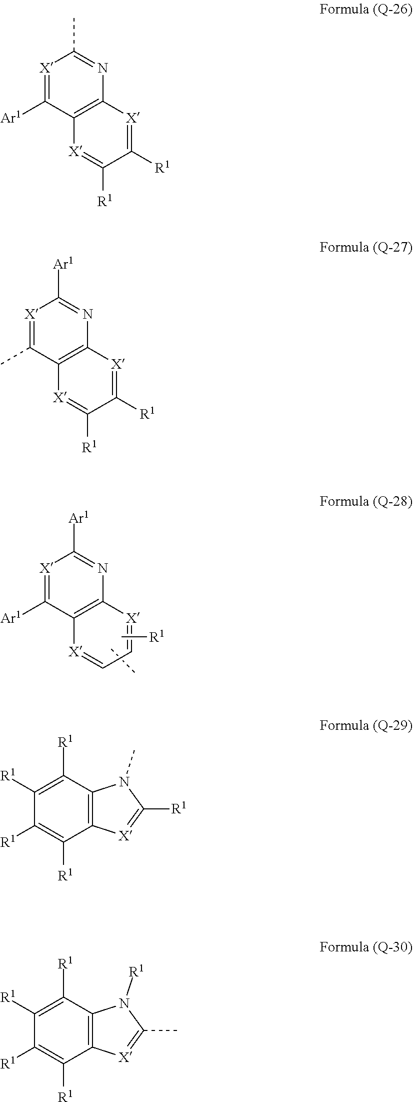

where symbols Ar¹ and R¹ have the definition given above for formula (I) or (Ia) inter alia, X¹ is N or CR¹ and the dotted bond marks the position of attachment. Preferably, in the structures of the formulae (Q-26), (Q-27) and (Q-28), exactly one X¹ is a nitrogen atom.

Preferably, the Q group shown in the formula (QL) inter alia, or the electron transport group, may be selected from structures of the formulae (Q-31), (Q-32), (Q-33), (Q-34), (Q-35), (Q-36), (Q-37), (Q-38), (Q-39), (Q-40), (Q-41), (Q-42), (Q-43) and/or (Q-44)

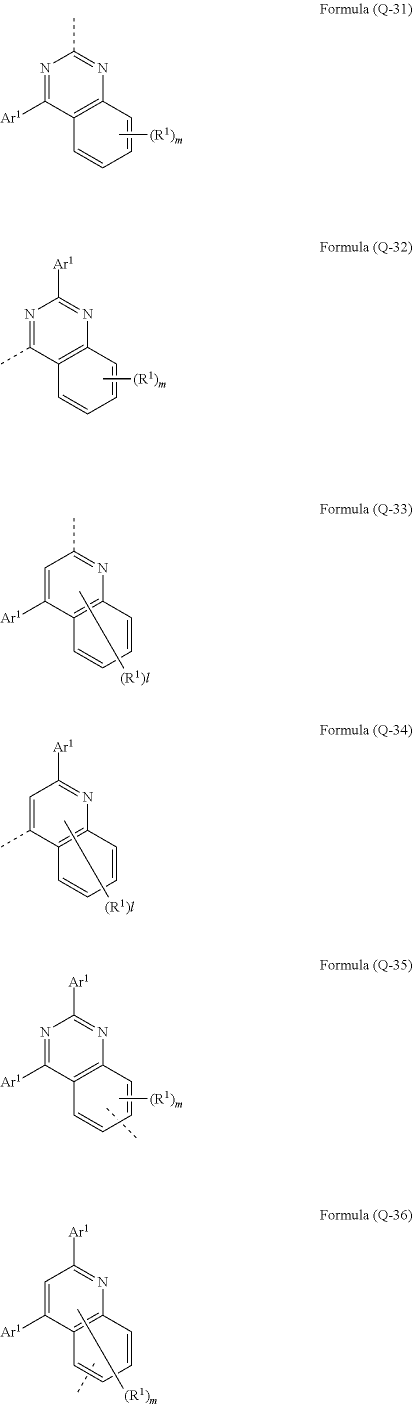

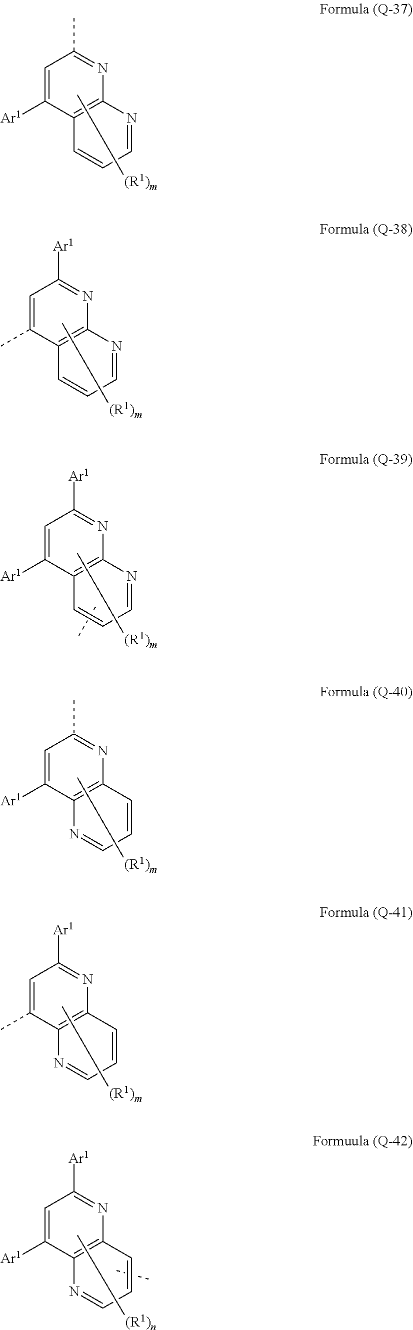

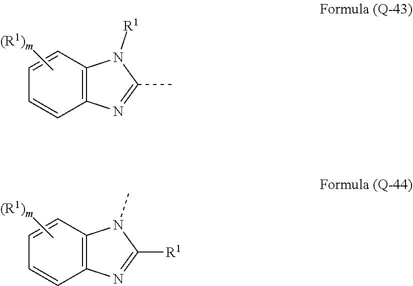

in which the symbols Ar¹ and R¹ have the definition set out above for formula (I) or (Ia) inter alia, the dotted bond marks the position of attachment and m is 0, 1, 2, 3 or 4, preferably 0, 1 or 2, n is 0, 1, 2 or 3, preferably 0 or 1, n is 0, 1, 2 or 3, preferably 0, 1 or 2, and l is 1, 2, 3, 4 or 5, preferably 0, 1 or 2.

The substituents R¹ in the structures of the formula (Q-11) to (Q-44) should be replaced in structures of formulae (II), (IIa) and (IVa) to (IVh) by substituents R².

In a further preferred embodiment of the invention, Ar¹ is the same or different at each instance and is an aromatic or heteroaromatic ring system, preferably an aryl or heteroaryl radical having 5 to 24 aromatic ring atoms, preferably having 6 to 18 aromatic ring atoms, and is more preferably an aromatic ring system, preferably an aryl radical having 6 to 12 aromatic ring atoms, or a heteroaromatic ring system, preferably a heteroaryl group having 5 to 13 aromatic ring atoms, each of which may be substituted by one or more R² radicals, but is preferably unsubstituted, where R² may have the definition detailed above, especially in formula (I).

Preferably, the symbol Ar¹ is an aryl or heteroaryl radical, such that an aromatic or heteroaromatic group of an aromatic or heteroaromatic ring system is bonded directly, i.e. via an atom of the aromatic or heteroaromatic group, to the respective atom of the further group, for example a carbon or nitrogen atom of the (H-1) to (H-26) or (Q-26) to (Q-44) groups shown above.

Advantageously, Ar¹ in the formulae (H-1) to (H-26) or (Q-26) to (Q-44) is an aromatic ring system which has 6 to 12 aromatic ring atoms and may be substituted by one or more R² radicals, but is preferably unsubstituted, where R² may have the definition detailed above, especially for formula (I).

Preferably, the R¹ or R² radicals in the formulae (H-1) to (H-26) or (Q-1) to (Q-44) do not form a fused ring system with the ring atoms of the aryl group or heteroaryl group Ar¹, Ar², Ar³ and/or Ar⁴ to which the R¹ or R² radicals are bonded. This includes the formation of a fused ring system with possible substituents R², R³ which may be bonded to the R¹ or R² radicals.

It may also be the case that the Ar, Ar¹, Ar², Ar³ and/or Ar⁴ group is selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl, pyrenyl, triazinyl, imidazolyl, benzimidazolyl, benzoxazolyl, benzothiazolyl, 1-, 2-, 3- or 4-carbazolyl, indenocarbazolyl, 1- or 2-naphthyl, anthracenyl, preferably 9-anthracenyl, phenanthrenyl and/or triphenylenyl, each of which may be substituted by one or more R¹ and/or R² radicals, but are preferably unsubstituted, particular preference being given to phenyl, spirobifluorene, fluorene, dibenzofuran, dibenzothiophene, anthracene, phenanthrene, triphenylene groups.

In a further configuration, it may be the case that the organofunctional compound comprises at least one group that leads to with wide bandgap materials. The expression “group that leads to with wide bandgap materials” sets out that the compounds can be used as wide bandgap materials, and so the compounds have corresponding groups. Wide bandgap materials are discussed in detail later on.

It may further be the case that the organofunctional compound comprises at least one group that leads to materials that are used as host material. The expression “group that leads to materials that are used as host material” sets out that the compounds can be used as host materials, and so the compounds have corresponding groups. Host materials will be discussed in detail later on.

In a further configuration, it may be the case that the compound usable for production of functional layers of electronic devices comprises a fused aromatic or heteroaromatic ring system having at least 2, preferably three, fused rings that may optionally be substituted.

In a further embodiment, it may be the case that the compound usable for production of functional layers of electronic devices comprises at least one aromatic or heteroaromatic ring system having at least two, preferably having three, fused aromatic or heteroaromatic rings.

In a preferred embodiment, it may be the case that the aromatic or heteroaromatic ring system is joined to at least one bullvalene structure by at least two, preferably by three, fused aromatic or heteroaromatic rings via one or two linkages represented as a dotted bond, for example in formula (I) or (Ia).

In addition, at least one of the R and/or R¹ groups in a structure of the formulae (I), (Ia), (II), (IIa) (IIIa) to (IIIh) and/or (IVa) to (IVh) comprises, preferably represents, at least one aromatic or heteroaromatic ring system having at least two, preferably having three, fused aromatic or heteroaromatic rings.

It may preferably be the case that the aromatic or heteroaromatic ring system having two, preferably having three, fused aromatic or heteroaromatic rings is selected from the groups of the formulae (Ar-1) to (Ar-11)

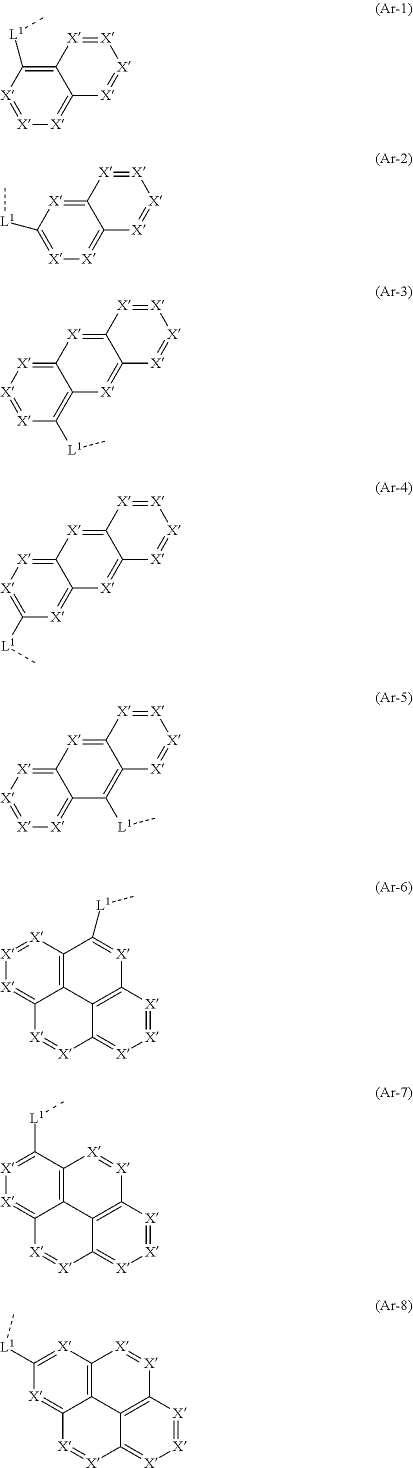

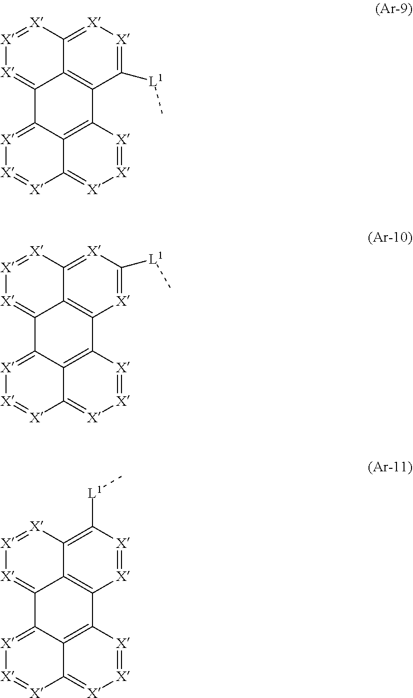

where X′ is N or CR¹, preferably CR¹, L¹ represents a bond or an aromatic or heteroaromatic ring system which has 5 to 40, preferably 5 to 30, aromatic ring atoms and may be substituted by one or more R¹ radicals, where R¹ has the definition set out above, especially for formula (I) or (Ia), and the dotted bond marks the position of attachment. The substructures of the formulae (Ar-1) to (Ar-11) preferably form a bond with structural elements of the formula (I) or (II).

It may further be the case that the structural element of the formula (Ia) or (IIa) is fused to an aromatic or heteroaromatic ring system which has 5 to 60 carbon atoms and is selected from the groups of the formulae (Ar-12) to (Ar-58)

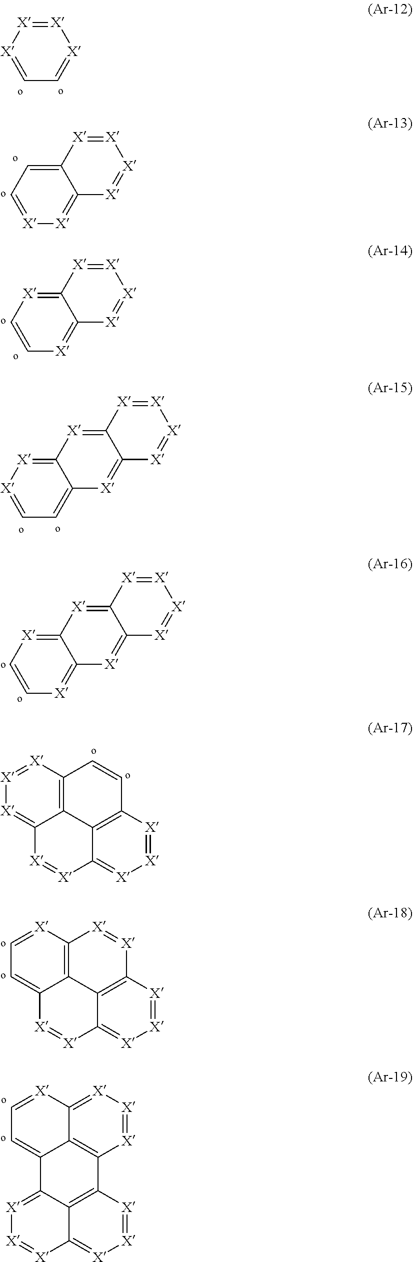

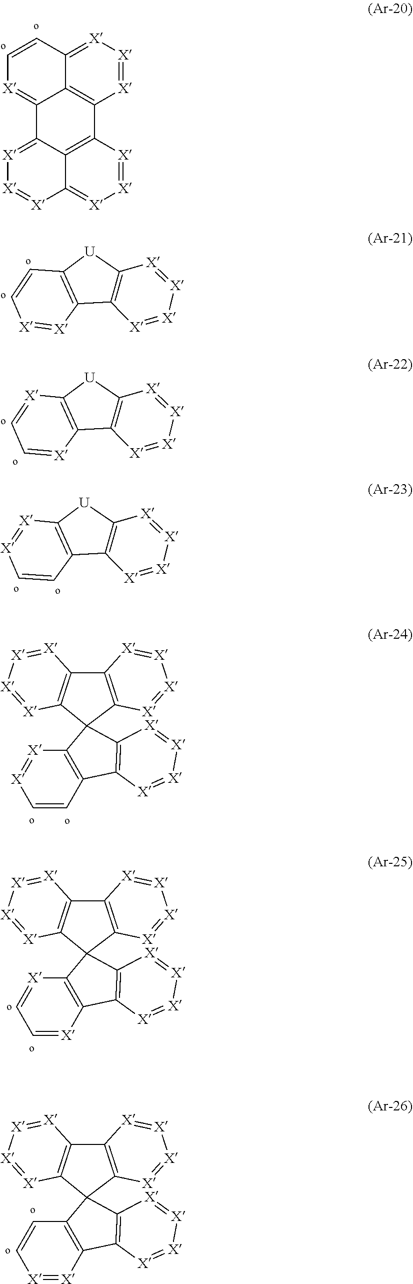

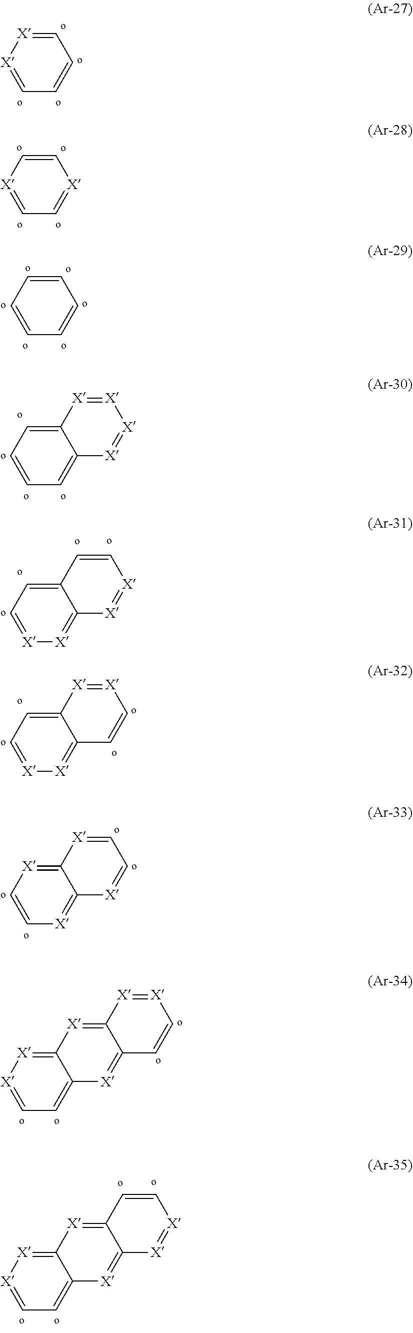

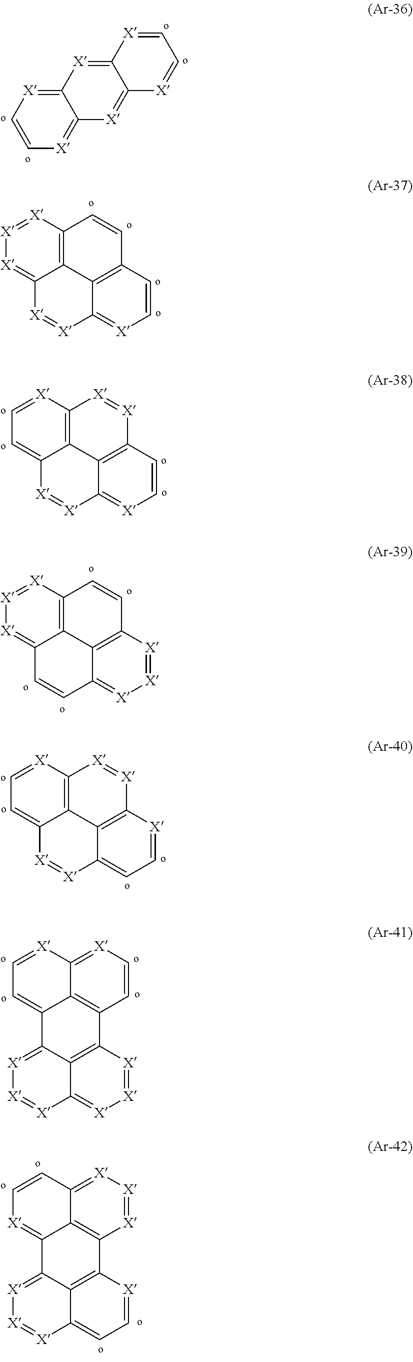

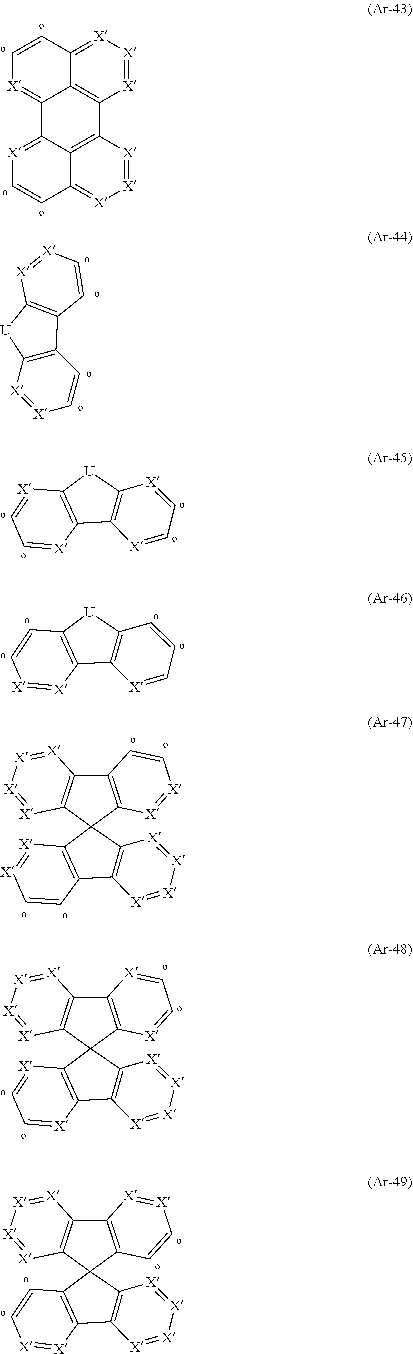

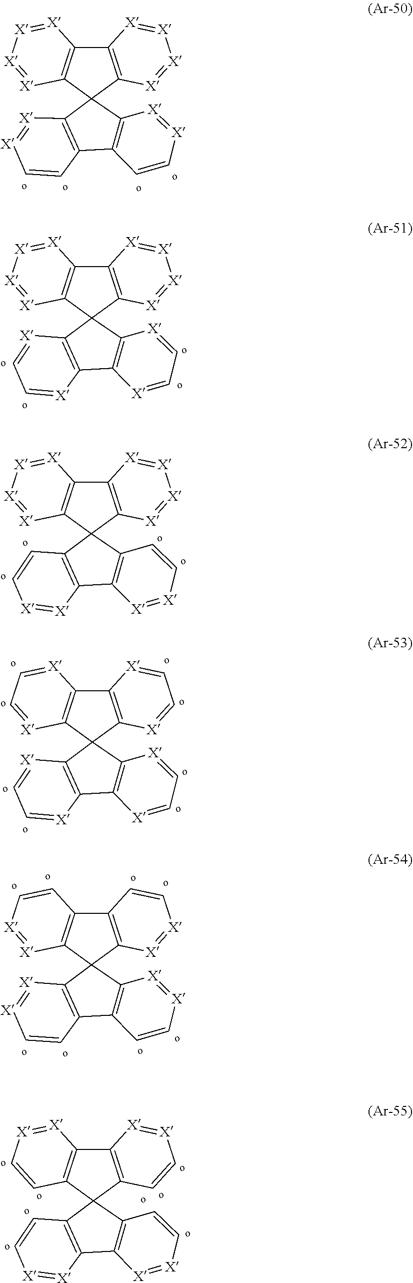

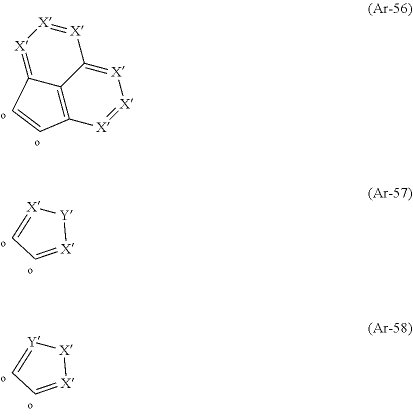

where X¹ is N or CR¹, preferably CR¹, Y¹ is selected from O, S, C(R¹)₂, Si(R¹)₂, Ge(R¹)₂, NR¹ and NAr¹, preferably O, S, NAr¹, more preferably NAr¹, U is selected from O, S, C(R¹)₂, N(R¹), B(R¹), Si(R¹)₂, C═O, S═O, SO₂, P(R¹) and P(═O)R¹, where R¹ has the definition set out above, especially for formula (I) or (Ia), and the nonaromatic or nonheteroaromatic polycyclic ring system having at least 3 rings binds in each case to the positions identified by o in the structural element of the formula (Ia) and/or (IIa) to form a ring. Preference is given here to structures of the formulae (Ar-2) to (Ar-54) and particular preference to structures of the formulae (Ar-4) to (Ar-15) and (Ar-23) to (Ar-44).

The double bond marked by the dotted bond that is shown in the structures of formula (Ia) or (IIa) may be regarded here as part of the aromatic or heteroaromatic ring system having 5 to 60 carbon atoms to which the structural element of formula (Ia) or (IIa) is fused.

Preference is also given to compounds having substructures of the formulae (Ar-1) to (Ar-58) in which not more than two X¹ groups per ring are N, preferably all X¹ groups per ring are CR¹, and preferably at least one, more preferably at least two, of the X¹ groups per ring are selected from C—H and C-D.

Furthermore, preference is given to compounds having substructures of formulae (Ar-1) to (Ar-58) in which not more than four, preferably not more than two, X′ groups are N, and more preferably all the X′ groups are CR¹, where preferably not more than four, more preferably not more than three and especially preferably not more than two of the CR¹ groups that X′ represents are not the CH group.

It may most preferably be the case that the aromatic or heteroaromatic ring system having two, preferably having three, fused aromatic or heteroaromatic rings is selected from the groups of the formulae (AR′-1) to (Ar′-11)

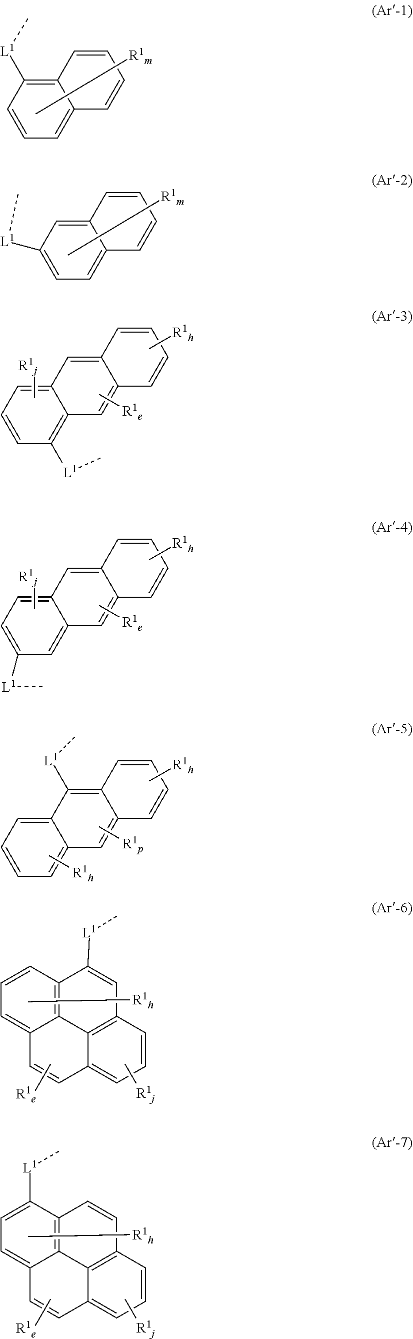

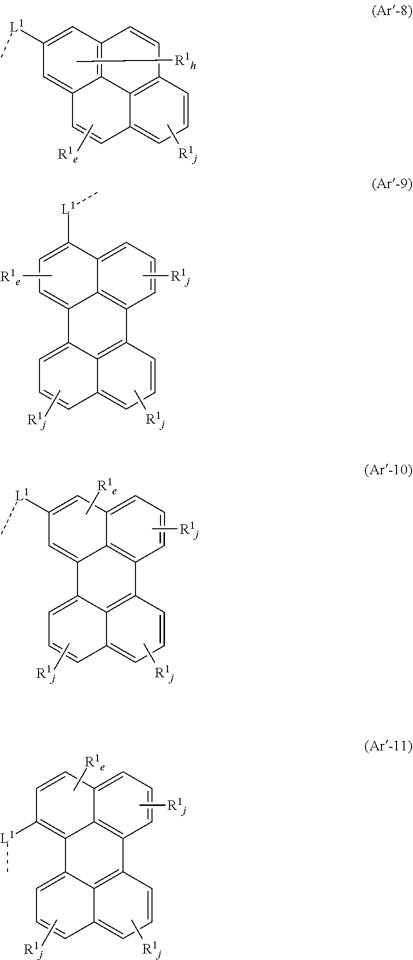

where L¹ represents a bond or an aromatic or heteroaromatic ring system which has 5 to 40, preferably 5 to 30, aromatic ring atoms and may be substituted by one or more R¹ radicals, where R¹ has the definition set out above, especially for formula (I) or (Ia), the dotted bond marks the position of attachment and the indices are as follows:
p is 0 or 1;
e is 0, 1 or 2, preferably 0 or 1;
j at each instance is independently 0, 1, 2 or 3, preferably 0, 1 or 2, more preferably preferably 0 or 1;
h at each instance is independently 0, 1, 2, 3 or 4, preferably 0, 1 or 2, more preferably preferably 0 or 1;
i at each instance is independently 0, 1 or 2;
m is an integer in the range from 0 to 7, preferably 0, 1, 2, 3, 4, 5 or 6, particularly preferably 0, 1, 2, 3 or 4, especially preferably 0, 1 or 2.

It is preferable that the sum total of the indices p, e, i, j, h and m in the structures of the formula (AR′-1) to (Ar′-11) in each case is not more than 3, preferably not more than 2 and more preferably not more than 1.

It may further be the case that the structural element of the formula (Ia) or (IIa) is fused to an aromatic or heteroaromatic ring system which has 5 to 60 carbon atoms and is selected from the groups of the formulae (Ar′-12) to (Ar′-57)

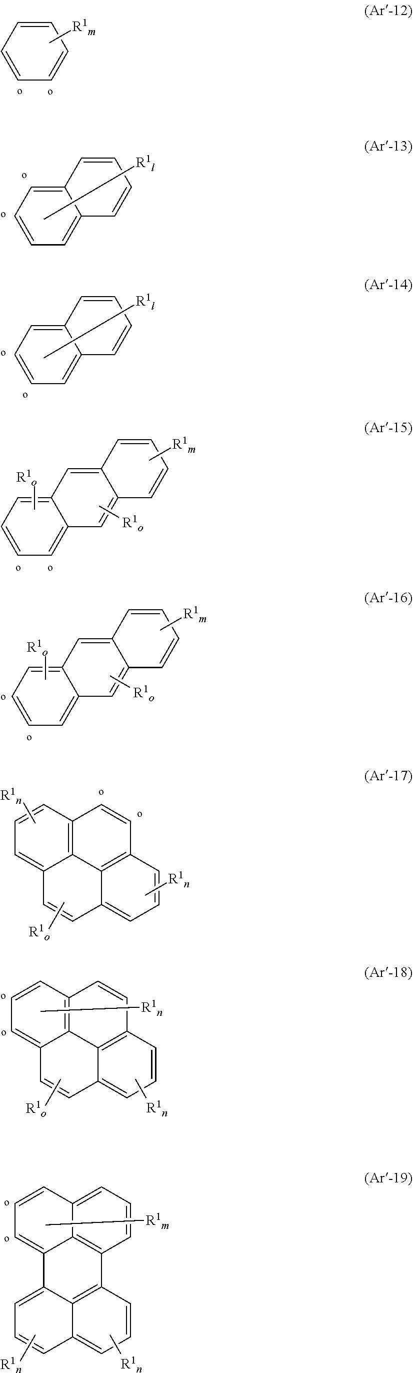

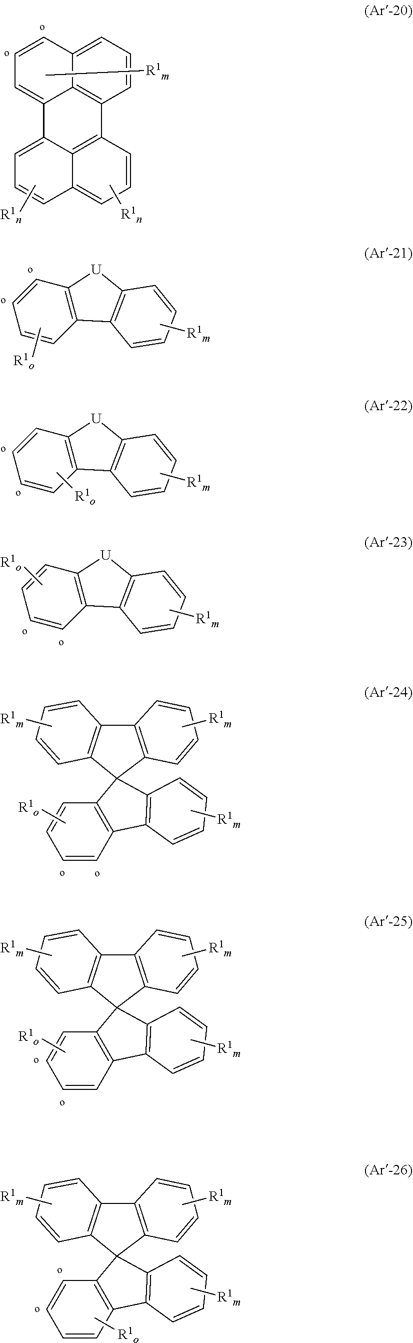

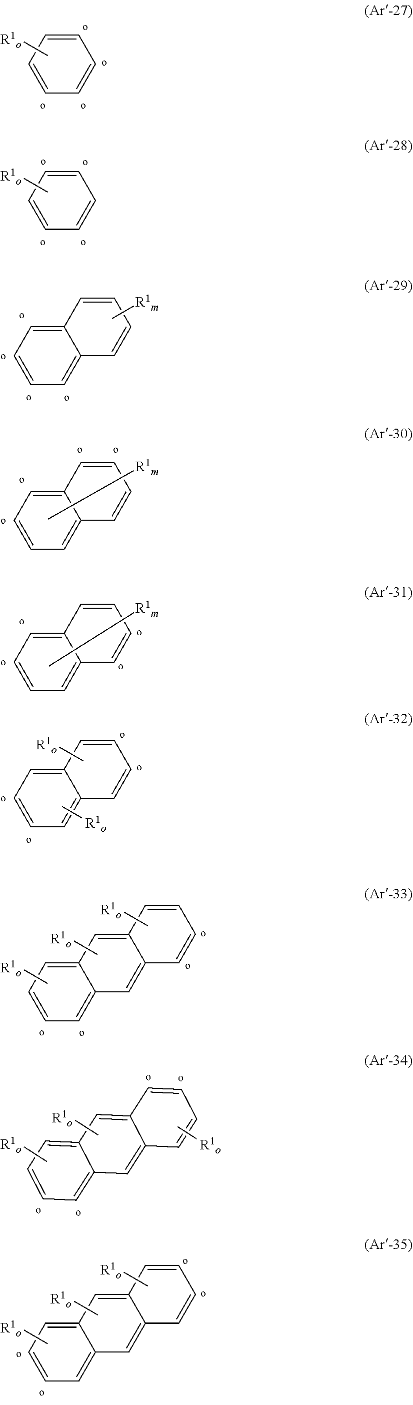

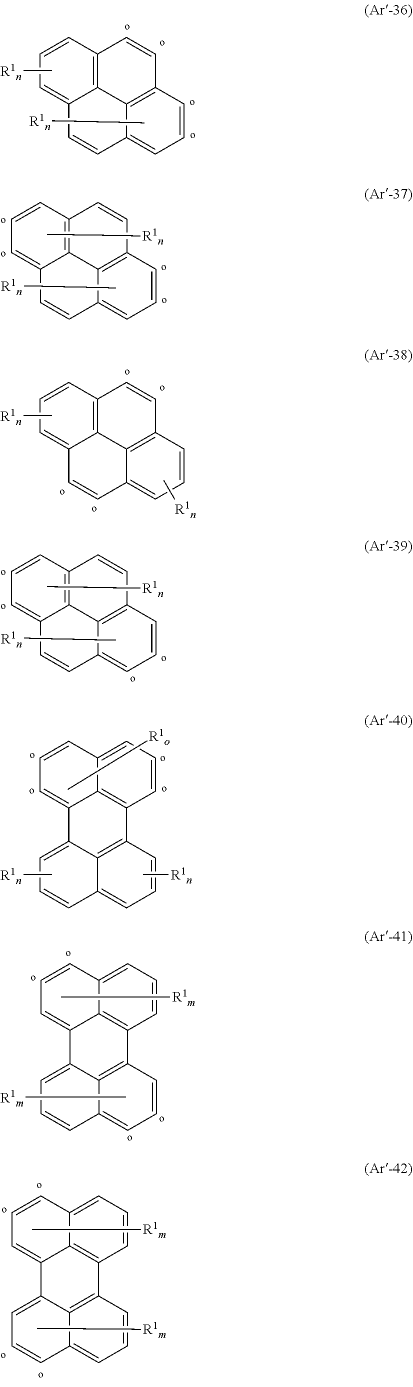

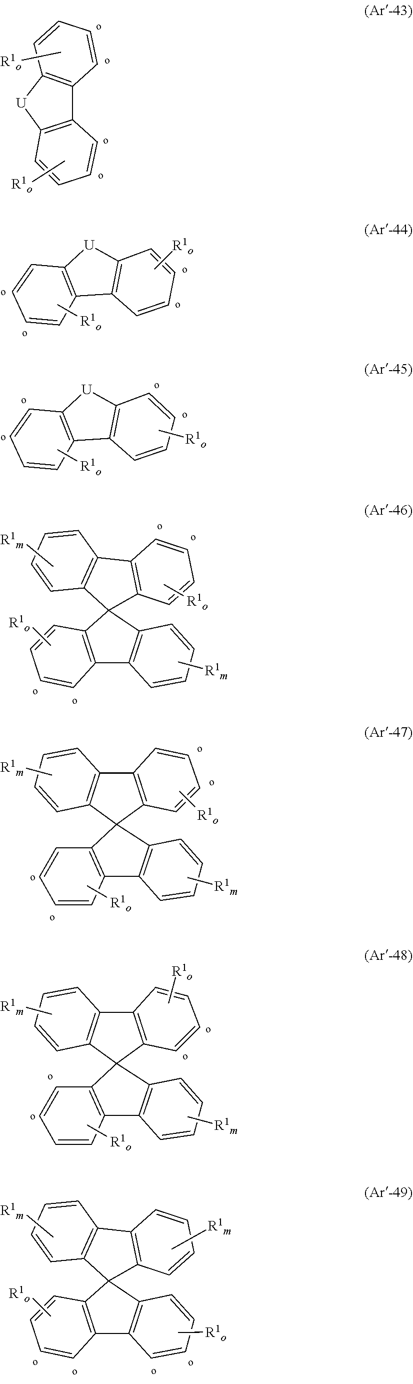

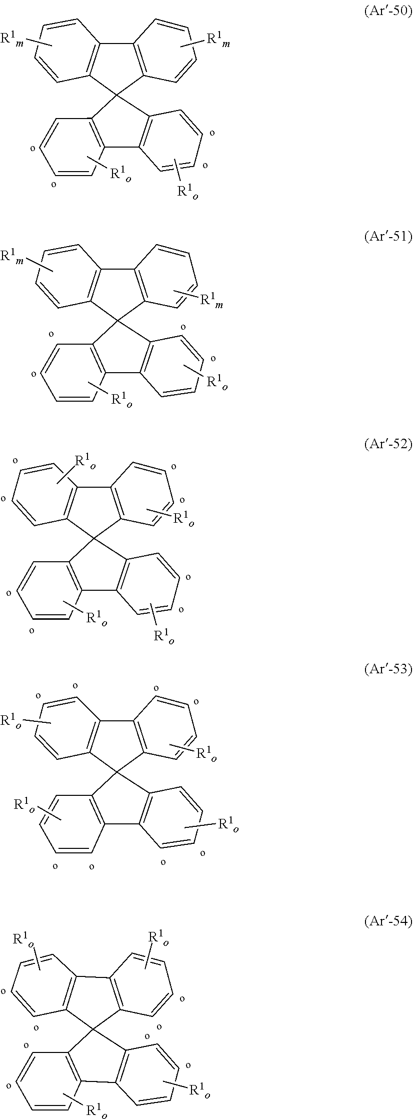

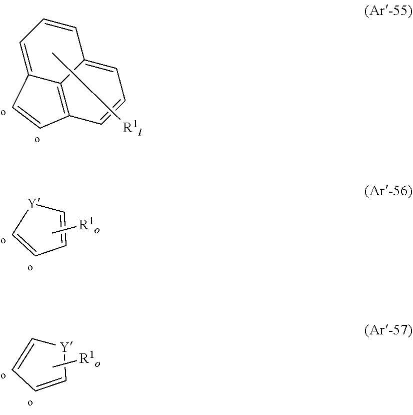

where R¹ has the definition given above, especially for formula (I) or (Ia), and the symbols Y′ and U have the definition given above, especially for formulae (Ar-12) to (Ar-58), the index 0 is 0, 1 or 2, preferably 0 or 1, the index n is 0, 1, 2 or 3, preferably 0, 1 or 2, and the index m is 0, 1, 2, 3 or 4, preferably 0, 1 or 2, and the index I is 0, 1, 2, 3, 4, 5 or 6, preferably 0, 1 or 2, and the structural element of the formula (Ia) or (IIa) binds to the aromatic or heteroaromatic ring system having 5 to 60 carbon atoms at the respective positions identified by o to form a ring. Preference is given here to structures of the formulae (AR′-2) to (Ar′-53) and particular preference to structures of the formulae (Ar′-4) to (Ar′-15) and (Ar′-22) to (Ar′-43).

In addition, in the substructures of the formulae (Ar′-12) to (Ar′-57), it may be the case that the sum total of the indices o, n, m and l is not more than 6, preferably not more than 4 and more preferably not more than 2.

In addition, the structures of the formulae (Ar-1) to (Ar-158) and/or (Ar′-1) to (Ar′-57) may comprise radicals comprising hole transport groups, preferably hole transport groups of the formulae (H-1) to (H-26) and/or electron transport groups, preferably electron transport group-comprising radicals of formula (QL), where the electron transport group can preferably be represented by the formulae (Q-1) to (Q-44). The substituents R¹ here in the structures of the formulae (H-1) to (H-26) and/or (Q-1) to (Q-44) should be replaced by substituents R².

It may further be the case that the organofunctional compound comprises at least one solubilizing group.

Preferably, a solubilizing group or solubilizing structural element may comprise, preferably constitute, a relatively long alkyl group (about 4 to 20 carbon atoms), especially a branched alkyl group, or an optionally substituted aryl group. The preferred aryl groups include a xylyl, mesityl, terphenyl or quaterphenyl group, particular preference being given to branched terphenyl or quaterphenyl groups.

It may further be the case that the compound contains at least one solubilizing structural element or solubilizing group and contains at least one functional structural element or functional group, the functional structural element or the functional group being selected from hole transport groups, electron transport groups, structural elements or groups which lead to host materials, or structural elements or groups having wide bandgap properties.

When X or X¹ is CR¹ or when the aromatic and/or heteroaromatic groups are substituted by substituents R¹, these substituents R¹ are preferably selected from the group consisting of H, D, F, CN, N(Ar¹)₂, C(═O)Ar¹, P(═O)(Ar¹)₂ a straight-chain alkyl or alkoxy group having 1 to 10 carbon atoms or a branched or cyclic alkyl or alkoxy group having 3 to 10 carbon atoms or an alkenyl group having 2 to 10 carbon atoms, each of which may be substituted by one or more R² radicals, where one or more nonadjacent CH₂ groups may be replaced by O and where one or more hydrogen atoms may be replaced by D or F, an aromatic or heteroaromatic ring system which has 5 to 24 aromatic ring atoms and may be substituted in each case by one or more R² radicals, but is preferably unsubstituted, or an aralkyl or heteroaralkyl group which has 5 to 25 aromatic ring atoms and may be substituted by one or more R² radicals; at the same time, it is optionally possible for two substituents R¹ preferably bonded to adjacent carbon atoms to form a monocyclic or polycyclic, aliphatic, aromatic or heteroaromatic ring system which may be substituted by one or more R¹ radicals; where the Ar¹ group has the definition given above, especially for formula (I).

More preferably, these substituents R¹ are selected from the group consisting of H, D, F, CN, N(Ar¹)₂, a straight-chain alkyl group having 1 to 8 carbon atoms, preferably having 1, 2, 3 or 4 carbon atoms, or a branched or cyclic alkyl group having 3 to 8 carbon atoms, preferably having 3 or 4 carbon atoms, or an alkenyl group having 2 to 8 carbon atoms, preferably having 2, 3 or 4 carbon atoms, each of which may be substituted by one or more R² radicals, but is preferably unsubstituted, or an aromatic or heteroaromatic ring system which has 5 to 24 aromatic ring atoms, preferably 6 to 18 aromatic ring atoms, more preferably 6 to 13 aromatic ring atoms, and may be substituted in each case by one or more nonaromatic R¹ radicals, but is preferably unsubstituted; at the same time, two substituents R¹ preferably bonded to adjacent carbon atoms may optionally form a monocyclic or polycyclic aliphatic ring system which may be substituted by one or more R² radicals, but is preferably unsubstituted, where Ar¹ may have the definition set out above.

Most preferably, the substituents R¹ are selected from the group consisting of H or an aromatic or heteroaromatic ring system which has 6 to 18 aromatic ring atoms, preferably 6 to 13 aromatic ring atoms, and may be substituted in each case by one or more nonaromatic R² radicals, but is preferably unsubstituted. Examples of suitable substituents R¹ are selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 1-, 2-, 3- or 4-spirobifluorenyl, pyridyl, pyrimidinyl, 1-, 2-, 3- or 4-dibenzofuranyl, 1-, 2-, 3- or 4-dibenzothienyl, 1-, 2-, 3- or 4-carbazolyl and indenocarbazolyl, each of which may be substituted by one or more R² radicals, but are preferably unsubstituted.

It may further be the case that the substituents R¹ of an aromatic or heteroaromatic ring system do not form a fused aromatic or heteroaromatic ring system, preferably any fused ring system, with further ring atoms of the aromatic or heteroaromatic ring system. This includes the formation of a fused ring system with possible substituents R², R³ which may be bonded to the R¹ radicals.

It may further be the case that the organofunctional compound comprises at least one group, preferably that, in the structure of formula (IIIa) to (IIIh) and/or (IVa) to (IVh), at least one structural element A^{a} or at least one Ar¹, Ar², Ar³, Ar⁴ and/or R¹ radical comprises a group, preferably is a group, selected from the formulae (R¹-1) to (R¹-92)

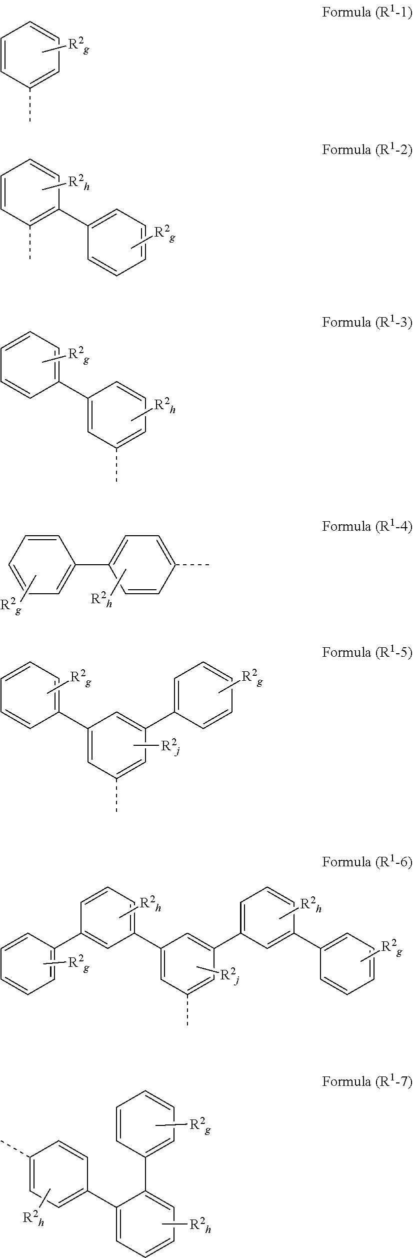

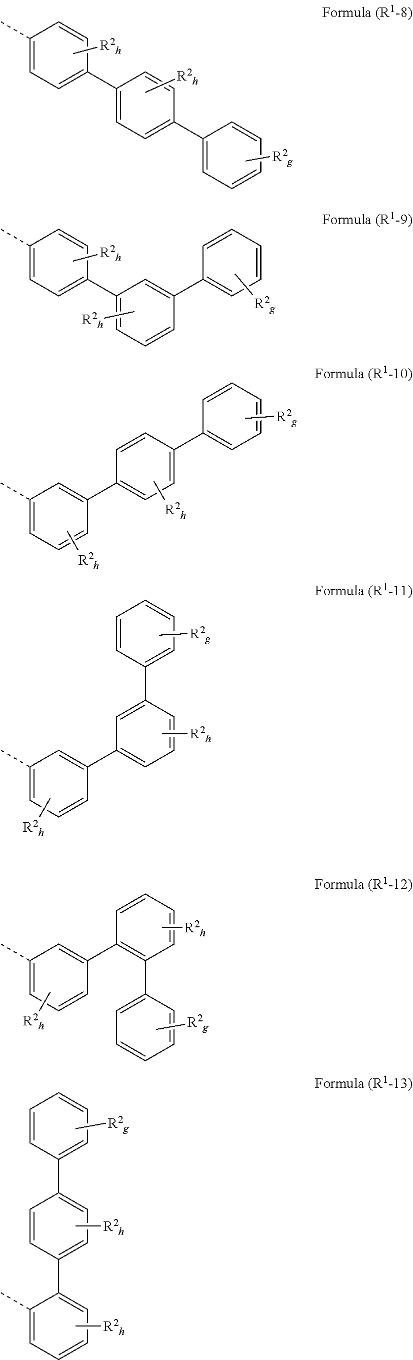

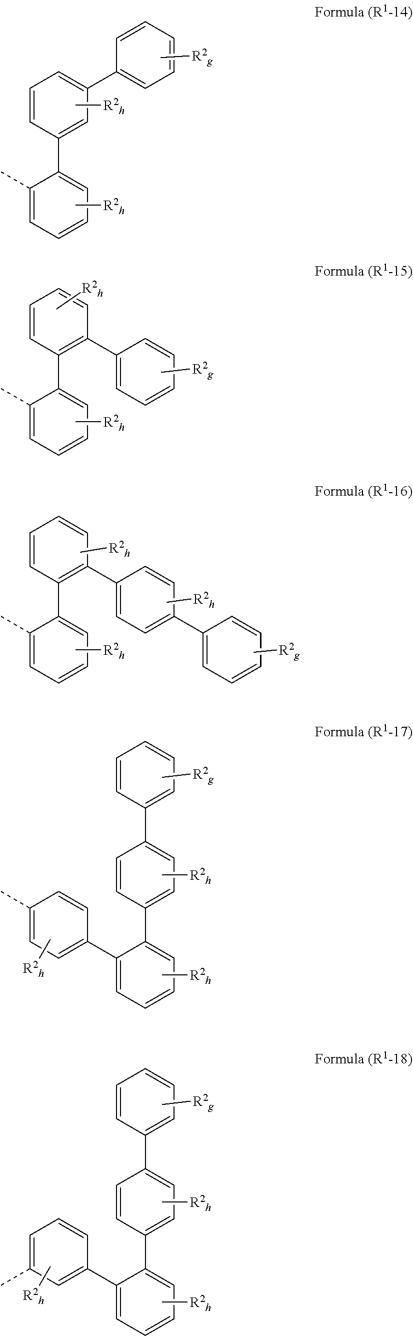

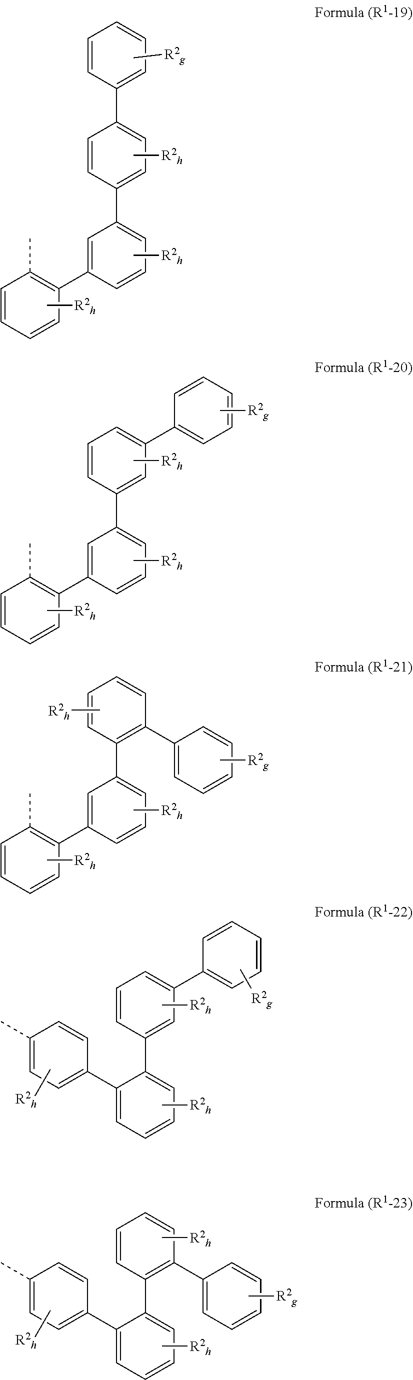

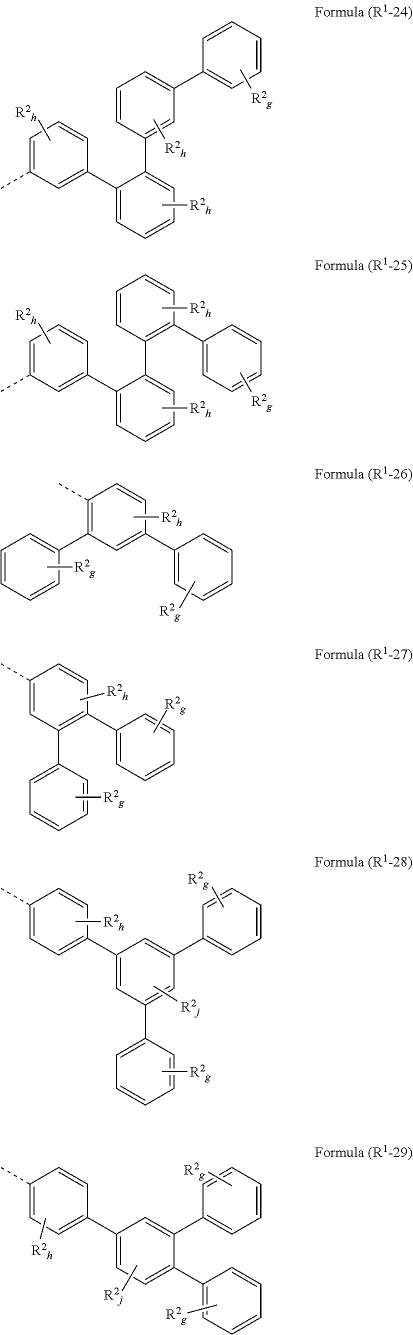

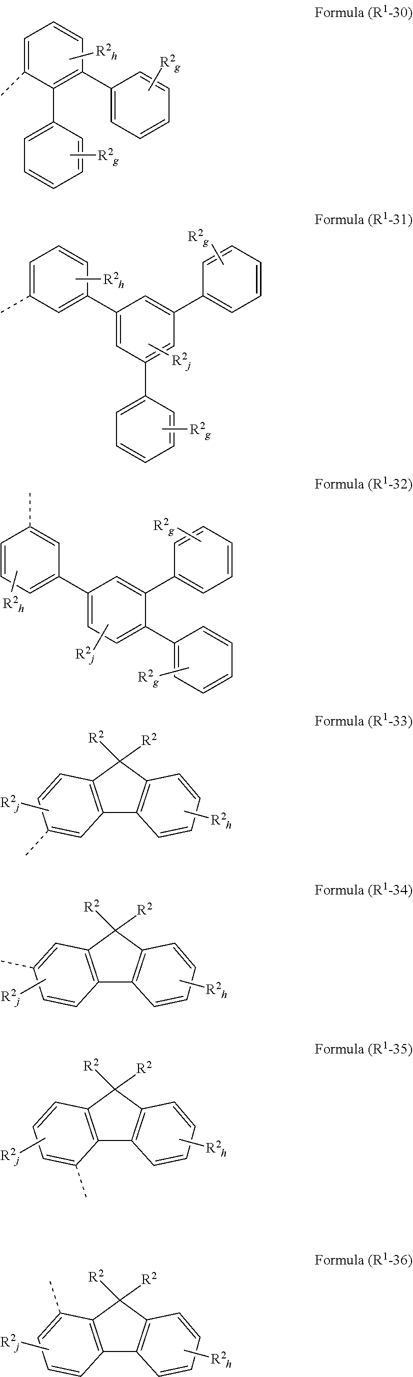

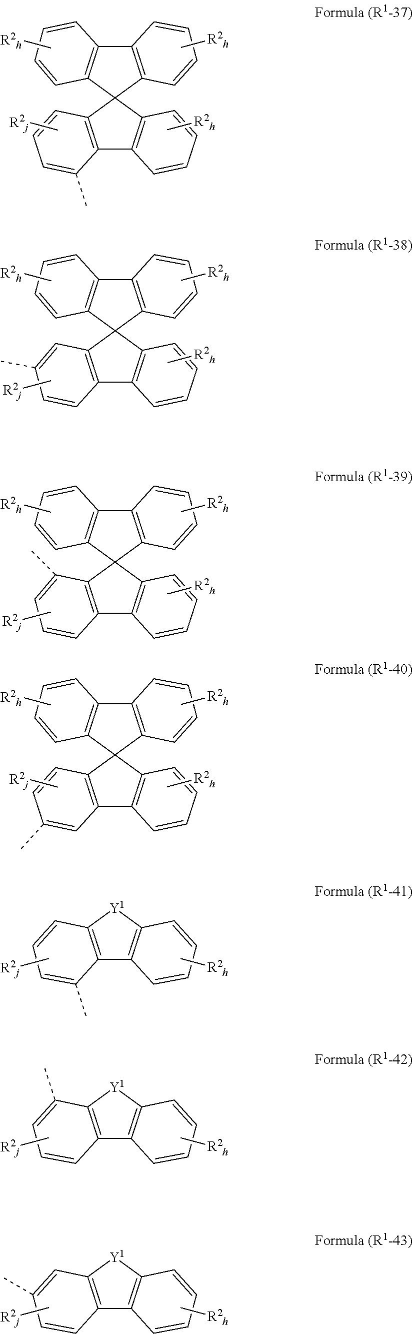

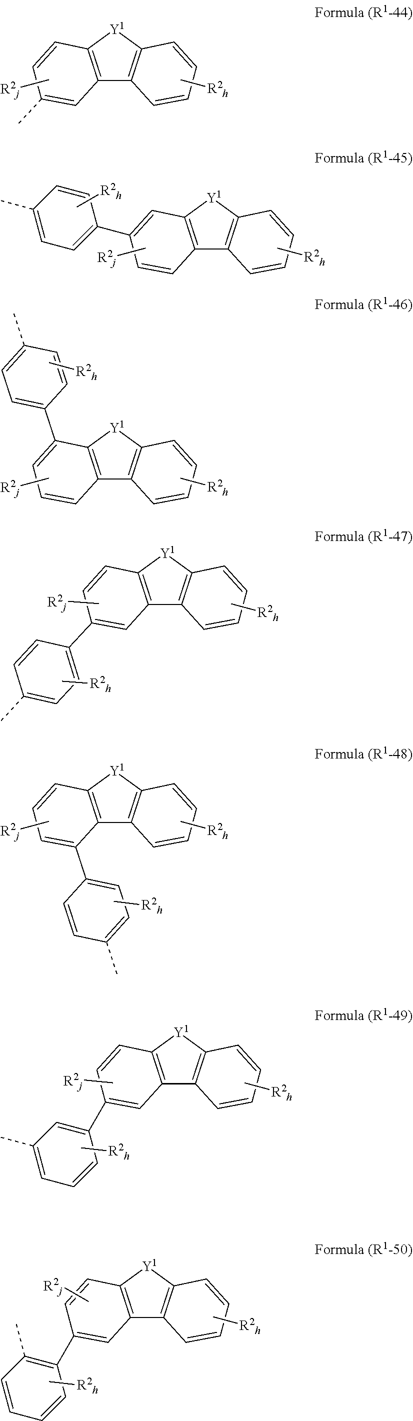

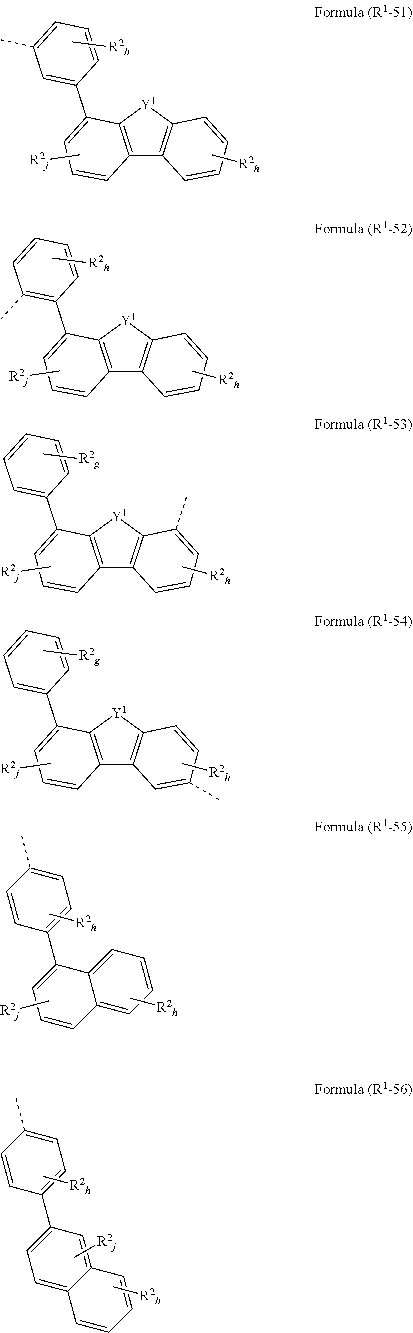

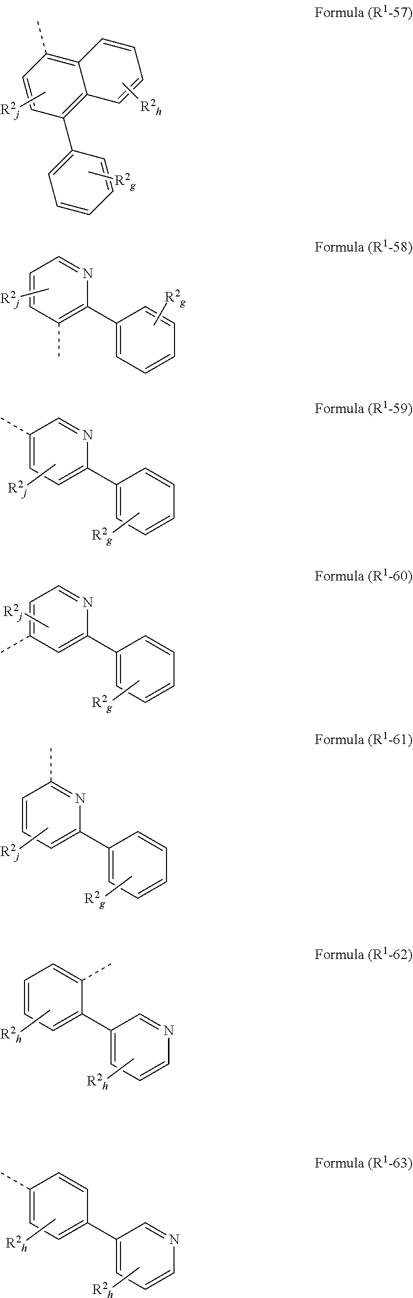

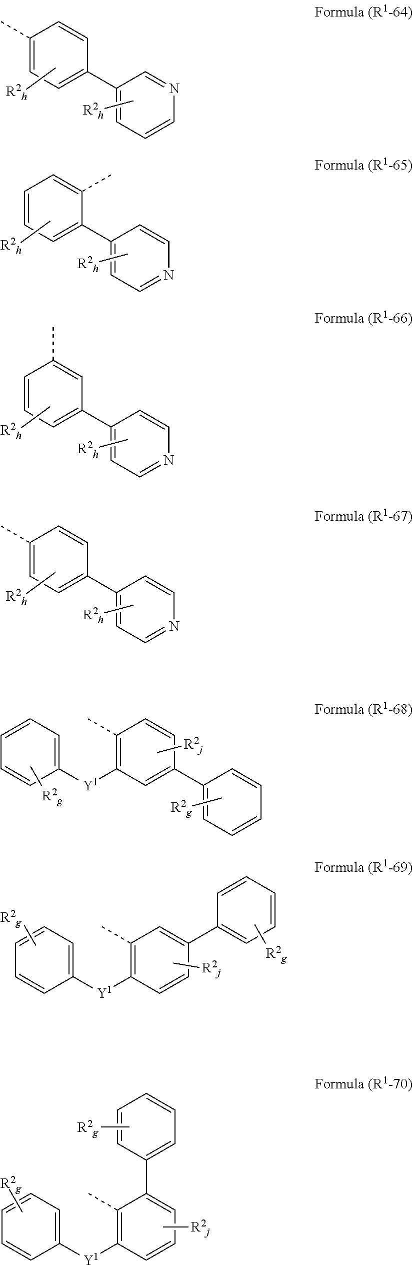

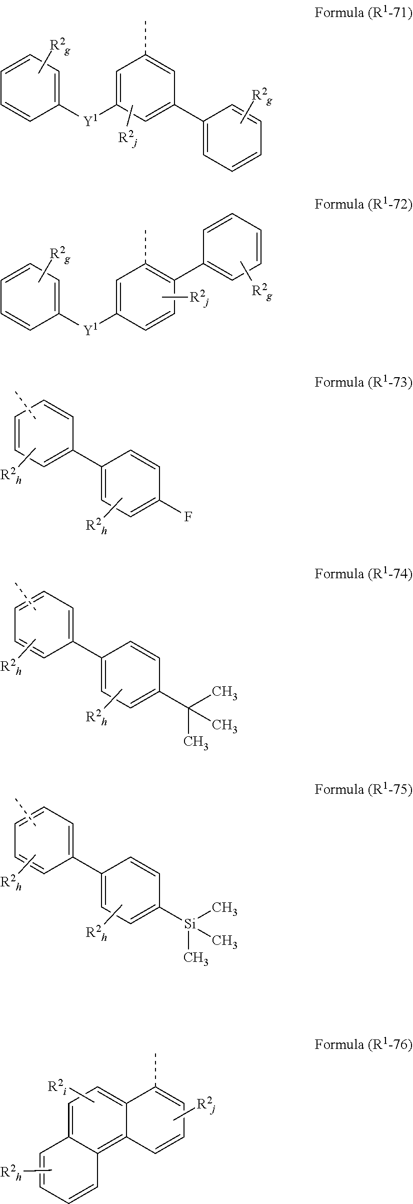

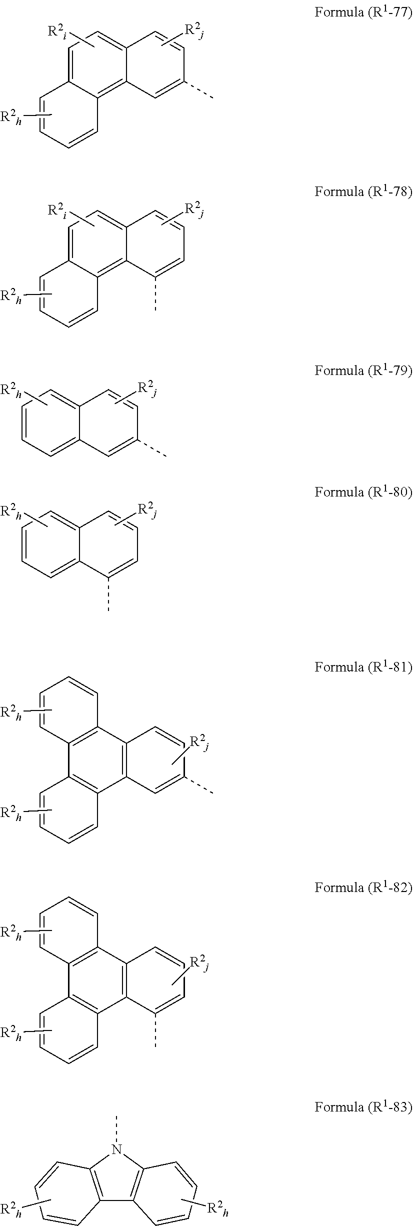

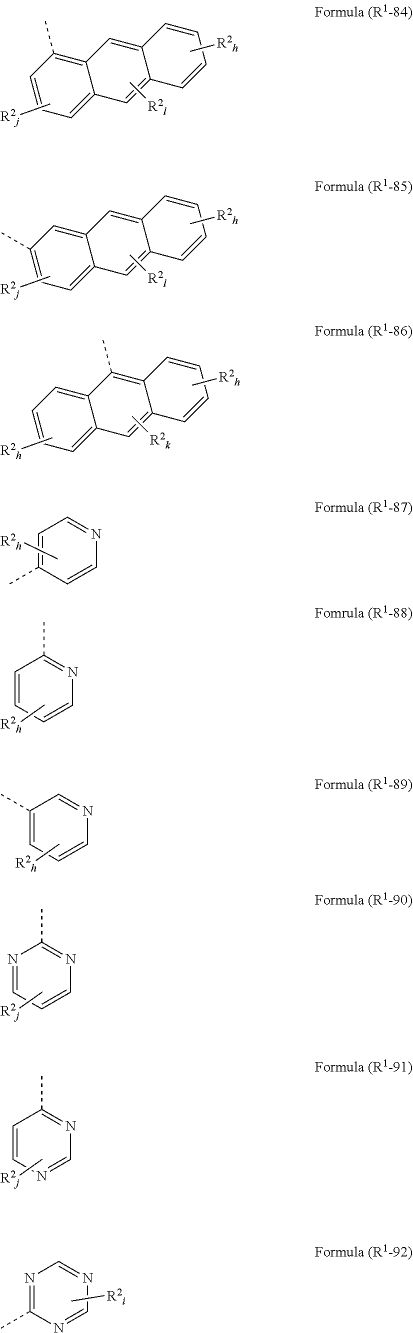

where the symbols used are as follows:
Y¹ is O, S or NR², preferably O or S;
k at each instance is independently 0 or 1;
i at each instance is independently 0, 1 or 2;
j at each instance is independently 0, 1, 2 or 3;
h at each instance is independently 0, 1, 2, 3 or 4;
g at each instance is independently 0, 1, 2, 3, 4 or 5;
R² may have the definition given above, especially for formula (I) or (Ia), and
   the dotted bond marks the position of attachment.

Preference is given here to the groups of the formulae R¹-1 to R¹-54, particular preference to the R¹-1, R¹-3, R¹-5, R¹-6, R¹-15, R¹-29, R¹-30, R¹-31, R¹-32, R¹-33, R¹-38, R¹-39, R¹-40, R¹-41, R¹-42, R¹-43, R¹-44 and/or R¹-45 groups.

It may preferably be the case that the sum total of the indices k, i, j, h and g in the structures of the formula (R¹-1) to (R¹-92) in each case is not more than 3, preferably not more than 2 and more preferably not more than 1. Preferably, the R² radicals in the formulae (R¹-1) to (R¹-92) do not form a fused aromatic or heteroaromatic ring system, and preferably do not form any fused ring system, with the ring atoms of the aryl group or heteroaryl group to which the R² radicals are bonded. This includes the formation of a fused ring system with possible substituents R³ which may be bonded to the R² radicals.

The above-detailed radicals of the formulae (R¹-1) to (R¹-92) are preferred Ar radicals of formula (I) or Ar³, Ar⁴ radicals of formulae (H-1) to (H-3) or preferred embodiments of these formulae, where, in this case, the R² groups shown in the formulae (R¹-1) to (R¹-92) are to be replaced by R¹ radicals. The preferences detailed above with regard to the formulae (R¹-1) to (R¹-92) are correspondingly applicable.

It may preferably be the case that the compound comprises at least one linking group selected from the formulae (L¹-1) to (L¹-108); preferably, in the structure of formulae (H-1) to (H-26), the Ar² group is selected from the formulae (L¹-1) to (L¹-108) or the electron-conducting group is linked to further structural elements via a linking group selected from the formulae (L¹-1) to (L¹-108), or the L¹ radical in formulae (QL), (Ar-1) to (Ar-11) and/or (Ar′-1) to (Ar′-11) is a group selected from the formulae (L¹-1) to (L¹-108)

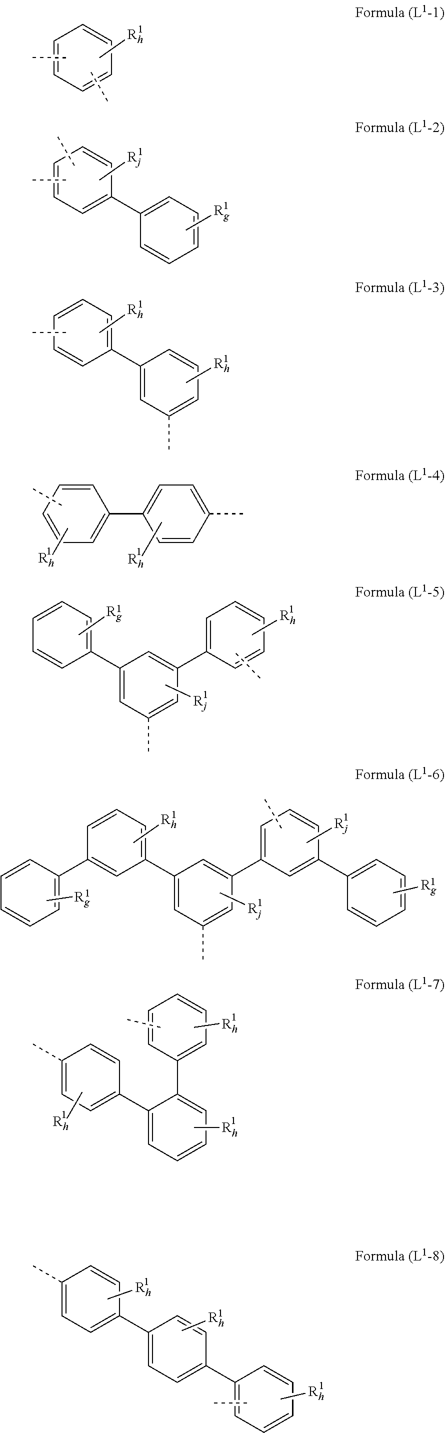

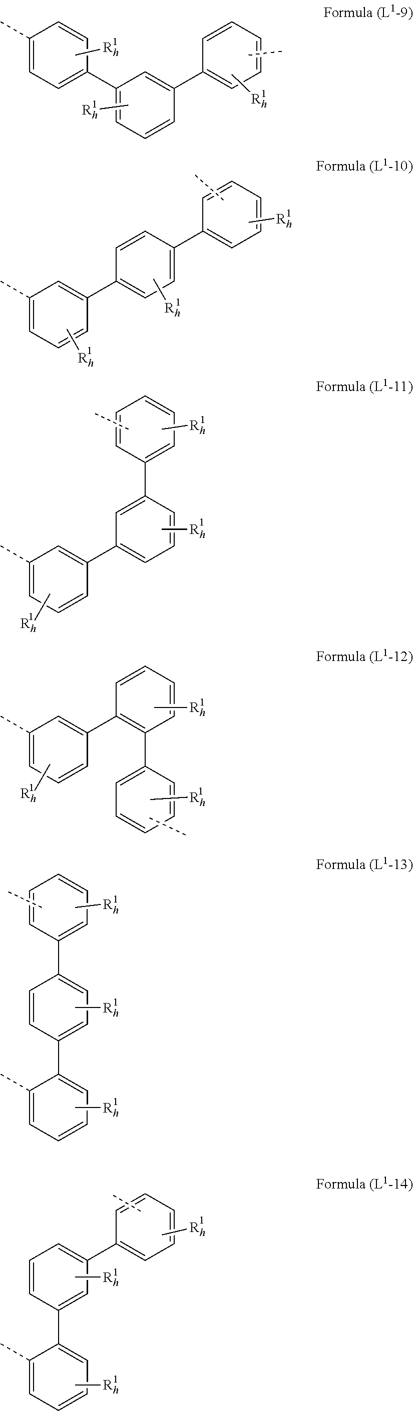

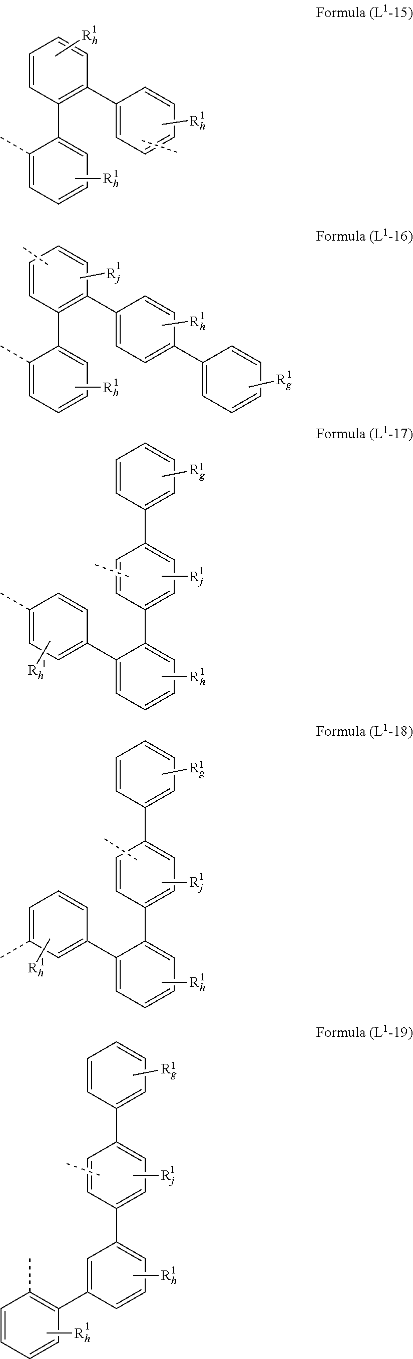

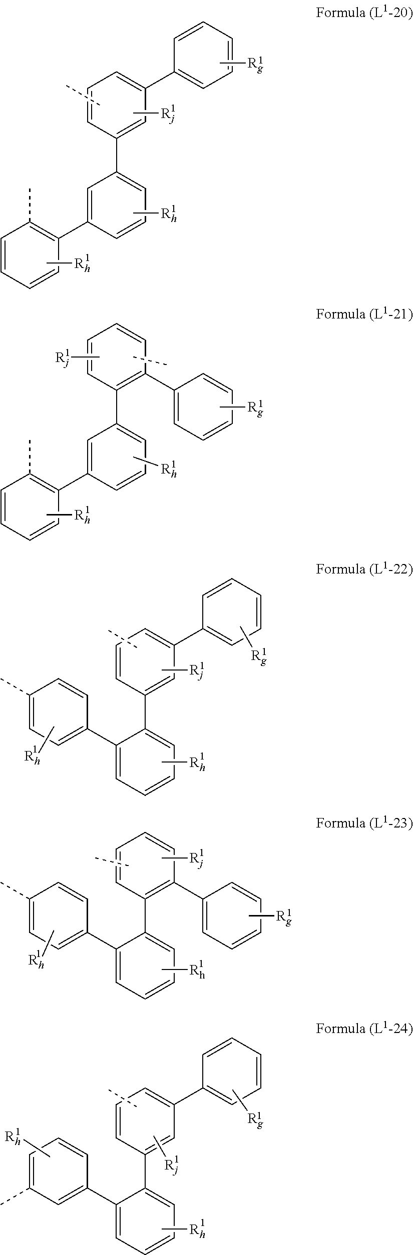

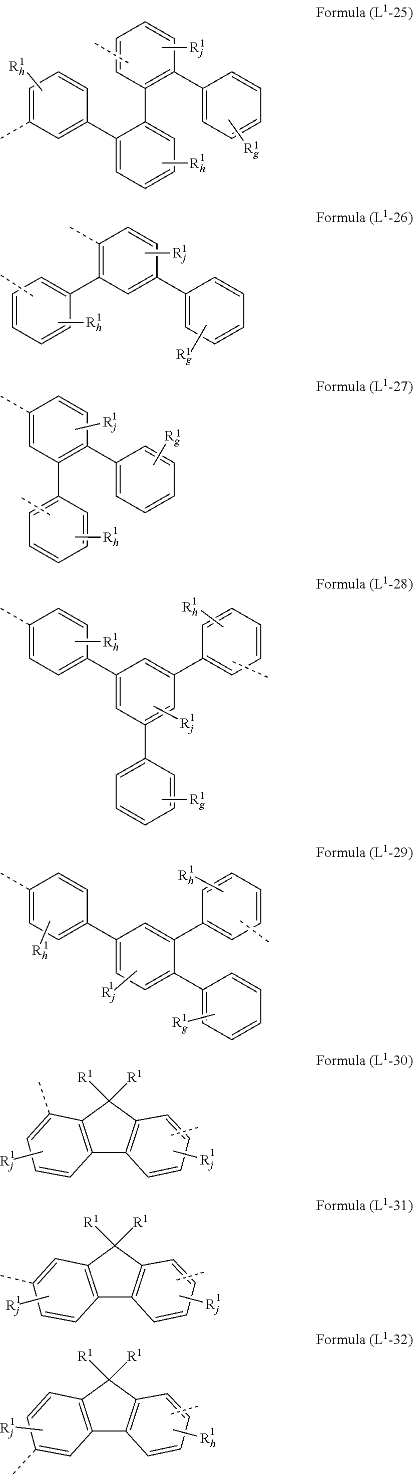

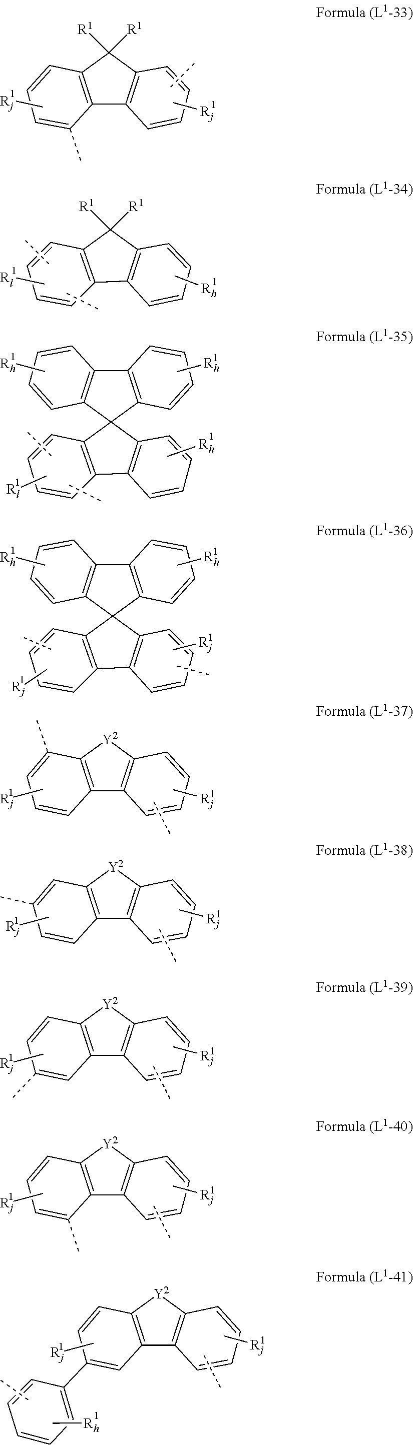

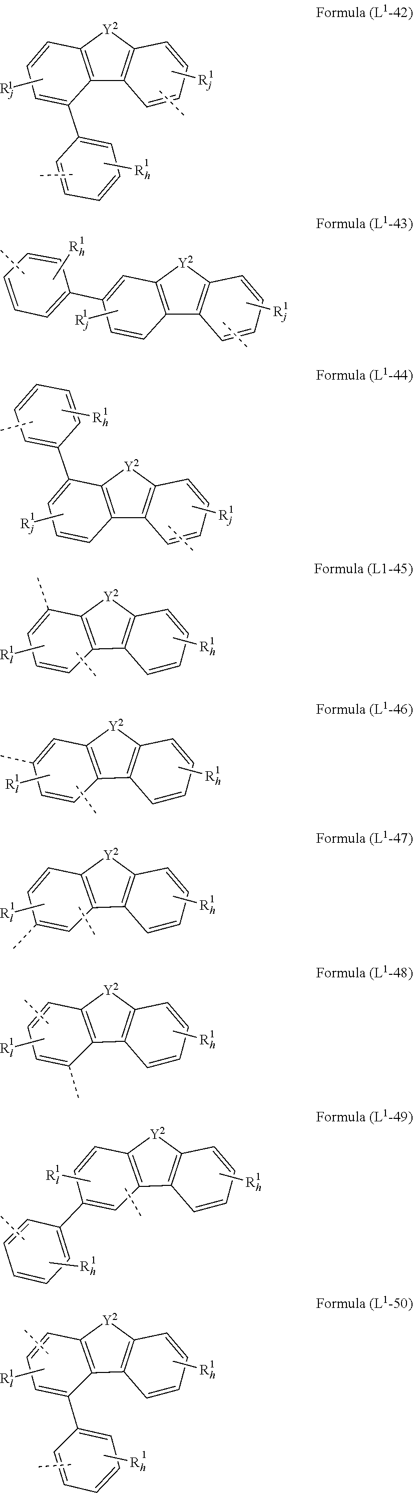

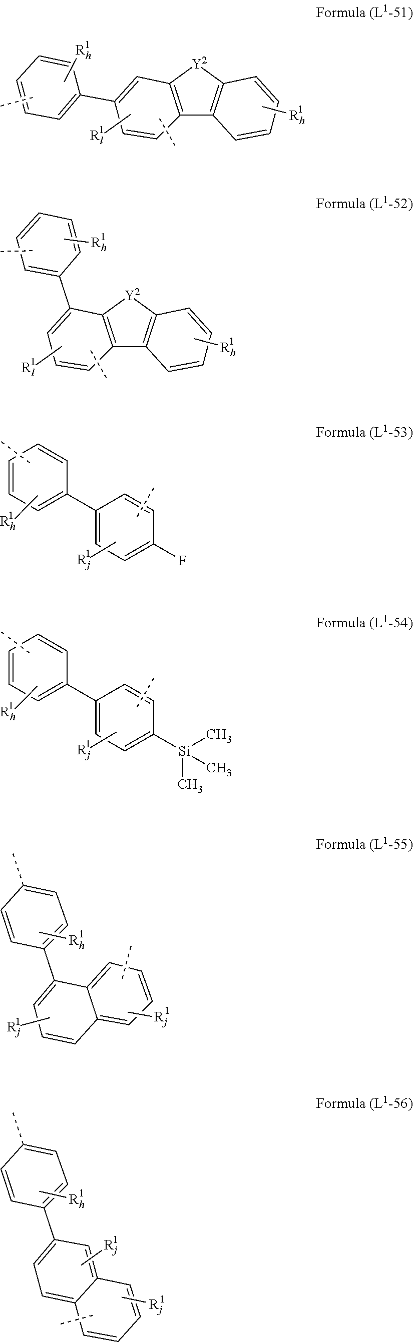

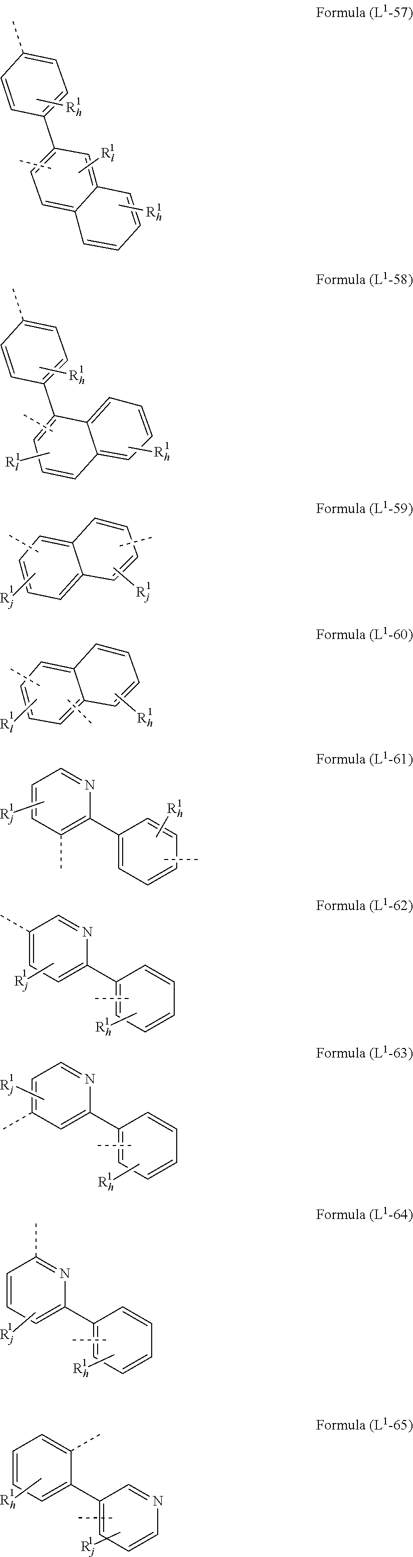

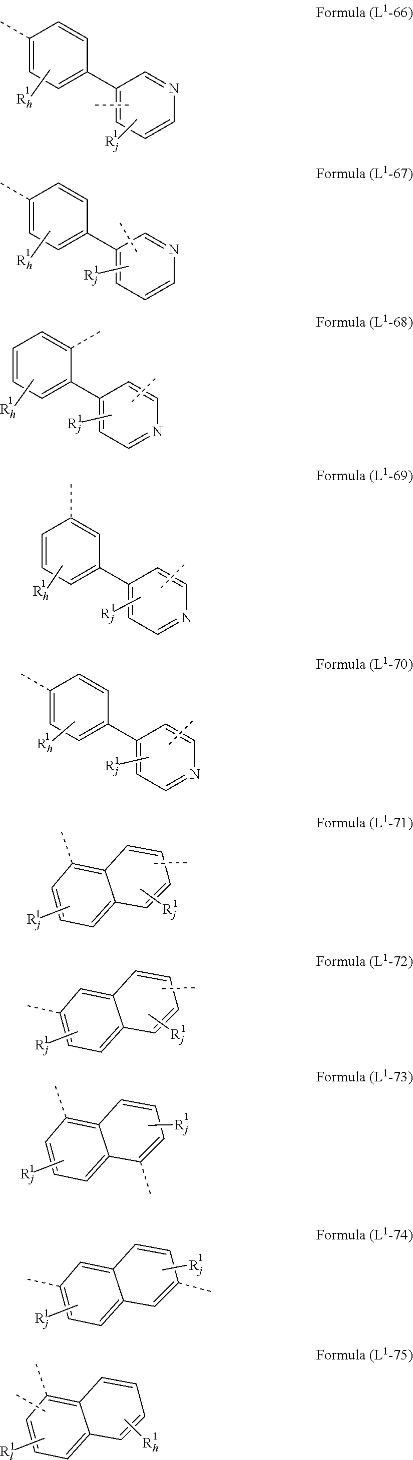

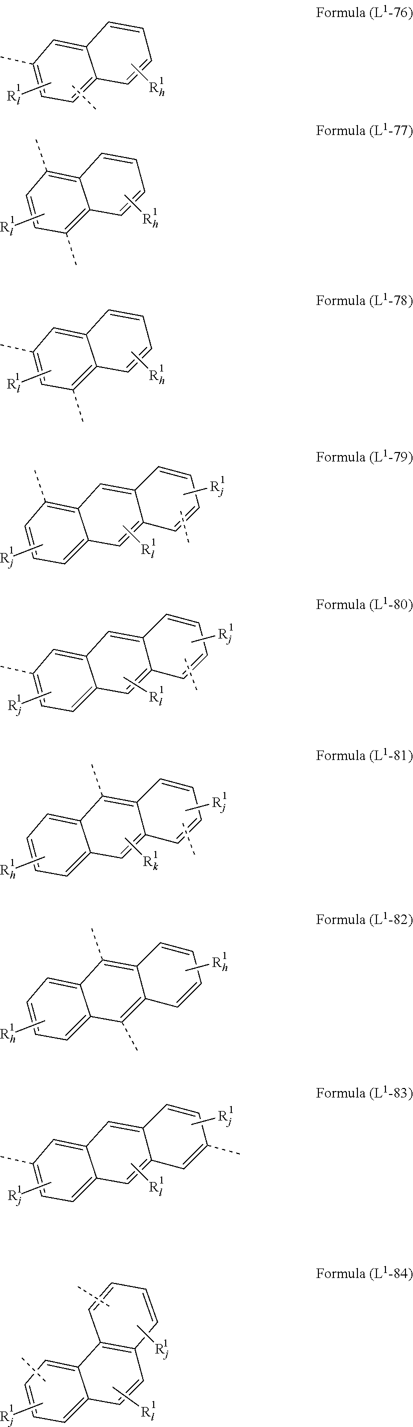

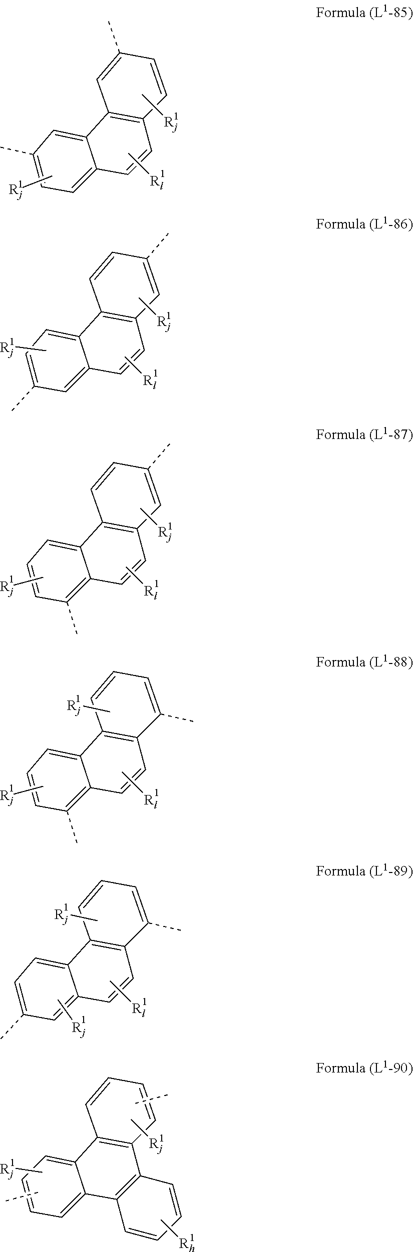

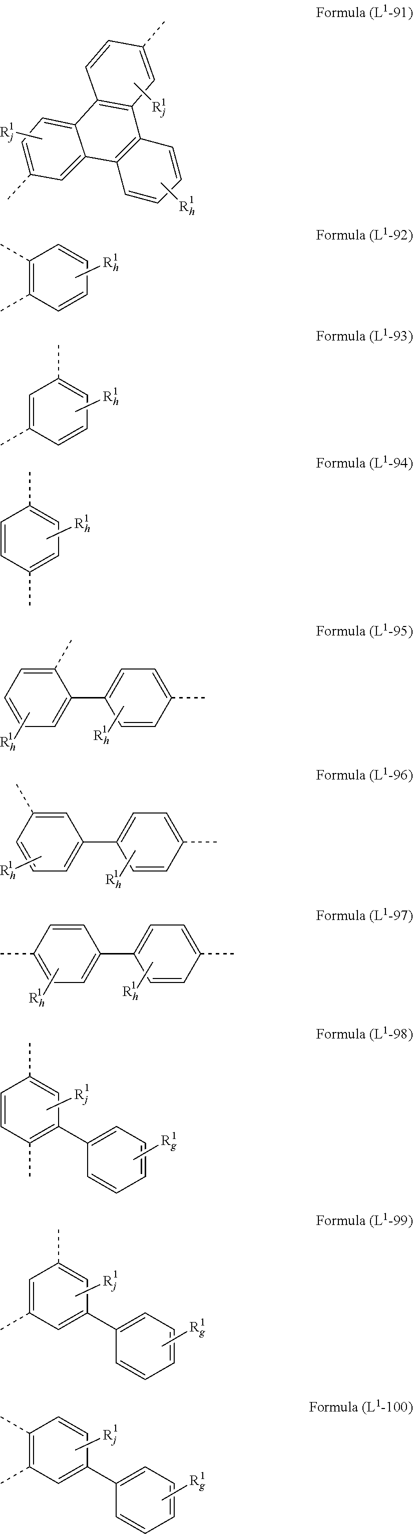

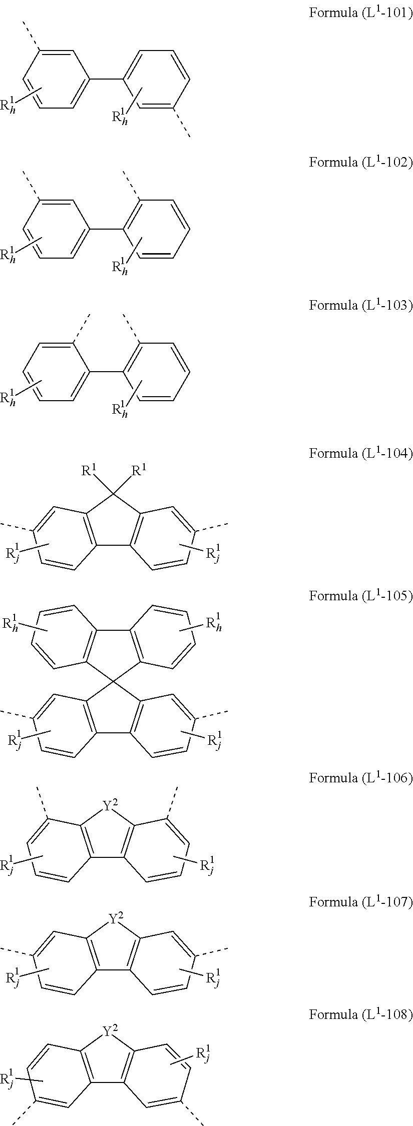

where the dotted bonds in each case mark the positions of attachment, the index k is 0 or 1, the index I is 0, 1 or 2, the index j at each instance is independently 0, 1, 2 or 3; the index h at each instance is independently 0, 1, 2, 3 or 4, the index g is 0, 1, 2, 3, 4 or 5; the symbol Y² is O, S or NR¹, preferably O or S; and the symbol R¹ has the definition given above, especially for formula (I) or (Ia).

It may preferably be the case that the sum total of the indices k, l, g, h and j in the structures of the formula (L¹-1) to (L¹-108) is at most 3 in each case, preferably at most 2 and more preferably at most 1.

Preferred compounds of the invention having a group of the formulae (H-1) to (H-26) comprise an Ar² group selected from one of the formulae (L¹-1) to (L¹-78) and/or (L¹-92) to (L¹-108), preferably of the formula (L¹-1) to (L¹-54) and/or (L¹-92) to (L¹-108), especially preferably of the formula (L¹-1) to (L¹-29) and/or (L¹-92) to (L¹-103). Advantageously, the sum total of the indices k, l, g, h and j in the structures of the formulae (L¹-1) to (L¹-78) and/or (L¹-92) to (L¹-108), preferably of the formula (L¹-1) to (L¹-54) and/or (L¹-92) to (L¹-108), especially preferably of the formula (L¹-1) to (L¹-29) and/or (L¹-92) to (L¹-103), may in each case be not more than 3, preferably not more than 2 and more preferably not more than 1.

Preferred compounds of the invention having a group of the formula (QL) comprise an L¹ group which represents a bond or which is selected from one of the formulae (L¹-1) to (L¹-78) and/or (L¹-92) to (L¹-108), preferably of the formula (L¹-1) to (L¹-54) and/or (L¹-92) to (L¹-108), especially preferably of the formula (L¹-1) to (L¹-29) and/or (L¹-92) to (L¹-103). Advantageously, the sum total of the indices k, l, g, h and j in the structures of the formulae (L¹-1) to (L¹-78) and/or (L¹-92) to (L¹-108), preferably of the formula (L¹-1) to (L¹-54) and/or (L¹-92) to (L¹-108), especially preferably of the formula (L¹-1) to (L¹-29) and/or (L¹-92) to (L¹-103), may in each case be not more than 3, preferably not more than 2 and more preferably not more than 1.

Preferred compounds of the invention that have a group of the formulae (Ar-1) to (Ar-11) and/or (Ar′-1) to (Ar′-11) comprise an L¹ group which is a bond or which is selected from one of the formulae (L¹-1) to (L¹-78) and/or (L¹-92) to (L¹-108), preferably of the formula (L¹-1) to (L¹-54) and/or (L¹-92) to (L¹-108), especially preferably of the formula (L¹-1) to (L¹-29) and/or (L¹-92) to (L¹-103). Advantageously, the sum total of the indices k, l, g, h and j in the structures of the formulae (L¹-1) to (L¹-78) and/or (L¹-92) to (L¹-108), preferably of the formula (L¹-1) to (L¹-54) and/or (L¹-92) to (L¹-108), especially preferably of the formula (L¹-1) to (L¹-29) and/or (L¹-92) to (L¹-103), may in each case be not more than 3, preferably not more than 2 and more preferably not more than 1.

Preferably, the R² radicals in the formulae (L¹-1) to (L¹-108) do not form a fused aromatic or heteroaromatic ring system, and preferably do not form any fused ring system, with the ring atoms of the aryl group or heteroaryl group to which the R² radicals are bonded. This includes the formation of a fused ring system with possible substituents R³ which may be bonded to the R² radicals.

In a preferred configuration, compounds of the invention that are usable for production of functional layers of electronic devices are selected from the group of the phenyls, fluorenes, indenofluorenes, spirobifluorenes, carbazoles, indenocarbazoles, indolocarbazoles, spirocarbazoles, pyrimidines, triazines, lactams, triarylamines, dibenzofurans, dibenzothienes, imidazoles, benzimidazoles, benzoxazoles, benzothiazoles, 5-arylphenanthridin-6-ones, 9,10-dehydrophenanthrenes, fluoranthenes, anthracenes, benzanthracenes, fluoradenes.

Preferably, compounds usable for production of functional layers of electronic devices, preferably compounds comprising structures of the formulae (I), (Ia), (II), (IIa), (IIIa) to (IIIh) and/or (IVa) to (IVh), have a molecular weight of not more than 5000 g/mol, preferably not more than 4000 g/mol, particularly preferably not more than 3000 g/mol, especially preferably not more than 2000 g/mol and very particularly preferably not more than 1200 g/mol. In a preferred configuration, compounds of the invention are defined by structures of the formulae (IIIa) to (IIIh) and/or (IVa) to (IVh).

In addition, it is a feature of preferred compounds of the invention that they are sublimable. These compounds generally have a molar mass of less than about 1200 g/mol.

When the compound of the invention is substituted by aromatic or heteroaromatic R¹ or R² groups, it is preferable when these do not have any aryl or heteroaryl groups having more than two aromatic six-membered rings fused directly to one another. More preferably, the substituents do not have any aryl or heteroaryl groups having six-membered rings fused directly to one another at all. The reason for this preference is the low triplet energy of such structures. Fused aryl groups which have more than two aromatic six-membered rings fused directly to one another but are nevertheless also suitable in accordance with the invention are phenanthrene and triphenylene, since these also have a high triplet level.

In the case of configuration of the compounds of the invention that are usable as active compound in an organic electronic device for use as fluorescent emitters or as blue OLED materials, preferred compounds may contain corresponding groups, for example fluorene, anthracene and/or pyrene groups which may be substituted by R¹ or R² groups or which are formed by corresponding substitution of the (R¹-1) to (R¹-92) groups, preferably (R¹-33) to (R¹-57) and (R¹-76) to (R¹-86), or (L¹-1) to (L¹-109), preferably (L¹-30) to (L¹-60) and (L¹-71) to (L¹-91), by the substituents R².

In a further preferred embodiment of the invention, R², for example in a structure of formula (I) and preferred embodiments of this structure or the structures where reference is made to these formulae, is the same or different at each instance and is selected from the group consisting of H, D, an aliphatic hydrocarbyl radical having 1 to 10 carbon atoms, preferably having 1, 2, 3 or 4 carbon atoms, or an aromatic or heteroaromatic ring system which has 5 to 30 aromatic ring atoms, preferably 5 to 24 aromatic ring atoms, more preferably 5 to 13 aromatic ring atoms, and may be substituted by one or more alkyl groups each having 1 to 4 carbon atoms, but is preferably unsubstituted.

Preferably, the R² radicals do not form a fused aromatic or heteroaromatic ring system, and preferably do not form any fused ring system, with the ring atoms of the aryl group or heteroaryl group to which the R² radicals are bonded. This includes the formation of a fused ring system with possible substituents R³ which may be bonded to the R² radicals.

In a further preferred embodiment of the invention, R³, for example in a structure of formulae (I), (Ia), (II), (IIa), (III) to (IIIh), (IVa) to (IVh) and preferred embodiments of this structure or the structures where reference is made to these formulae, is the same or different at each instance and is selected from the group consisting of H, D, F, CN, an aliphatic hydrocarbyl radical having 1 to 10 carbon atoms, preferably having 1, 2, 3 or 4 carbon atoms, or an aromatic or heteroaromatic ring system which has 5 to 30 aromatic ring atoms, preferably 5 to 24 aromatic ring atoms, more preferably 5 to 13 aromatic ring atoms, and may be substituted by one or more alkyl groups each having 1 to 4 carbon atoms, but is preferably unsubstituted.

In a preferred embodiment, preference is given inter alia to the combinations of structural elements of the formula II with aromatic or heteroaromatic ring systems that have the following properties:

| | | | Sum total of the indices |
|---|---|---|---|
| | | Index v in the | p, e, i, j, h and m in the |
| | | substructures of | structures of the |
| Substructure | L¹ in | the formula | formula (Ar′-1) to |
| of the formula | substructure | (IIa) | (Ar′-11) |
| Ar′-1 to | bond or L¹-1 to | 0 to 6, preferably 0 | 0 to 4, preferably |
| Ar′-11 | L¹-108, preferably | to 3, more preferably | 1 or 2, more |
| | bond or L¹-1 to L¹-4 | 0 or 1, especially | preferably 1 |
| | | preferably 0 | |
| Ar′-1 | bond or L¹-1 to | 0 to 6, preferably 0 | 0 to 4, preferably |
| | L¹-108, preferably | to 3, more preferably | 1 or 2, more |
| | bond or L¹-1 to L¹-4 | 0 or 1, especially | preferably 1 |
| | | preferably 0 | |
| Ar′-2 | bond or L¹-1 to | 0 to 6, preferably 0 | 0 to 4, preferably |
| | L¹-108, preferably | to 3, more preferably | 1 or 2, more |
| | bond or L¹-1 to L¹-4 | 0 or 1, especially | preferably 1 |
| | | preferably 0 | |
| Ar′-3 | bond or L¹-1 to | 0 to 6, preferably 0 | 0 to 4, preferably |
| | L¹-108, preferably | to 3, more preferably | 1 or 2, more |
| | bond or L¹-1 to L¹-4 | 0 or 1, especially | preferably 1 |
| | | preferably 0 | |
| Ar′-4 | bond or L¹-1 to | 0 to 6, preferably 0 | 0 to 4, preferably |
| | L¹-108, preferably | to 3, more preferably | 1 or 2, more |
| | bond or L¹-1 to L¹-4 | 0 or 1, especially | preferably 1 |
| | | preferably 0 | |
| Ar′-5 | bond or L¹-1 to | 0 to 6, preferably 0 | 0 to 4, preferably |
| | L¹-108, preferably | to 3, more preferably | 1 or 2, more |
| | bond or L¹-1 to L¹-4 | 0 or 1, especially | preferably 1 |
| | | preferably 0 | |
| Ar′-6 | bond or L¹-1 to | 0 to 6, preferably 0 | 0 to 4, preferably |
| | L¹-108, preferably | to 3, more preferably | 1 or 2, more |
| | bond or L¹-1 to L¹-4 | 0 or 1, especially | preferably 1 |
| | | preferably 0 | |
| Ar′-7 | bond or L¹-1 to | 0 to 6, preferably 0 | 0 to 4, preferably |
| | L¹-108, preferably | to 3, more preferably | 1 or 2, more |
| | bond or L¹-1 to L¹-4 | 0 or 1, especially | preferably 1 |
| | | preferably 0 | |
| Ar′-8 | bond or L¹-1 to | 0 to 6, preferably 0 | 0 to 4, preferably |
| | L¹-108, preferably | to 3, more preferably | 1 or 2, more |
| | bond or L¹-1 to L¹-4 | 0 or 1, especially | preferably 1 |
| | | preferably 0 | |
| Ar′-9 | bond or L¹-1 to | 0 to 6, preferably 0 | 0 to 4, preferably |
| | L¹-108, preferably | to 3, more preferably | 1 or 2, more |
| | bond or L¹-1 to L¹-4 | 0 or 1, especially | preferably 1 |
| | | preferably 0 | |
| Ar′-10 | bond or L¹-1 to | 0 to 6, preferably 0 | 0 to 4, preferably |
| | L¹-108, preferably | to 3, more preferably | 1 or 2, more |
| | bond or L¹-1 to L¹-4 | 0 or 1, especially | preferably 1 |
| | | preferably 0 | |
| Ar′-11 | bond or L¹-1 to | 0 to 6, preferably 0 | 0 to 4, preferably |
| | L¹-108, preferably | to 3, more preferably | 1 or 2, more |
| | bond or L¹-1 to L¹-4 | 0 or 1, especially | preferably 1 |
| | | preferably 0 | |

In a preferred embodiment, preference is given inter alia to the combinations of structural elements of the formula IIa with aromatic or heteroaromatic ring systems that have the following properties:

| Substructure | | Index u in the | Sum total of the |
|---|---|---|---|
| of the | L¹ in | substructures of | indices o, n, m and l |
| formula | substructure | the formula (IIa) | (Ar′-12) to (Ar′-57) |
| Ar′-12 to | bond or L¹-1 to | 0 to 6, | 0 to 4, |
| Ar′-57 | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-12 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-13 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-14 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-15 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-16 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-17 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-18 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-19 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-20 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-21 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-22 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-23 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-24 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-25 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-26 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-27 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-28 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-29 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-30 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-31 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-32 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-33 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-34 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-35 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-36 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-37 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-38 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-39 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-40 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-41 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-42 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-43 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-44 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-45 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-46 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-47 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-48 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-49 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-50 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-51 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-52 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-53 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-54 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-55 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-56 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |
| Ar′-57 | bond or L¹-1 to | 0 to 6, | 0 to 4, |
| | L¹-108, | preferably 0 to | preferably 1 or |
| | preferably bond | 3, more | 2, more |
| | or L¹-1 to L¹-4 | preferably 0 or | preferably 1 |
| | | 1, especially | |
| | | preferably 0 | |

In a preferred embodiment, preference is given inter alia to the combinations of the substructure of the formula II with electron transport group-comprising radicals according to the following table:

| Substructure of | Substructure of | Sum total of the | Index v in the |
|---|---|---|---|
| the formula QL | the formula QL | indices k, l, g, h | substructures of |
| with Q | with L¹ | and j | the formula (II) |
| Q-1 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-2 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-3 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-4 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-5 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-6 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-7 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-8 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-9 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-10 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-11 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-12 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-13 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-14 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-15 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-16 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-17 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-18 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-19 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-20 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-21 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-22 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-23 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-24 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-25 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-26 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-27 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-28 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-29 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-30 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-31 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-32 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-33 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-34 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-35 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-36 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-37 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-38 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-39 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-40 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-41 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-42 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-43 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |
| Q-44 | bond or L¹-1 to | 0 to 3, | 0 to 6, |
| | L¹-108, | preferably 0 or | preferably 0 to |
| | preferably bond | 1, more | 3, more |
| | or L¹-1 to L¹-4 | preferably 0 | preferably 0 or |
| | | | 1, especially |
| | | | preferably 0 |

In a preferred embodiment, preference is given inter alia to the combinations of the substructure of the formula II having hole transport groups according to the following table:

| Substructure | | | Index v in the |
|---|---|---|---|
| of the | with Ar¹ or with | | substructures of |
| formula | Ar³ or Ar⁴ | with Ar² | the formula (II) |
| H-1 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-2 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-3 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-4 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-5 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-6 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-7 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-8 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-9 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-10 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-11 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-12 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-13 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-14 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-15 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-16 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-17 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-18 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-19 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-20 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-21 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-22 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-23 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-24 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-25 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |
| H-26 | R¹-1 to R¹-92, | bond or L¹-1 to | 0 to 6, |
| | preferably | L¹-108, | preferably 0 to |
| | R¹-1 to R¹-4 | preferably bond | 3, more |
| | | or L¹-1 to L¹-4 | preferably 0 or |
| | | | 1, especially |
| | | | preferably 0 |

Preference is further given to compounds of the invention that have structures of the formulae (IIIa) to (IIIh) and (IVa) to (IVh) and have the following properties, where the W group is more preferably O, S or NR, more preferably NR:

| at least one A^{a} | | | |
|---|---|---|---|
| radical | | | |
| comprises one | | | |
| of the groups | where Ar³, Ar⁴ | Ar² | R¹ (if present) |
| H-1 to H-3 | R¹-1 to R¹-92 | L¹-1 to L¹-108 | R¹-1 to R¹-92 |
| H-1 to H-3 | R¹-1 to R¹-92 | bond | R¹-1 to R¹-92 |
| H-1 to H-3 | R¹-1 to R¹-92 | L¹-1 to L¹-4 | R¹-1 to R¹-92 |
| H-1 | R¹-2 to R¹-32 | bond, | R¹-1 to R¹-92 |
| | | L¹-1 to L¹-4 | |
| H-1 | R¹-2 to R¹-32 | L¹-41 to L¹-54 | R¹-1 to R¹-92 |
| H-1 | R¹-1 | bond, | R¹-1 to R¹-92 |
| | | L¹-1 to L¹-4 | |
| H-1 | R¹-1 | L¹-41 to L¹-54 | R¹-1 to R¹-92 |
| H-2 | R¹-2 to R¹-32 | bond, | R¹-1 to R¹-92 |
| | | L¹-1 to L¹-4 | |
| H-2 | R¹-2 to R¹-32 | L¹-41 to L¹-54 | R¹-1 to R¹-92 |
| H-2 | R¹-1 | bond, | R¹-1 to R¹-92 |
| | | L¹-1 to L¹-4 | |
| H-2 | R¹-1 | L¹-41 to L¹-54 | R¹-1 to R¹-92 |
| H-3 | R¹-2 to R¹-32 | bond, | R¹-1 to R¹-92 |
| | | L¹-1 to L¹-4 | |
| H-3 | R¹-2 to R¹-32 | L¹-41 to L¹-54 | R¹-1 to R¹-92 |
| H-3 | R¹-1 | bond, | R¹-1 to R¹-92 |
| | | L¹-1 to L¹-4 | |
| H-3 | R¹-1 | L¹-41 to L¹-54 | R¹-1 to R¹-92 |

Preference is further given to compounds of the invention that have structures of the formulae (IIIa) to (IIIh) and (IVa) to (IVh) and have the following properties, where the W group is more preferably O, S or NR, more preferably NR:

| at least one A^{a} | | | |
|---|---|---|---|
| comprises one | | | Ar¹ or R¹ (if |
| of the groups | Ar² | Ar² (preferred) | present) |
| H-4 to H-26 | L¹-1 to L¹-108 | L¹-1 to L¹-54, | R¹-1 to R¹-92 |
| | | L¹-92 to L¹-108 | |
| H-4 to H-26 | bond or | bond | R¹-1 to R¹-92 |
| | L¹-1 to L¹-54, | | |
| | L¹-92 to L¹-108 | | |
| H-4 to H-26 | bond or | L¹-1 to L¹-4 | R¹-1 to R¹-92 |
| | L¹-1 to L¹-4, | | |
| | L¹-92 to L¹-103 | | |
| H-4 to H-26 | L¹-30 to L¹-52 or | L¹-37 to L¹-52 or | R¹-1 to R¹-92 |
| | L¹-104 to L¹-108 | L¹-106 to L¹-108 | |
| H-4 | L¹-1 to L¹-108 | L¹-1 to L¹-54, | R¹-1 to R¹-92 |
| | | L¹-92 to L¹-108 | |
| H-4 | bond or | bond | R¹-1 to R¹-92 |
| | L¹-1 to L¹-54, | | |
| | L¹-92 to L¹-108 | | |
| H-4 | bond or | L¹-1 to L¹-4 | R¹-1 to R¹-92 |
| | L¹-1 to L¹-4, | | |
| | L¹-92 to L¹-103 | | |
| H-4 | L¹-30 to L¹-52 or | L¹-37 to L¹-52 or | R¹-1 to R¹-92 |
| | L¹-104 to L¹-108 | L¹-106 to L¹-108 | |
| H-5 | L¹-1 to L¹-108 | L¹-1 to L¹-54, | R¹-1 to R¹-92 |
| | | L¹-92 to L¹-108 | |
| H-5 | bond or | bond | R¹-1 to R¹-92 |
| | L¹-1 to L¹-54, | | |
| | L¹-92 to L¹-108 | | |
| H-5 | bond or | L¹-1 to L¹-4 | R¹-1 to R¹-92 |
| | L¹-1 to L¹-4, | | |
| | L¹-92 to L¹-103 | | |
| H-5 | L¹-30 to L¹-52 or | L¹-37 to L¹-52 or | R¹-1 to R¹-92 |
| | L¹-104 to L¹-108 | L¹-106 to L¹-108 | |
| H-6 | L¹-1 to L¹-108 | L¹-1 to L¹-54, | R¹-1 to R¹-92 |
| | | L¹-92 to L¹-108 | |
| H-6 | bond or | bond | R¹-1 to R¹-92 |
| | L¹-1 to L¹-54, | | |
| | L¹-92 to L¹-108 | | |
| H-6 | bond or | L¹-1 to L¹-4 | R¹-1 to R¹-92 |
| | L¹-1 to L¹-4, | | |
| | L¹-92 to L¹-103 | | |
| H-6 | L¹-30 to L¹-52 or | L¹-37 to L¹-52 or | R¹-1 to R¹-92 |
| | L¹-104 to L¹-108 | L¹-106 to L¹-108 | |
| H-7 | L¹-1 to L¹-108 | L¹-1 to L¹-54, | R¹-1 to R¹-92 |
| | | L¹-92 to L¹-108 | |
| H-7 | bond or | bond | R¹-1 to R¹-92 |
| | L¹-1 to L¹-54, | | |
| | L¹-92 to L¹-108 | | |
| H-7 | bond or | L¹-1 to L¹-4 | R¹-1 to R¹-92 |
| | L¹-1 to L¹-4, | | |
| | L¹-92 to L¹-103 | | |
| H-7 | L¹-30 to L¹-52 or | L¹-37 to L¹-52 or | R¹-1 to R¹-92 |
| | L¹-104 to L¹-108 | L¹-106 to L¹-108 | |
| H-8 | L¹-1 to L¹-108 | L¹-1 to L¹-54, | R¹-1 to R¹-92 |
| | | L¹-92 to L¹-108 | |
| H-8 | bond or | bond | R¹-1 to R¹-92 |
| | L¹-1 to L¹-54, | | |
| | L¹-92 to L¹-108 | | |
| H-8 | bond or | L¹-1 to L¹-4 | R¹-1 to R¹-92 |
| | L¹-1 to L¹-4, | | |
| | L¹-92 to L¹-103 | | |
| H-8 | L¹-30 to L¹-52 or | L¹-37 to L¹-52 or | R¹-1 to R¹-92 |
| | L¹-104 to L¹-108 | L¹-106 to L¹-108 | |

Preference is further given to compounds of the invention that have structures of the formulae (IIIa) to (IIIh) and (IVa) to (IVh) and have the following properties, where the W group is more preferably O, S or NR, more preferably NR:

| at least one A^{a} | | | |
|---|---|---|---|
| radical | | | |
| comprises one | | | Ar¹ or R¹ (if |
| of the groups | L¹ | L¹ (preferred) | present) |
| Q-1 to Q-44 | L¹-1 to L¹-108 | L¹-1 to L¹-54, | R¹-1 to R¹-92 |
| | | L¹-92 to L¹-108 | |
| Q-1 to Q-44 | bond or | bond | R¹-1 to R¹-92 |
| | L¹-1 to L¹-54, | | |
| | L¹-92 to L¹-108 | | |
| Q-1 to Q-44 | bond or | L¹-1 to L¹-4 | R¹-1 to R¹-92 |
| | L¹-1 to L¹-4, | | |
| | L¹-92 to L¹-103 | | |
| Q-1 to Q-44 | L¹-30 to L¹-52 or | L¹-37 to L¹-52 or | R¹-1 to R¹-92 |
| | L¹-104 to L¹-108 | L¹-106 to L¹-108 | |
| Q-11 to Q-25 | L¹-1 to L¹-108 | L¹-1 to L¹-54, | R¹-1 to R¹-92 |
| | | L¹-92 to L¹-108 | |
| Q-11 to Q-25 | bond or | bond | R¹-1 to R¹-92 |
| | L¹-1 to L¹-54, | | |
| | L¹-92 to L¹-108 | | |
| Q-11 to Q-25 | bond or | L¹-1 to L¹-4 | R¹-1 to R¹-92 |
| | L¹-1 to L¹-4, | | |
| | L¹-92 to L¹-103 | | |
| Q-11 to Q-25 | L¹-30 to L¹-52 or | L¹-37 to L¹-52 or | R¹-1 to R¹-92 |
| | L¹-104 to L¹-108 | L¹-106 to L¹-108 | |
| Q-26 to Q-44, | L¹-1 to L¹-108 | L¹-1 to L¹-54, | R¹-1 to R¹-92 |
| preferably Q-26 | | L¹-92 to L¹-108 | |
| to Q-42 | | | |
| Q-26 to Q-44, | bond or | bond | R¹-1 to R¹-92 |
| preferably Q-26 | L¹-1 to L¹-54, | | |
| to Q-42 | L¹-92 to L¹-108 | | |
| Q-26 to Q-44, | bond or | L¹-1 to L¹-4 | R¹-1 to R¹-92 |
| preferably Q-26 | L¹-1 to L¹-4, | | |
| to Q-42 | L¹-92 to L¹-103 | | |
| Q-26 to Q-44, | L¹-30 to L¹-52 or | L¹-37 to L¹-52 or | R¹-1 to R¹-92 |
| preferably Q-26 | L¹-104 to L¹-108 | L¹-106 to L¹-108 | |
| to Q-42 | | | |
| Q-23 to Q-25 | L¹-1 to L¹-108 | L¹-1 to L¹-54, | R¹-1 to R¹-92 |
| | | L¹-92 to L¹-108 | |
| Q-23 to Q-25 | bond or | bond | R¹-1 to R¹-92 |
| | L¹-1 to L¹-54, | | |
| | L¹-92 to L¹-108 | | |
| Q-23 to Q-25 | bond or | L¹-1 to L¹-4 | R¹-1 to R¹-92 |
| | L¹-1 to L¹-4, | | |
| | L¹-92 to L¹-103 | | |
| Q-23 to Q-25 | L¹-30 to L¹-52 or | L¹-37 to L¹-52 or | R¹-1 to R¹-92 |
| | L¹-104 to L¹-108 | L¹-106 to L¹-108 | |
| Q-23 | L¹-1 to L¹-108 | L¹-1 to L¹-54, | R¹-1 to R¹-92 |
| | | L¹-92 to L¹-108 | |
| Q-23 | bond or | bond | R¹-1 to R¹-92 |
| | L¹-1 to L¹-54, | | |
| | L¹-92 to L¹-108 | | |
| Q-23 | bond or | L¹-1 to L¹-4 | R¹-1 to R¹-92 |
| | L¹-1 to L¹-4, | | |
| | L¹-92 to L¹-103 | | |
| Q-23 | L¹-30 to L¹-52 or | L¹-37 to L¹-52 or | R¹-1 to R¹-92 |
| | L¹-104 to L¹-108 | L¹-106 to L¹-108 | |

It may further be the case that the compound usable for production of functional layers of electronic devices is a ligand in a metal complex.

These metal complexes are novel and have unexpected technical advantages over known metal complexes. The present invention therefore further provides a metal complex comprising one or more one structural elements of the formula (I) and/or (Ia) or preferred embodiments of these structural elements.

The present invention accordingly further provides a metal complex comprising at least one structure of the general formula (1)

M(L)ₙ(L′)ₘ Formula (1)

where the symbols and indices used are as follows:
M is a transition metal, preferably copper, molybdenum, tungsten, rhenium, ruthenium, osmium, rhodium, iridium, palladium, platinum, silver, gold or europium, more preferably iridium or platinum;
L is the same or different at each instance and is a bidentate ligand;
L′ is the same or different at each instance and is a ligand;
n is 1, 2 or 3, preferably 2 or 3, more preferably 2;
m is 0, 1, 2, 3 or 4, preferably 0, 1 or 2, more preferably 0 or 1, especially preferably 0;
   at the same time, it is also possible for two or more ligands L to be joined to one another or for L to be joined to L′ by a single bond or a bivalent or trivalent bridge, thus forming a tridentate, tetradentate, pentadentate or hexadentate ligand system, characterized in that the metal complex contains at least one substructure of the formula (2) and/or (2a):

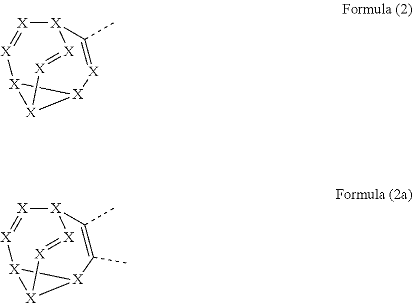

where the dotted bond represents the linkage of this group to a further part of the metal complex of the formula (1), and the symbol X has the definition set out above for formula (I) or (Ia) inter alia.

It may further be the case that the metal complex contains at least one substructure of the formula (2-1) and/or (2a-1):

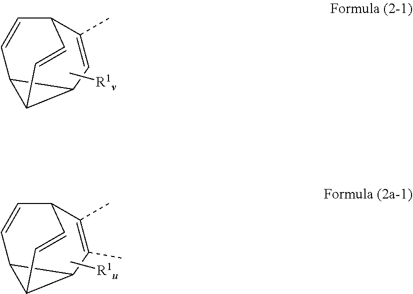

where the dotted bond represents the linkage of this group to a further part of the metal complex of the formula (1), and the symbols u, v and R¹ have the definition set out above for formula (I), (Ia), (II) or (IIa) inter alia.

In a preferred embodiment, the linkage, represented by a dotted bond in formula (2), (2a) (2-1) or (2a-1), of the substructure of formula (2), (2a) (2-1) or (2a-1) is bonded to an aromatic or heteroaromatic ring system, preferably an aryl or heteroaryl radical having preferably 5 to 40 ring atoms. It is possible here for the aromatic or heteroaromatic ring system, preferably the aryl or heteroaryl radical having 5 to 40 ring atoms, preferably 5 to 24 ring atoms and especially preferably 6 to 12 ring atoms, to be substituted by one or more R radicals as defined above for formula (2); however, this radical is preferably unsubstituted. At the same time, the aromatic or heteroaromatic ring system or the aryl or heteroaryl radical is preferably part of a ligand L and coordinates directly to the metal M.

It may further be the case that the substructure of formula (2), (2a), (2-1) and/or (2a-1) is bonded directly to the metal atom M. This may preferably take place via one of the R or R¹ radicals.

The inventive metal complexes of the formula (1) may contain one, two, three or more of the substructures of the formula (2), (2a) further detailed above or preferred embodiments thereof. In a specific embodiment, an inventive metal complex of the formula (1) may comprise exactly one substructure of the formula (2) or (2a). Preferably, metal complexes of the formula (1) may contain two, more preferably three or more, of the substructures of the formula (2) and/or (2a) further detailed above or preferred embodiments thereof. Especially preferably, the inventive metal complexes of the formula (1) comprise one, two, three or six substructures of the formula (2) and/or (2a) or preferred embodiments thereof.

There follows a description of the bidentate ligands that are identified by the symbol L in formula (1) and bonded to M. The metal complexes may preferably have one, two or three bidentate ligands. The coordinating atoms of the bidentate ligands here may be the same or different at each instance and may be selected from C, N, P, O, S and/or B, more preferably C, N and/or O and most preferably C and/or N. The bidentate ligands preferably have one carbon atom and one nitrogen atom or two carbon atoms or two nitrogen atoms or two oxygen atoms or one oxygen atom and one nitrogen atom as coordinating atoms. In this case, the coordinating atoms of each of the ligands may be the same, or they may be different. Preferably at least one of the bidentate ligands has, more preferably all the bidentate ligands have, one carbon atom and one nitrogen atom or two carbon atoms as coordinating atoms, especially one carbon atom and one nitrogen atom. More preferably at least two of the bidentate ligands and most preferably, when M=Ir, all three bidentate ligands have one carbon atom and one nitrogen atom or two carbon atoms as coordinating atoms, especially one carbon atom and one nitrogen atom. Particular preference is thus given to an iridium complex in which all three bidentate ligands are ortho-metalated, i.e. form a metalacycle with the iridium in which at least one iridium-carbon bond is present.

More preferably, the metal complex does not comprise any monodentate ligands, and all bidentate ligands, identically or differently at each instance, have at least one carbon atom as coordinating atom. It should be emphasized once again that the bidentate ligands may be joined to one another and may have further coordination sites, and so the term “bidentate ligand” refers to a ligand having at least two coordination sites. In the case that a bidentate ligand has exactly two coordination sites, this is stated explicitly. In this connection, it should also be noted that the index n in formula (1) may be 1 and the index m may simultaneously be 0, where, in this case, for example, the bidentate ligands L are joined to one another and form a hexadentate ligand system. In this case, the three ligands bonded to one another may also be regarded as sub-ligands.

The indices in the above-detailed formula (1) or the preferred embodiments of this formula are dependent on the type of metal and possible linkage of the ligands. For unbridged iridium complexes (M=Ir), n is more preferably 3 and m is 0. Since platinum in preferred complexes is only tetracoordinated, for unbridged platinum complexes (M=Pt), n is more preferably 2 and m=0. In the case of bridged complexes, the bidentate ligands can be regarded as sub-ligands, and so, when considered in this way, the details given above are applicable. Otherwise, according to the degree of bridging, n in particularly preferred embodiments as described above and hereinafter is 1 in each case, particular preference being given to formation of a metal complex containing iridium and a hexadentate tripodal ligand or a metal complex containing platinum and a tetradentate ligand.

Preferably, the metal complex of formula (1) comprises three bidentate ligands L which may optionally also be joined. The three bidentate ligands may be the same or different. When the bidentate ligands are the same, they preferably also have the same substitution. When all three bidentate ligands chosen are the same, the result in the case of polypodal complexes is C₃-symmetric iridium complexes. It may also be advantageous to select the three bidentate ligands differently or to select two identical ligands and a different third ligand, so as to give rise to C₁-symmetric metal complexes, because this permits greater possible variation of the ligands, such that the desired properties of the complex, for example the HOMO and LUMO position or the emission color, can be varied more easily. Moreover, the solubility of the complexes can thus also be improved without having to attach long aliphatic or aromatic solubility-imparting groups.

In a preferred embodiment of the invention, either the three bidentate ligands are selected identically or two of the bidentate ligands are selected identically and the third bidentate ligand is different from the first two bidentate ligands.

It may further be the case that the metal complex has three bidentate ligands, where all three ligands chosen are the same or two of the bidentate ligands chosen are the same and the third bidentate ligand is different from the first two bidentate ligands.

It may further be the case that the metal is Ir(III) and the metal complex has three bidentate ligands, where two of the bidentate ligands respectively coordinate to the iridium via one carbon atom and one nitrogen atom, and the third of the bidentate ligands coordinates to the iridium via one carbon atom and one nitrogen atom or via two carbon atoms or via two nitrogen atoms, where preferably the third of the bidentate ligands coordinates to the iridium via one carbon atom and one nitrogen atom or via two carbon atoms.

In a further configuration, it may be the case that the metal is Pt coordinated to two bidentate ligands.

It is further preferable when the metalacycle which is formed from the metal and the bidentate ligand is a five-membered ring, which is preferable particularly when the coordinating atoms are C and N, C and C, N and N, or N and O. When the coordinating atoms are O, a six-membered metalacyclic ring may also be preferred. This is shown schematically hereinafter:

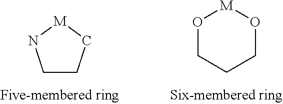

where N is a coordinating nitrogen atom, C is a coordinating carbon atom and O represents coordinating oxygen atoms, and the carbon atoms shown are atoms of the bidentate ligand.

In a preferred embodiment of the invention, at least one of the bidentate ligands, more preferably at least two of the bidentate ligands, most preferably, when M=Ir, all three bidentate ligands of the metal complex shown in formula (1), are the same or different at each instance and are selected from the structures of the following formulae (L-1), (L-2), (L-3), (L-4) and/or (L-5):

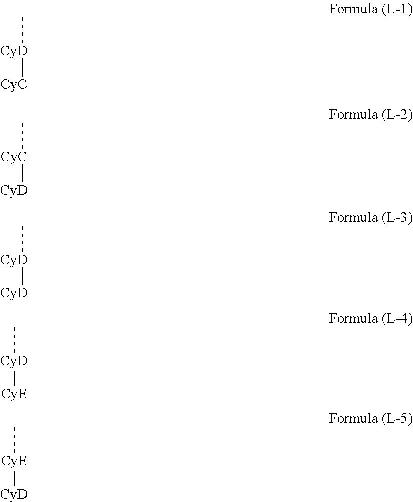

where the symbols and indices used are as follows:
CyC is the same or different at each instance and is a substituted or unsubstituted aryl or heteroaryl group which has 5 to 14 aromatic ring atoms and coordinates in each case to the metal via a carbon atom and which is bonded to CyD via a covalent bond in each case;
CyD is the same or different at each instance and is a substituted or unsubstituted heteroaryl group which has 5 to 14 aromatic ring atoms and coordinates to the metal via a nitrogen atom or via a carbene carbon atom and which is bonded to CyC via a covalent bond;
CyE is the same or different at each instance and is a substructure of formula (2) and/or (2a) which coordinates to the metal in each case via a carbon atom and which is bonded in each case to CyD via a covalent bond;
   at the same time, it is also possible for two or more ligands L to be joined to one another, or for L to be joined to L′, via a single bond or a bivalent or trivalent bridge, thus forming a tridentate, tetradentate, pentadentate or hexadentate ligand system; where these optional bonds to a bridge are indicated by the dotted bond; at the same time, two or more of the optional substituents together may form a ring system; at the same time, a substituent may also additionally coordinate to M; in addition, the optional radicals are preferably selected from the abovementioned R radicals and/or the substructure of the formula (2) and/or (2a).

At the same time, CyD in the ligands of the formulae (L-1) and (L-2) preferably coordinates via an uncharged nitrogen atom or via a carbene carbon atom. Further preferably, one of the two CyD groups in the ligand of the formula (L-3) coordinates via an uncharged nitrogen atom and the other of the two CyD groups via an anionic nitrogen atom. Further preferably, CyC in the ligands of the formulae (L-1) and (L-2) coordinates via anionic carbon atoms.

When two or more of the substituents, especially two or more R radicals, together form a ring system, it is possible for a ring system to be formed from substituents bonded to directly adjacent carbon atoms. In addition, it is also possible that the substituents on CyC and CyD in the formulae (L-1) and (L-2) or the substituents on the two CyD groups in formula (L-3) together form a ring, as a result of which CyC and CyD or the two CyD groups or the two CyC groups may also together form a single fused aryl or heteroaryl group as bidentate ligand.

In a preferred embodiment of the present invention, CyC is an aryl or heteroaryl group having 6 to 13 aromatic ring atoms, more preferably having 6 to 10 aromatic ring atoms, most preferably having 6 aromatic ring atoms, which coordinates to the metal via a carbon atom, which may be substituted by one or more R radicals and which is bonded to CyD via a covalent bond.

Preferred embodiments of the CyC group are the structures of the following formulae (CyC-1) to (CyC-20):

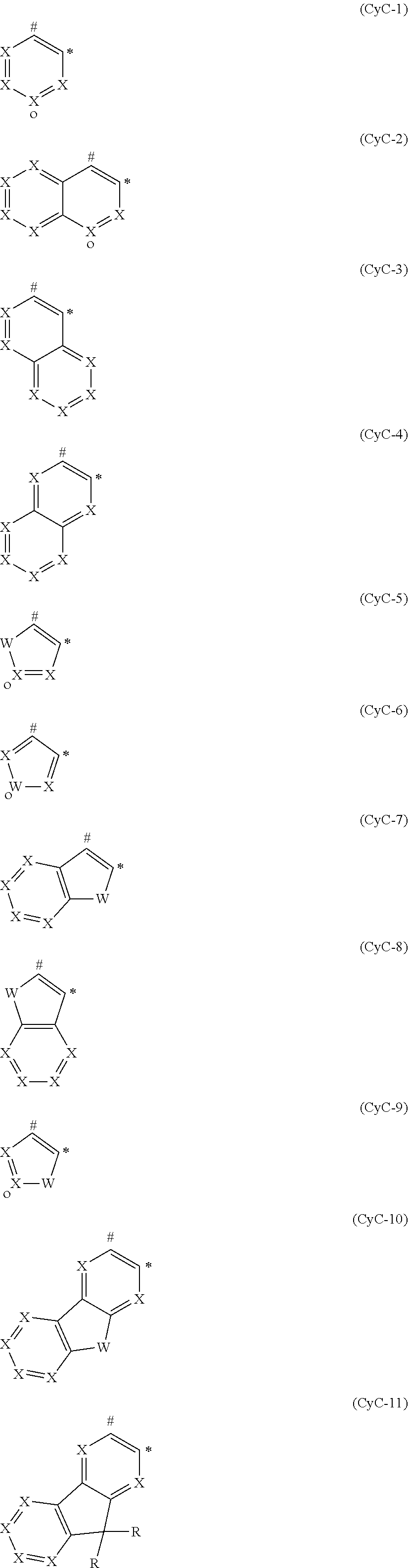

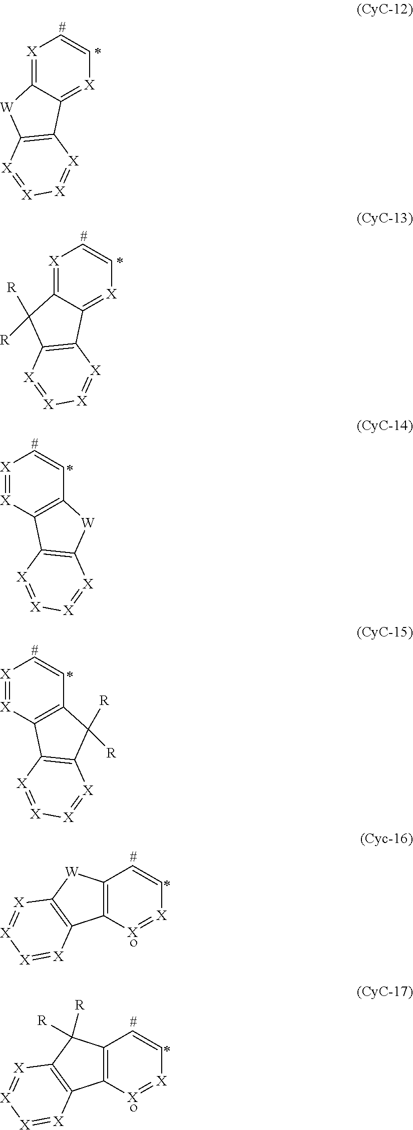

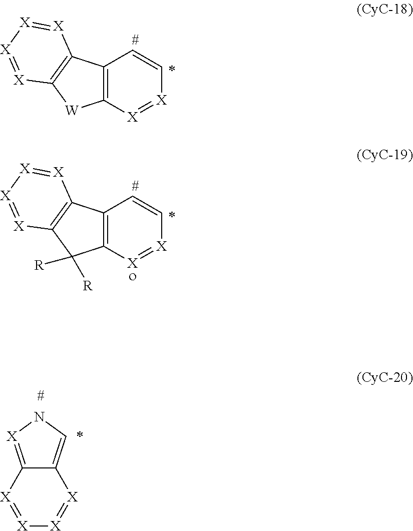

where CyC binds in each case to the position in CyD indicated by # and coordinates to the metal at the position indicated by *, R has the definition given above, especially for formula (I) or (Ia), and the further symbols used are as follows:
X is the same or different at each instance and is CR or N, where preferably not more than two X symbols per cycle are N;
W is NR, O or S;
   where the ligands may optionally be bonded by a bridge via the CyC group, where the bond to the bridge may preferably be via the position marked “o”, where the position marked “o” represents a carbon atom if it constitutes a bridgehead site. When the CyC group is bonded to a bridge, the bond is preferably via the position marked “o” in the formulae depicted above, and so the symbol X marked “o” in that case is preferably C. The above-depicted structures which do not contain any symbol X marked “o” are preferably not bonded directly to a bridge, since such a bond to the bridge is not advantageous for steric reasons.

Preferably, a total of not more than two symbols X in CyC are N, more preferably not more than one symbol X in CyC is N, and especially preferably all symbols X are CR, with the proviso that, when CyC is bonded to a bridge, one symbol X is C and the bridge is bonded to this carbon atom.

Particularly preferred CyC groups are the groups of the following formulae (CyC-1a) to (CyC-20a):

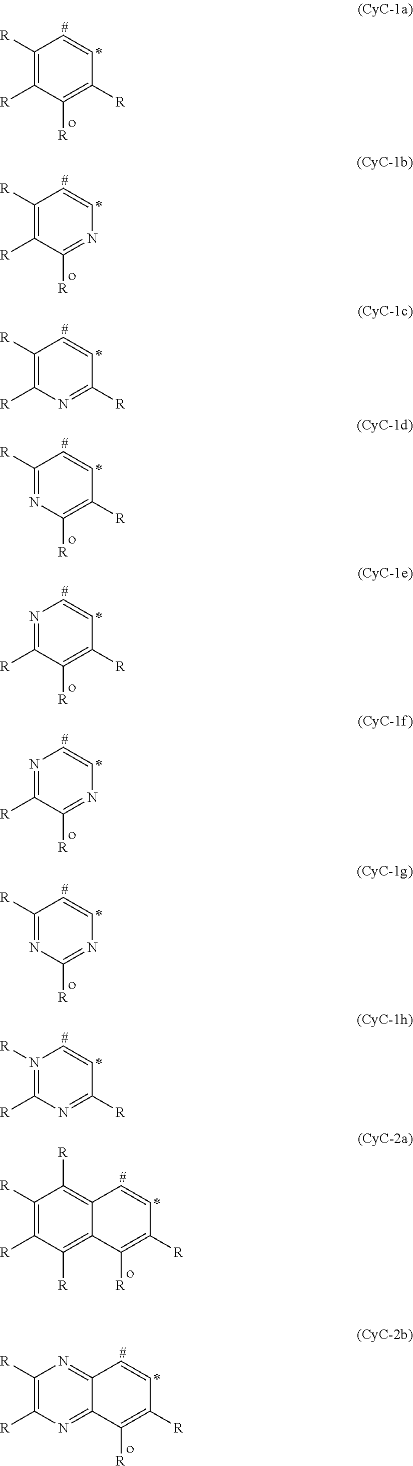

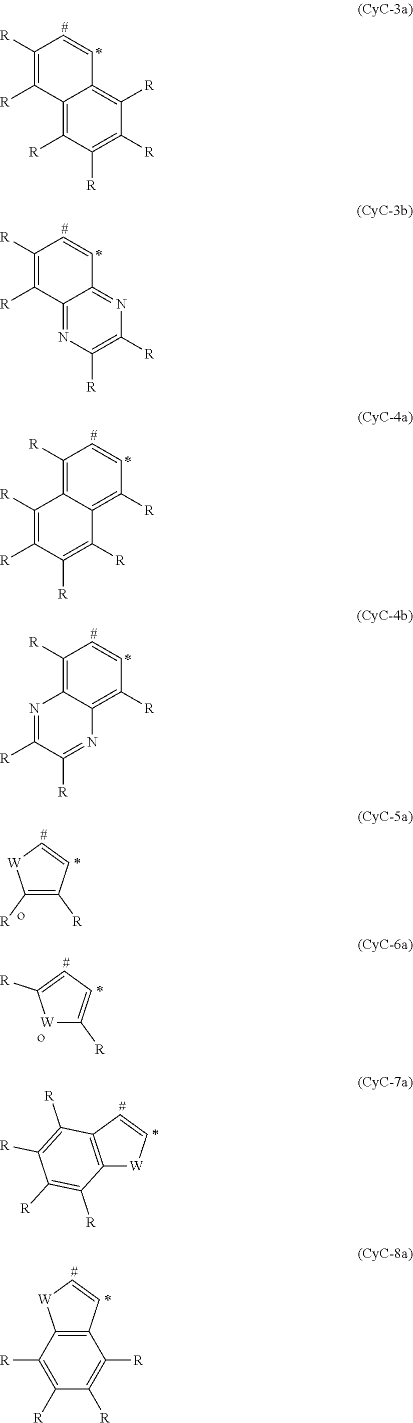

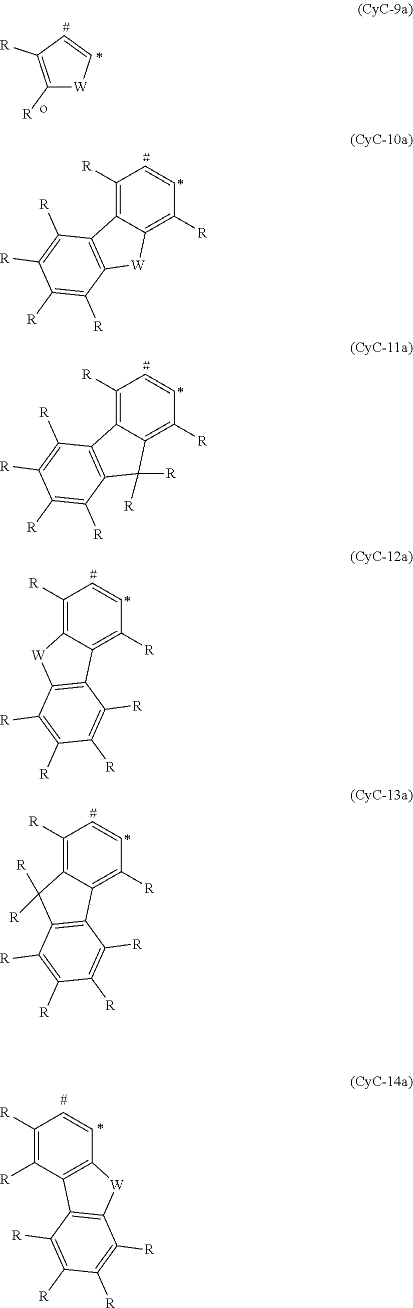

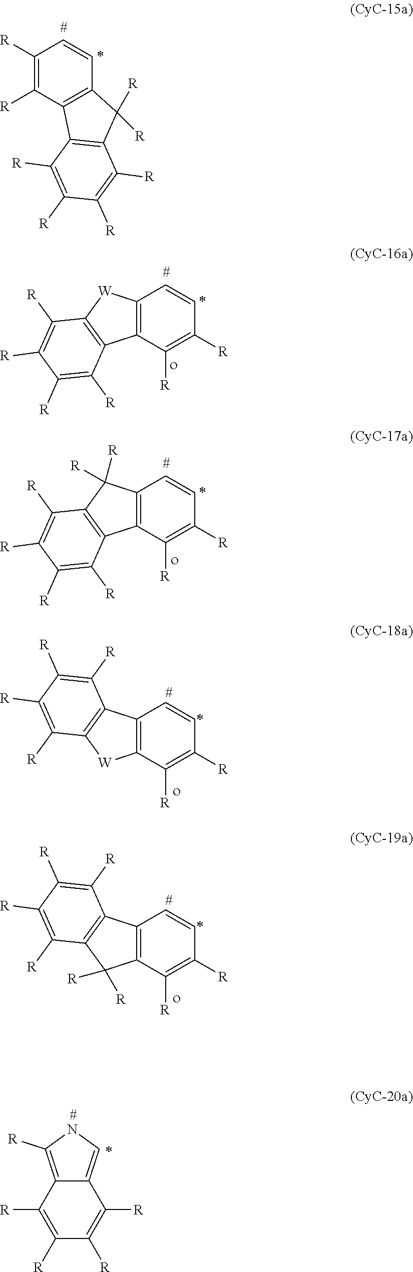

where the symbols have the definitions given above and, when a bridge is bonded to CyC, one R radical is absent and the bridge is bonded to the corresponding carbon atom. When a CyC group is bonded to a bridge, the bond is preferably via the position marked “o” in the formulae depicted above, and so the R radical in this position in that case is preferably absent. The above-depicted structures which do not contain any carbon atom marked “o” are preferably not bonded directly to a bridge.

The position of the bond by which the (sub)ligands may be bonded to one another via a bridge may be different in the case of different metals, where the preference shown in the structures of the formulae (CyC-1) to (CyC-20) and (CyC-1a) to (CyC-20a) is applicable to iridium, for example. With regard to the position for platinum and similar metals, the examples give valuable pointers, where the bridge is preferably via a position adjacent to the coordination or binding site to the metal atom.

Preferred groups among the (CyC-1) to (CyC-19) groups are the (CyC-1), (CyC-3), (CyC-8), (CyC-10), (CyC-12), (CyC-13) and (CyC-16) groups, and particular preference is given to the (CyC-1a), (CyC-3a), (CyC-8a), (CyC-10a), (CyC-12a), (CyC-13a) and (CyC-16a) groups.

It may further be the case that CyC comprises a substructure of the formula (2), (2a), (2-1) and/or (2a-1) or the preferred embodiment of this substructure or is formed by suitable substitution by R radicals, where the X groups in formula (2) or (2a) in this case are CR¹. More preferably, one R radical in the above-detailed embodiments of the CyC group represents a substructure of the formula (2) or (2a), such that the bonding site shown by a dotted bond in formula (2) is bonded directly to the aromatic or heteroaromatic ring system shown in the CyC group. In the case of the configurations shown in formula (2a) or the preferred embodiments thereof, the CyC group is bonded directly by two binding sites to the aromatic or heteroaromatic ring system shown in the CyC group, such that the double bond shown in the structures of formula (2a) which is marked by the dotted bond constitutes part of the aromatic or heteroaromatic ring system.

In a further preferred embodiment of the invention, CyD is a heteroaryl group having 5 to 13 aromatic ring atoms, more preferably having 6 to 10 aromatic ring atoms, which coordinates to the metal via an uncharged nitrogen atom or via a carbene carbon atom and which may be substituted by one or more R radicals and which is bonded via a covalent bond to CyC.

Preferred embodiments of the CyD group are the structures of the following formulae (CyD-1) to (CyD-14):

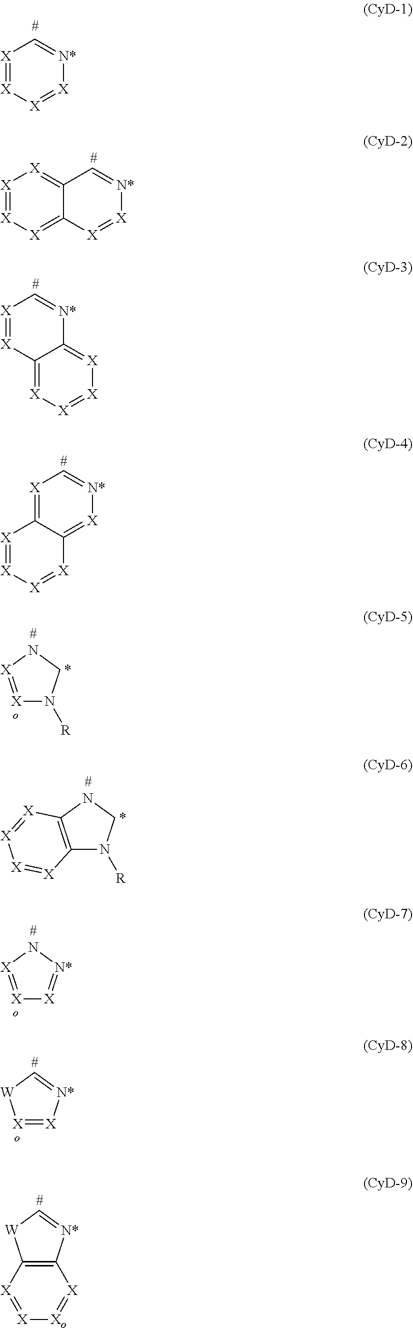

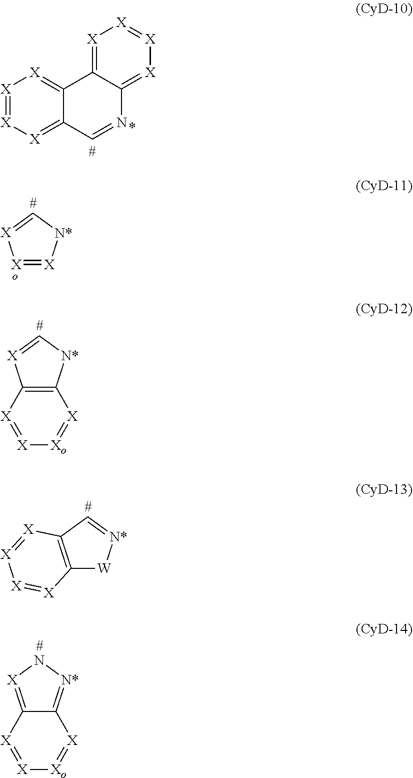

where the CyD group binds in each case to the position in CyC indicated by # and coordinates to the metal at the position indicated by *, and the symbols used are as follows:
X is the same or different at each instance and is CR or N, with the proviso that at most two symbols X per ring are N;
W is the same or different at each instance and is NR, O or S;
R has the definition given above, especially formula (I) or (Ia);
   where X, W and R have the definitions given above and where the ligands via the CyD group may optionally be bonded by a bridge, where the bond to the bridge may preferably be via the position marked “o”, where the position marked “o” represents a carbon atom if it constitutes a bridgehead site. When the CyD group is bonded to a bridge, the bond is preferably via the position marked “o” in the formulae depicted above, and so the symbol X marked “o” in that case is preferably C. The above-depicted structures which do not contain any symbol X marked “o” are preferably not bonded directly to a bridge, since such a bond to the bridge is not advantageous for steric reasons.

In this case, the (CyD-1) to (CyD-4), (CyD-7) to (CyD-10), (CyD-13) and (CyD-14) groups coordinate to the metal via an uncharged nitrogen atom, the (CyD-5) and (CyD-6) groups via a carbene carbon atom and the (CyD-11) and (CyD-12) groups via an anionic nitrogen atom.

Preferably, a total of not more than two symbols X in CyD are N, more preferably not more than one symbol X in CyD is N, and especially preferably all symbols X are CR, with the proviso that, when CyD is bonded to a bridge, one symbol X is C and the bridge is bonded to this carbon atom.

Particularly preferred CyD groups are the groups of the following formulae (CyD-1a) to (CyD-14b):

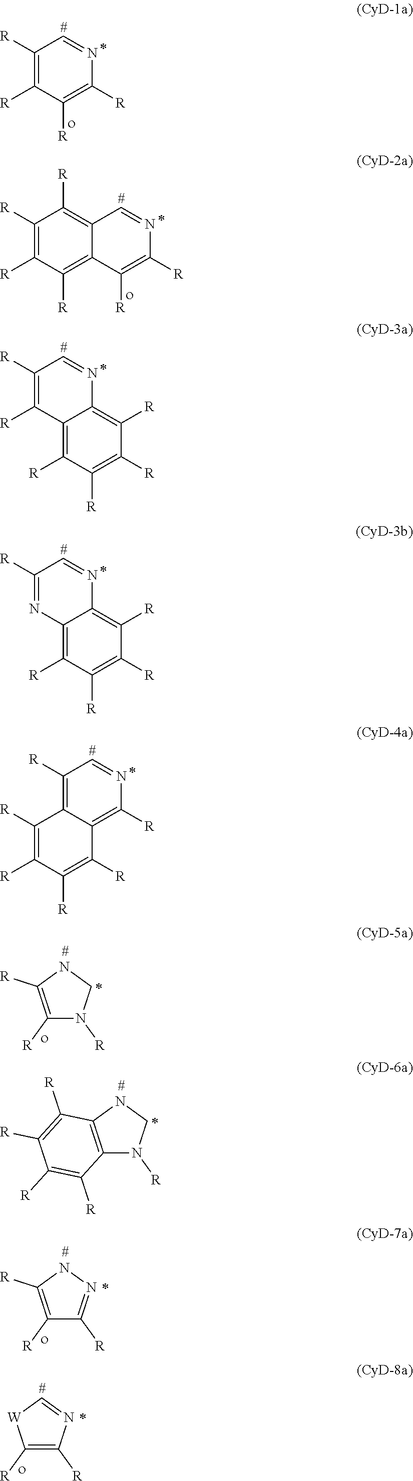

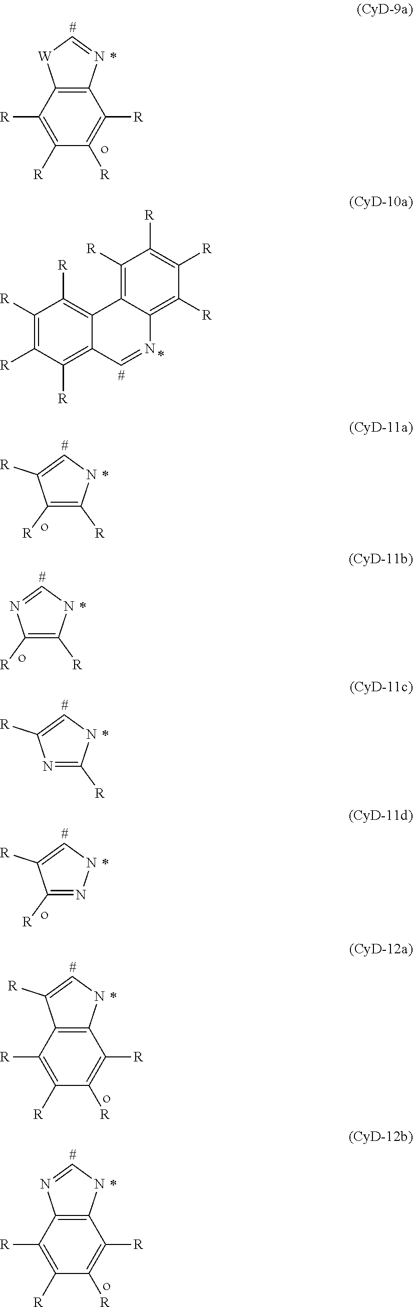

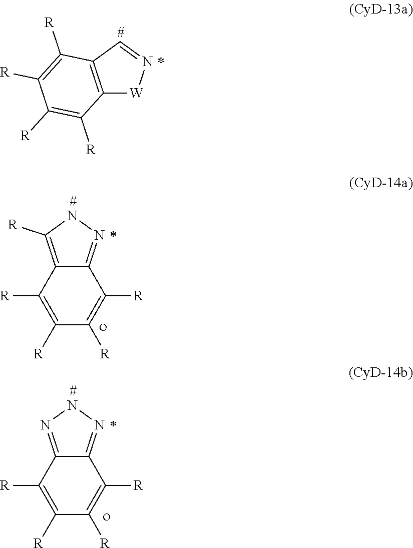

where the symbols used have the definitions given above and, when a bridge is bonded to CyD, one R radical is absent and the bridge is bonded to the corresponding carbon atom. When the CyD group is bonded to a bridge, the bond is preferably via the position marked “o” in the formulae depicted above, and so the R radical in this position in that case is preferably absent. The above-depicted structures which do not contain any carbon atom marked “o” are preferably not bonded directly to a bridge.

The position of the bond by which the (sub)ligands may be bonded to one another via a bridge may be different in the case of different metals, where the preference shown in the structures of the formulae (CyD-1) to (CyD-14) and (CyD-1a) to (CyD-14b) is applicable to iridium, for example. With regard to the position for platinum and similar metals, the examples give valuable pointers, where the bridge is preferably via a position adjacent to the coordination or binding site to the metal atom.

Preferred groups among the (CyD-1) to (CyD-10) groups are the (CyD-1), (CyD-2), (CyD-3), (CyD-4), (CyD-5) and (CyD-6) groups, especially (CyD-1), (CyD-2) and (CyD-3), and particular preference is given to the (CyD-1a), (CyD-2a), (CyD-3a), (CyD-4a), (CyD-5a) and (CyD-6a) groups, especially (CyD-1a), (CyD-2a) and (CyD-3a).

In a preferred embodiment of the present invention, CyC is an aryl or heteroaryl group having 6 to 13 aromatic ring atoms, and at the same time CyD is a heteroaryl group having 5 to 13 aromatic ring atoms. More preferably, CyC is an aryl or heteroaryl group having 6 to 10 aromatic ring atoms, and at the same time CyD is a heteroaryl group having 5 to 10 aromatic ring atoms. Most preferably, CyC is an aryl or heteroaryl group having 6 aromatic ring atoms, and CyD is a heteroaryl group having 6 to 10 aromatic ring atoms. At the same time, CyC and CyD may be substituted by one or more R radicals.

It may further be the case that CyD comprises a substructure of the formula (2), (2a), (2-1) and/or (2a-1) or the preferred embodiment of this substructure or is formed by suitable substitution by R radicals, where the X groups in formula (2) or (2a) in this case are CR¹. More preferably, one R radical in the above-detailed embodiments of the CyD group represents a substructure of the formula (2) or (2a), such that the bonding site shown by a dotted bond in formula (2) is bonded directly to the aromatic or heteroaromatic ring system shown in the CyD group. In the case of the configurations shown in formula (2a) or the preferred embodiments thereof, the CyD group is bonded directly by two binding sites to the aromatic or heteroaromatic ring system shown in the CyD group, such that the double bond shown in the structures of formula (2a) which is marked by the dotted bond constitutes part of the aromatic or heteroaromatic ring system.

The abovementioned preferred groups (CyC-1) to (CyC-20) and (CyD-1) to (CyD-14) may be combined with one another as desired in the ligands of the formulae (L-1) and (L-2). In this case, at least one of the CyC or CyD groups may have a suitable attachment site to a bridge, where suitable attachment sites in the abovementioned formulae are identified by “o”. It is especially preferable when the CyC and CyD groups mentioned as particularly preferred above, i.e. the groups of the formulae (CyC-1a) to (CyC-20a) and the groups of the formulae (CyD1-a) to (CyD-14b), are combined with one another. Combinations in which neither CyC nor CyD has such a suitable attachment site for a bridge are therefore not preferred.

It is very particularly preferable when one of the (CyC-1), (CyC-3), (CyC-8), (CyC-10), (CyC-12), (CyC-13) and (CyC-16) groups and especially the (CyC-1a), (CyC-3a), (CyC-8a), (CyC-10a), (CyC-12a), (CyC-13a) and (CyC-16a) groups is combined with one of the (CyD-1), (CyD-2) and (CyD-3) groups and especially with one of the (CyD-1a), (CyD-2a) and (CyD-3a) groups.

The abovementioned preferred (CyD-1) to (CyD-14) groups may be combined as desired with groups of the formulae (2), (2a), (2-1) and (2a-1) in the ligands of the formulae (L-4) and (L-5). In this case, at least one of the CyD groups or a substructure of the formulae (2), (2a), (2-1) and (2a-1) may have a suitable attachment site to a bridge, where suitable attachment sites in the abovementioned formulae are identified by “o”. It is especially preferable when the CyD groups mentioned as particularly preferred above, i.e. the groups of the formulae (CyD1-a) to (CyD-14b), are combined with a substructure of the formulae (2), (2a), (2-1) and (2a-1).

Preferred ligands (L-1) are the structures of the following formulae (L-1-1) and (L-1-2), and preferred ligands (L-2) are the structures of the following formulae (L-2-1) to (L-2-3):

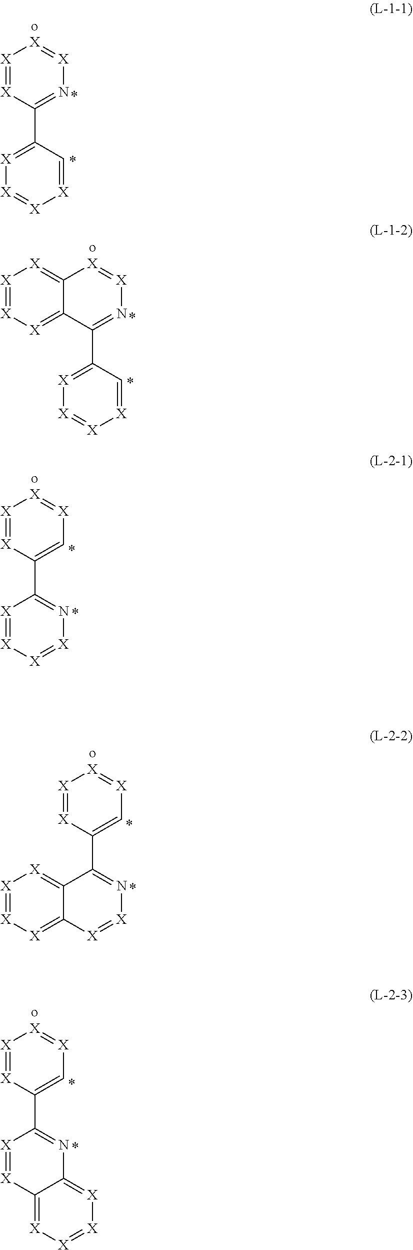

where the symbols used have the definitions given above and the ligands may optionally be bonded by a bridge, where the bond to the bridge may preferably be via the position marked “o”, where the position marked “o” represents a carbon atom if it constitutes a bridgehead site.

Particularly preferred ligands (L-1) are the structures of the following formulae (L-1-1a) and (L-1-2b), and particularly preferred ligands (L-2) are the structures of the following formulae (L-2-1a) to (L-2-3a):

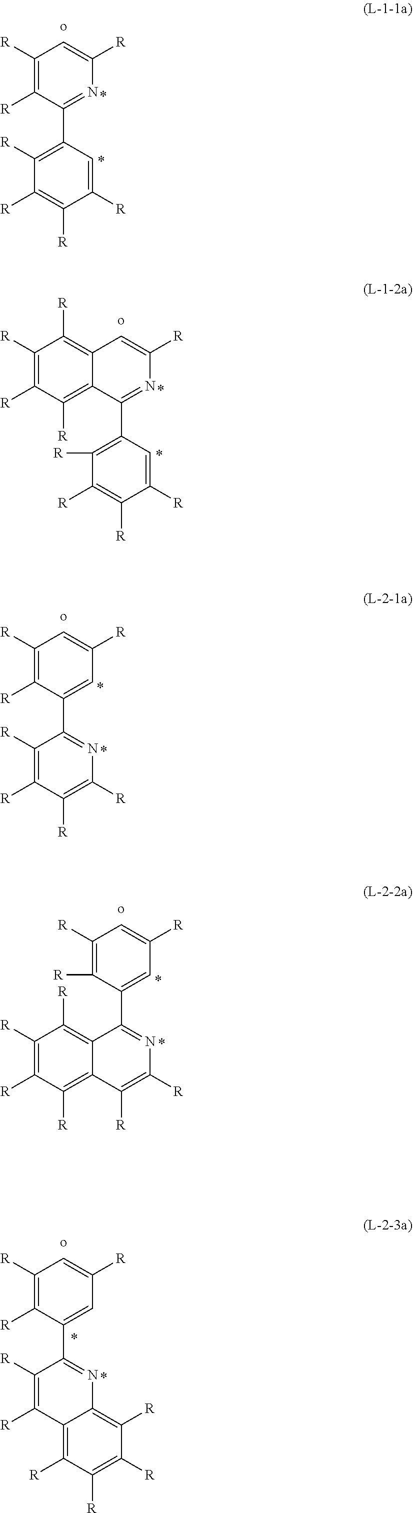

where the symbols used have the definitions given above and the ligands may optionally be bonded by a bridge, where the bond to the bridge may preferably be via the position marked “o”, where the position marked “o” represents a carbon atom if it constitutes a bridgehead site. If the ligands are unbridged, the position marked “o” may also be substituted by an R radical.

It is likewise possible for the abovementioned preferred CyD groups in the ligands of the formula (L-3) to be combined with one another as desired, it being preferable to combine an uncharged CyD group, i.e. a (CyD-1) to (CyD-10), (CyD-13) or (CyD-14) group, with an anionic CyD group, i.e. a (CyD-11) or (CyD-12) group, where the ligands may optionally be bonded by a bridge, where the bond to the bridge may preferably be via the position marked “o”, where suitable attachment sites in the abovementioned formula are identified by “o”.

The position of the bond by which the (sub)ligands may be bonded to one another via a bridge may be different in the case of different metals, where the preference shown in the structures of the formulae (L-1-1) to (L-2-3) and (L-1-1a) to (L-2-3a) or the preferred embodiments of these structures that are described hereinafter is applicable to iridium, for example. With regard to the position for platinum and similar metals, the examples give valuable pointers, where the bridge is preferably via a position adjacent to the coordination or binding site to the metal atom.

When two R radicals, one of them bonded to CyC and the other to CyD in the formulae (L-1) and (L-2) or one of them bonded to one CyD group and the other to the other CyD group in formula (L-3) or one of them bonded to one CyD group and the other to a substructure of the formula (2) or (2-1), form an aromatic ring system with one another, this may result in bridged ligands and, for example, also in ligands which constitute a single larger heteroaryl group overall, for example benzo[h]quinoline, etc. The ring formation between the substituents on CyC and CyD in the formulae (L-1) and (L-2) or between the substituents on the two CyD groups in formula (L-3) or between the substituents on CyD and a substructure of the formula (2) or (2-1) in formula (L-4) or (L-5) is preferably via a group according to one of the following formulae (RB-1) to (RB-10):

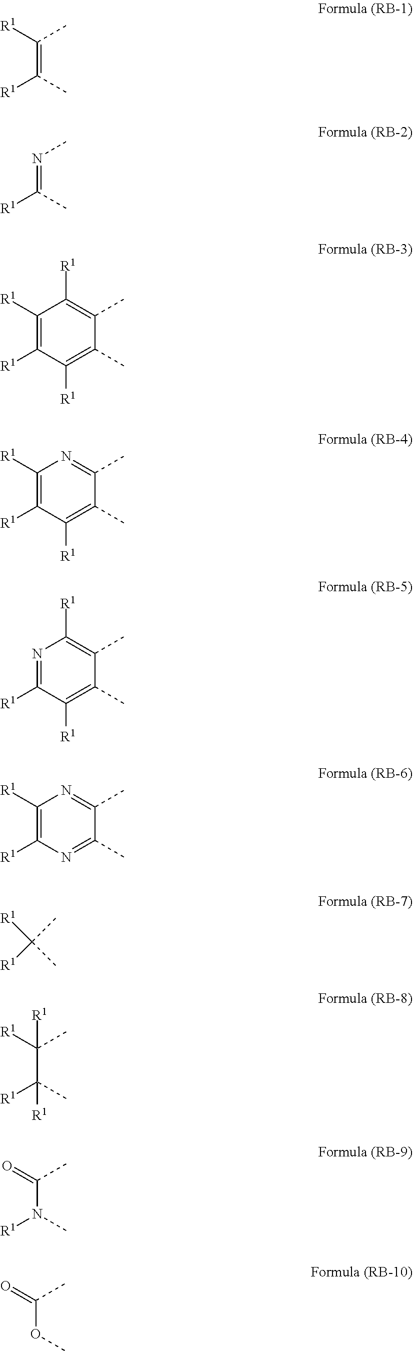

where R¹ has the definitions given above and the dotted bonds signify the bonds to CyC or CyD. At the same time, the unsymmetric groups among those mentioned above may be incorporated in each of the two options; for example, in the group of the formula (RB-10), the oxygen atom may bind to the CyC group and the carbonyl group to the CyD group, or the oxygen atom may bind to the CyD group and the carbonyl group to the CyC group.

At the same time, the group of the formula (RB-7) is preferred particularly when this results in ring formation to give a six-membered ring, as shown below, for example, by the formulae (L-23) and (L-24).

Preferred ligands which arise through ring formation between two R radicals on the different cycles are the structures of the formulae (L-5) to (L-32) shown below:

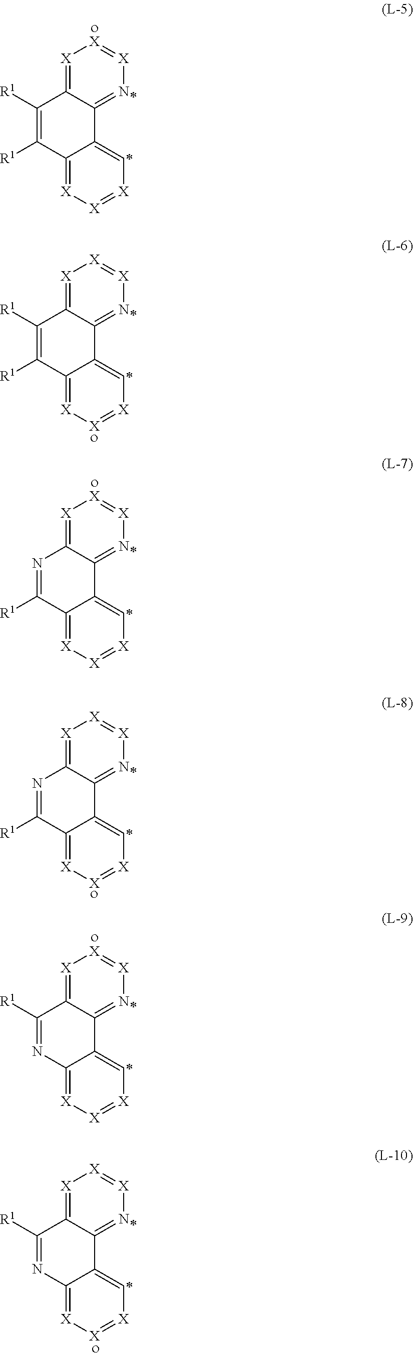

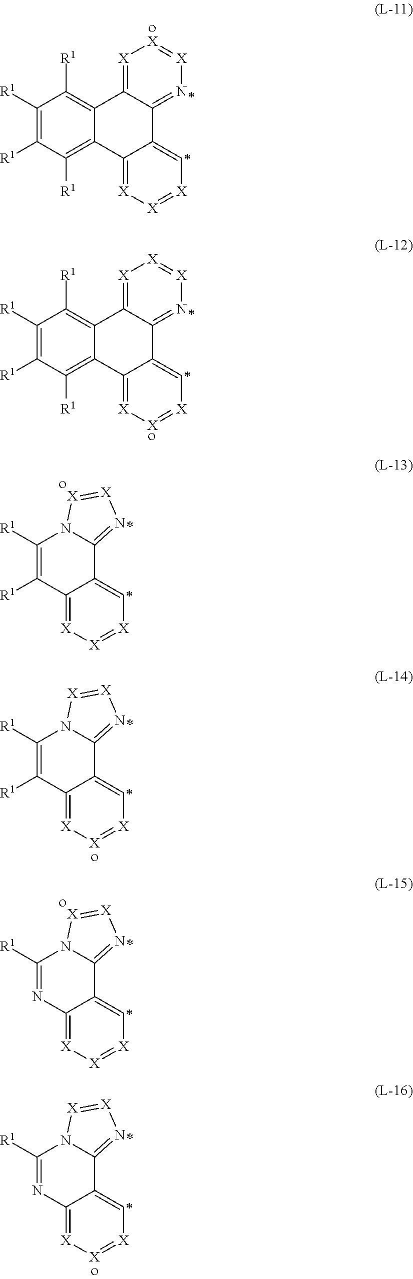

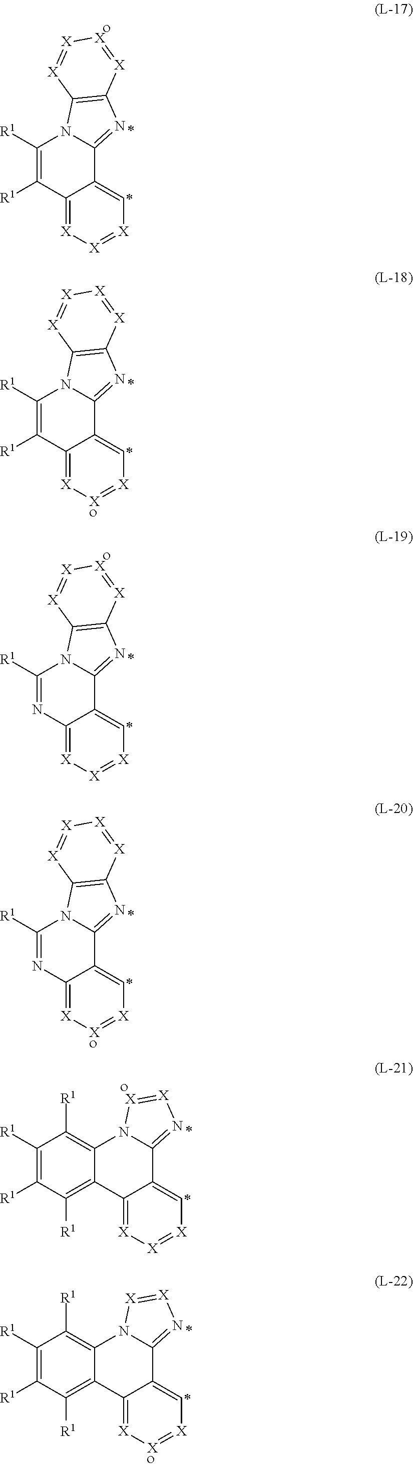

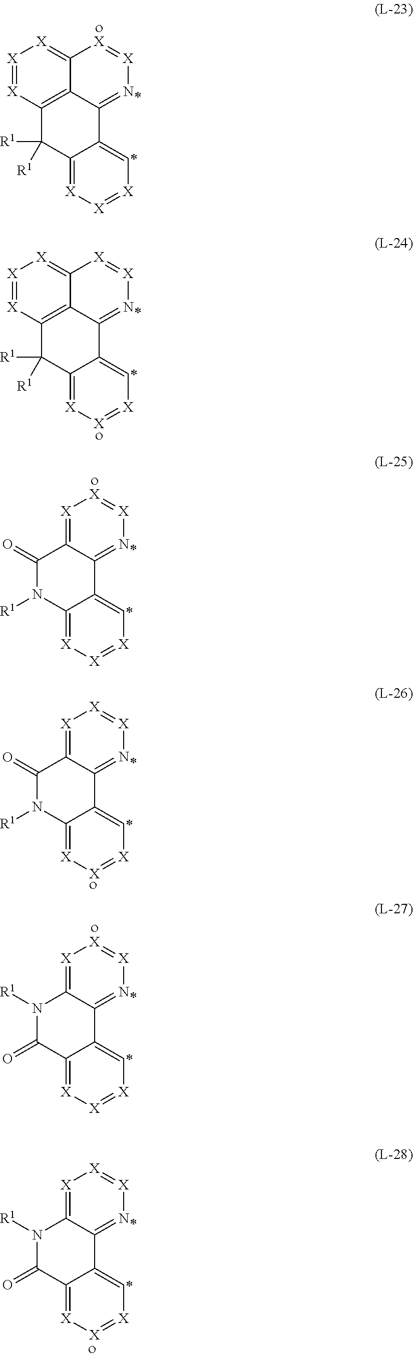

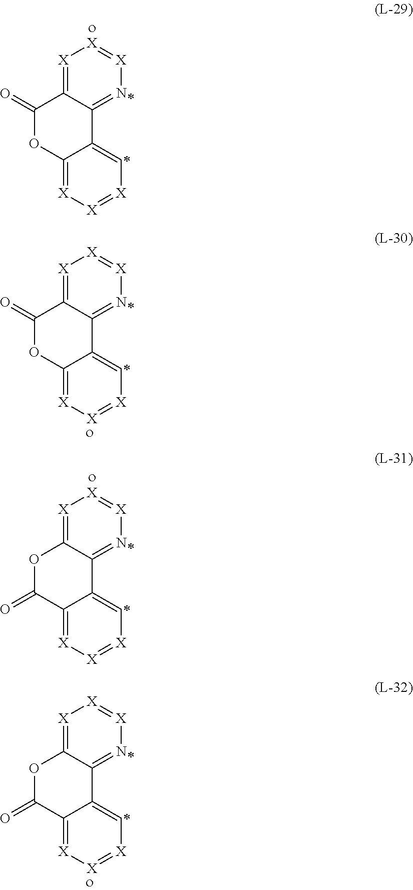

where the symbols used have the definitions given above, where the ligands may optionally be bonded by a bridge, where the bond to the bridge may preferably be via the position marked “o”, where the position marked “o” represents a carbon atom if it constitutes a bridgehead site.

In a preferred embodiment of the ligands of the formulae (L-5) to (L-32), overall, one symbol X is N and the other symbols X are CR, or all symbols X are CR, with the proviso that, when these ligands are bonded via a bridge, one symbol X is C and the bridge is bonded to this carbon atom.

In a further embodiment of the invention, it is preferable if, in the groups (CyC-1) to (CyC-20) or (CyD-1) to (CyD-14) or in the ligands (L-5) to (L-3), one of the atoms X is N when an R group bonded as a substituent adjacent to this nitrogen atom is not hydrogen or deuterium. This applies analogously to the preferred structures (CyC-1a) to (CyC-20a) or (CyD-1a) to (CyD-14b) in which a substituent bonded adjacent to a non-coordinating nitrogen atom is preferably an R group which is not hydrogen or deuterium. This substituent R is preferably a group selected from CF₃, OCF₃, alkyl or alkoxy groups having 1 to 10 carbon atoms, especially branched or cyclic alkyl or alkoxy groups having 3 to 10 carbon atoms, a dialkylamino group having 2 to 10 carbon atoms, aromatic or heteroaromatic ring systems or aralkyl or heteroaralkyl groups. These groups are sterically demanding groups. Further preferably, this R radical may also form a cycle with an adjacent R radical.

A further suitable bidentate ligand is the ligand of the following formula (L-33) or (L-34)

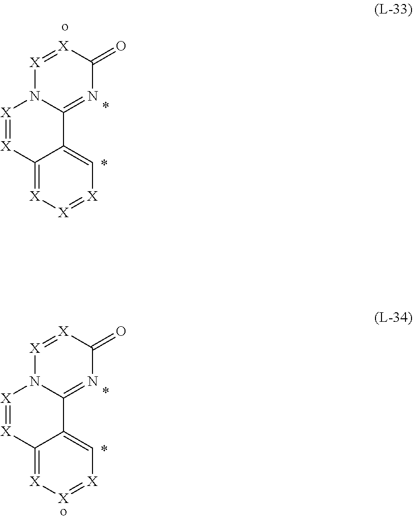

where R has the definitions given above, * represents the position of coordination to the metal, where the ligands may optionally be bonded by a bridge, where the bond to the bridge may preferably be via the position marked “0”, and the further symbols used are as follows:
X is the same or different at each instance and is CR or N, with the proviso that not more than one X symbol per cycle is N, where X is C if the ligand at this position is bonded to a bridge.

When two R radicals bonded to adjacent carbon atoms in the ligands (L-33) and (L-34) form an aromatic cycle with one another, this cycle together with the two adjacent carbon atoms is preferably a structure of the following formula (BR-11):

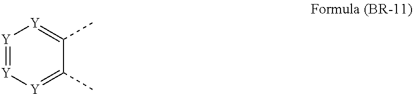

where the dotted bonds symbolize the linkage of this group within the ligand and Y is the same or different at each instance and is CR¹ or N and preferably not more than one symbol Y is N.

In a preferred embodiment of the ligand (L-33) or (L-34), not more than one group of the formula (BR-11) is present. The ligands are thus preferably ligands of the following formulae (L-35) to (L-40):

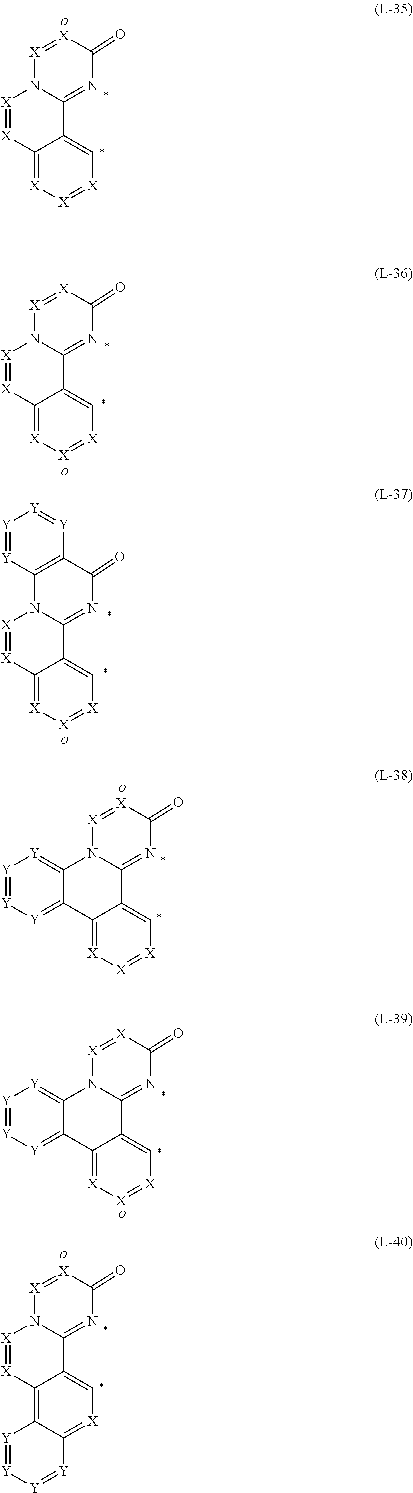

where R has the definition given above, * represents the position of coordination to the metal, where the ligands may optionally be bonded by a bridge, where the bond to the bridge may preferably be via the position marked “o”, and the further symbols used are as follows:
X is the same or different at each instance and is CR or N, with the proviso that not more than one X symbol per cycle is N, where X is C if the ligand at this position is bonded to a bridge;
Y is the same or different at each instance and is CR¹ or N and preferably not more than one symbol Y is N, where R¹ has the definition given above.

In a preferred embodiment of the invention, in the ligand of the formulae (L-33) to (L-40), a total of 0, 1 or 2 of the symbols X and, if present, Y are N. More preferably, a total of 0 or 1 of the symbols X and, if present, Y are N.

In a preferred embodiment of the invention, the X group in the ortho position to the coordination to the metal is CR. In this radical, R bonded in the ortho position to the coordination to the metal is preferably selected from the group consisting of H, D, F and methyl.

In a further embodiment of the invention, it is preferable, if one of the atoms X or, if present, Y is N, when a substituent bonded adjacent to this nitrogen atom is an R group which is not hydrogen or deuterium. This substituent R is preferably a group selected from CF₃, OCF₃, alkyl or alkoxy groups having 1 to 10 carbon atoms, especially branched or cyclic alkyl or alkoxy groups having 3 to 10 carbon atoms, a dialkylamino group having 2 to 10 carbon atoms, aromatic or heteroaromatic ring systems or aralkyl or heteroaralkyl groups. These groups are sterically demanding groups. Further preferably, this R radical may also form a cycle with an adjacent R radical.

Further suitable bidentate ligands are the structures of the following formulae (L-41) to (L-45), where preferably not more than one of the three bidentate ligands is one of these structures,

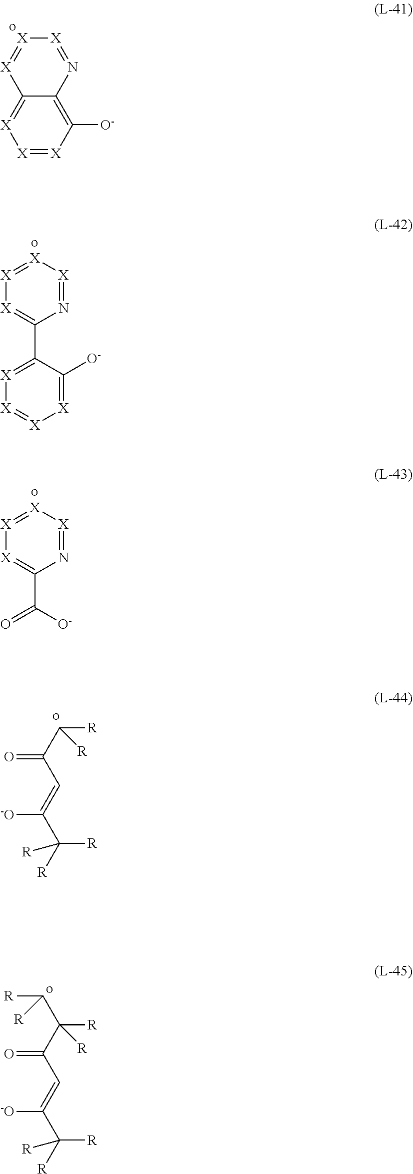

where the ligands (L-41) to (L-43) each coordinate to the metal via the nitrogen atom shown explicitly and the negatively charged oxygen atom and the ligands (L-44) and (L-45) via the two oxygen atoms, R and X have the definitions given above, especially for formula (I) or (Ia), where the ligands may optionally be bonded by a bridge, where the bond to the bridge may preferably be via the position marked by “o”, where X is C if the ligand is bonded to a bridge at this position, or, in formula (L-44) or (L-45), the carbon atom may have a substituent R if the ligand is not bonded to a bridge at this position.

The above-recited preferred embodiments of X are also preferred for the ligands of the formulae (L-41) to (L-43).

Preferred ligands of the formulae (L-41) to (L-43) are therefore the ligands of the following formulae (L-41a) to (L-43a):

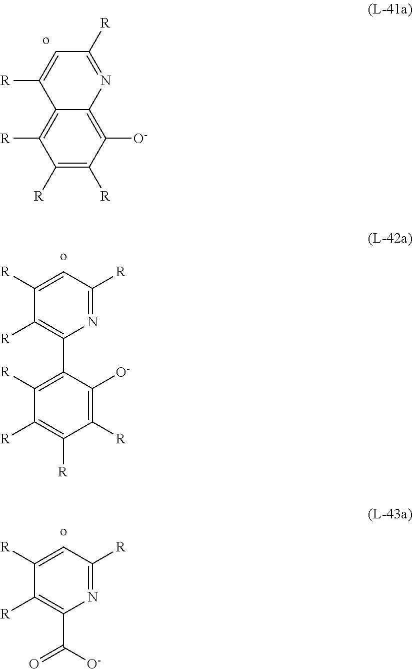

where the symbols used have the definitions given above and one R group is absent, where the ligands may optionally be bonded by a bridge, where the bond to the bridge may preferably be via the position marked “o” or, in formula (L-41a), (L-42a) or (L-43a), the carbon atom may have a substituent R if the ligand at this position is not bonded to a bridge.

More preferably, in these formulae, R is hydrogen, where the ligands may optionally be bonded by a bridge, where the bond to the bridge may preferably be via the position marked “o”, and so the structures are those of the following formulae (L-41 b) to (L-43b):

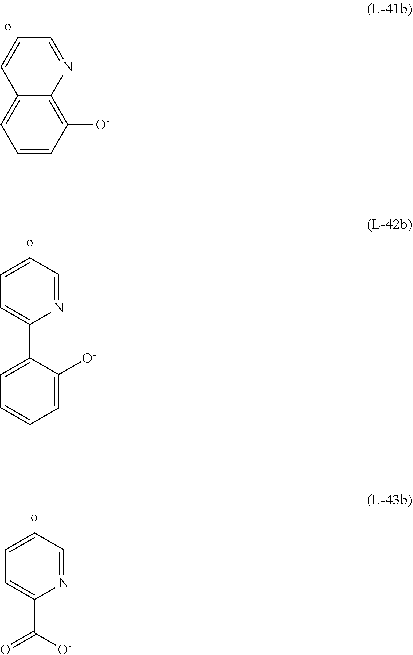 [SMILES: [O-]c1cccc2cccnc12 ; [O-]c1ccccc1-c1ccccn1 ; O=C([O-])c1ccccn1]

where the symbols used have the definitions given above.

Further preferred bidentate ligands are the structures of the following formula (L-46):

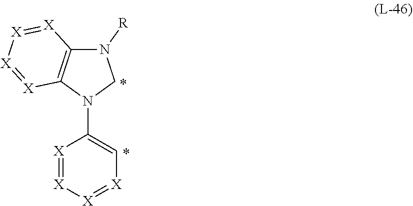

where X and R have the definitions given above, * represents the position of coordination to the metal, where the ligands may optionally be bonded by a bridge. In this case, the R group bonded to N is preferably not H, but is an alkyl, heteroalkyl, aryl or heteroaryl group as detailed above for R. Preferably, not more than two X per ring are N; more preferably, all X are CR, where the ligands may be bonded via an R radical.

Preferred ligands of the formula (L-46) are therefore the ligands of the following formulae (L-46a):

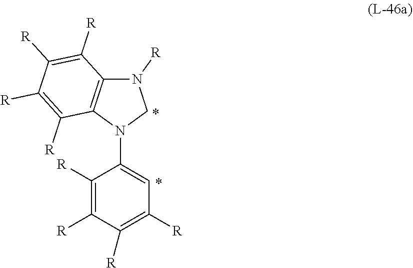

where the symbols used have the definitions given above.

The ligands of the formulae (L-1) to (L-46) or preferred configurations thereof preferably comprise at least one substructure of the formula (2) and/or (2a), where this substructure is preferably formed by suitable substitution by R or R¹ radicals, in which case the X groups in formula (2) or (2a) are CR¹. More preferably, one R radical in the above-detailed embodiments of the ligands of the formulae (L-1) to (L-46) represents a substructure of the formula (2) or (2a), such that the bonding site shown by a dotted bond in formula (2) is bonded directly to the aromatic or heteroaromatic ring system. In the case of the configurations shown in formula (2a) or the preferred embodiments thereof, ligands of the formulae (L-1) to (L-46) are bonded directly by two binding sites to the aromatic or heteroaromatic ring system, such that the double bond shown in the structures of formula (2a) which is marked by the dotted bond constitutes part of the aromatic or heteroaromatic ring system.

In a preferred embodiment, the metal complexes conform to the general formula

M(L)ₙ(L′)ₘ Formula (Ia)

where the symbol M and the ligands L and/or L′ have the definitions given above, especially for formula (1), and at least some of the ligands are joined via a bridge, so as to form a tridentate, tetradentate, pentadentate or hexadentate ligand system, and preferably to form a metal complex containing iridium and a hexadentate tripodal ligand, with the proviso that the metal complex contains at least one substructure of the formula (2) or (2a)

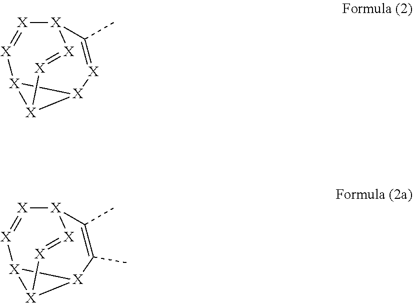

where the symbols have the definitions given above, especially for formula (1) and (2), where the preferences mentioned above are applicable thereto as well. In this case, the ligands L and L¹ may be regarded as three bidentate sub-ligands that coordinate to a metal. Preferably, the bridge may be an aryl or heteroaryl group which has 5 to 36 aromatic ring atoms and may be substituted by one or more R radicals. A metal complex of the general formula (Ia) may preferably contain structures of the above-detailed formulae (2-1) and/or (2a-1).

In the case of Pt, in a structure of formula (Ia), preferably a tetradentate ligand system is formed.

There follows a description of preferred iridium and platinum complexes. The same applies to the further metal atoms listed for M in formula (1). As described above, these are organometallic complexes. An organometallic complex in the context of the present invention is a complex having at least one metal-carbon bond to the ligand.

In a preferred embodiment of the invention, the iridium or platinum complex is uncharged, i.e. electrically neutral. Therefore, the iridium complex preferably contains either three bidentate monoanionic ligands or one tripodal hexadentate trianionic ligand, and the platinum complex contains either two bidentate monoanionic ligands or one tetradentate dianionic ligand.

The bond of the ligand to the iridium or the platinum may either be a coordinate bond or a covalent bond, or the covalent fraction of the bond may vary according to the ligand. When it is said in the present application that the ligand or ligand coordinates or binds to iridium or the platinum, this refers in the context of the present application to any kind of bond of the ligand to the iridium or the platinum, irrespective of the covalent component of the bond.

In a further preferred embodiment of the invention, M is platinum, and so an organometallic platinum complex comprises a substructure of the formula (2) or (2a). When M is platinum, this complex preferably comprises two bidentate ligands that may be joined to one another. In this case, these ligands are the same or different and are preferably selected from the above-depicted ligands of the formulae (L-1), (L-2) and (L-3), where the abovementioned preferences are applicable thereto as well.

When M is platinum and the platinum complex comprises a tetradentate ligand, this can be shown schematically by the following formula (Lig′):

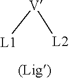

where V′ is selected from CR₂, NR, O, S and BR, preferably CR₂ and NR, where R has the definitions given above, and L1 and L2 are the same or different at each instance and are each bidentate ligands, preferably monoanionic bidentate ligands. Since the ligand has two bidentate ligands, the overall result is a tetradentate ligand, i.e. a ligand which coordinates or binds to the platinum via four coordination sites.

The platinum complex formed with this ligand of the formula (Lig′) can thus be represented schematically by the following formula:

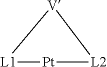

where the symbols used have the definitions given above.

The position of the bond by which the (sub)ligands may be bonded to one another via a bridge may be different in the case of different metals, where the preference shown is applicable to iridium, for example. With regard to the position for platinum and similar metals, the examples give valuable pointers, where the bridge is preferably via a position adjacent to the coordination or binding site to the metal atom.

In a preferred embodiment of the invention, M is iridium. It may be the case here that the metal is Ir(III) and the metal complex has three bidentate ligands, where two of the bidentate ligands coordinate to the iridium via one carbon atom and one nitrogen atom in each case or via two carbon atoms, and the third of the bidentate ligands coordinates to the iridium via one carbon atom and one nitrogen atom or via two carbon atoms or via two nitrogen atoms, where preferably the third of the bidentate ligands coordinates to the iridium via one carbon atom and one nitrogen atom or via two carbon atoms.

Particular preference is given to an iridium complex having a tripodal hexadentate ligand as described hereinafter. This tripodal hexadentate ligand contains three bidentate sub-ligands which may be the same or different and coordinate to an iridium atom, where the three bidentate sub-ligands are joined via a bridge of the following formula (3) or formula (4):

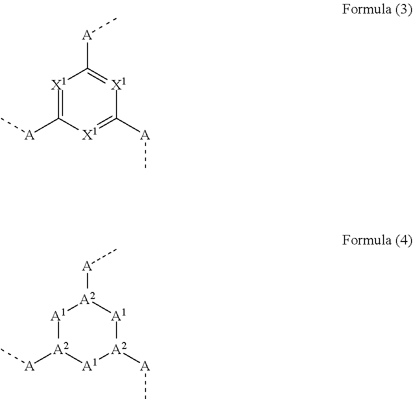

where the dotted bond constitutes the bond of the bidentate ligands to this structure, R, R¹ and R² have the definitions given above and in addition:
X¹ is the same or different at each instance and is CR or N;
A¹ is the same or different at each instance and is C(R)₂ or O;
A² is the same or different at each instance and is CR, P(═O), B or SiR, with the proviso that, when A²=P(═O), B or SiR, the symbol A¹ is O and the symbol A bonded to this A² is not —C(═O)—NR′— or —C(═O)—O—;
A is the same or different at each instance and is —CR═CR—, —C(═O)—NR′—, —C(═O)—O— or a group of the following formula (5):

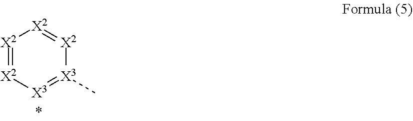

where the dotted bond represents the position of the bond of the bidentate ligands to this structure and * represents the position of the linkage of the unit of the formula (5) to the central cyclic group;
X² is the same or different at each instance and is CR or N, or two adjacent X² groups together are NR, O or S, thus forming a five-membered ring, and the remaining X² are the same or different at each instance and are CR or N; or two adjacent X² groups together are CR or N when one of the X³ groups in the cycle is N, thus forming a five-membered ring; with the proviso that not more than two adjacent X² groups are N;
X³ is C at each instance or one X³ group is N and the other X³ group in the same cycle is C; with the proviso that two adjacent X² groups together are CR or N when one of the X³ groups in the cycle is N;
R′ is the same or different at each instance and is H, D, a straight-chain alkyl group having 1 to 20 carbon atoms or a branched or cyclic alkyl group having 3 to 20 carbon atoms, where the alkyl group in each case may be substituted by one or more R¹ radicals and where one or more nonadjacent CH₂ groups may be replaced by Si(R¹)₂, or an aromatic or heteroaromatic ring system which has 5 to 40 aromatic ring atoms and may be substituted in each case by one or more R¹ radicals;
   at the same time, the three bidentate sub-ligands, apart from by the bridge of the formula (3) or (4), may also be closed by a further bridge to form a cryptate.

When two R or R¹ or R² radicals together form a ring system, it may be mono- or polycyclic, and aliphatic, heteroaliphatic, aromatic or heteroaromatic. In this case, the radicals which together form a ring system may be adjacent, meaning that these radicals are bonded to the same carbon atom or to carbon atoms directly bonded to one another, or they may be further removed from one another.

The structure of the hexadentate tripodal ligands can be shown in schematic form by the following formula (Lig):

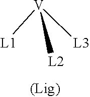

where V represents the bridge of formula (3) or (4) and L1, L2 and L3 are the same or different at each instance and are each bidentate sub-ligands, preferably monoanionic bidentate sub-ligands. “Bidentate” means that the particular ligand in the complex M coordinates or binds to the iridium via two coordination sites. “Tripodal” means that the ligand has three sub-ligands bonded to the bridge V or the bridge of the formula (3) or (4). Since the ligand has three bidentate sub-ligands, the overall result is a hexadentate ligand, i.e. a ligand which coordinates or binds to the iridium via six coordination sites. The expression “bidentate sub-ligand” in the context of this application means that this unit would be a bidentate ligand if the bridge of the formula (3) or (4) were not present. However, as a result of the formal abstraction of a hydrogen atom in this bidentate ligand and the attachment to the bridge of the formula (3) or (4), it is not a separate ligand but a portion of the hexadentate ligand which thus arises, and so the term “sub-ligand” is used therefor.

The iridium complex formed with this ligand of the formula (Lig) can thus be represented schematically by the following formula:

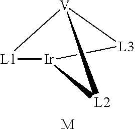

where V represents the bridge of formula (3) or (4) and L1, L2 and L3 are the same or different at each instance and are each bidentate sub-ligands.

Preferred embodiments of the bridge of the formula (3) or (4) are detailed hereinafter. Suitable embodiments of the group of the formula (3) are the structures of the following formulae (6) to (9), and suitable embodiments of the group of the formula (4) are the structures of the following formulae (10) to (14):

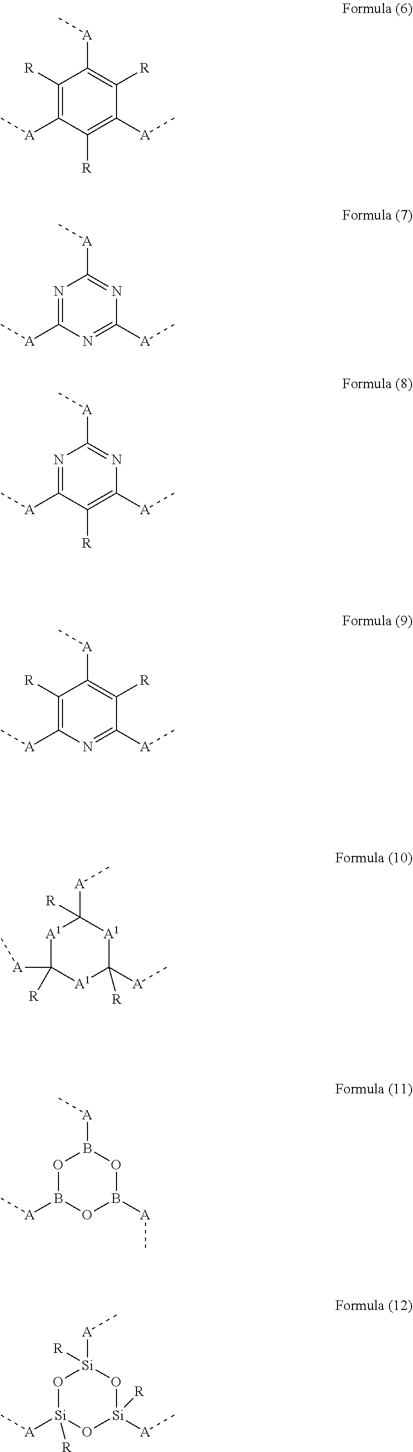

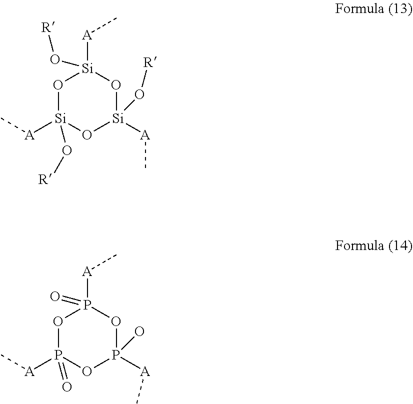

where the symbols have the definitions given above.

The following is applicable in respect of preferred R radicals on the trivalent central benzene ring of the formula (6), on the pyrimidine ring of the formula (8), on the pyridine ring of the formula (9) and on the central (hetero)aliphatic ring of the formulae (10) to (14):
R is the same or different at each instance and is H, D, F, CN, a straight-chain alkyl or alkoxy group having 1 to 10 carbon atoms or an alkenyl group having 2 to 10 carbon atoms or a branched or cyclic alkyl or alkoxy group having 3 to 10 carbon atoms, each of which may be substituted by one or more R¹ radicals, or an aromatic or heteroaromatic ring system which has 5 to 24 aromatic ring atoms and may be substituted in each case by one or more R¹ radicals;
R¹ is the same or different at each instance and is H, D, F, CN, a straight-chain alkyl or alkoxy group having 1 to 10 carbon atoms or an alkenyl group having 2 to 10 carbon atoms or a branched or cyclic alkyl or alkoxy group having 3 to 10 carbon atoms, each of which may be substituted by one or more R² radicals, or an aromatic or heteroaromatic ring system which has 5 to 24 aromatic ring atoms and may be substituted in each case by one or more R² radicals; at the same time, two or more adjacent R¹ radicals together may form a ring system;
R² is the same or different at each instance and is H, D, F or an aliphatic, aromatic and/or heteroaromatic organic radical having 1 to 20 carbon atoms, in which one or more hydrogen atoms may also be replaced by F.

The following is applicable in respect of particularly preferred R radicals on the trivalent central benzene ring of the formula (6), on the pyrimidine ring of the formula (8), on the pyridine ring of the formula (9) and on the central (hetero)aliphatic ring of the formulae (10) to (14):
R is the same or different at each instance and is H, D, F, CN, a straight-chain alkyl group having 1 to 4 carbon atoms or a branched or cyclic alkyl group having 3 to 6 carbon atoms, each of which may be substituted by one or more R¹ radicals, or an aromatic or heteroaromatic ring system which has 6 to 12 aromatic ring atoms and may be substituted in each case by one or more R¹ radicals;
R¹ is the same or different at each instance and is H, D, F, CN, a straight-chain alkyl group having 1 to 4 carbon atoms or a branched or cyclic alkyl group having 3 to 6 carbon atoms, each of which may be substituted by one or more R² radicals, or an aromatic or heteroaromatic ring system which has 6 to 12 aromatic ring atoms and may be substituted in each case by one or more R² radicals; at the same time, two or more adjacent R¹ radicals together may form a ring system;
R² is the same or different at each instance and is H, D, F or an aliphatic or aromatic hydrocarbyl radical having 1 to 12 carbon atoms.

In a preferred embodiment of the invention, all X¹ groups in the group of the formula (3) are CR, and so the central trivalent cycle of the formula (3) is a benzene. More preferably, all X¹ groups are CH. In a further preferred embodiment of the invention, all X¹ groups are a nitrogen atom, and so the central trivalent cycle of the formula (3) is a triazine. Preferred embodiments of the formula (3) are thus the structures of the formulae (6) and (7). More preferably, the structure of the formula (6) is a structure of the following formula (6′):

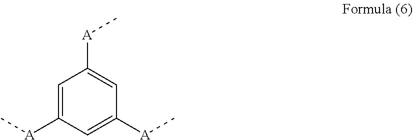

where the symbols have the definitions given above.

In a further preferred embodiment of the invention, all A² groups in the group of the formula (4) are CR. More preferably, all A² groups are CH. Preferred embodiments of the formula (4) are thus the structures of the formula (10). More preferably, the structure of the formula (10) is a structure of the following formula (10′) or (10″):

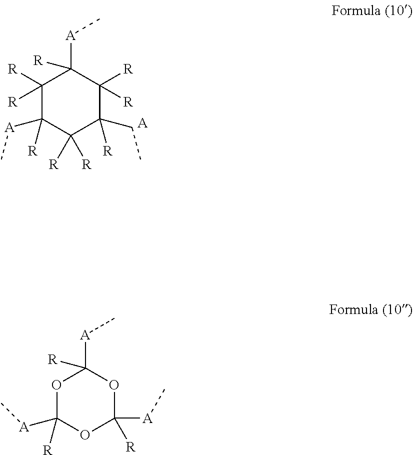

where the symbols have the definitions given above and R is preferably H.

Preferred embodiments of the group of the formula (5) are described hereinafter. The group of the formula (5) may represent a heteroaromatic five-membered ring or an aromatic or heteroaromatic six-membered ring. In a preferred embodiment of the invention, the group of the formula (5) contains not more than two heteroatoms in the aromatic or heteroaromatic unit, more preferably not more than one heteroatom. This does not mean that any substituents bonded to this group cannot also contain heteroatoms. In addition, this definition does not mean that formation of rings by substituents does not give rise to fused aromatic or heteroaromatic structures, for example naphthalene, benzimidazole, etc.

When both X³ groups in formula (5) are carbon atoms, preferred embodiments of the group of the formula (5) are the structures of the following formulae (15) to (31), and, when one X³ group is a carbon atom and the other X³ group in the same cycle is a nitrogen atom, preferred embodiments of the group of the formula (5) are the structures of the following formulae (32) to (39):

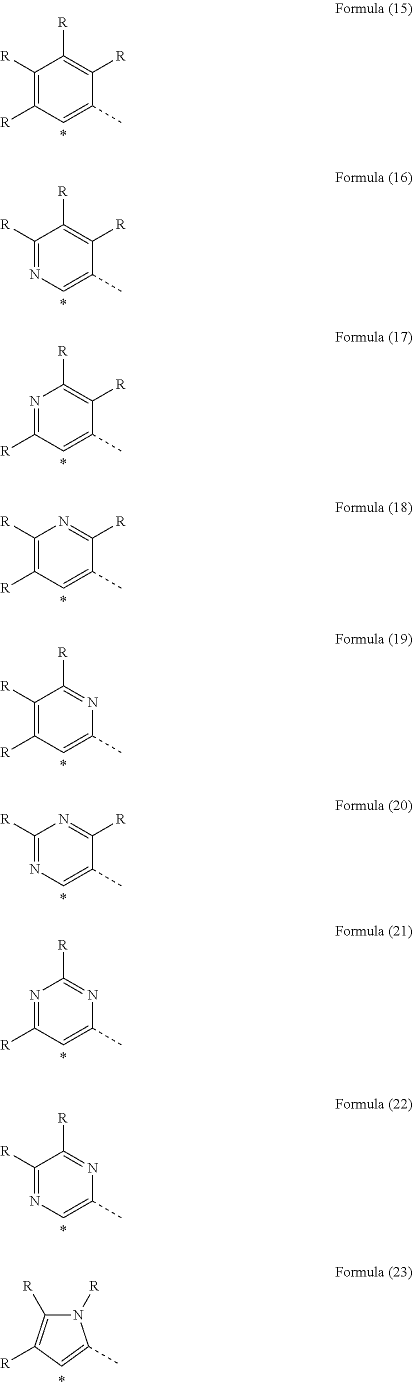

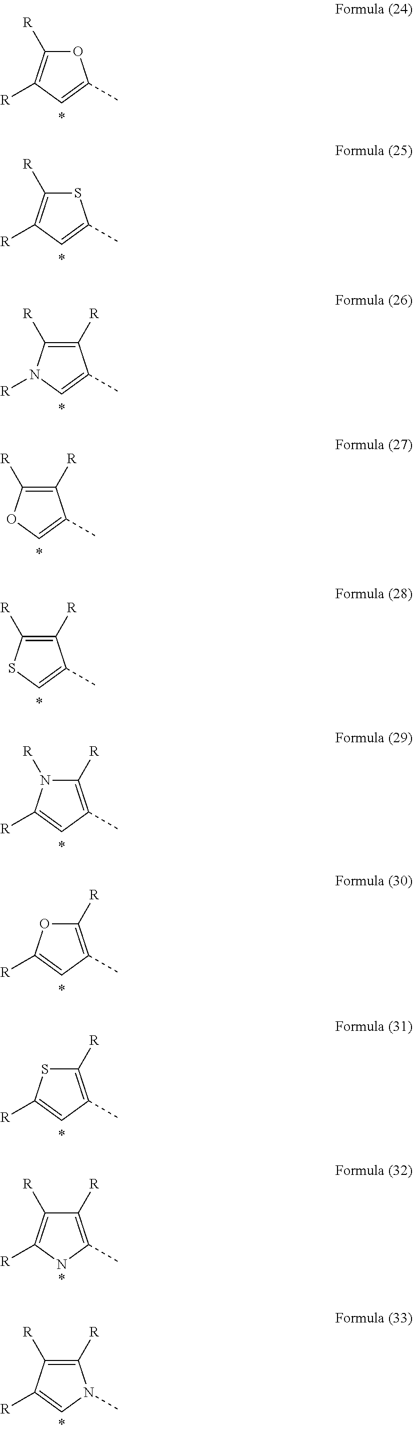

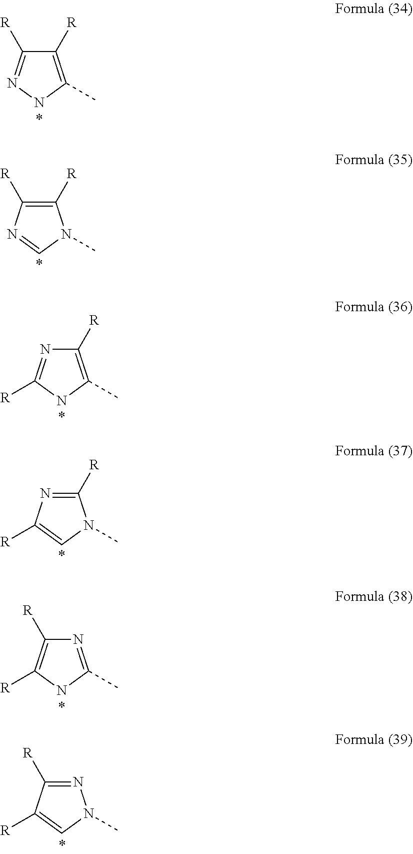

where the symbols have the definitions given above.

Particular preference is given to the six-membered aromatic rings and heteroaromatic rings of the formulae (15) to (19) depicted above. Very particular preference is given to ortho-phenylene, i.e. a group of the abovementioned formula (15).

At the same time, it is also possible for adjacent R substituents together to form a ring system, such that it is possible to form fused structures, including fused aryl and heteroaryl groups, for example naphthalene, quinoline, benzimidazole, carbazole, dibenzofuran or dibenzothiophene. Such ring formation is shown schematically below in groups of the abovementioned formula (15), which leads to groups of the following formulae (15a) to (15j):

where the symbols have the definitions given above.

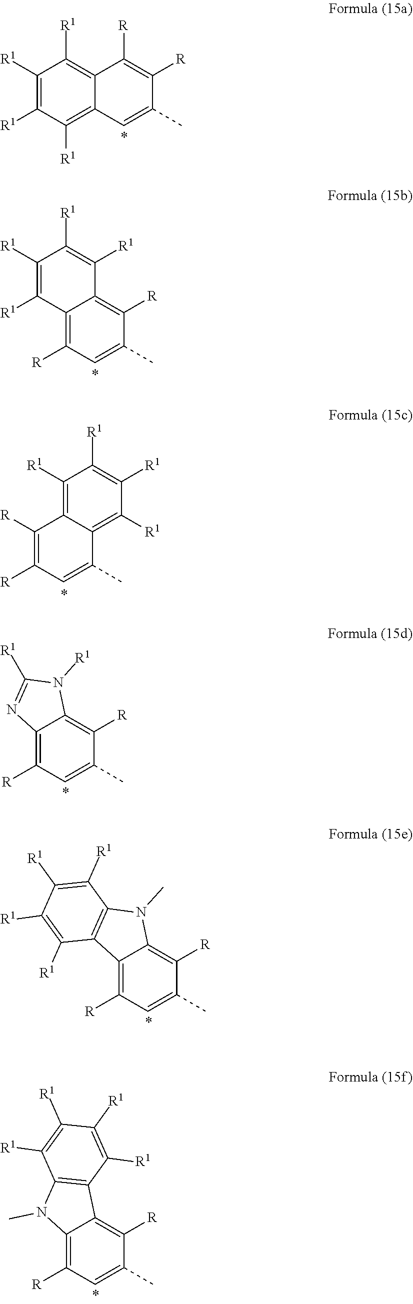

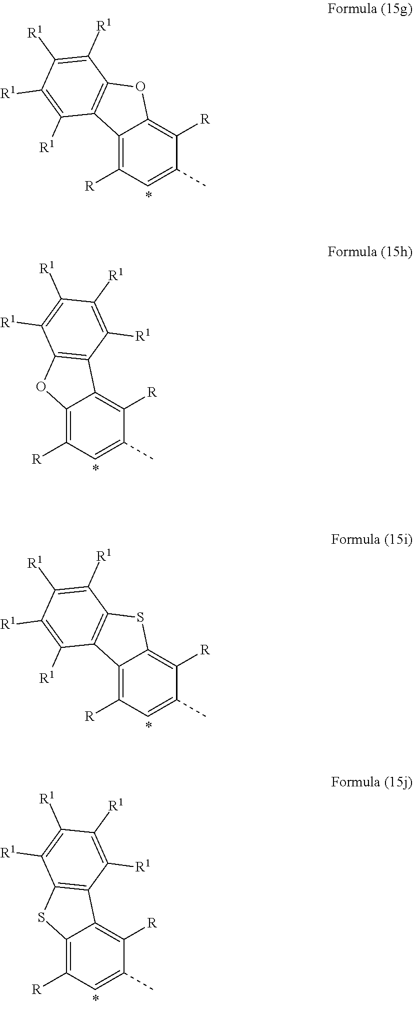

In general, the groups fused on may be fused onto any position in the unit of formula (5), as shown by the fused-on benzo group in the formulae (15a) to (15c). The groups as fused onto the unit of the formula (5) in the formulae (15d) to (15j) may therefore also be fused onto other positions in the unit of the formula (5).

The group of the formula (3) can more preferably be represented by the following formulae (3a) to (3m), and the group of the formula (4) can more preferably be represented by the following formulae (4a) to (4m):

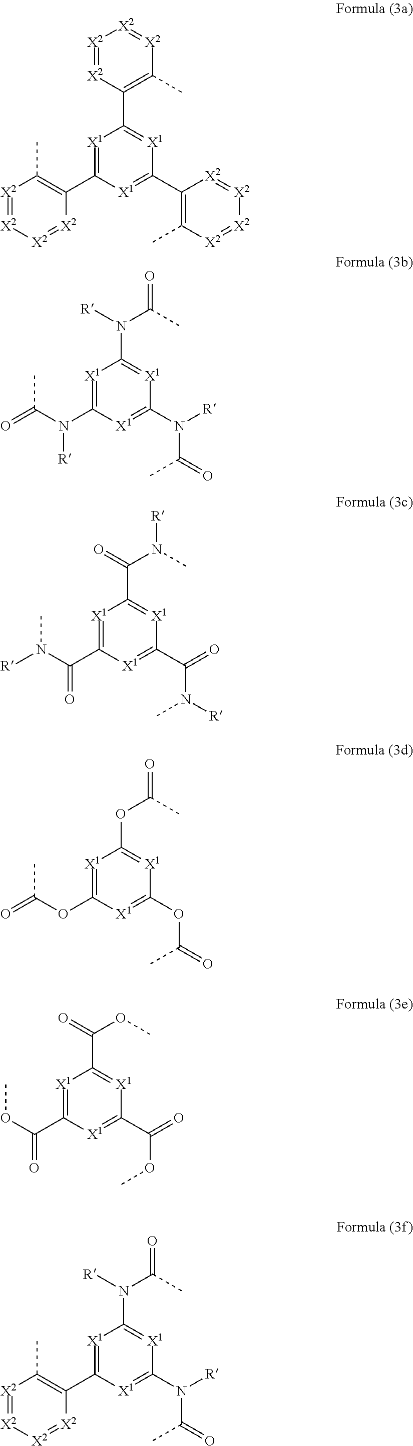

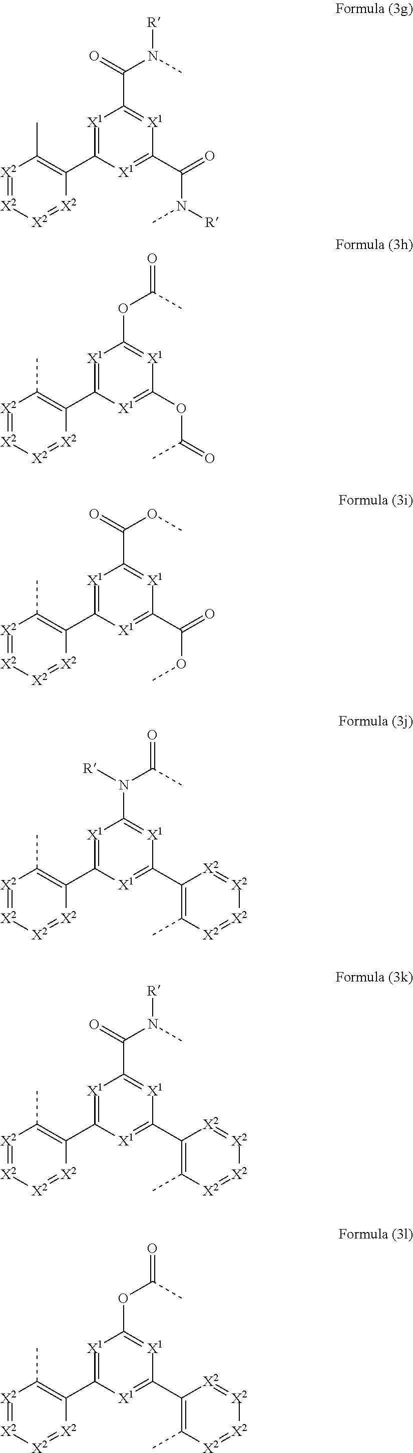

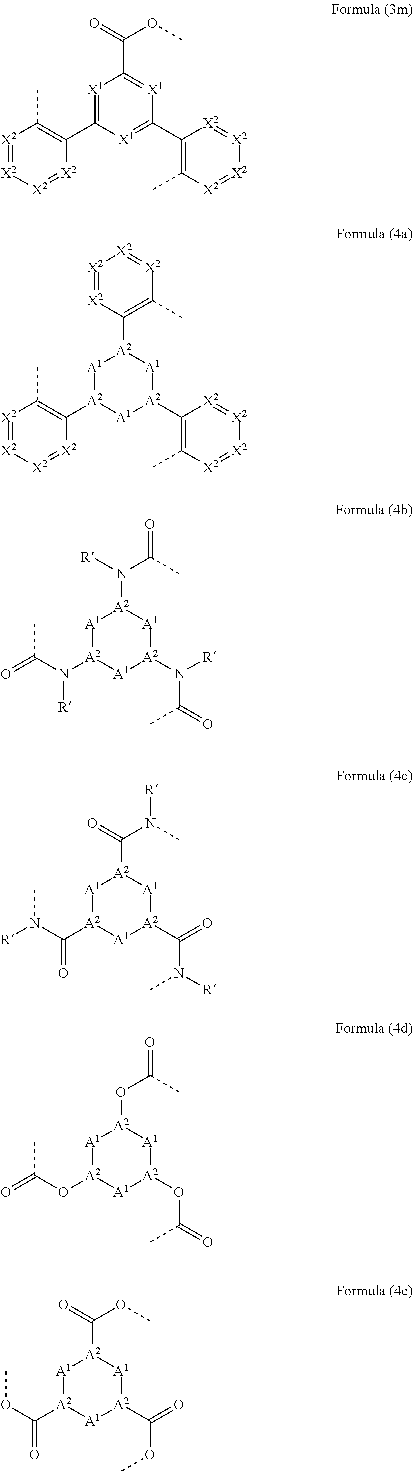

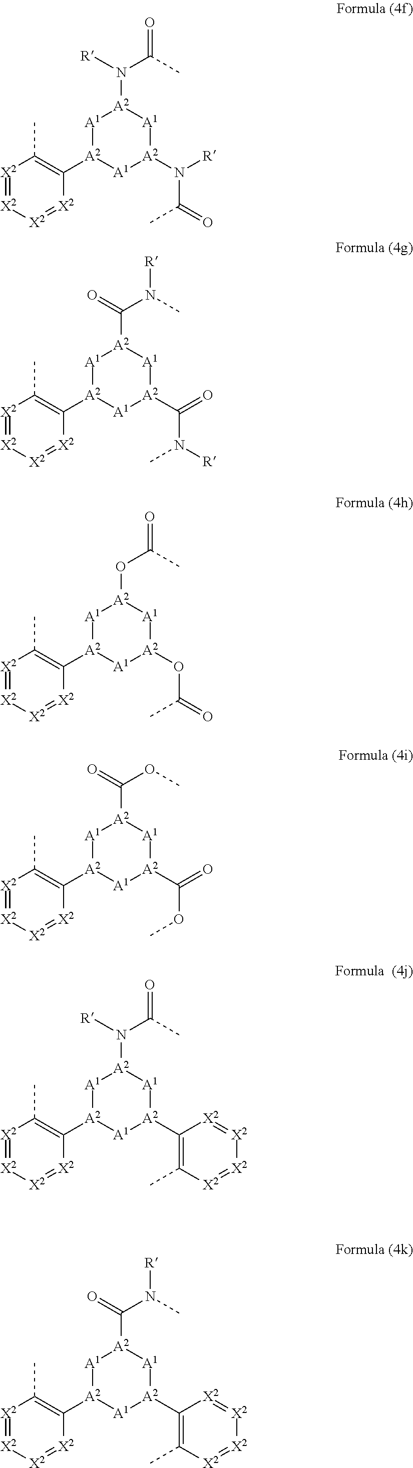

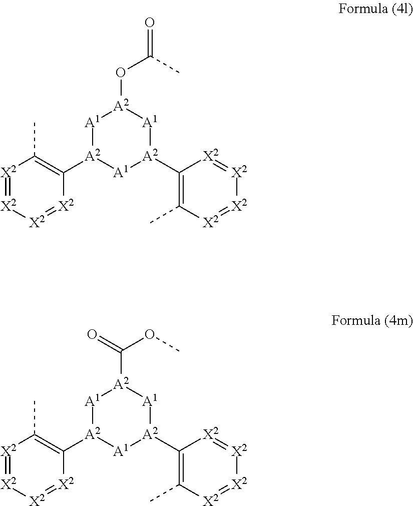

where the symbols have the definitions given above. Preferably, X² is the same or different at each instance and is CR.

In a preferred embodiment of the invention, the group of the formulae (3a) to (3m) is selected from the groups of the formulae (6a′) to (6 m′), and the group of the formulae (4a) to (4m) from the groups of the formulae (10a′) to (10m′):

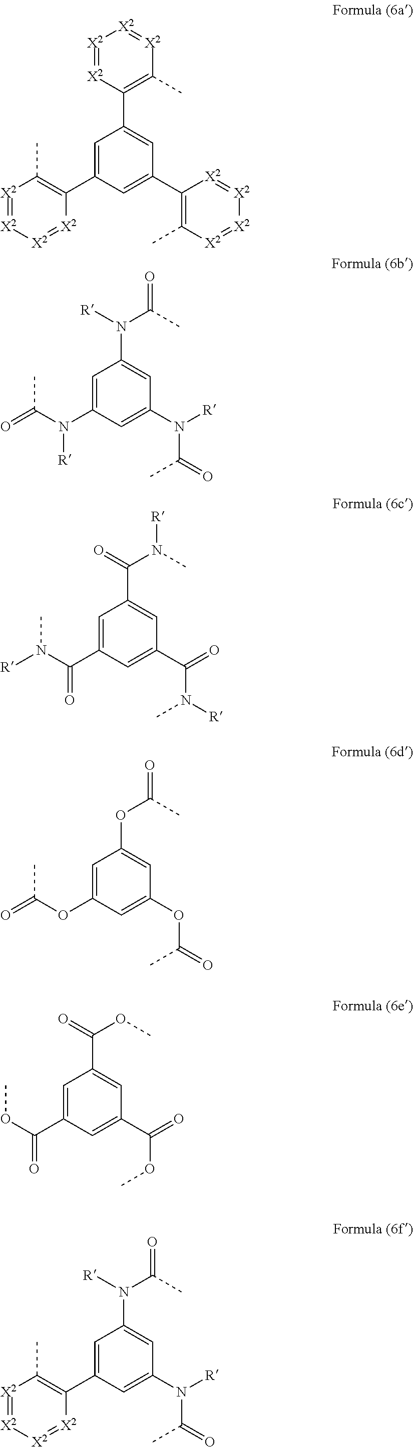

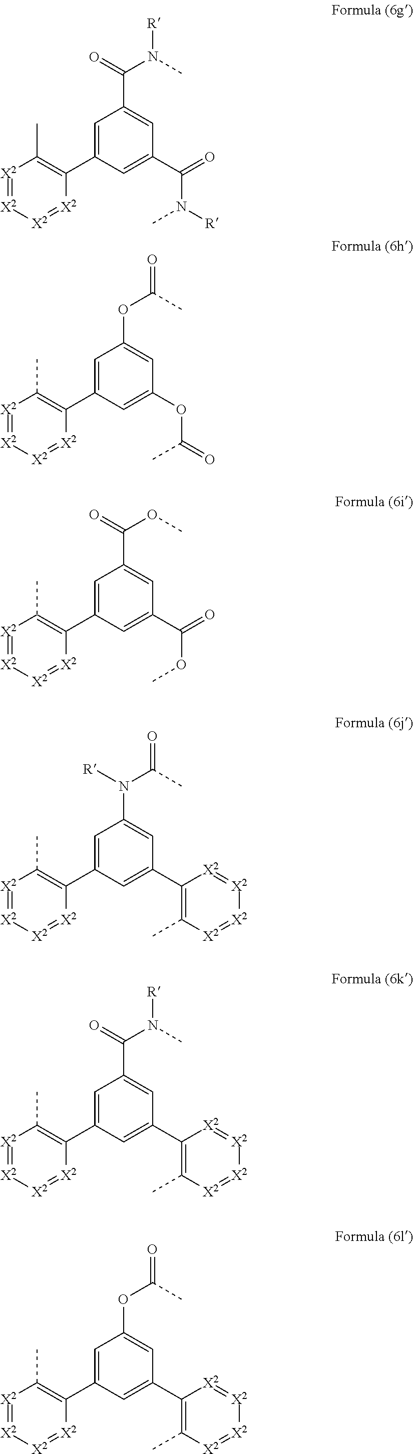

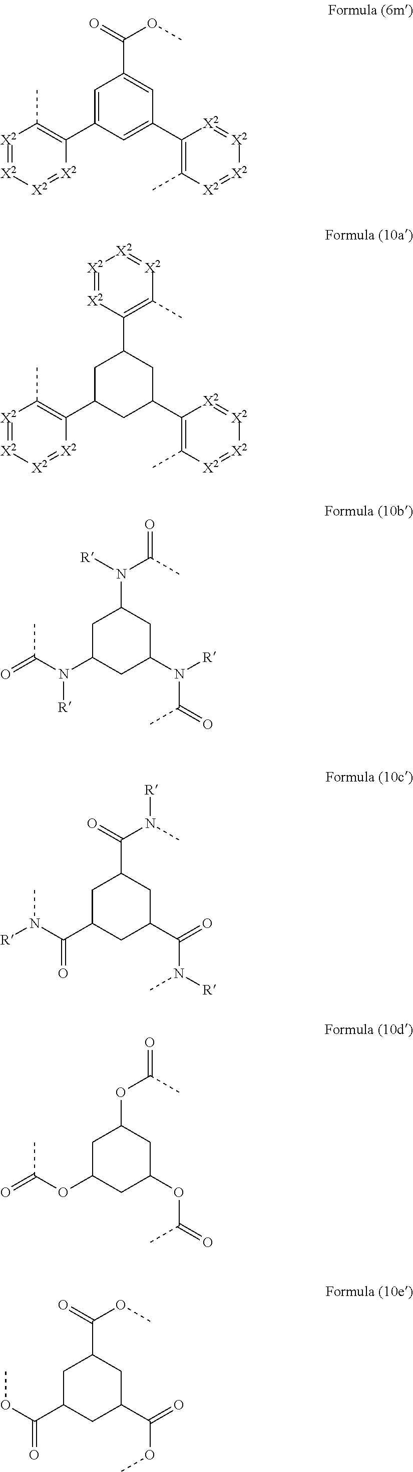

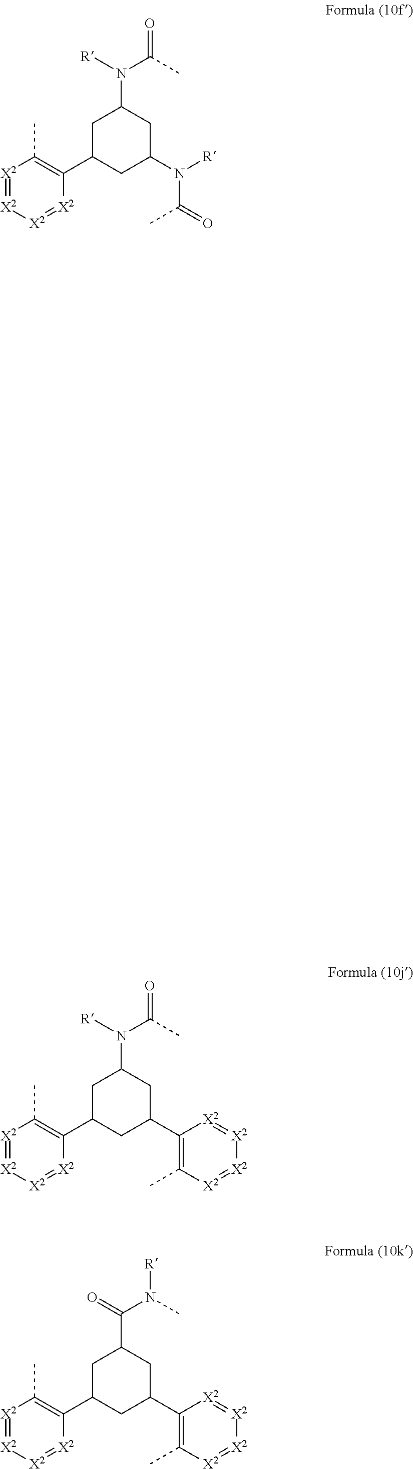

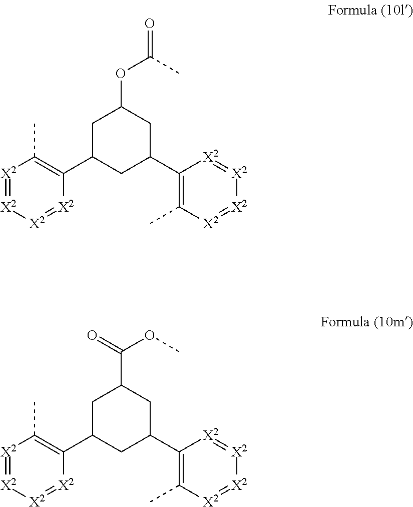

where the symbols have the definitions given above. Preferably, X² is the same or different at each instance and is CR.

A particularly preferred embodiment of the group of the formula (3) is the group of the following formula (6a″):

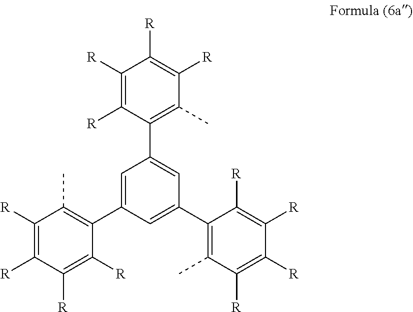

where the symbols have the definitions given above.

More preferably, the R groups in the abovementioned formulae are the same or different and are H, D or an alkyl group having 1 to 4 carbon atoms. Most preferably, R═H. Very particular preference is thus given to the structure of the following formula (6a′″):

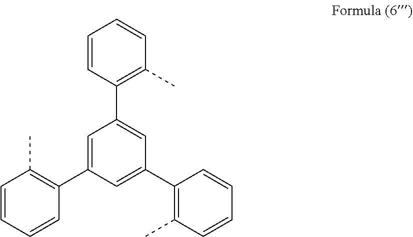

where the symbols have the definitions given above.

There follows a description of preferred substituents as may be present on the above-described sub-ligands and ligands, but also on the bivalent arylene or heteroarylene group in the structure of the formula (5).

In a preferred embodiment of the invention, the metal complex of the invention contains two R substituents or two R¹ substituents which are bonded to adjacent carbon atoms and together form an aliphatic ring according to one of the formulae described hereinafter. In this case, the two R substituents which form this aliphatic ring may be present on the bridge of the formulae (3) or (4) or the preferred embodiments and/or on one or more of the bidentate ligands. The aliphatic ring which is formed by the ring formation by two R substituents together or by two R¹ substituents together is preferably described by one of the following formulae (40) to (46):

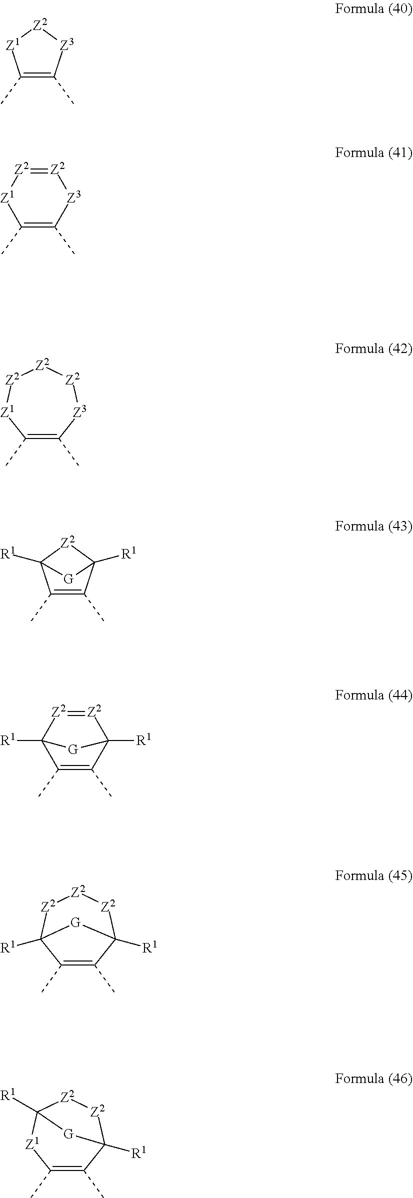

where R¹ and R² have the definitions given above, the dotted bonds signify the attachment of the two carbon atoms in the ligand, and in addition:
Z¹, Z³ is the same or different at each instance and is C(R³)₂, O, S, NR³ or C(═O);
Z² is C(R¹)₂, O, S, NR³ or C(═O);
G is an alkylene group which has 1, 2 or 3 carbon atoms and may be substituted by one or more R² radicals, —CR²═CR²- or an ortho-bonded arylene or heteroarylene group which has 5 to 14 aromatic ring atoms and may be substituted by one or more R² radicals;
R³ is the same or different at each instance and is H, F, a straight-chain alkyl or alkoxy group having 1 to 10 carbon atoms, a branched or cyclic alkyl or alkoxy group having 3 to 10 carbon atoms, where the alkyl or alkoxy group may be substituted in each case by one or more R² radicals, where one or more nonadjacent CH₂ groups may be replaced by R²C═CR², C≡C, Si(R²)₂, C═O, NR², O, S or CONR², or an aromatic or heteroaromatic ring system which has 5 to 24 aromatic ring atoms and may be substituted in each case by one or more R² radicals, or an aryloxy or heteroaryloxy group which has 5 to 24 aromatic ring atoms and may be substituted by one or more R² radicals; at the same time, two R³ radicals bonded to the same carbon atom together may form an aliphatic or aromatic ring system and thus form a spiro system; in addition, R³ with an adjacent R or R¹ radical may form an aliphatic ring system;
   with the proviso that no two heteroatoms in these groups are bonded directly to one another and no two C═O groups are bonded directly to one another.

In a preferred embodiment of the invention, R³ is not H.

In the above-depicted structures of the formulae (40) to (46) and the further embodiments of these structures specified as preferred, a double bond is depicted in a formal sense between the two carbon atoms. This is a simplification of the chemical structure when these two carbon atoms are incorporated into an aromatic or heteroaromatic system and hence the bond between these two carbon atoms is formally between the bonding level of a single bond and that of a double bond. The drawing of the formal double bond should thus not be interpreted so as to limit the structure; instead, it will be apparent to the person skilled in the art that this is an aromatic bond.

When adjacent radicals in the structures of the invention form an aliphatic ring system, it is preferable when the latter does not have any acidic benzylic protons. Benzylic protons are understood to mean protons which bind to a carbon atom bonded directly to the ligand. This can be achieved by virtue of the carbon atoms in the aliphatic ring system which bind directly to an aryl or heteroaryl group being fully substituted and not containing any bonded hydrogen atoms. Thus, the absence of acidic benzylic protons in the formulae (40) to (42) is achieved by virtue of Z¹ and Z³, when they are C(R³)₂, being defined such that R³ is not hydrogen. This can additionally also be achieved by virtue of the carbon atoms in the aliphatic ring system which bind directly to an aryl or heteroaryl group being the bridgeheads in a bi- or polycyclic structure. The protons bonded to bridgehead carbon atoms, because of the spatial structure of the bi- or polycycle, are significantly less acidic than benzylic protons on carbon atoms which are not bonded within a bi- or polycyclic structure, and are regarded as non-acidic protons in the context of the present invention. Thus, the absence of acidic benzylic protons in formulae (43) to (46) is achieved by virtue of this being a bicyclic structure, as a result of which R¹, when it is H, is much less acidic than benzylic protons since the corresponding anion of the bicyclic structure is not mesomerically stabilized. Even when R¹ in formulae (43) to (46) is H, this is therefore a non-acidic proton in the context of the present application.

In a preferred embodiment of the structure of the formulae (40) to (46), not more than one of the Z¹, Z² and Z³ groups is a heteroatom, especially O or NR³, and the other groups are C(R³)₂ or C(R¹)₂, or Z¹ and Z³ are the same or different at each instance and are O or NR³ and Z² is C(R¹)₂. In a particularly preferred embodiment of the invention, Z¹ and Z³ are the same or different at each instance and are C(R³)₂, and Z² is C(R¹)₂ and more preferably C(R³)₂ or CH₂.

Preferred embodiments of the formula (40) are thus the structures of the formulae (40-A), (40-B), (40-C) and (40-D), and a particularly preferred embodiment of the formula (40-A) is the structures of the formulae (40-E) and (40-F):

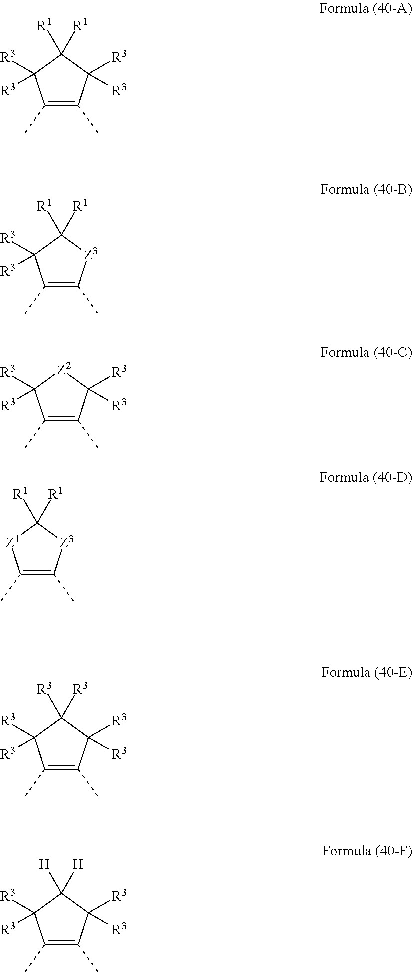

where R¹ and R³ have the definitions given above and Z¹, Z² and Z³ are the same or different at each instance and are O or NR³.

Preferred embodiments of the formula (41) are the structures of the following formulae (41-A) to (41-F):

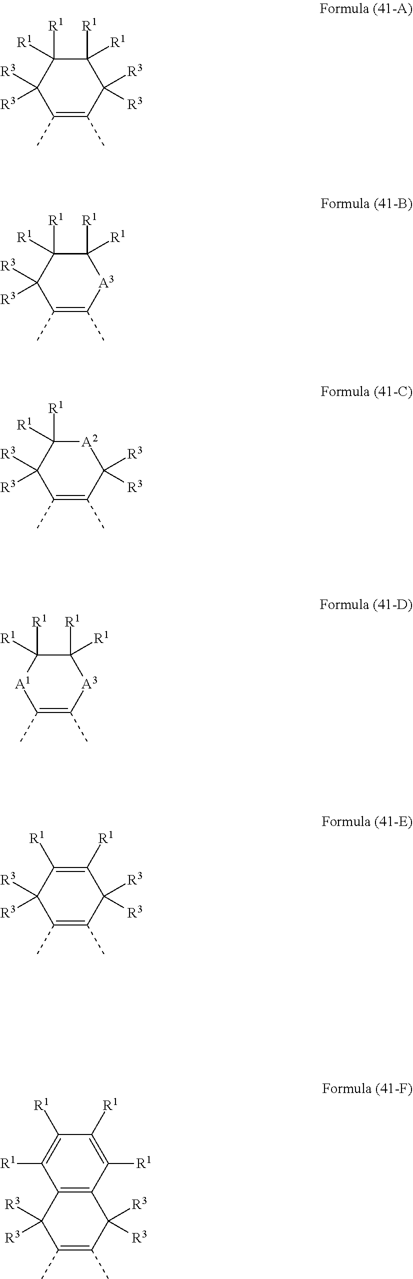

where R¹ and R³ have the definitions given above and Z¹, Z² and Z³ are the same or different at each instance and are O or NR³.

Preferred embodiments of the formula (42) are the structures of the following formulae (42-A) to (42-E):

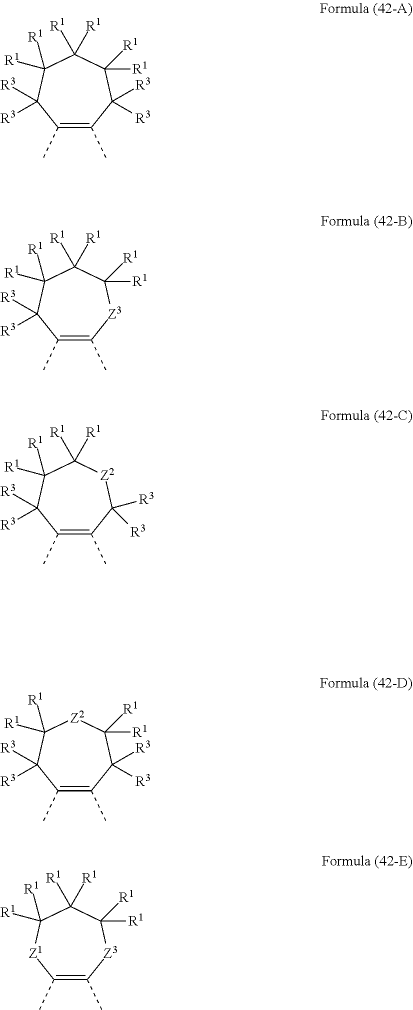

where R¹ and R³ have the definitions given above and Z¹, Z² and Z³ are the same or different at each instance and are O or NR³.

In a preferred embodiment of the structure of formula (43), the R¹ radicals bonded to the bridgehead are H, D, F or CH₃. Further preferably, Z² is C(R¹)₂ or O, and more preferably C(R³)₂. Preferred embodiments of the formula (43) are thus structures of the formulae (43-A) and (43-B), and a particularly preferred embodiment of the formula (43-A) is a structure of the formula (43-C):

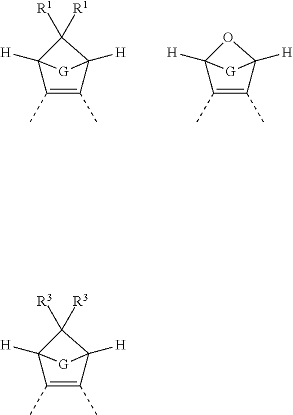

where the symbols used have the definitions given above.

In a preferred embodiment of the structure of formulae (44), (45) and (46), the R¹ radicals bonded to the bridgehead are H, D, F or CH₃. Further preferably, Z² is C(R¹)₂. Preferred embodiments of the formula (44), (45) and (46) are thus the structures of the formulae (44-A), (45-A) and (46-A):

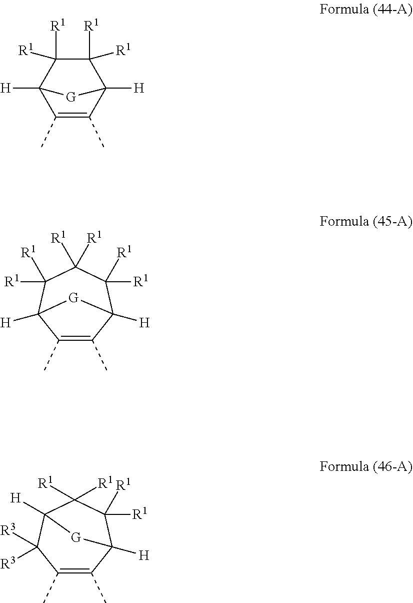

where the symbols used have the definitions given above.

Further preferably, the G group in the formulae (43), (43-A), (43-B), (43-C), (44), (44-A), (45), (45-A), (46) and (46-A) is a 1,2-ethylene group which may be substituted by one or more R² radicals, where R² is preferably the same or different at each instance and is H or an alkyl group having 1 to 4 carbon atoms, or an ortho-arylene group which has 6 to 10 carbon atoms and may be substituted by one or more R² radicals, but is preferably unsubstituted, especially an ortho-phenylene group which may be substituted by one or more R² radicals, but is preferably unsubstituted.

In a further preferred embodiment of the invention, R³ in the groups of the formulae (40) to (46) and in the preferred embodiments is the same or different at each instance and is F, a straight-chain alkyl group having 1 to 10 carbon atoms or a branched or cyclic alkyl group having 3 to 20 carbon atoms, where one or more nonadjacent CH₂ groups in each case may be replaced by R²C═CR² and one or more hydrogen atoms may be replaced by D or F, or an aromatic or heteroaromatic ring system which has 5 to 14 aromatic ring atoms and may be substituted in each case by one or more R² radicals; at the same time, two R³ radicals bonded to the same carbon atom may together form an aliphatic or aromatic ring system and thus form a spiro system; in addition, R³ may form an aliphatic ring system with an adjacent R or R¹ radical.

In a particularly preferred embodiment of the invention, R³ in the groups of the formulae (40) to (46) and in the preferred embodiments is the same or different at each instance and is F, a straight-chain alkyl group having 1 to 3 carbon atoms, especially methyl, or an aromatic or heteroaromatic ring system which has 5 to 12 aromatic ring atoms and may be substituted in each case by one or more R² radicals, but is preferably unsubstituted; at the same time, two R³ radicals bonded to the same carbon atom may together form an aliphatic or aromatic ring system and thus form a spiro system; in addition, R³ may form an aliphatic ring system with an adjacent R or R¹ radical.

Examples of particularly suitable groups of the formula (40) are the groups depicted below:

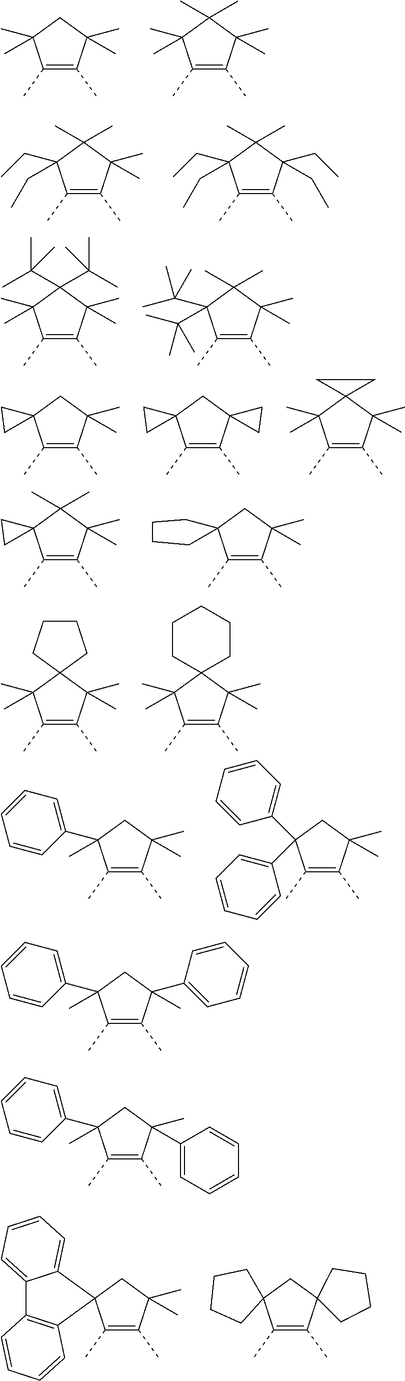

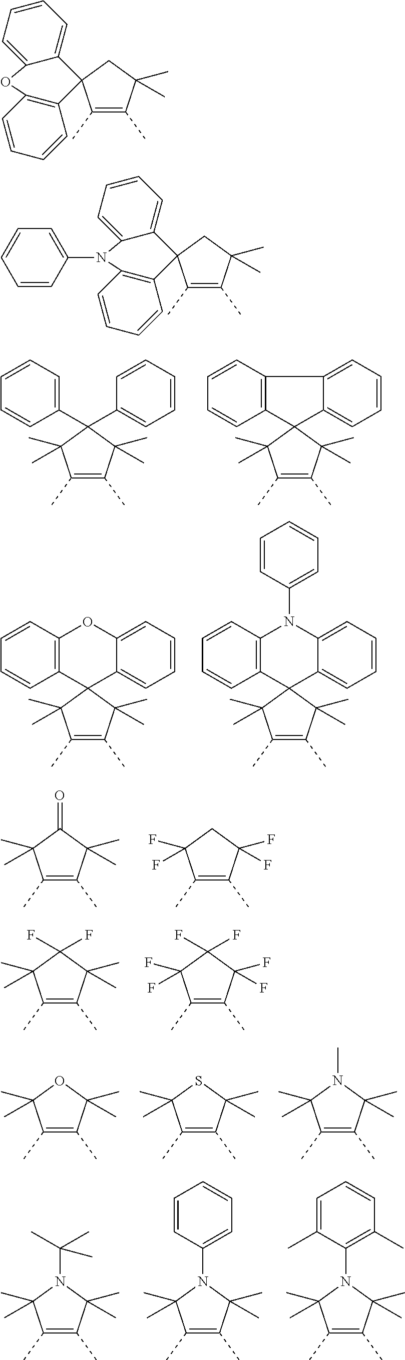

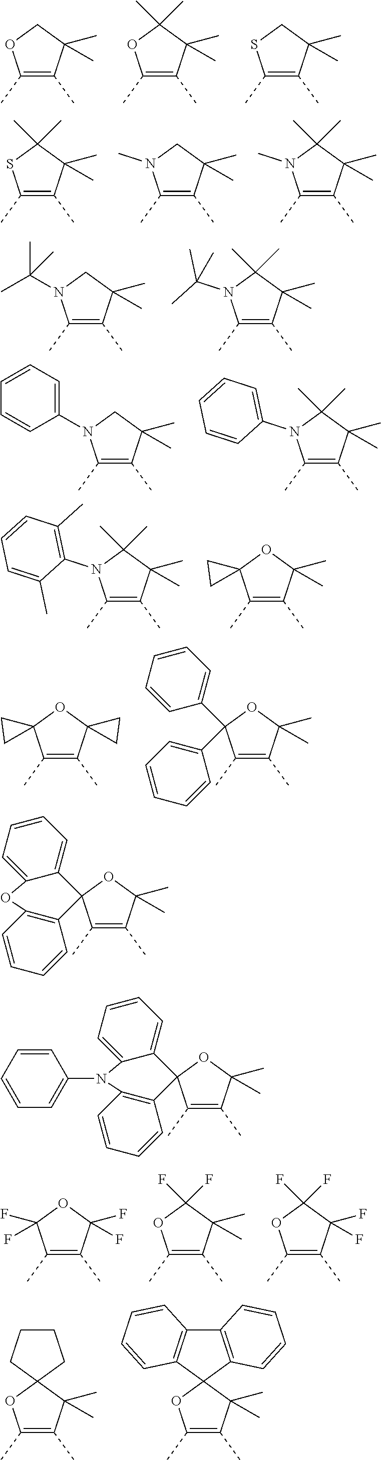

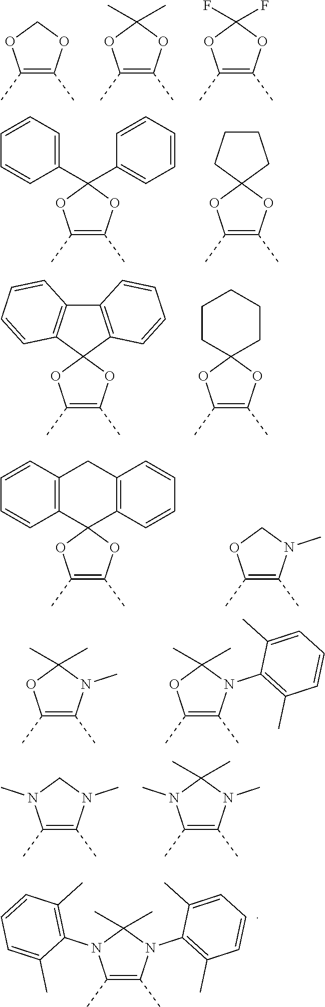

Examples of particularly suitable groups of the formula (41) are the groups depicted below:

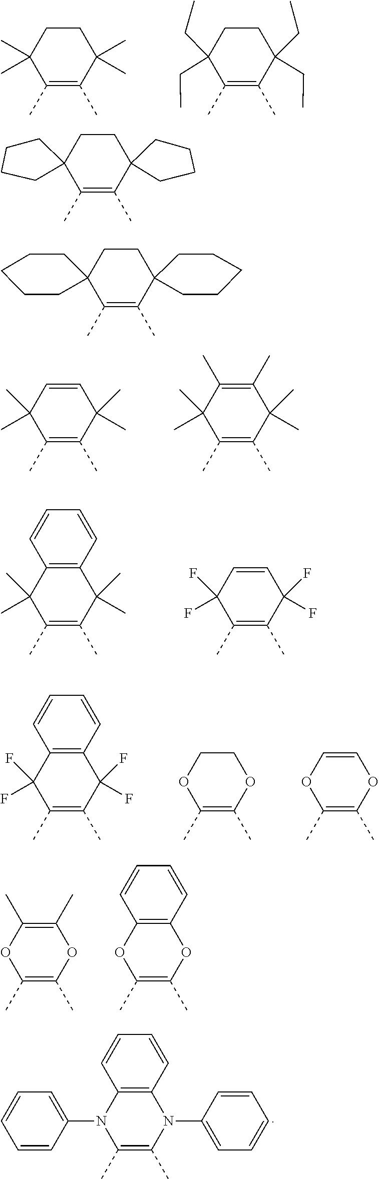

Examples of particularly suitable groups of the formulae (42), (45) and (46) are the groups depicted below:

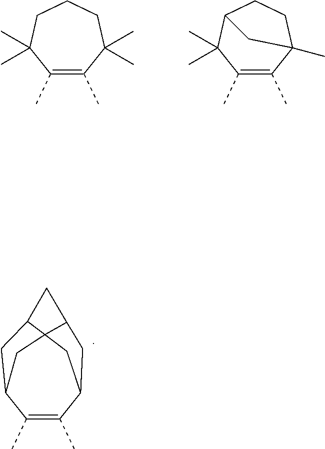

Examples of particularly suitable groups of the formula (43) are the groups depicted below:

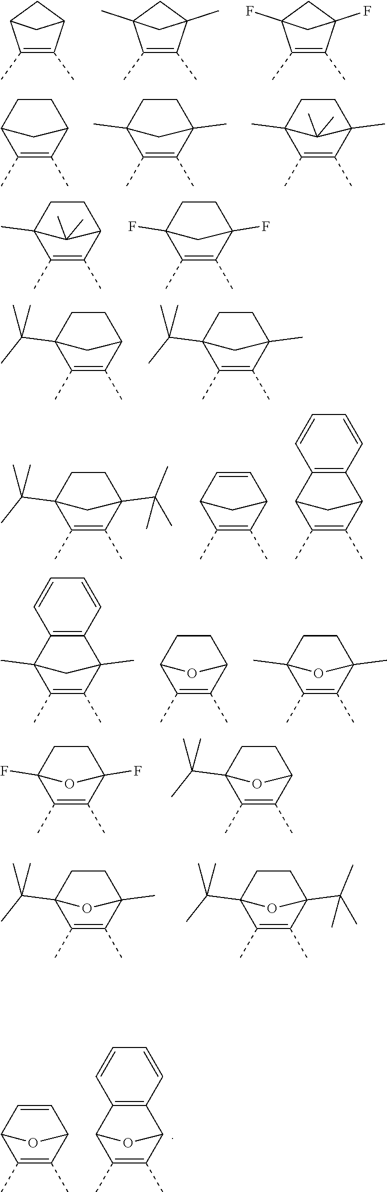

Examples of particularly suitable groups of the formula (44) are the groups depicted below:

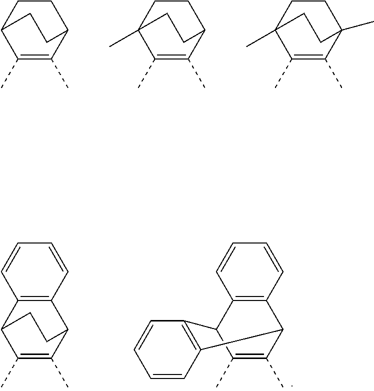

When R radicals are bonded within the substructures of the formulae (2), (2a), (2-1) and/or (2a-1) or or within the bidentate sub-ligands or ligands or within the bivalent arylene or heteroarylene groups of the formula (5) bonded within the formulae (3) or (4) or the preferred embodiments, these R radicals are the same or different at each instance and are preferably selected from the group consisting of H, D, F, Br, I, N(R¹)₂, CN, Si(R¹)₃, B(OR¹)₂, C(═O)R¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may be substituted in each case by one or more R¹ radicals, or an aromatic or heteroaromatic ring system which has 5 to 30 aromatic ring atoms and may be substituted in each case by one or more R¹ radicals; at the same time, two adjacent R radicals together or R together with R¹ may also form a ring system. More preferably, these R radicals are the same or different at each instance and are selected from the group consisting of H, D, F, N(R¹)₂, a straight-chain alkyl group having 1 to 6 carbon atoms or a branched or cyclic alkyl group having 3 to 10 carbon atoms, where one or more hydrogen atoms may be replaced by D or F, or an aromatic or heteroaromatic ring system which has 5 to 24 aromatic ring atoms and may be substituted in each case by one or more R¹ radicals; at the same time, two adjacent R radicals together or R together with R¹ may also form a mono- or polycyclic ring system.

Examples of suitable compounds of the invention are the structures of the following formulae (I-1) to (I-14) shown below:

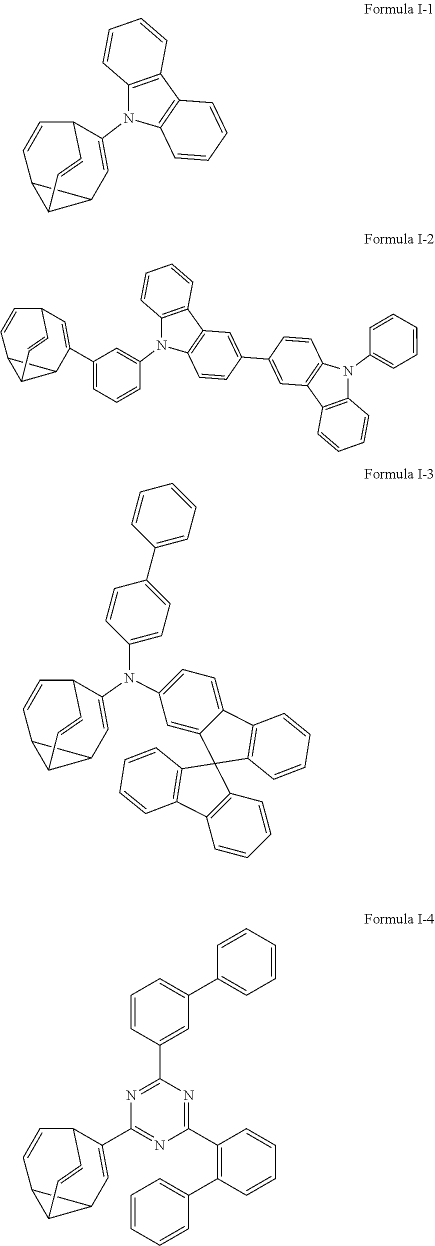 [SMILES: C1=CC2C3C=CC1C(n1c4ccccc4c4ccccc41)=CC23 ; C1=CC2C3C=CC1C=C(c1cccc(-n4c5ccccc5c5cc(-c6ccc7c(c6)c6ccccc6n7-c6ccccc6)ccc54)c1)C23 ; C1=CC2C3C=CC1C(N(c1ccc(-c4ccccc4)cc1)c1ccc4c(c1)C1(c5ccccc5-c5ccccc51)c1ccccc1-4)=CC23 ; C1=CC2C3C=CC1C(c1nc(-c4cccc(-c5ccccc5)c4)nc(-c4ccccc4-c4ccccc4)n1)=CC23]

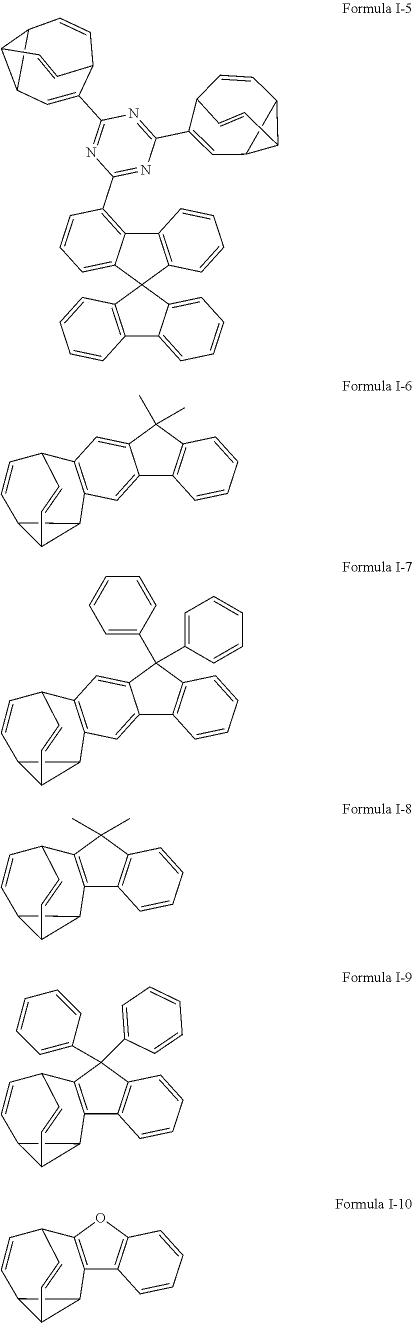

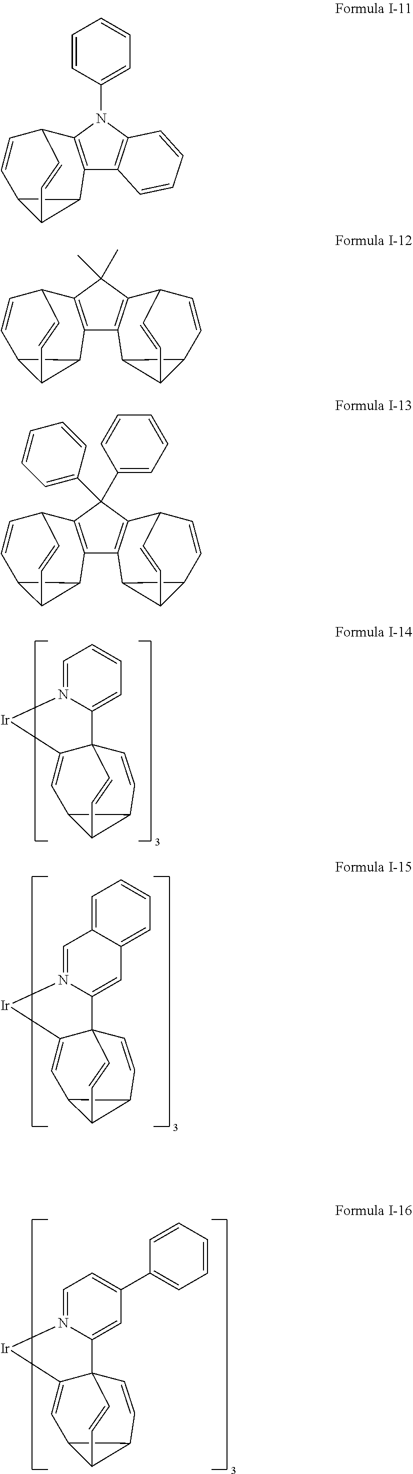

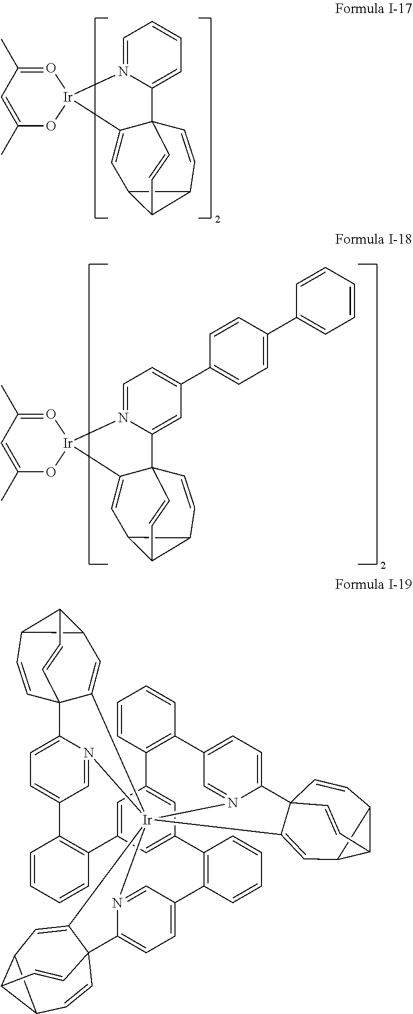

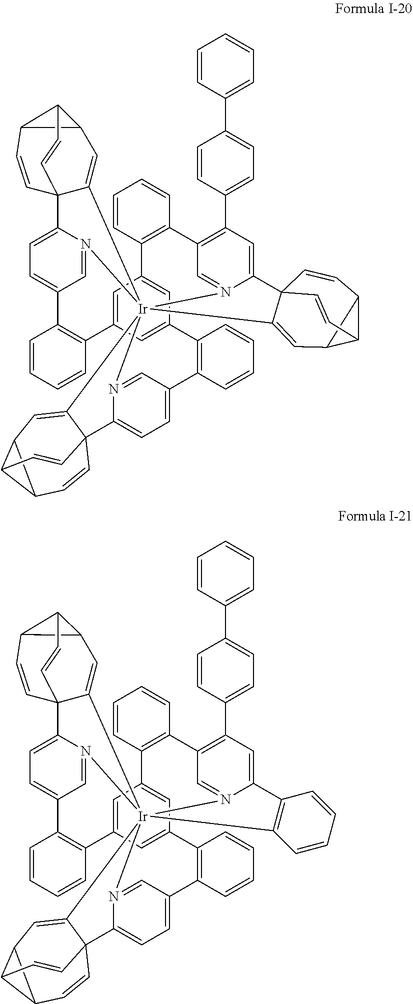

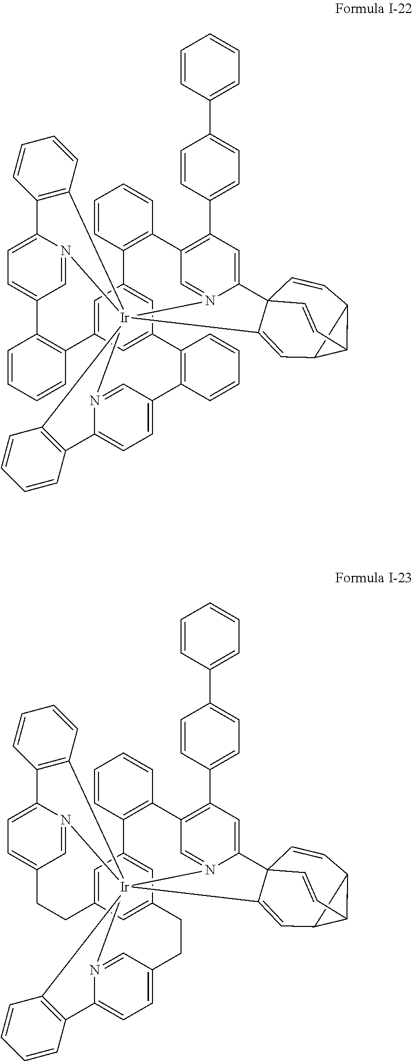

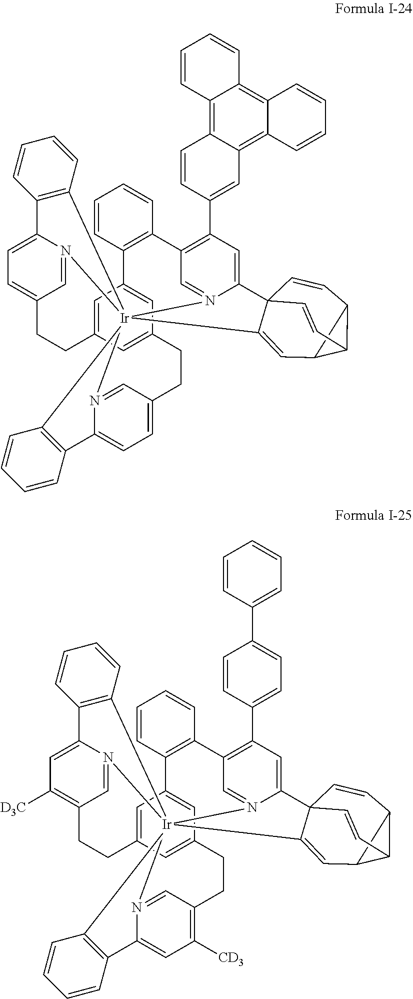

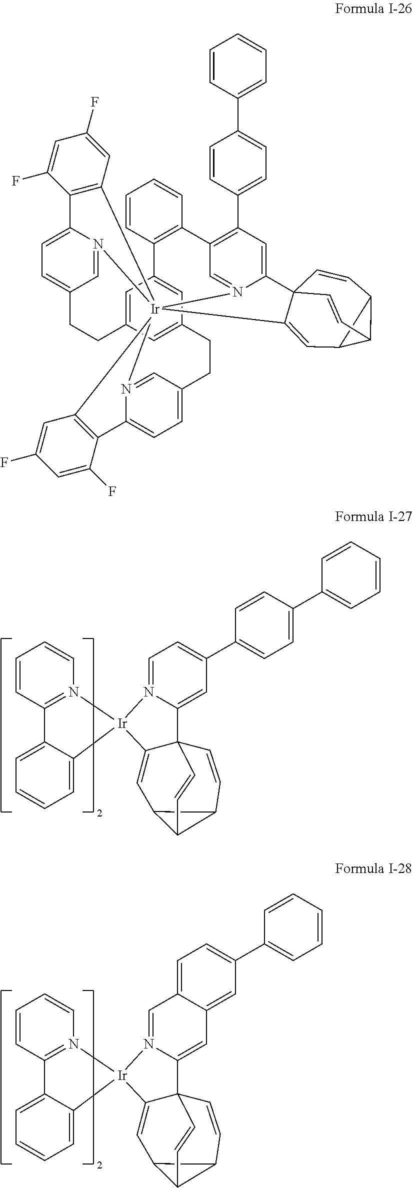

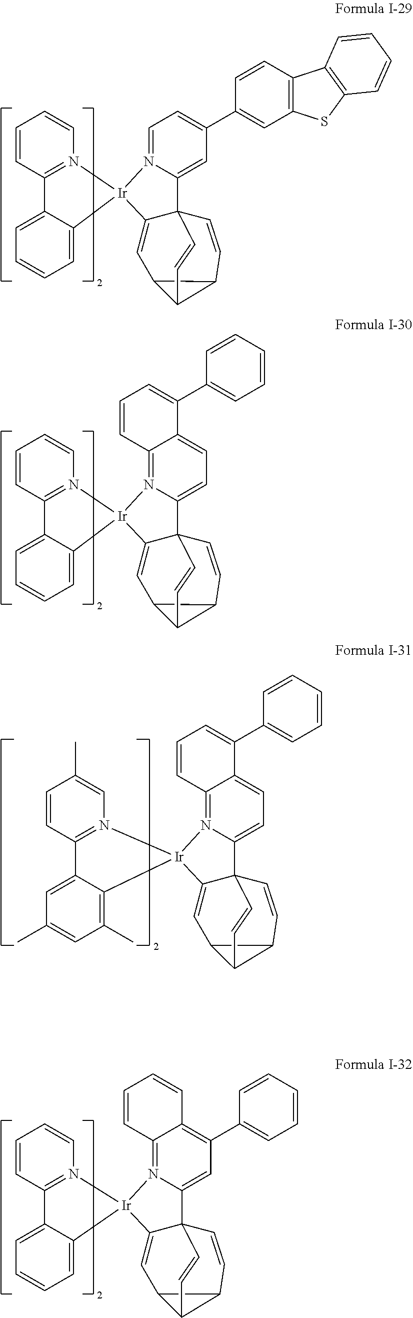

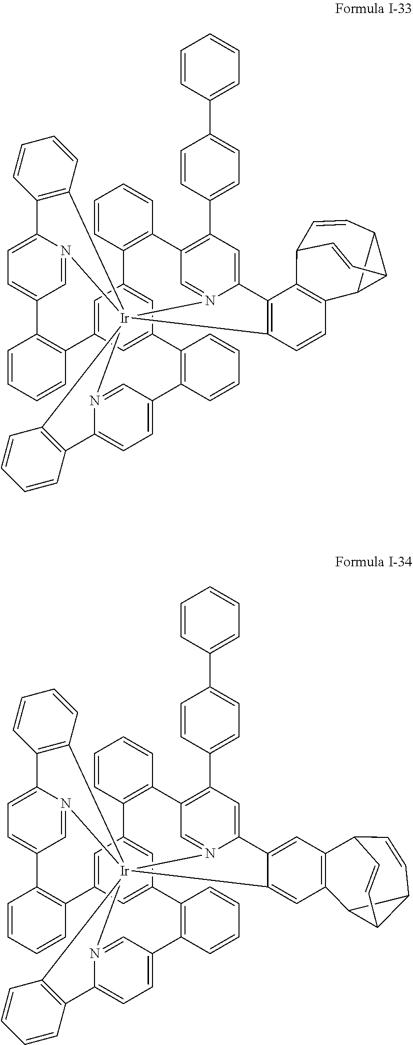

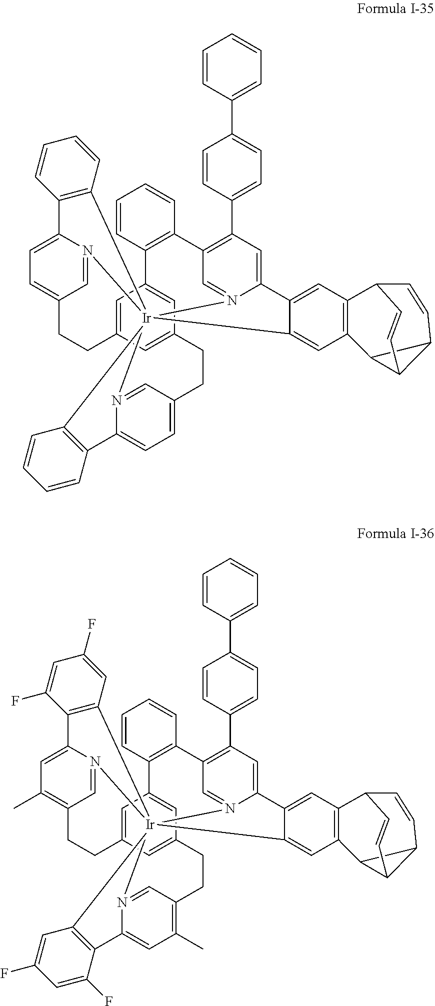

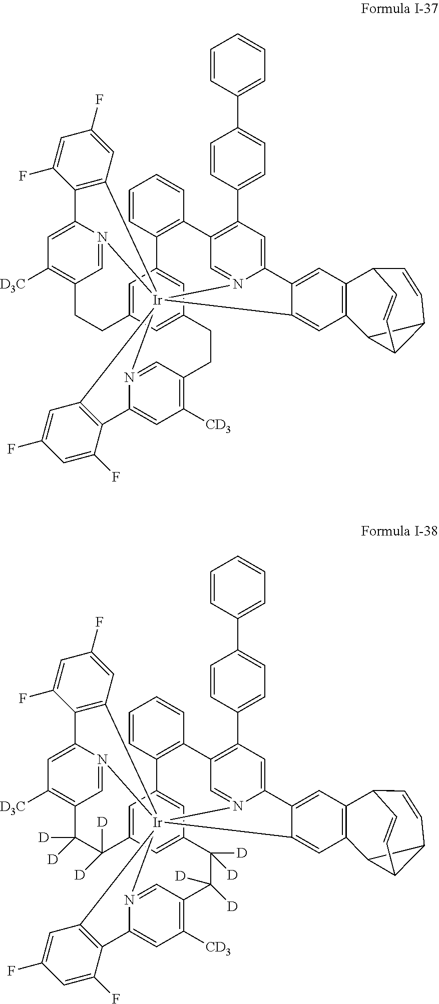

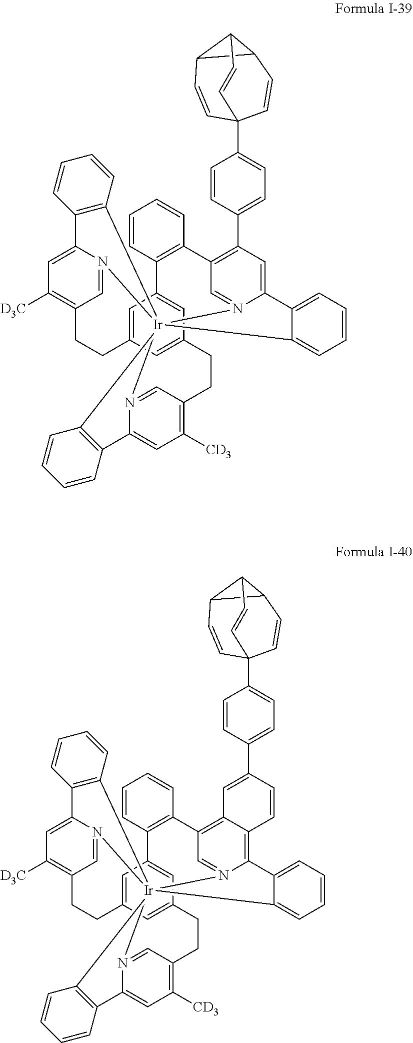

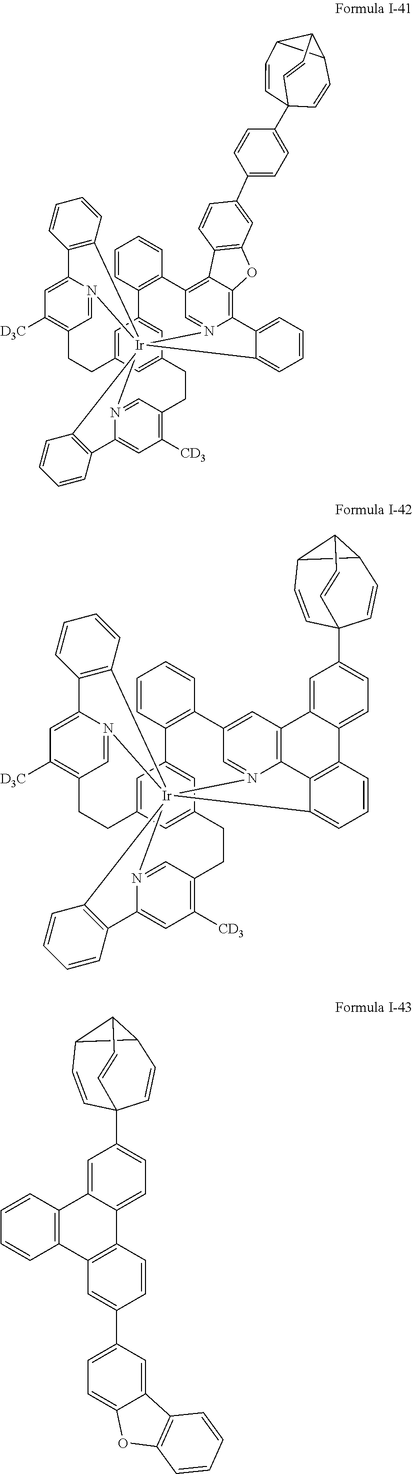

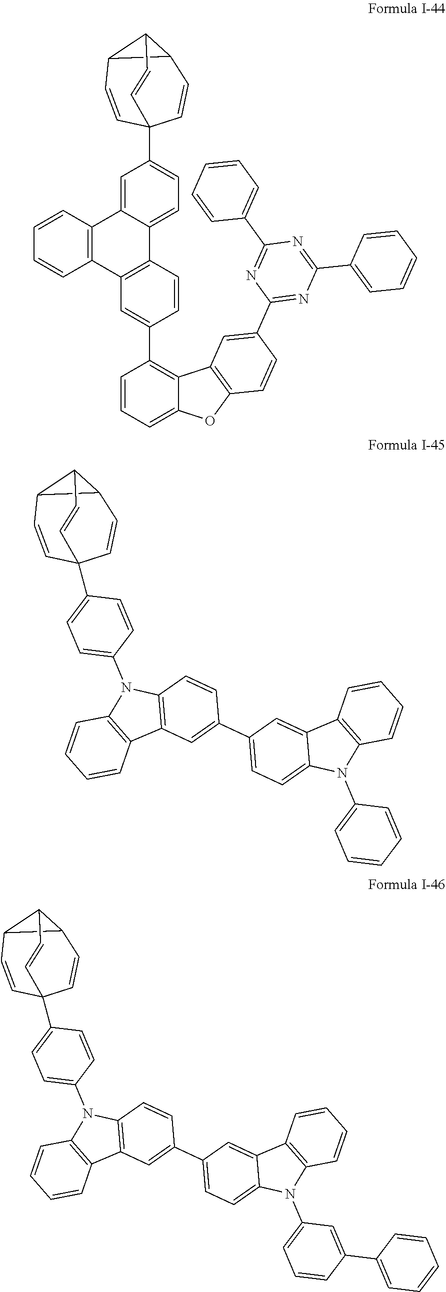 [SMILES: C1=CC2(c3ccc4c5ccc(-c6cccc7oc8ccc(-c9nc(-c%10ccccc%10)nc(-c%10ccccc%10)n9)cc8c67)cc5c5ccccc5c4c3)C=CC3C1C3C=C2 ; C1=CC2(c3ccc(-n4c5ccccc5c5cc(-c6ccc7c(c6)c6ccccc6n7-c6ccccc6)ccc54)cc3)C=CC3C1C3C=C2 ; C1=CC2(c3ccc(-n4c5ccccc5c5cc(-c6ccc7c(c6)c6ccccc6n7-c6cccc(-c7ccccc7)c6)ccc54)cc3)C=CC3C1C3C=C2]

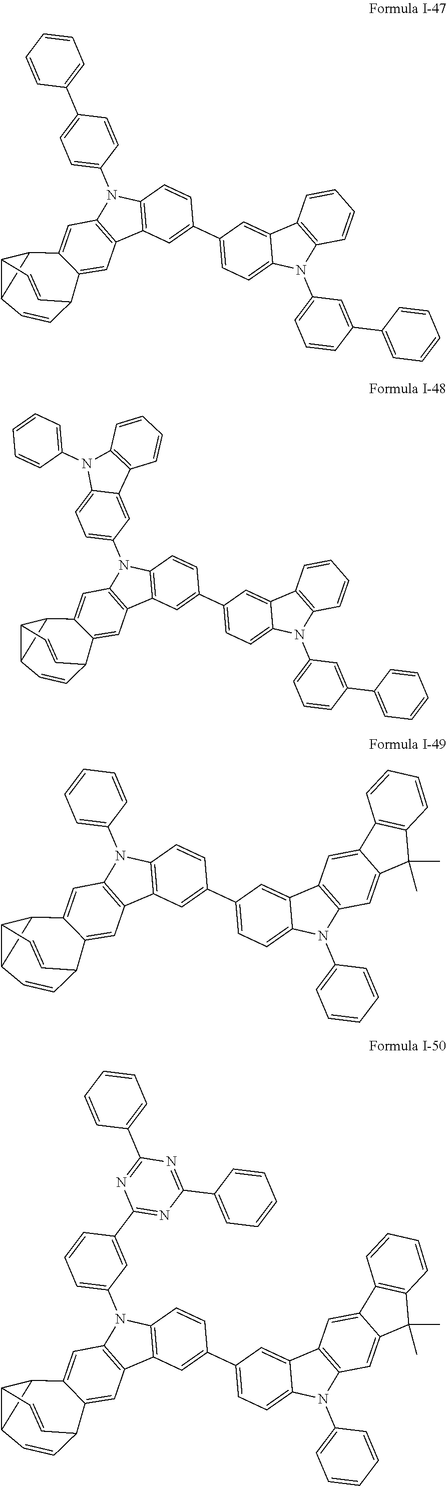 [SMILES: C1=CC2C3C=CC1c1cc4c5cc(-c6ccc7c(c6)c6ccccc6n7-c6cccc(-c7ccccc7)c6)ccc5n(-c5ccc(-c6ccccc6)cc5)c4cc1C23 ; C1=CC2C3C=CC1c1cc4c5cc(-c6ccc7c(c6)c6ccccc6n7-c6cccc(-c7ccccc7)c6)ccc5n(-c5ccc6c(c5)c5ccccc5n6-c5ccccc5)c4cc1C23 ; CC1(C)c2ccccc2-c2cc3c4cc(-c5ccc6c(c5)c5cc7c(cc5n6-c5ccccc5)C5C6C=CC7C=CC65)ccc4n(-c4ccccc4)c3cc21 ; CC1(C)c2ccccc2-c2cc3c4cc(-c5ccc6c(c5)c5cc7c(cc5n6-c5cccc(-c6nc(-c8ccccc8)nc(-c8ccccc8)n6)c5)C5C6C=CC7C=CC65)ccc4n(-c4ccccc4)c3cc21]

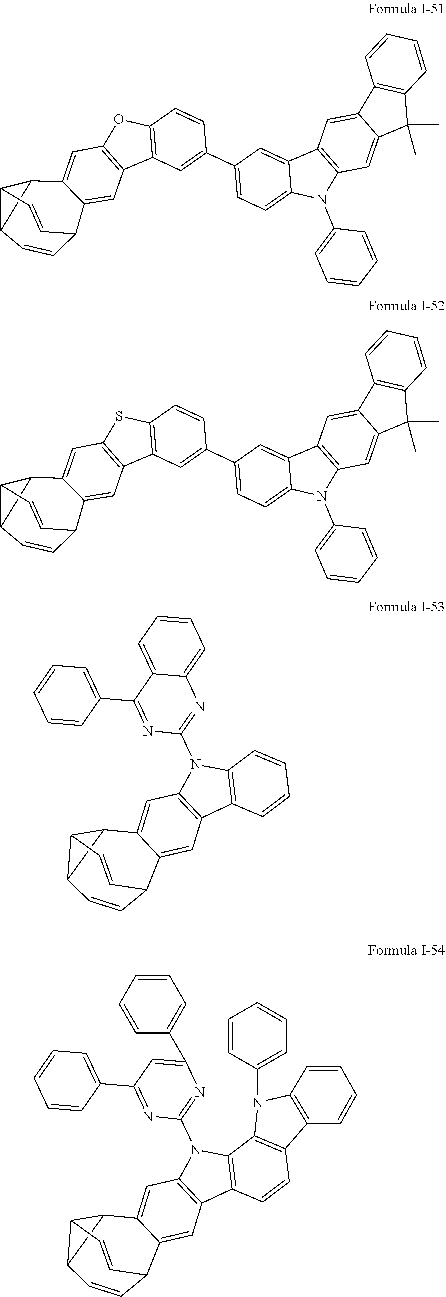 [SMILES: CC1(C)c2ccccc2-c2cc3c4cc(-c5ccc6oc7cc8c(cc7c6c5)C5C=CC6C(C=C5)C86)ccc4n(-c4ccccc4)c3cc21 ; CC1(C)c2ccccc2-c2cc3c4cc(-c5ccc6sc7cc8c(cc7c6c5)C5C=CC6C(C=C5)C86)ccc4n(-c4ccccc4)c3cc21 ; C1=CC2C3C=CC1c1cc4c5ccccc5n(-c5nc(-c6ccccc6)c6ccccc6n5)c4cc1C23 ; C1=CC2C3C=CC1c1cc4c5ccc6c7ccccc7n(-c7ccccc7)c6c5n(-c5nc(-c6ccccc6)cc(-c6ccccc6)n5)c4cc1C23]

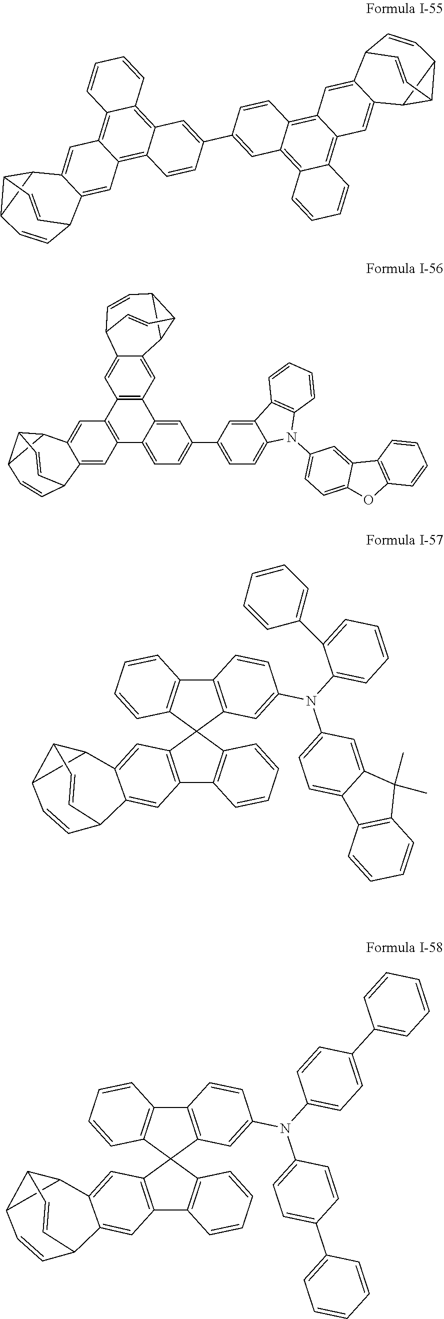

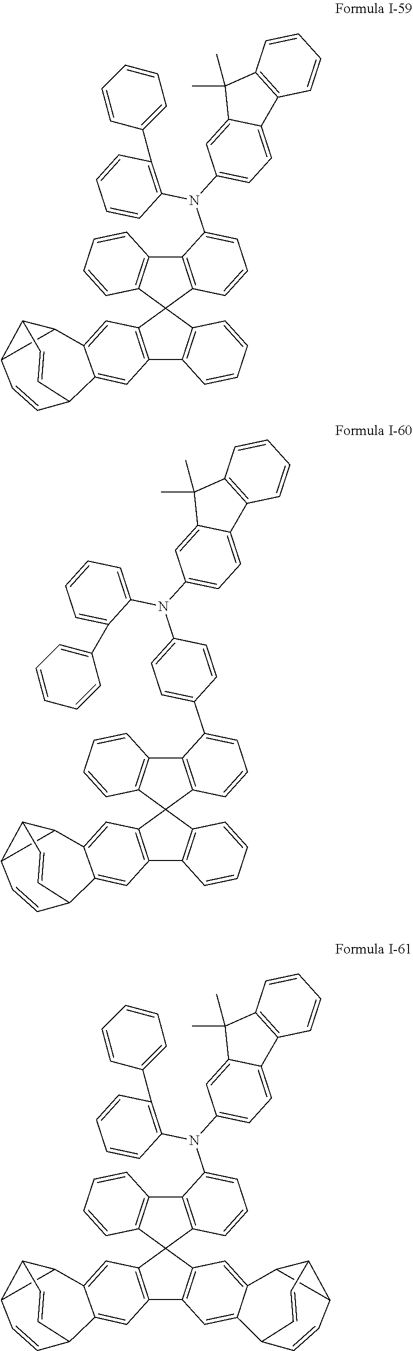 [SMILES: CC1(C)c2ccccc2-c2ccc(N(c3ccccc3-c3ccccc3)c3cccc4c3-c3ccccc3C43c4ccccc4-c4cc5c(cc43)C3C4C=CC5C=CC43)cc21 ; CC1(C)c2ccccc2-c2ccc(N(c3ccc(-c4cccc5c4-c4ccccc4C54c5ccccc5-c5cc6c(cc54)C4C5C=CC6C=CC54)cc3)c3ccccc3-c3ccccc3)cc21 ; CC1(C)c2ccccc2-c2ccc(N(c3ccccc3-c3ccccc3)c3cccc4c3-c3ccccc3C43c4cc5c(cc4-c4cc6c(cc43)C3C4C=CC6C=CC43)C3C=CC4C(C=C3)C54)cc21]

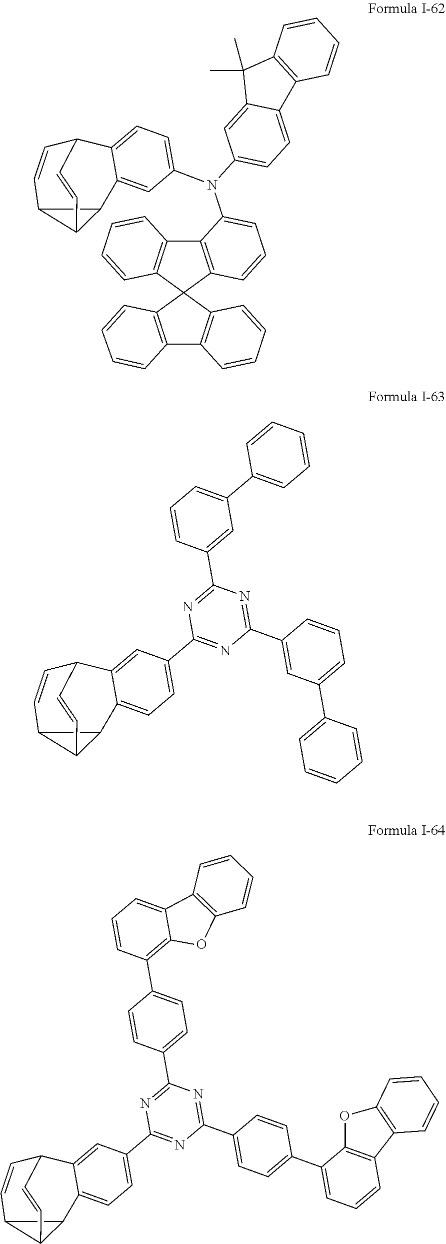 [SMILES: CC1(C)c2ccccc2-c2ccc(N(c3ccc4c(c3)C3C5C=CC4C=CC53)c3cccc4c3-c3ccccc3C43c4ccccc4-c4ccccc43)cc21 ; C1=CC2C3C=CC1c1cc(-c4nc(-c5cccc(-c6ccccc6)c5)nc(-c5cccc(-c6ccccc6)c5)n4)ccc1C23 ; C1=CC2C3C=CC1c1cc(-c4nc(-c5ccc(-c6cccc7c6oc6ccccc67)cc5)nc(-c5ccc(-c6cccc7c6oc6ccccc67)cc5)n4)ccc1C23]

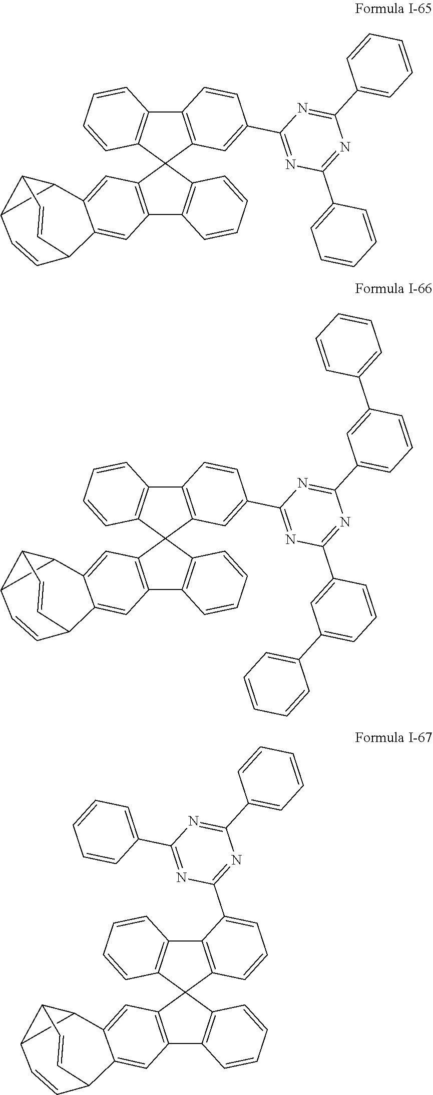

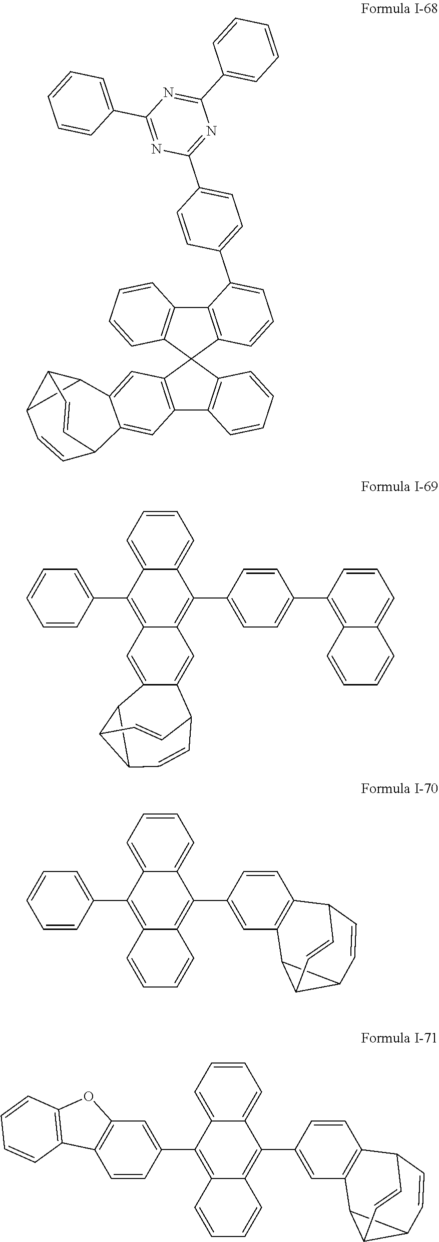

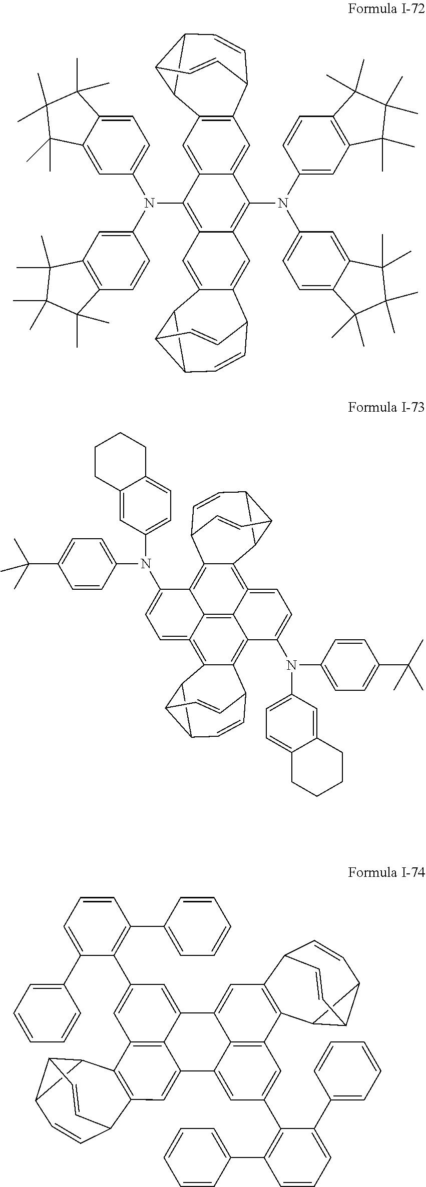

Preferred embodiments of compounds of the invention are recited in detail in the examples, these compounds being usable alone or in combination with further compounds for all purposes of the invention.

Provided that the conditions specified in claim 1 are met, the abovementioned preferred embodiments can be combined with one another as desired. In a particularly preferred embodiment of the invention, the abovementioned preferred embodiments apply simultaneously.

The compounds of the invention are preparable in principle by various processes. However, the processes described hereinafter have been found to be particularly suitable.

Therefore, the present invention further provides a process for preparing the compounds of the invention, preferably compounds comprising at least one structure of the formulae (I), (Ia), (II), (IIa), (IIIa) to (IIIh), (IVa) to (IVh), and/or metal complexes comprising at least one substructure of the formulae (2), (2a), (2-1), (2a-1), in which a compound comprising a structure of formula (I), (Ia), (II) and/or (IIa) is bonded to a compound comprising at least one aromatic or heteroaromatic group in a coupling reaction.

Suitable compounds comprising at least one structure of formula (I), (Ia), (II) and/or (IIa) are in many cases commercially available, with the starting compounds detailed in the examples being obtainable by known processes, and so reference is made thereto.

These compounds can be reacted with further compounds comprising at least one aromatic or heteroaromatic group by known coupling reactions, the necessary conditions for this purpose being known to the person skilled in the art, and detailed specifications in the examples assisting the person skilled in the art in conducting these reactions.

Particularly suitable and preferred coupling reactions which all lead to C—C bond formations and/or C—N bond formations are those according to BUCHWALD, SUZUKI, YAMAMOTO, STILLE, HECK, NEGISHI, SONOGASHIRA and HIYAMA. These reactions are widely known, and the examples will provide the person skilled in the art with further pointers.

In all the synthesis schemes which follow, the compounds are shown with a small number of substituents to simplify the structures. This does not rule out the presence of any desired further substituents in the processes.

The principles of the preparation processes detailed above are known in principle from the literature for similar compounds and can be adapted easily by the person skilled in the art for the preparation of the compounds of the invention. Further information can be found in the examples.

It is possible by these processes, if necessary followed by purification, for example recrystallization or sublimation, to obtain the compounds of the invention comprising structures of formula (I) in high purity, preferably more than 99% (determined by means of ¹H NMR and/or HPLC).

The compounds of the invention may also have suitable substituents, for example by relatively long alkyl groups (about 4 to 20 carbon atoms), especially branched alkyl groups, or optionally substituted aryl groups, for example xylyl, mesityl or branched terphenyl or quaterphenyl groups, which bring about solubility in standard organic solvents, such that the compounds are soluble at room temperature in toluene or xylene, for example, in sufficient concentration to be able to process the compounds from solution. These soluble compounds are of particularly good suitability for processing from solution, for example by printing methods. It should also be emphasized that the compounds of the invention that comprise at least one structural element of the formulae (I), (Ia), (II), (IIa), a structure of the formulae (IIIa) to (IIIh), (IVa) to (IVh) and/or metal complexes of the invention that comprise at least one substructure of the formulae (2), (2a), (2-1) and/or (2a-1) already have enhanced solubility in these solvents.

In addition, the compounds of the present invention may contain one or more crosslinkable groups. “Crosslinkable group” means a functional group capable of reacting irreversibly. This forms a crosslinked material which is insoluble. The crosslinking can usually be promoted by means of heat or by means of UV radiation, microwave radiation, x-radiation or electron beams. In this case, there is little by-product formation in the crosslinking. In addition, the crosslinkable groups that may be present in the functional compounds crosslink very readily, such that relatively small amounts of energy are required for the crosslinking (for example <200° C. in the case of thermal crosslinking).

Examples of crosslinkable groups are units containing a double bond, a triple bond, a precursor capable of in situ formation of a double or triple bond, or a heterocyclic addition-polymerizable radical. Crosslinkable groups include vinyl, alkenyl, preferably ethenyl and propenyl, C₄₋₂₀-cycloalkenyl, azide, oxirane, oxetane, di(hydrocarbyl)amino, cyanate ester, hydroxyl, glycidyl ether, C₁₋₁₀-alkyl acrylate, C₁₋₁₀-alkyl methacrylate, alkenyloxy, preferably ethenyloxy, perfluoroalkenyloxy, preferably perfluoroethenyloxy, alkynyl, preferably ethynyl, maleimide, cyclobutylphenyl, tri(C₁₋₄)-alkylsiloxy and tri(C₁₋₄)-alkylsilyl. Particular preference is given to cyclobutylphenyl, vinyl and alkenyl.

The compounds of the invention may also be mixed with a polymer. It is likewise possible to incorporate these compounds covalently into a polymer. This is especially possible with compounds substituted by reactive leaving groups such as bromine, iodine, chlorine, boronic acid or boronic ester, or by reactive polymerizable groups such as olefins or oxetanes. These may find use as monomers for production of corresponding oligomers, dendrimers or polymers. The oligomerization or polymerization is preferably effected via the halogen functionality or the boronic acid functionality or via the polymerizable group. It is additionally possible to crosslink the polymers via groups of this kind. The compounds and polymers of the invention may be used in the form of a crosslinked or uncrosslinked layer.

The invention therefore further provides oligomers, polymers or dendrimers containing one or more of the above-detailed structures of the formulae (I), (Ia), (II), (IIa), (IIIa) to (IIIh), (IVa) to (IVh) or compounds or metal complexes of the invention, wherein there are one or more bonds of the compounds or metal complexes of the invention or of the structures of the formulae (I), (Ia), (II), (IIa), (IIIa) to (IIIh), (IVa) to (IVh) to the polymer, oligomer or dendrimer. According to the linkage of the structures of the formulae (I), (Ia), (II), (IIa), (IIIa) bis (IIIh), (IVa) to (IVh) or of the compounds or metal complexes, these therefore form a side chain of the oligomer or polymer or are bonded within the main chain. The polymers, oligomers or dendrimers may be conjugated, partly conjugated or nonconjugated. The oligomers or polymers may be linear, branched or dendritic. For the repeat units of the compounds of the invention in oligomers, dendrimers and polymers, the same preferences apply as described above.

For preparation of the oligomers or polymers, the monomers of the invention are homopolymerized or copolymerized with further monomers. Preference is given to copolymers wherein the units having structural elements of the formulae (I), (Ia), (II), (IIa) or units of formulae (IIIa) to (IIIh), (IVa) to (IVh), or the preferred embodiments recited above and hereinafter, are present to an extent of 0.01 to 99.9 mol %, preferably 5 to 90 mol %, more preferably 20 to 80 mol %. Suitable and preferred comonomers which form the polymer base skeleton are chosen from fluorenes (for example according to EP 842208 or WO 2000/022026), spirobifluorenes (for example according to EP 707020, EP 894107 or WO 2006/061181), paraphenylenes (for example according to WO 92/18552), carbazoles (for example according to WO 2004/070772 or WO 2004/113468), thiophenes (for example according to EP 1028136), dihydrophenanthrenes (for example according to WO 2005/014689), cis- and trans-indenofluorenes (for example according to WO 2004/041901 or WO 2004/113412), ketones (for example according to WO 2005/040302), phenanthrenes (for example according to WO 2005/104264 or WO 2007/017066) or else a plurality of these units. The polymers, oligomers and dendrimers may contain still further units, for example hole transport units, especially those based on triarylamines, and/or electron transport units.

Additionally of particular interest are compounds of the invention which feature a high glass transition temperature. In this connection, preference is given in particular to compounds of the invention that are usable for production of functional layers of electronic devices, preferably compounds comprising at least one structural element of the formulae (I), (Ia), (II), (IIa), a structure of the formulae (IIIa) to (IIIh), (IVa) to (IVh), and/or a metal complex comprising at least one substructure of the formulae (2), (2a), (2-1) and (2-1a), or the preferred embodiments recited above and hereinafter, that have a glass transition temperature of at least 70° C., more preferably of at least 110° C., even more preferably of at least 125° C. and especially preferably of at least 150° C., determined to DIN 51005 (2005-08 version).

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (—)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, a-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, hexamethylindane or mixtures of these solvents.

The present invention therefore further provides a formulation comprising a compound of the invention and at least one further compound. The further compound may, for example, be a solvent, especially one of the abovementioned solvents or a mixture of these solvents. The further compound may alternatively be at least one further organic or inorganic compound which is likewise used in the electronic device, for example an emitting compound, for example a fluorescent dopant, a phosphorescent dopant or a compound that exhibits TADF (thermally activated delayed fluorescence), especially a phosphorescent dopant, and/or a further matrix material. This further compound may also be polymeric. With regard to the metal complexes of the invention, it should be emphasized that these are of course likewise compounds of the invention.

The present invention therefore still further provides a composition comprising a compound of the invention and at least one further organically functional material. Functional materials are generally the organic or inorganic materials introduced between the anode and cathode. Preferably, the organically functional material is selected from the group consisting of fluorescent emitters, phosphorescent emitters, emitters that exhibit TADF (thermally activated delayed fluorescence), host materials, electron transport materials, electron injection materials, hole conductor materials, hole injection materials, electron blocker materials, hole blocker materials, wide bandgap materials and n-dopants.

The present invention therefore also relates to a composition comprising at least one compound of the invention, preferably a compound comprising at least one structural element of the formulae (I), (Ia), (II), (IIa), a structure of the formulae (IIIa) to (IIIh), (IVa) to (IVh), and/or a metal complex comprising at least one substructure of the formulae (2), (2a), (2-1) and (2a-1), or the preferred embodiments recited above and hereinafter, and at least one further matrix material. According to a particular aspect of the present invention, the further matrix material has hole-transporting properties.

The present further provides a composition comprising at least one compound of the invention, preferably a compound comprising at least one structural element of the formulae (I), (Ia), (II), (IIa), a structure of the formulae (IIIa) to (IIIh), (IVa) to (IVh), and/or a metal complex comprising at least one substructure of the formulae (2), (2a), (2-1) and (2a-1), or the preferred embodiments recited above and hereinafter, and at least one wide bandgap material, a wide bandgap material being understood to mean a material in the sense of the disclosure of U.S. Pat. No. 7,294,849. These systems exhibit exceptional advantageous performance data in electroluminescent devices.

Preferably, the additional compound may have a band gap of 2.5 eV or more, preferably 3.0 eV or more, very preferably of 3.5 eV or more. One way of calculating the band gap is via the energy levels of the highest occupied molecular orbital (HOMO) and the lowest unoccupied molecular orbital (LUMO).

Molecular orbitals, especially also the highest occupied molecular orbital (HOMO) and the lowest unoccupied molecular orbital (LUMO), the energy levels thereof and the energy of the lowest triplet state T₁ and that of the lowest excited singlet state ST of the materials are determined via quantum-chemical calculations. For calculation of organic substances without metals, an optimization of geometry is first conducted by the “Ground State/Semi-empirical/Default Spin/AM1/Charge 0/Spin Singlet” method. Subsequently, an energy calculation is effected on the basis of the optimized geometry. This is done using the “TD-SCF/DFT/Default Spin/B3PW91” method with the “6-31 G(d)” basis set (charge 0, spin singlet). For metal-containing compounds, the geometry is optimized via the “Ground State/Hartree-Fock/Default Spin/LanL2 MB/Charge 0/Spin Singlet” method. The energy calculation is effected analogously to the above-described method for the organic substances, except that the “LanL2DZ” basis set is used for the metal atom and the “6-31 G(d)” basis set for the ligands. The HOMO energy level HEh or LUMO energy level LEh is obtained from the energy calculation in Hartree units. This is used to determine the HOMO and LUMO energy levels in electron volts, calibrated by cyclic voltammetry measurements, as follows:

### HOMO(eV)=((HEh*27.212)−0.9899)/1.1206

### LUMO(eV)=((LEh*27.212)−2.0041)/1.385

These values are to be regarded as HOMO and LUMO energy levels of the materials in the context of this application.

The lowest triplet state T₁ is defined as the energy of the triplet state having the lowest energy, which is apparent from the quantum-chemical calculation described.

The lowest excited singlet state ST is defined as the energy of the excited singlet state having the lowest energy, which is apparent from the quantum-chemical calculation described.

The method described herein is independent of the software package used and always gives the same results. Examples of frequently utilized programs for this purpose are “Gaussian09W” (Gaussian Inc.) and Q-Chem4.1 (Q-Chem, Inc.).

The present invention also relates to a composition comprising at least one compound comprising at least one structural element of the formulae (I), (Ia), (II), (IIa) or a structure of the formulae (IIIa) to (IIIh), (IVa) to (IVh), and/or a metal complex comprising at least one substructure of the formulae (2), (2a), (2-1) and (2a-1), or the preferred embodiments recited above and hereinafter, and at least one phosphorescent emitter, the term “phosphorescent emitter” also being understood to mean phosphorescent dopants.

A dopant in a system comprising a matrix material and a dopant is understood to mean that component having the smaller proportion in the mixture. Correspondingly, a matrix material in a system comprising a matrix material and a dopant is understood to mean that component having the greater proportion in the mixture.

Preferred phosphorescent dopants for use in matrix systems, preferably mixed matrix systems, are the preferred phosphorescent dopants specified hereinafter.

The term “phosphorescent dopants” typically encompasses compounds where the emission of light is effected through a spin-forbidden transition, for example a transition from an excited triplet state or a state having a higher spin quantum number, for example a quintet state.

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum. In the context of the present invention, all luminescent compounds containing the abovementioned metals are regarded as phosphorescent compounds.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2016124304, WO 2017032439, WO 2018019687, WO 2018019688, WO 2018041769, WO 2018054798, WO 2018069196, WO 2018069197, WO 2018069273. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Explicit examples of phosphorescent dopants are adduced in the following table:

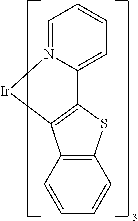

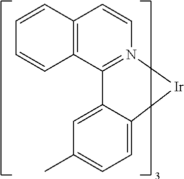

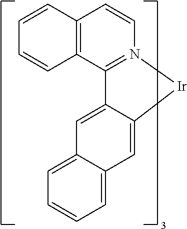

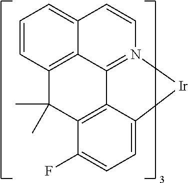

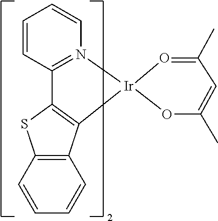

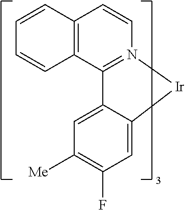

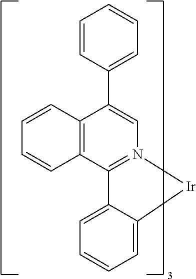

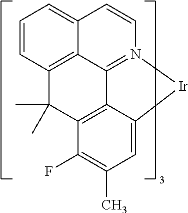

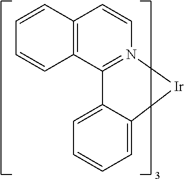

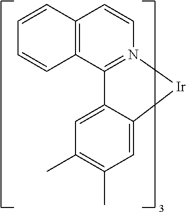

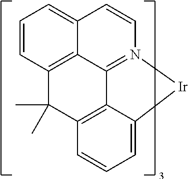

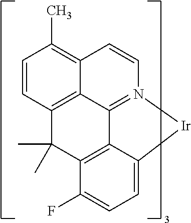

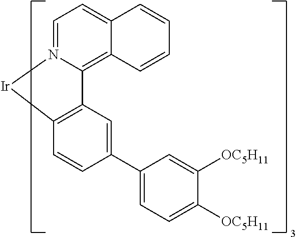

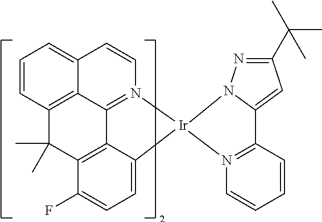

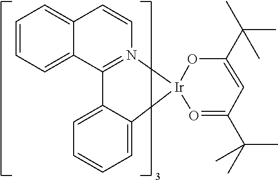

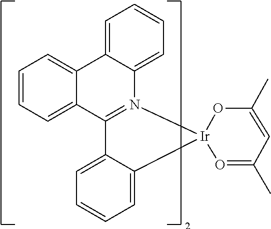

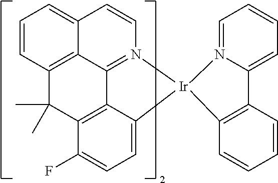

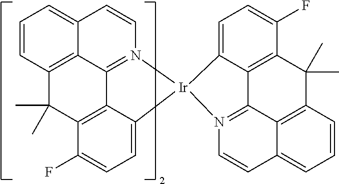

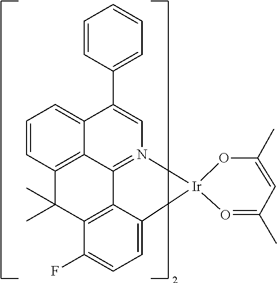

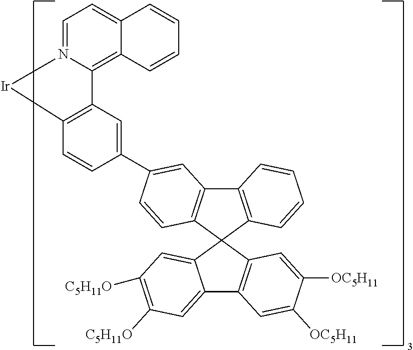

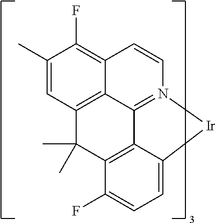

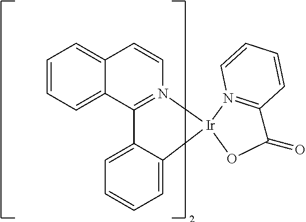

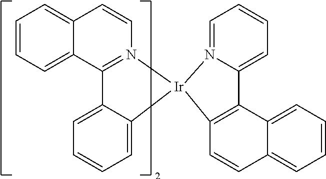

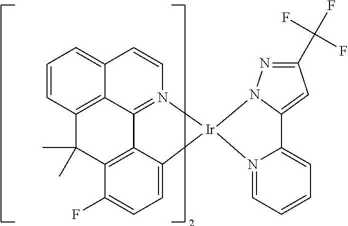

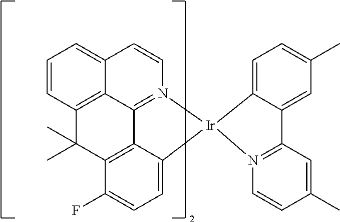

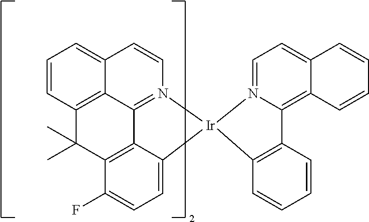

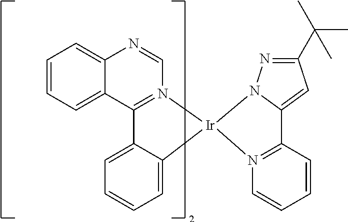

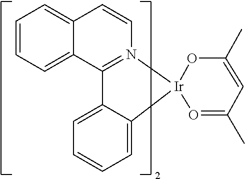

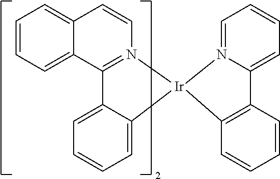

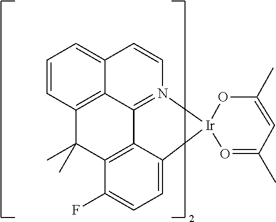

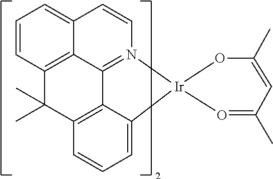

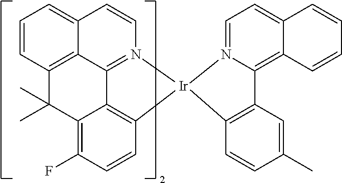

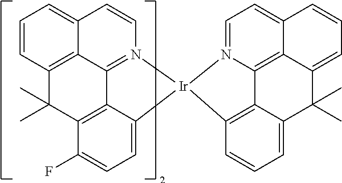

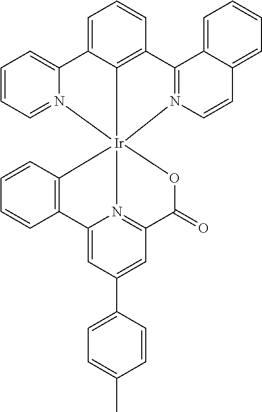

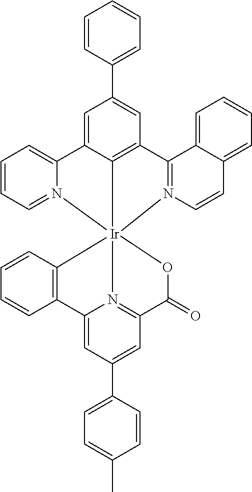

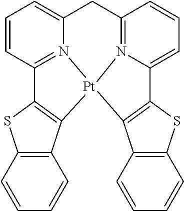

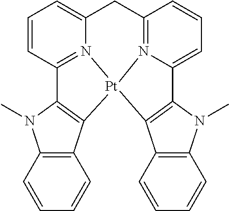

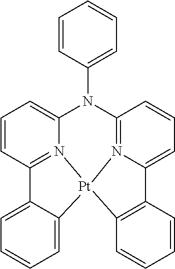

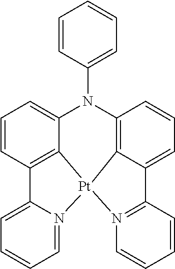

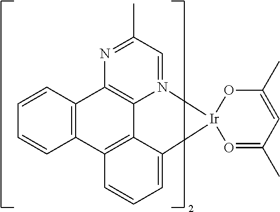

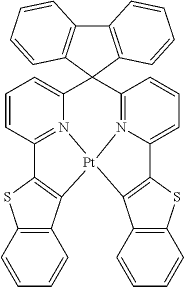

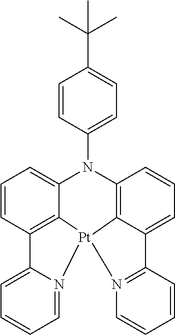

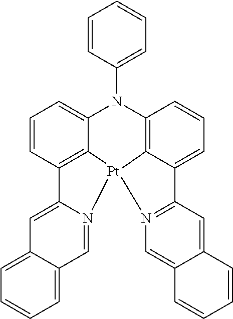

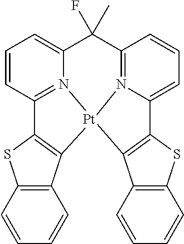

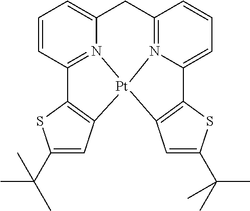

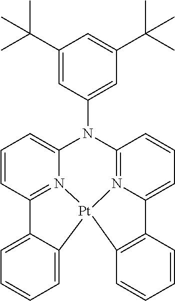

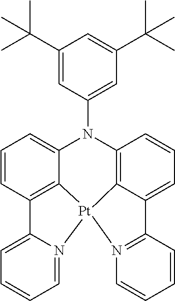

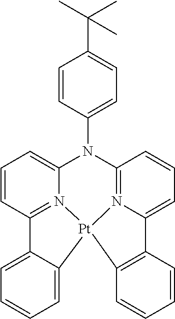

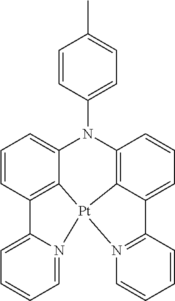

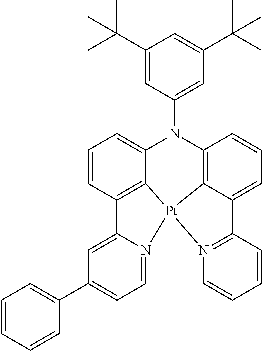

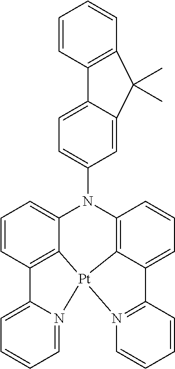

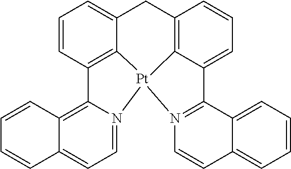

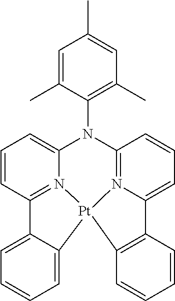

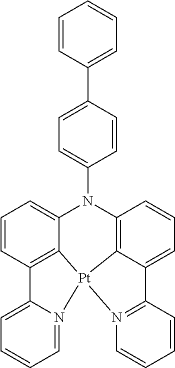

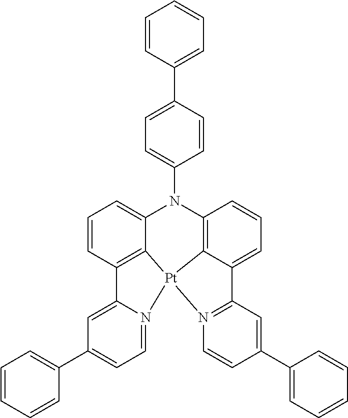

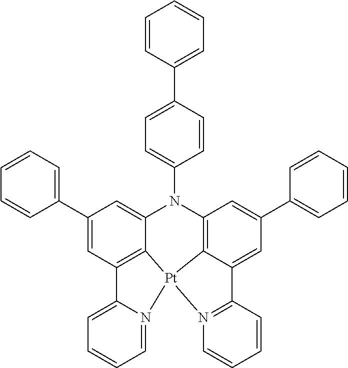

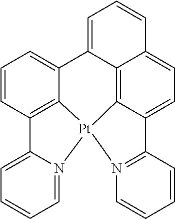

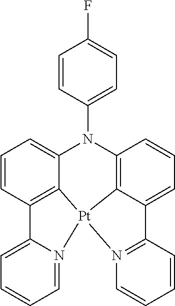

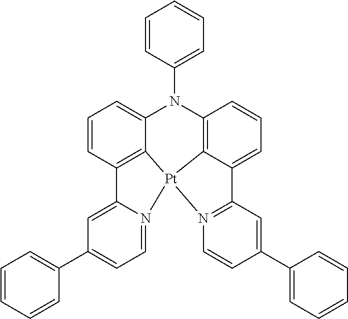

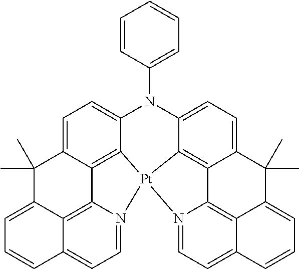

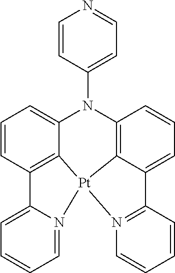

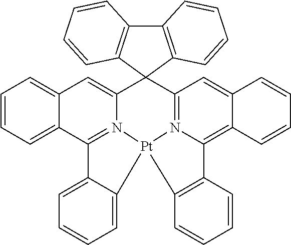

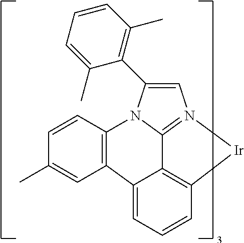

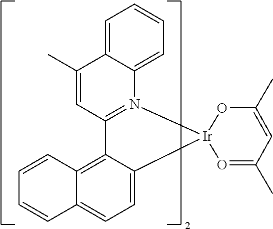

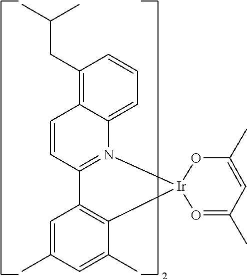

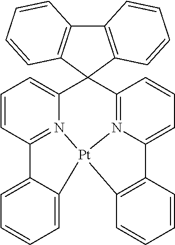

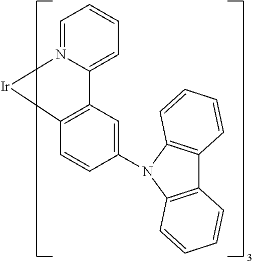

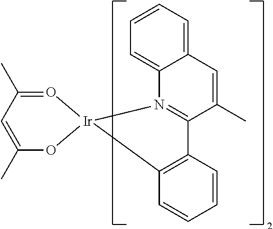

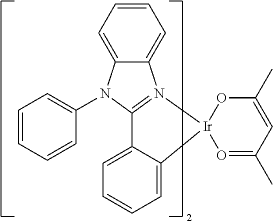

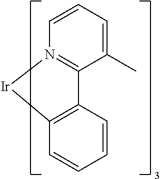

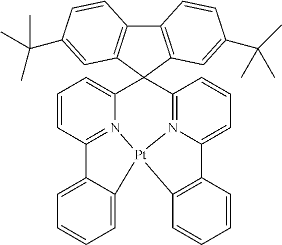

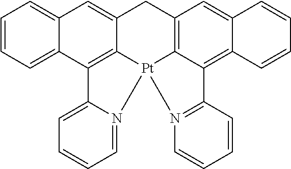

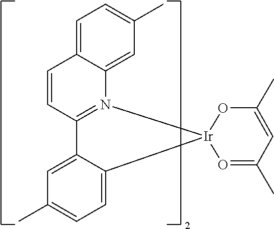

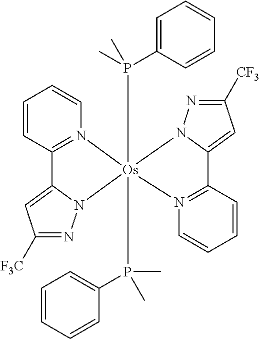

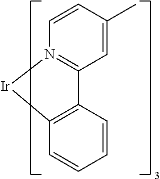

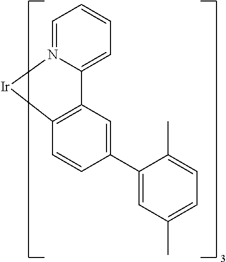

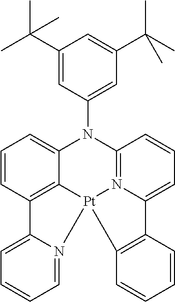

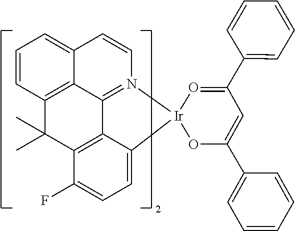

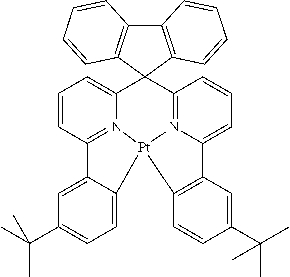

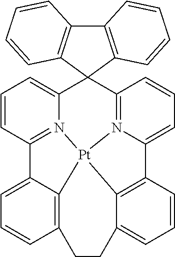

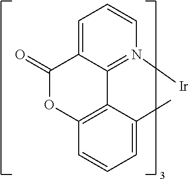

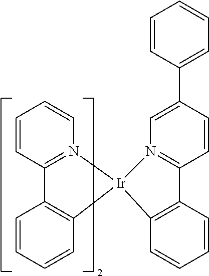

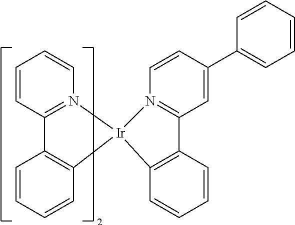

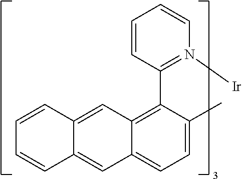

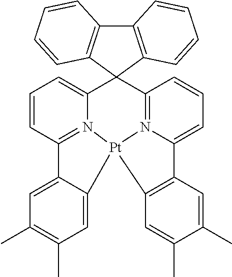

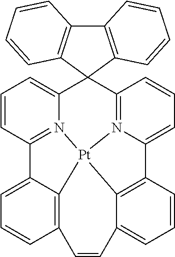

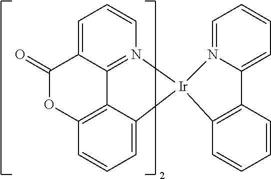

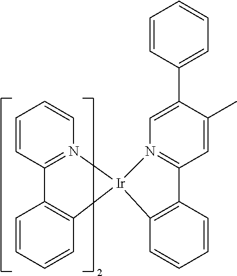

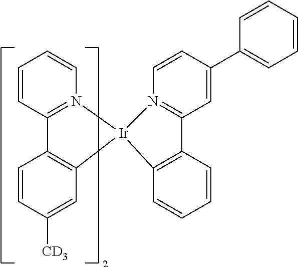

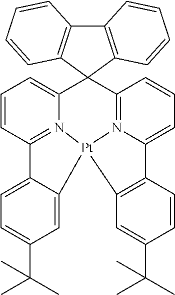

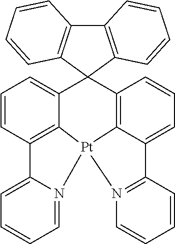

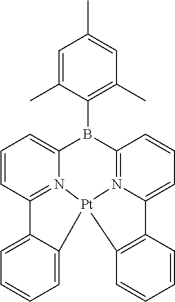

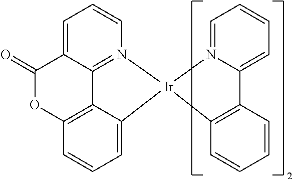

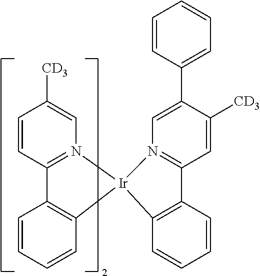

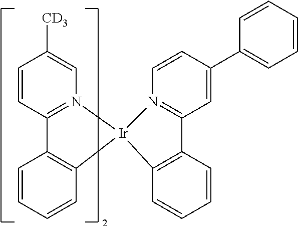

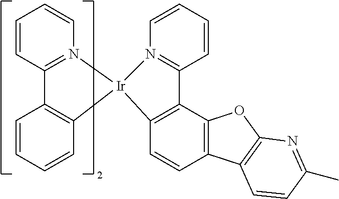

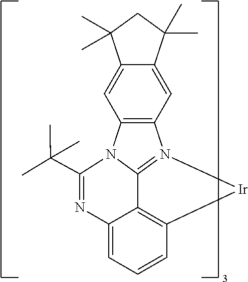

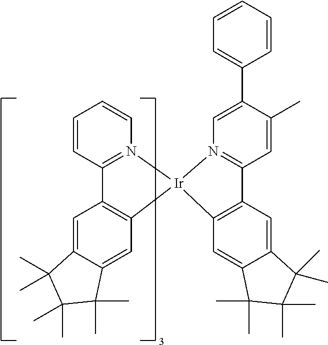

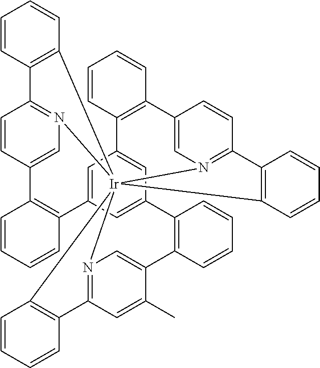

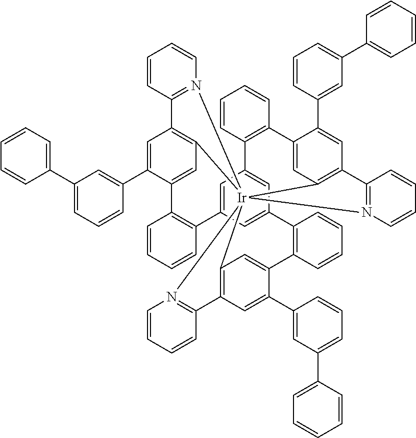

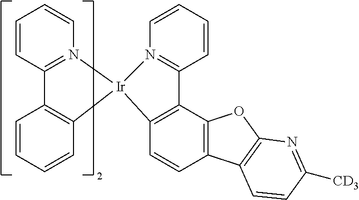

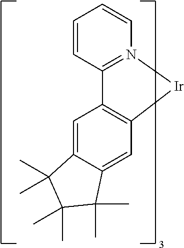

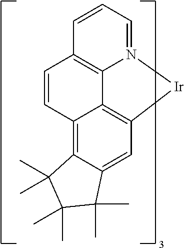

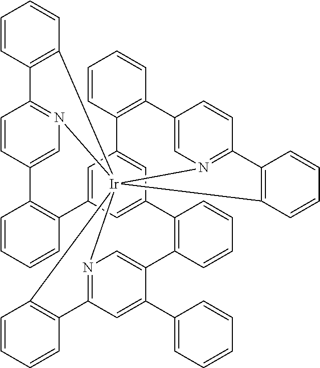

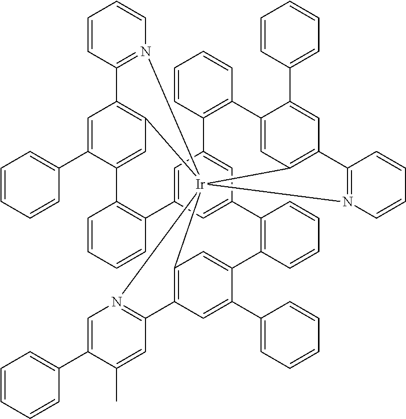

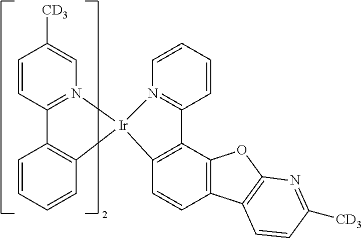

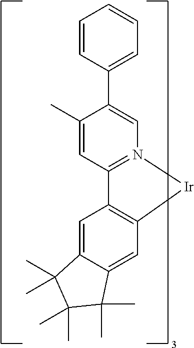

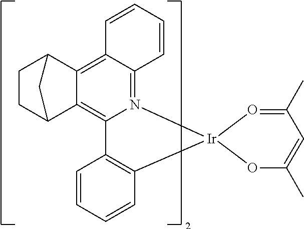

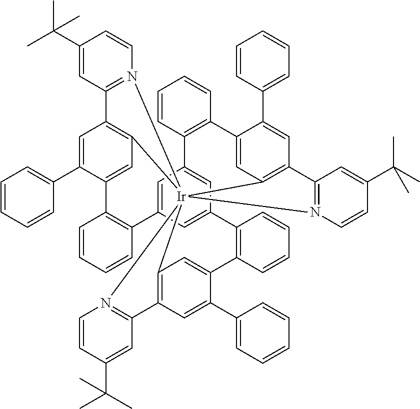

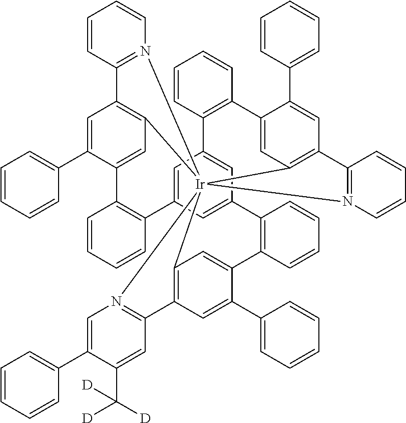

The above-described compounds comprising at least one structural element of the formulae (I), (Ia), (II), (IIa), a structure of the formulae (IIIa) to (IIIh), (IVa) to (IVh), and/or a metal complex comprising at least one substructure of the formulae (2), (2a), (2-1) and (2a-1), or the preferred embodiments adduced above, may preferably be used as active component in an electronic device. An electronic device is understood to mean any device comprising anode, cathode and at least one layer between anode and cathode, said layer comprising at least one organic or organometallic compound. The electronic device of the invention thus comprises anode, cathode and at least one intervening layer containing at least one compound comprising structures of the formula (I). Preferred electronic devices here are selected from the group consisting of organic electroluminescent devices (OLEDs, PLEDs), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs), organic electrical sensors, light-emitting electrochemical cells (LECs), organic laser diodes (O-lasers) and organic plasmon emitting devices (D. M. Koller et al, Nature Photonics 2008, 1-4), preferably organic electroluminescent devices (OLEDs, PLEDs), especially phosphorescent OLEDs, containing at least one compound comprising structures of the formula (I) and/or (Ia) in at least one layer. Particular preference is given to organic electroluminescent devices. Active components are generally the organic or inorganic materials introduced between the anode and cathode, for example charge injection, charge transport or charge blocker materials, but especially emitters and matrix materials.

A preferred embodiment of the invention is organic electroluminescent devices. The organic electroluminescent device comprises cathode, anode and at least one emitting layer. Apart from these layers, it may comprise still further layers, for example in each case one or more hole injection layers, hole transport layers, hole blocker layers, electron transport layers, electron injection layers, exciton blocker layers, electron blocker layers, charge generation layers and/or organic or inorganic p/n junctions. At the same time, it is possible that one or more hole transport layers are p-doped, for example with metal oxides such as MoO₃ or WO₃ or with (per)fluorinated electron-deficient aromatic systems, and/or that one or more electron transport layers are n-doped. It is likewise possible for interlayers to be introduced between two emitting layers, these having, for example, an exciton-blocking function and/or controlling the charge balance in the electroluminescent device. However, it should be pointed out that not necessarily every one of these layers need be present.

In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are three-layer systems where the three layers exhibit blue, green and orange or red emission (for the basic construction see, for example, WO 2005/011013), or systems having more than three emitting layers. Preference is further given to tandem OLEDs as well. The system may also be a hybrid system wherein one or more layers fluoresce and one or more other layers phosphoresce.

In a preferred embodiment of the invention, the organic electroluminescent device contains the compound of the invention, preferably a compound comprising at least one structural element of the formulae (I), (Ia), (II), (IIa) and/or a structure of the formulae (IIIa) to (IIIh), (IVa) to (IVh), or the above-detailed preferred embodiments, as matrix material, preferably as electron-conducting matrix material, in one or more emitting layers, preferably in combination with a further matrix material, preferably a hole-conducting matrix material. In a further preferred embodiment of the invention, the further matrix material is an electron-transporting compound. In yet a further preferred embodiment, the further matrix material is a compound having a large band gap which is not involved to a significant degree, if at all, in the hole and electron transport in the layer. An emitting layer comprises at least one emitting compound.

In a further particularly preferred embodiment of the present invention, an organic electroluminescent device of the invention comprises the compound of the invention, preferably a compound comprising at least one structural element of the formulae (I), (Ia), (II), (IIa) and/or a structure of the formulae (IIIa) to (IIIh), (IVa) to (IVh), or the above-detailed preferred embodiments, in a hole conductor layer or an electron conductor layer.

Suitable matrix materials which can be used in combination with the compounds comprising at least one structural element of the formulae (I), (Ia), (II), (IIa) and/or a structure of the formulae (IIIa) to (IIIh), (IVa) to (IVh), or according to the preferred embodiments, are aromatic ketones, aromatic phosphine oxides or aromatic sulfoxides or sulfones, for example according to WO 2004/013080, WO 2004/093207, WO 2006/005627 or WO 2010/006680, triarylamines, especially monoamines, for example according to WO 2014/015935, carbazole derivatives, e.g. CBP (N,N-biscarbazolylbiphenyl) or the carbazole derivatives disclosed in WO 2005/039246, US 2005/0069729, JP 2004/288381, EP 1205527 or WO 2008/086851, indolocarbazole derivatives, for example according to WO 2007/063754 or WO 2008/056746, indenocarbazole derivatives, for example according to WO 2010/136109 and WO 2011/000455, azacarbazole derivatives, for example according to EP 1617710, EP 1617711, EP 1731584, JP 2005/347160, bipolar matrix materials, for example according to WO 2007/137725, silanes, for example according to WO 005/111172, azaboroles or boronic esters, for example according to WO 2006/117052, triazine derivatives, for example according to WO 2010/015306, WO 2007/063754 or WO 2008/056746, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to US 2009/0136779, WO 2010/050778, WO 2011/042107, WO 2011/088877 or WO 2012/143080, triphenylene derivatives, for example according to WO 2012/048781, lactams, for example according to WO 2011/116865, WO 2011/137951 or WO 2013/064206, 4-spirocarbazole derivatives, for example according to WO 2014/094963 or WO 2015/192939, or dibenzofuran derivatives, for example according to WO 2015/169412, WO 2016/015810, WO 2016/023608 or the as yet unpublished applications EP16158460.2 and EP16159829.7. It is likewise possible for a further phosphorescent emitter which emits at a shorter wavelength than the actual emitter to be present as co-host in the mixture.

Preferred co-host materials are triarylamine derivatives, especially monoamines, indenocarbazole derivatives, 4-spirocarbazole derivatives, lactams and carbazole derivatives.

It may also be preferable to use a plurality of different matrix materials as a mixture, especially at least one electron-conducting matrix material and at least one hole-conducting matrix material. Preference is likewise given to the use of a mixture of a charge-transporting matrix material and an electrically inert matrix material having no significant involvement, if any, in the charge transport, as described, for example, in WO 2010/108579.

It is further preferable to use a mixture of two or more triplet emitters together with a matrix. In this case, the triplet emitter having the shorter-wave emission spectrum serves as co-matrix for the triplet emitter having the longer-wave emission spectrum.

More preferably, a compound of the invention comprising at least one structural element of the formulae (I), (Ia), (II), (IIa) and/or a structure of the formulae (IIIa) to (IIIh), (IVa) to (IVh), in a preferred embodiment, can be used as matrix material in an emission layer of an organic electronic device, especially in an organic electroluminescent device, for example in an OLED or OLEC. In this case, the matrix material containing compound comprising at least one structural element of the formulae (I), (Ia), (II), (IIa) and/or a structure of the formulae (IIIa) to (IIIh), (IVa) to (IVh), or the preferred embodiments recited above and hereinafter, is present in the electronic device in combination with one or more dopants, preferably phosphorescent dopants.

The proportion of the matrix material in the emitting layer in this case is between 50.0% and 99.9% by volume, preferably between 80.0% and 99.5% by volume, and more preferably between 92.0% and 99.5% by volume for fluorescent emitting layers and between 85.0% and 97.0% by volume for phosphorescent emitting layers.

Correspondingly, the proportion of the dopant is between 0.1% and 50.0% by volume, preferably between 0.5% and 20.0% by volume, and more preferably between 0.5% and 8.0% by volume for fluorescent emitting layers and between 3.0% and 15.0% by volume for phosphorescent emitting layers.

An emitting layer of an organic electroluminescent device may also comprise systems comprising a plurality of matrix materials (mixed matrix systems) and/or a plurality of dopants. In this case too, the dopants are generally those materials having the smaller proportion in the system and the matrix materials are those materials having the greater proportion in the system. In individual cases, however, the proportion of a single matrix material in the system may be less than the proportion of a single dopant.

In a further preferred embodiment of the invention, the compounds comprising at least one structural element of the formulae (I), (Ia), (II), (IIa) and/or a structure of the formulae (IIIa) to (IIIh), (IVa) to (IVh), or the preferred embodiments recited above and hereinafter, are used as a component of mixed matrix systems. The mixed matrix systems preferably comprise two or three different matrix materials, more preferably two different matrix materials. Preferably, in this case, one of the two materials is a material having hole-transporting properties and the other material is a material having electron-transporting properties. The desired electron-transporting and hole-transporting properties of the mixed matrix components may, however, also be combined mainly or entirely in a single mixed matrix component, in which case the further mixed matrix component(s) fulfill(s) other functions. The two different matrix materials may be present here in a ratio of 1:50 to 1:1, preferably 1:20 to 1:1, more preferably 1:10 to 1:1 and most preferably 1:4 to 1:1. Preference is given to using mixed matrix systems in phosphorescent organic electroluminescent devices. One source of more detailed information about mixed matrix systems is the application WO 2010/108579.

The present invention further provides an electronic device, preferably an organic electroluminescent device, comprising one or more compounds of the invention and/or at least one oligomer, polymer or dendrimer of the invention in one or more electron-conducting layers, as electron-conducting compound.

Preferred cathodes are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag, in which case combinations of the metals such as Mg/Ag, Ca/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). Likewise useful for this purpose are organic alkali metal complexes, e.g. Liq (lithium quinolinate). The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOx, Al/PtOₓ) may also be preferred. For some applications, at least one of the electrodes has to be transparent or partly transparent in order to enable either the irradiation of the organic material (O—SC) or the emission of light (OLED/PLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers, for example PEDOT, PANI or derivatives of these polymers. It is further preferable when a p-doped hole transport material is applied to the anode as hole injection layer, in which case suitable p-dopants are metal oxides, for example MoO₃ or WO₃, or (per)fluorinated electron-deficient aromatic systems. Further suitable p-dopants are HAT-CN (hexacyanohexaazatriphenylene) or the compound NPD9 from Novaled. Such a layer simplifies hole injection into materials having a low HOMO, i.e. a large HOMO in terms of magnitude.

In the further layers, it is generally possible to use any materials as used according to the prior art for the layers, and the person skilled in the art is able, without exercising inventive skill, to combine any of these materials with the materials of the invention in an electronic device.

The device is correspondingly (according to the application) structured, contact-connected and finally hermetically sealed, since the lifetime of such devices is severely shortened in the presence of water and/or air.

Additionally preferred is an electronic device, especially an organic electroluminescent device, which is characterized in that one or more layers are coated by a sublimation process. In this case, the materials are applied by vapor deposition in vacuum sublimation systems at an initial pressure of typically less than 10⁻⁵ mbar, preferably less than 10⁻⁶ mbar. It is also possible that the initial pressure is even lower or even higher, for example less than 10⁻⁷ mbar.

Preference is likewise given to an electronic device, especially an organic electroluminescent device, which is characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al, Appl. Phys. Lett. 2008, 92, 053301).

Preference is additionally given to an electronic device, especially an organic electroluminescent device, which is characterized in that one or more layers are produced from solution, for example by spin-coating, or by any printing method, for example screen printing, flexographic printing, offset printing or nozzle printing, but more preferably LITI (light-induced thermal imaging, thermal transfer printing) or inkjet printing. For this purpose, soluble compounds are needed, which are obtained, for example, through suitable substitution.

The electronic device, especially the organic electroluminescent device, can also be produced as a hybrid system by applying one or more layers from solution and applying one or more other layers by vapor deposition. For example, it is possible to apply an emitting layer comprising a compound of the invention comprising at least one structural element of the formulae (I), (Ia), (II), (IIa) and/or a structure of the formulae (IIIa) to (IIIh), (IVa) to (IVh), or a metal complex comprising structures of the formulae (2), (2a), (2-1) and/or (2a-1), and a matrix material from solution, and to apply a hole blocker layer and/or an electron transport layer thereto by vapor deposition under reduced pressure.

The person skilled in the art is generally aware of these processes and will be able to apply them without difficulty to electronic devices, especially organic electroluminescent devices comprising compounds of the invention comprising at least one structural element of the formulae (I), (Ia), (II), (IIa) and/or a structure of the formulae (IIIa) to (IIIh), (IVa) to (IVh), or metal complexes comprising structures of the formulae (2), (2a), (2-1) and/or (2a-1), or the preferred embodiments detailed above.

The electronic devices of the invention, especially organic electroluminescent devices, are notable for one or more of the following surprising advantages over the prior art:
1. Electronic devices, especially organic electroluminescent devices, comprising compounds, oligomers, polymers or dendrimers of the invention usable as active compound in an organic electronic device or the preferred embodiments recited above and hereinafter, especially as emitters, electron-conducting materials and/or hole conductor materials or as matrix materials, have a very good lifetime.
2. Electronic devices, especially organic electroluminescent devices, comprising compounds, oligomers, polymers or dendrimers of the invention that are usable as active compound in an organic electronic device, or the preferred embodiments recited above and hereinafter, especially as emitters, electron transport materials, hole conductor materials and/or as host materials, have excellent efficiency. More particularly, efficiency is much higher compared to analogous compounds containing no bullvalene structure. In this context, the effect of the compounds, oligomers, polymers or dendrimers of the invention that are usable as active compound in an organic electronic device, or the preferred embodiments recited above and hereinafter, is a low operating voltage when used in electronic devices. In this context, these compounds especially bring about low roll-off, i.e. a small drop in power efficiency of the device at high luminances.
3. Electronic devices, especially organic electroluminescent devices, comprising compounds, oligomers, polymers or dendrimers usable as active compound in an organic electronic device, or the preferred embodiments recited above and hereinafter, as emitters, electron transport materials, hole conductor materials and/or as host materials, have excellent color purity.
4. The compounds, oligomers, polymers or dendrimers of the invention that are usable as active compound in an organic electronic device, or the preferred embodiments recited above and hereinafter, exhibit very high thermal and photochemical stability and lead to compounds having a very long lifetime.
5. With compounds, oligomers, polymers or dendrimers that are usable as active compound in an organic electronic device, or the preferred embodiments recited above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
6. Compounds, oligomers, polymers or dendrimers that are usable as active compound in an organic electronic device, or the preferred embodiments recited above and hereinafter, have excellent glass film formation.
7. Compounds, oligomers, polymers or dendrimers that are usable as active compound in an organic electronic device, or the preferred embodiments recited above and hereinafter, form very good films from solutions.

These abovementioned advantages are not accompanied by a deterioration in the further electronic properties.

The compounds and mixtures of the invention are suitable for use in an electronic device. An electronic device is understood here to mean a device containing at least one layer containing at least one organic compound. The component may, however, also comprise inorganic materials or else layers formed entirely from inorganic materials.

The present invention therefore further provides for the use of the compounds or mixtures of the invention in an electronic device, especially in an organic electroluminescent device.

The present invention still further provides for the use of a compound of the invention and/or of an oligomer, polymer or dendrimer of the invention in an electronic device as phosphorescent emitter, fluorescent emitter, emitter that exhibits TADF (thermally activated delayed fluorescence), host material, electron transport material, electron injection material, hole conductor material, hole injection material, electron blocker material, hole blocker material and/or wide bandgap material, preferably as emitter, host material, hole conductor material and/or electron transport material.

The present invention still further provides an electronic device comprising at least one of the above-detailed compounds or mixtures of the invention. In this case, the preferences detailed above for the compound also apply to the electronic devices. More preferably, the electronic device is selected from the group consisting of organic electroluminescent devices (OLEDs, PLEDs), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs), organic electrical sensors, light-emitting electrochemical cells (LECs), organic laser diodes (O-lasers) and organic plasmon emitting devices (D. M. Koller et al, Nature Photonics 2008, 1-4), preferably organic electroluminescent devices (OLEDs, PLEDs), especially phosphorescent OLEDs.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In the further layers of the organic electroluminescent device of the invention, it is possible to use any materials as typically used according to the prior art. A person skilled in the art will therefore be able, without exercising inventive skill, to use all materials known for organic electroluminescent devices in combination with the compounds of the invention that are usable for production of functional layers of electronic devices, preferably compounds comprising at least one structural element of the formulae (I), (Ia), (II), (IIa), a structure of the formulae (IIIa) to (IIIh), (IVa) to (IVh), and/or metal complexes comprising structures of the formulae (2), (2a), (2-1) and/or (2a-1), or according to the preferred embodiments.

The compounds of the invention generally have very good properties on use in organic electroluminescent devices. Especially in the case of use of the compounds of the invention in organic electroluminescent devices, the lifetime is significantly better compared to similar compounds according to the prior art. At the same time, the further properties of the organic electroluminescent device, especially the efficiency and voltage, are likewise better or at least comparable.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

It should also be pointed out that many of the features, and especially those of the preferred embodiments of the present invention, should themselves be regarded as inventive and not merely as some of the embodiments of the present invention. For these features, independent protection may be sought in addition to or as an alternative to any currently claimed invention.

The technical teaching disclosed with the present invention may be abstracted and combined with other examples.

The invention is illustrated in more detail by the examples which follow, without any intention of restricting it thereby.

The person skilled in the art will be able to use the details given, without exercising inventive skill, to produce further electronic devices of the invention and hence to execute the invention over the entire scope claimed.

### EXAMPLES

The syntheses which follow, unless stated otherwise, are conducted under a protective gas atmosphere in dried solvents. The metal complexes are additionally handled with exclusion of light or under yellow light. The solvents and reagents can be purchased, for example, from Sigma-ALDRICH or ABCR. The respective figures in square brackets or the numbers quoted for individual compounds relate to the CAS numbers of the compounds known from the literature. In the case of compounds that can display multiple valence-isomeric or tautomeric forms, one valence-isomeric or tautomeric form is shown representatively.

### 1) Synthesis of the Synthons S

### Example S1

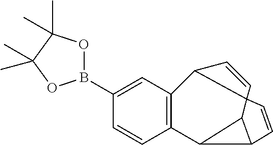 [SMILES: CC1(C)OB(c2ccc3c(c2)C2C=CC24C=CC2C3C24)OC1(C)C]

To 100 ml of n-heptane is added 330 mg (0.5 mmol) of bis[(1,2,5,6-η)-1,5-cyclooctadiene]di-μ-methoxydiiridium(I) [12148-71-9], then 268 mg (1 mmol) of 4,4′-di-tert-butyl-[2,2′]bipyridinyl [72914-19-3] and then 508 mg (2 mmol) of bis(pinacolato)diborane, and the mixture is stirred at room temperature for 15 min. Subsequently, 5.08 g (20 mmol) of bis(pinacolato)diborane [73183-34-3] and then 3.61 g (20 mmol) of 1,1a,4,8b-tetrahydro-1,4-ethenobenzo[a]-cyclopropa[c]cycloheptene [50653-71-9] are added, and the mixture is heated to 80° C. for 12 h. After cooling, 50 ml of ethyl acetate is added to the reaction mixture, which is filtered through a silica gel bed, and the filtrate is concentrated completely under reduced pressure. The crude product is subjected to flash chromatography (Combi-Flash Torrent from A. Semrau). Yield: 2.2 g (7 mmol), 35%; purity: about 95% by ¹H NMR.

The following compounds can be prepared analogously:

| Ex. | Reactants | Product | Yield |
|---|---|---|---|
| S10 S11 | 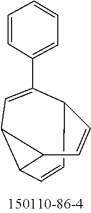 [SMILES: C1=CC2C3C=CC1C(c1ccccc1)=CC23] | 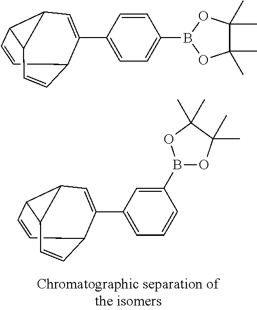 [SMILES: CC1(C)OB(c2ccc(C3=CC4C5C=CC3C=CC54)cc2)OC1(C)C ; CC1(C)OB(c2cccc(C3=CC4C5C=CC3C=CC54)c2)OC1(C)C] | 19% 36% |

### Example S2

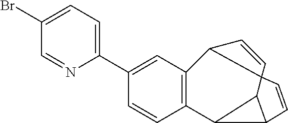 [SMILES: Brc1ccc(-c2ccc3c(c2)C2C=CC24C=CC2C3C24)nc1]

A mixture of 2.37 g (100 mmol) of 2,5-dibromopyridine [624-28-2], 3.06 g (10 mmol) of S1, 2.76 g (20 mmol) of potassium carbonate, 10 g of glass beads (diameter 3 mm), 52.6 mg (0.2 mmol) of triphenylphosphine, 22.5 mg (0.1 mmol) of palladium(II) acetate, 30 ml of acetonitrile and 15 ml of methanol is heated to 60° C. while stirring for 16 h. After cooling, the solvent is largely removed under reduced pressure, and the residue is taken up in 100 ml of ethyl acetate, washed three times with 30 ml each time of 3% by weight aqueous acetylcysteine solution, three times with 30 ml each time of water and once with 30 ml of saturated sodium chloride solution, and dried over magnesium sulfate. The desiccant is filtered off, the filtrate is concentrated to dryness and the solids are recrystallized from acetonitrile. Yield: 2.12 g (6.3 mmol), 63%; purity: about 95% by ¹H NMR.

### Example S3

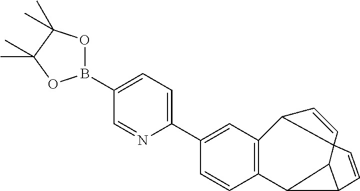 [SMILES: CC1(C)OB(c2ccc(-c3ccc4c(c3)C3C=CC35C=CC3C4C35)nc2)OC1(C)C]

To a mixture of 3.36 g (10 mmol) of S2, 2.80 g (11 mmol) of bis(pinacolato)diborane, 2.94 g (30 mmol) of potassium acetate (anhydrous), 10 g of glass beads (diameter 3 mm) and 50 ml of THF are added, with good stirring, 56.1 mg (0.2 mmol) of tricyclohexylphosphine and then 22.5 mg (0.11 mmol) of palladium(II) acetate, and the mixture is heated under gentle reflux for 16 h. After cooling, the salts and glass beads are removed by suction filtration through a Celite bed in the form of a THF slurry, which is washed through with a little THF, and the filtrate is concentrated to dryness. The residue is taken up in 50 ml of ethyl acetate, washed three times with 30 ml each time of 3% by weight aqueous acetylcysteine solution, three times with 30 ml each time of water and once with 30 ml of saturated sodium chloride solution, and dried over magnesium sulfate. The desiccant is filtered off, the filtrate is concentrated to dryness, the solids are stirred with 20 ml of warm methanol, and the crystallized product is filtered off with suction, washed twice with 5 ml each time of methanol and dried under reduced pressure. Yield: 2.49 g (6.5 mmol), 65%; purity: about 95% by ¹H NMR.

### Example S4

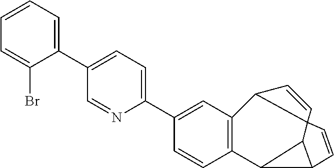 [SMILES: Brc1ccccc1-c1ccc(-c2ccc3c(c2)C2C=CC24C=CC2C3C24)nc1]

To a mixture of 3.83 g (10 mmol) of S3, 2.83 g (10 mmol) of 1-bromo-2-iodobenzene [583-55-1], 3.18 g (30 mmol) of sodium carbonate, 25 ml of toluene, 10 ml of ethanol and 25 ml of water are added, with very good stirring, 78.8 mg (0.3 mmol) of triphenylphosphine and then 22.5 mg (0.1 mmol) of palladium(II) acetate, and the mixture is heated to 75° C. for 48 h. After cooling, the organic phase is separated off, washed three times with 30 ml each time of 3% by weight aqueous acetylcysteine solution, three times with 30 ml each time of water and once with 30 ml of saturated sodium chloride solution, and dried over magnesium sulfate. The desiccant is filtered off and the filtrate is concentrated fully under reduced pressure. The residue is subjected to flash chromatography (CombiFlash Torrent from A. Semrau). Yield: 1.65 g (4 mmol), 40%; purity: about 97% by ¹H NMR.

### Example S5

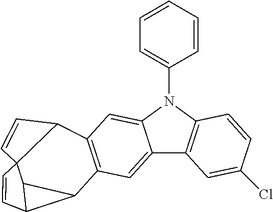 [SMILES: Clc1ccc2c(c1)c1cc3c(cc1n2-c1ccccc1)C1C=CC12C=CC1C3C12]

A well-stirred mixture of 2.83 g (10 mmol) of (2-bromo-4-chlorophenyl)phenylamine [2149611-39-0], 3.06 g (10 mmol) of S1, 6.37 g (30 mmol) of tripotassium phosphate, 183 mg (0.6 mmol) of tri-o-tolylphosphine, 22.5 mg (0.1 mmol) of palladium(II) acetate, 40 ml of toluene, 10 ml of dioxane and 40 ml of water is heated under reflux for 16 h. After cooling, the aqueous phase is separated off, washed three times with 30 ml each time of 3% by weight aqueous acetylcysteine solution, three times with 30 ml each time of water and once with 30 ml of saturated sodium chloride solution, and dried over magnesium sulfate. The desiccant is filtered off using a silica gel bed in the form of a toluene slurry, and the filtrate is concentrated to dryness. The residue is recrystallized from acetonitrile with addition of a little acetone. The secondary amine thus obtained is dissolved in 50 ml of DMAc (dimethylacetamide), 4.54 g (25 mmol) of copper(II) acetate and 22.5 mg (0.1 mmol) of palladium(II) acetate are added, and the mixture is stirred at 140° C. for 4 h. The DMAc is largely removed under reduced pressure, the residue is taken up in 100 ml of DCM, 30 ml of cone, ammonia solution is added, the mixture is stirred at room temperature for 1 h, and the organic phase is separated off and washed three times with 30 ml of cone, ammonia solution, three times with 30 ml each time of 3% by weight aqueous acetylcysteine solution, three times with 30 ml each time of water and once with 30 ml of saturated sodium chloride solution, and dried over magnesium sulfate. The magnesium sulfate is filtered off using a silica gel bed in the form of a DCM slurry, and the filtrate is concentrated to dryness and the residue is subjected to flash chromatography (CombiFlash Torrent from A. Semrau). Yield: 1.25 g (3.3 mmol) 33%. Purity by ¹H NMR about 95%.

### 2) Synthesis of the Ligands L

The following compounds can be prepared analogously to example S2:

| Ex. | Reactants | Product | Yield |
|---|---|---|---|
| L1 | 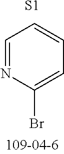 | 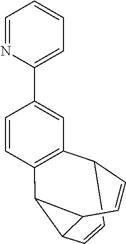 [SMILES: C1=CC2C3C=CC1c1cc(-c4ccccn4)ccc1C23] | 47% |
| L2 | 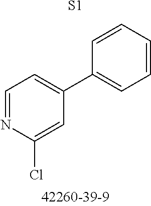 | 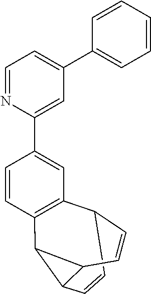 [SMILES: C1=CC2C3C=CC1c1cc(-c4cc(-c5ccccc5)ccn4)ccc1C23] | 33% |
| L3 | 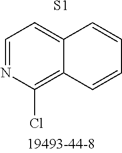 | 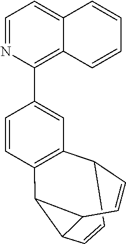 [SMILES: C1=CC2C3C=CC1c1cc(-c4nccc5ccccc45)ccc1C23] | 35% |
| L4 | 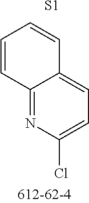 | 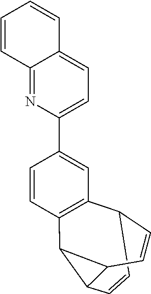 [SMILES: C1=CC2C3C=CC1c1cc(-c4ccc5ccccc5n4)ccc1C23] | 38% |

### Example L5

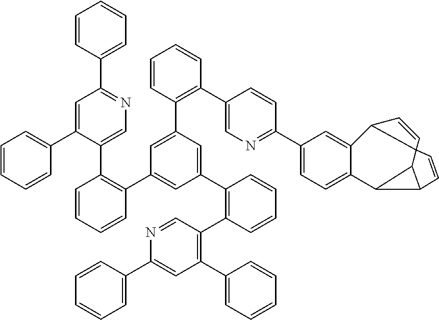 [SMILES: C1=CC23C=CC2c2cc(-c4ccc(-c5ccccc5-c5cc(-c6ccccc6-c6cnc(-c7ccccc7)cc6-c6ccccc6)cc(-c6ccccc6-c6cnc(-c7ccccc7)cc6-c6ccccc6)c5)cn4)ccc2C2C1C23]

To a mixture of 8.15 g (10 mmol) of 3,3′-[5′-(4,4,5,5-tetramethyl-1,3,2-dioxaborolan-2-yl)[1,1′:3′,1″-terphenyl]-2,2″-diyl]bis[4,6-diphenylpyridine [1989597-74-1], 4.12 g (10 mmol) of S4, 6.37 g (30 mmol) of tripotassium phosphate, 30 ml of toluene, 15 ml of dioxane and 30 ml of water are added, with good stirring, 164 mg (0.4 mmol) of SPhos and then 44.9 mg (0.2 mmol) of palladium(II) acetate, and then the mixture is heated under reflux for 24 h. After cooling, the organic phase is separated off, washed three times with 30 ml each time of 3% by weight aqueous acetylcysteine solution, three times with 30 ml each time of water and once with 30 ml of saturated sodium chloride solution, and dried over magnesium sulfate. The desiccant is filtered off, the filtrate is concentrated to dryness under reduced pressure and the glassy crude product is recrystallized at boiling from acetonitrile (˜10 ml) with addition of ethyl acetate (˜2 ml). Yield: 4.77 g (4.6 mmol), 46%; purity: about 95% by ¹H NMR.

The following compounds can be prepared analogously:

| Ex. | Reactants | Product | Yield |
|---|---|---|---|
| L6 | 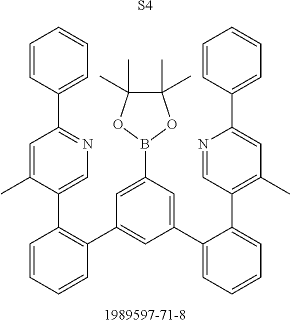 | 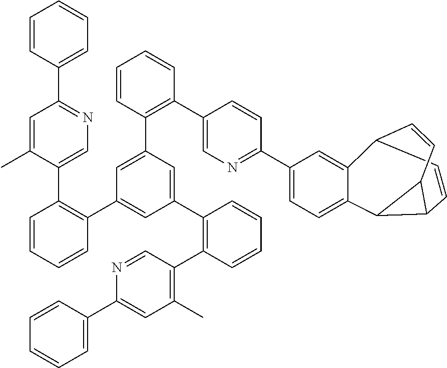 [SMILES: Cc1cc(-c2ccccc2)ncc1-c1ccccc1-c1cc(-c2ccccc2-c2ccc(-c3ccc4c(c3)C3C=CC5C(C=C3)C45)nc2)cc(-c2ccccc2-c2cnc(-c3ccccc3)cc2C)c1] | 40% |
| L7 | 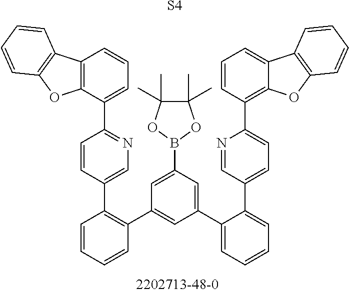 [SMILES: "S4" ; CC1(C)OB(c2cc(-c3ccccc3-c3ccc(-c4cccc5c4oc4ccccc45)nc3)cc(-c3ccccc3-c3ccc(-c4cccc5c4oc4ccccc45)nc3)c2)OC1(C)C] | 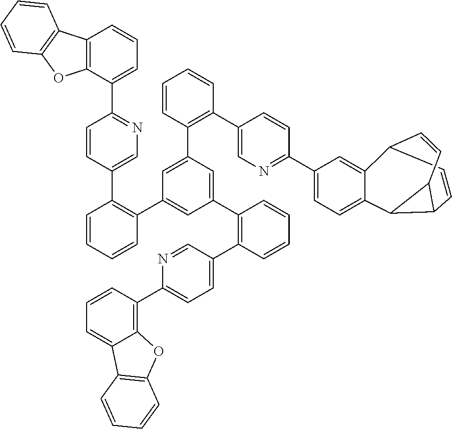 [SMILES: C1=CC2C3C=CC1c1cc(-c4ccc(-c5ccccc5-c5cc(-c6ccccc6-c6ccc(-c7cccc8c7oc7ccccc78)nc6)cc(-c6ccccc6-c6ccc(-c7cccc8c7oc7ccccc78)nc6)c5)cn4)ccc1C23] | 42% |

### Example L8

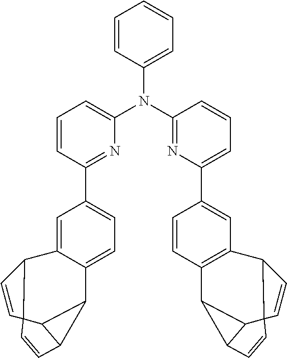 [SMILES: C1=CC23C=CC2c2cc(-c4cccc(N(c5ccccc5)c5cccc(-c6ccc7c(c6)C6C=CC68C=CC6C7C68)n5)n4)ccc2C2C1C23]

Preparation analogous to L1, except replacing 10 mmol of 2-bromopyridine [109-04-6] with 2.03 g (5 mmol) of 6-bromo-N-(6-bromo-2-pyridinyl)-N-phenyl-2-pyridinamine [894405-86-8], Yield: 1.75 g (2.8 mmol), 56%; purity: about 95% by ¹H NMR.

The following compounds can be prepared analogously:

| Ex. | Reactants | Product | Yield |
|---|---|---|---|
| L9 | 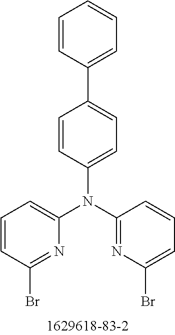 [SMILES: Brc1cccc(N(c2ccc(-c3ccccc3)cc2)c2cccc(Br)n2)n1] | 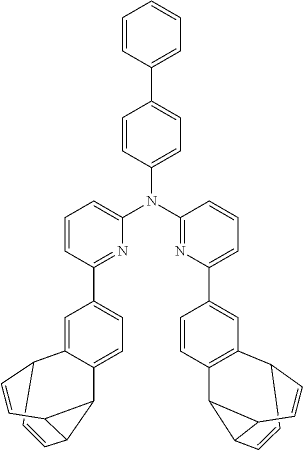 [SMILES: C1=CC2C3C=CC1c1cc(-c4cccc(N(c5ccc(-c6ccccc6)cc5)c5cccc(-c6ccc7c(c6)C6C=CC8C(C=C6)C78)n5)n4)ccc1C23] | 52% |
| L10 | 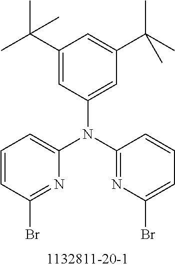 [SMILES: CC(C)(C)c1cc(N(c2cccc(Br)n2)c2cccc(Br)n2)cc(C(C)(C)C)c1] | 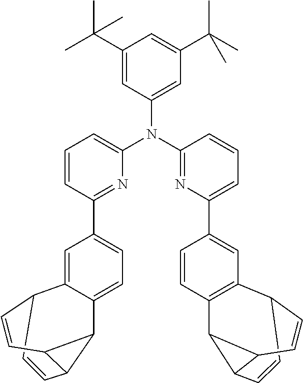 [SMILES: CC(C)(C)c1cc(N(c2cccc(-c3ccc4c(c3)C3C=CC5C(C=C3)C45)n2)c2cccc(-c3ccc4c(c3)C3C=CC5C(C=C3)C45)n2)cc(C(C)(C)C)c1] | 50% |
| L11 | 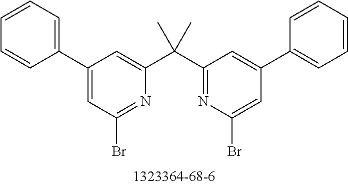 [SMILES: CC(C)(c1cc(-c2ccccc2)cc(Br)n1)c1cc(-c2ccccc2)cc(Br)n1] | 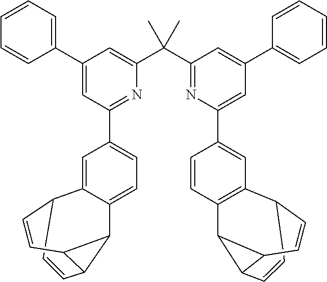 [SMILES: CC(C)(c1cc(-c2ccccc2)cc(-c2ccc3c(c2)C2C=CC4C(C=C2)C34)n1)c1cc(-c2ccccc2)cc(-c2ccc3c(c2)C2C=CC4C(C=C2)C34)n1] | 47% |
| L12 | 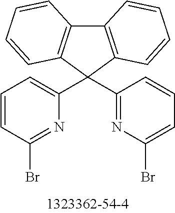 [SMILES: Brc1cccc(C2(c3cccc(Br)n3)c3ccccc3-c3ccccc32)n1] | 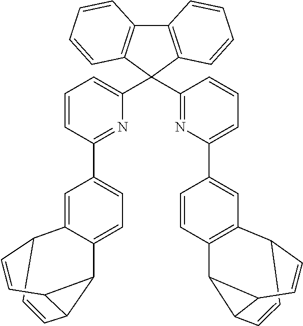 [SMILES: C1=CC2C3C=CC1c1cc(-c4cccc(C5(c6cccc(-c7ccc8c(c7)C7C=CC9C(C=C7)C89)n6)c6ccccc6-c6ccccc65)n4)ccc1C23] | 45% |

### 3) Synthesis of the Carbazoles, Amines, Triazines

### Example C1

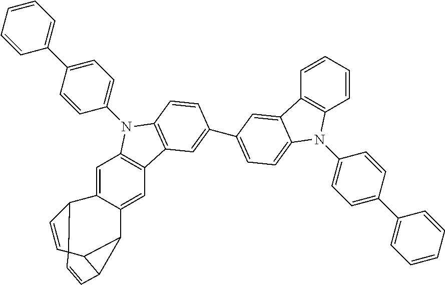 [SMILES: C1=CC23C=CC2c2cc4c(cc2C2C1C23)c1cc(-c2ccc3c(c2)c2ccccc2n3-c2ccc(-c3ccccc3)cc2)ccc1n4-c1ccc(-c2ccccc2)cc1]

A well-stirred mixture of 3.80 g (10 mmol) of S5, 3.69 g (10 mmol) of 9-phenyl-3-(4,4,5,5-tetramethyl-1,3,2-dioxaborolan-2-yl)-9H-carbazole [1126522-69-7], 6.37 g (30 mmol) of tripotassium phosphate, 183 mg (0.6 mmol) of tri-o-tolylphosphine, 22.5 mg (0.1 mmol) of palladium(II) acetate, 40 ml of toluene, 10 ml of dioxane and 40 ml of water is heated under reflux for 16 h. After cooling, the aqueous phase is separated off and the organic phase is concentrated to dryness. The residue is taken up in 50 ml of DCM, washed three times with 30 ml each time of 3% by weight aqueous acetylcysteine solution, three times with 30 ml each time of water and once with 30 ml of saturated sodium chloride solution, and dried over magnesium sulfate. The desiccant is filtered off using a silica gel bed in the form of a DCM slurry, and the filtrate is concentrated to dryness. The residue is extracted by stirring with hot butyl acetateliso-propanol, then extracted three times with hot toluene and purified by heating under reduced pressure (p ˜ 10⁻⁵ mbar, T ˜ 280° C.). Yield: 3.40 g (4.5 mmol) 45%. Purity by HPLC >99.9%.

The following compounds can be prepared analogously:

| Ex. | Reactants | Product | Yield |
|---|---|---|---|
| C2 | 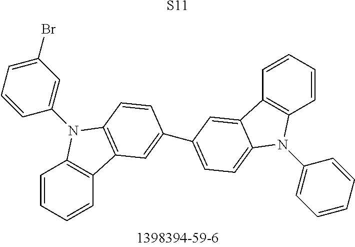 | 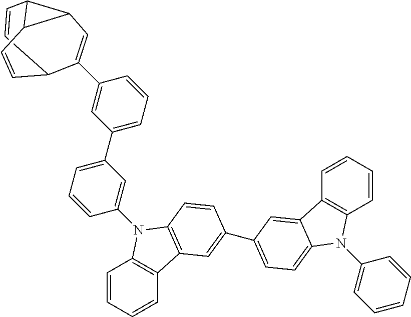 [SMILES: C1=CC2C3C=CC1C(c1cccc(-c4cccc(-n5c6ccccc6c6cc(-c7ccc8c(c7)c7ccccc7n8-c7ccccc7)ccc65)c4)c1)=CC23] | 38% |
| A1 | 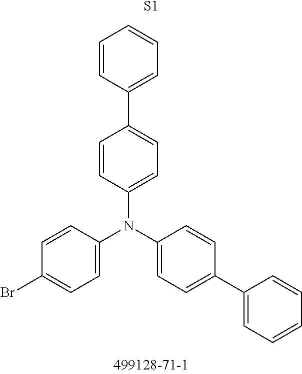 | 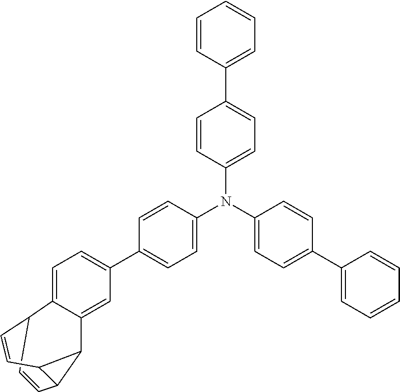 [SMILES: C1=CC2C3C=CC1c1ccc(-c4ccc(N(c5ccc(-c6ccccc6)cc5)c5ccc(-c6ccccc6)cc5)cc4)cc1C23] | 48% |
| T1 | 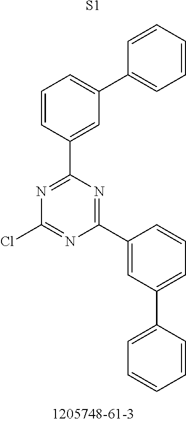 [SMILES: "S1" ; Clc1nc(-c2cccc(-c3ccccc3)c2)nc(-c2cccc(-c3ccccc3)c2)n1] | 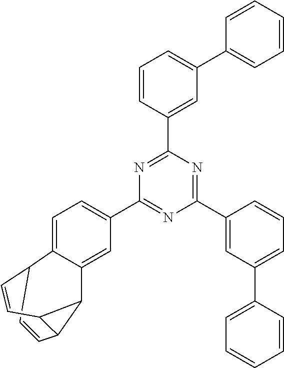 [SMILES: C1=CC2C3C=CC1c1ccc(-c4nc(-c5cccc(-c6ccccc6)c5)nc(-c5cccc(-c6ccccc6)c5)n4)cc1C23] | 44% |
| T2 | 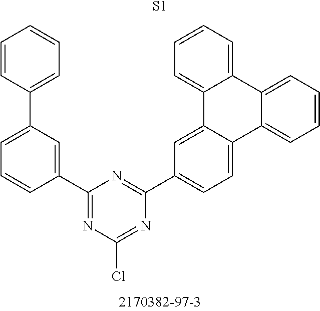 | 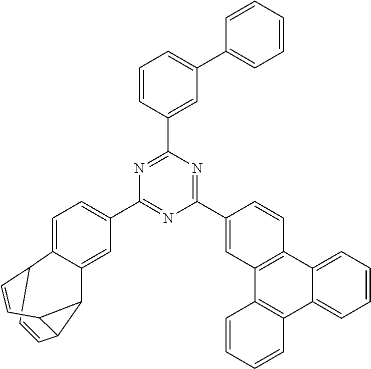 [SMILES: C1=CC2C3C=CC1c1ccc(-c4nc(-c5cccc(-c6ccccc6)c5)nc(-c5ccc6c7ccccc7c7ccccc7c6c5)n4)cc1C23] | 42% |
| T3 | 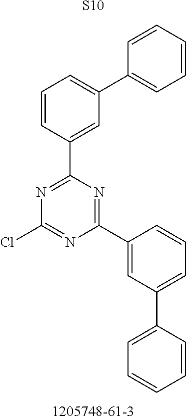 [SMILES: "S10" ; Clc1nc(-c2cccc(-c3ccccc3)c2)nc(-c2cccc(-c3ccccc3)c2)n1] | 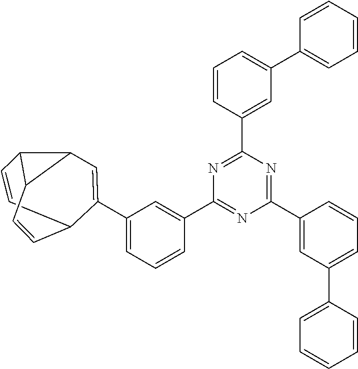 [SMILES: C1=CC2C3C=CC1C(c1cccc(-c4nc(-c5cccc(-c6ccccc6)c5)nc(-c5cccc(-c6ccccc6)c5)n4)c1)=CC23] | 35% |

### 4) Synthesis of the C3-Symmetric Iridium Complexes of Bidentate Ligands L

### Example Ir(L1)₃

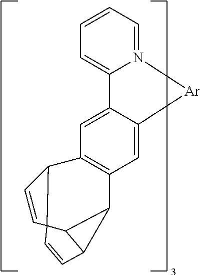

Preparation according to WO 2015/104045, Ir(LB₇₄)₃, see page 179. Yield: 41%; purity: >99.9% by HPLC.

The following compounds can be prepared analogously:

| Ex. | Ligand | Product | Yield |
|---|---|---|---|
| Ir(L2)₃ | L2 | 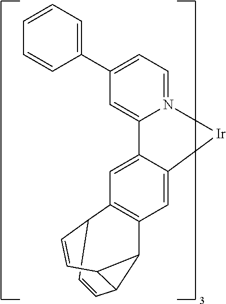 | 30% |
| Ir(L3)₃ | L3 | 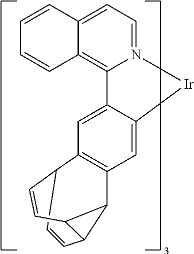 | 35% |

### 5) Synthesis of the C2-Symmetric N,N-Trans-Iridium Complexes of Bidentate Ligands L

### Example Ir(L4)₂(acac)

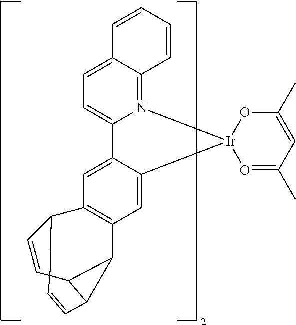

Preparation according to WO 2015/104045. First of all, the C1 dimer is prepared analogously to [Ir(L42)₂Cl)₂, see page 193. This is reacted with acetylacetone analogously to Ir538; see page 218. Yield over two stages: 39%; purity: >99.9% by HPLC.

### 6) Synthesis of the Tripodal Iridium Complexes

### Example IrL5

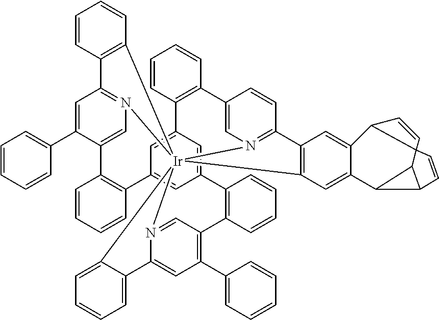

Preparation according to WO 2016/124304, Ir(L1) variant A, see page 218. Use of ligand L5. Purification as described therein by chromatography on silica gel, hot extraction three times with DCM/methanol (1:1, w) and three times with DCM/acetonitrile (2:1, w) and heating at T ˜ 200° C. and p ˜10⁻⁶ mbar. Yield: 38%; purity: >99.9% by HPLC.

The following compounds can be prepared analogously:

| Ex. | Ligand | Product | Yield |
|---|---|---|---|
| IrL6 | L6 | 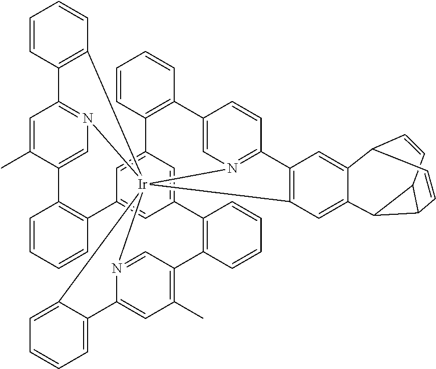 | 33% |
| IrL7 | L7 | 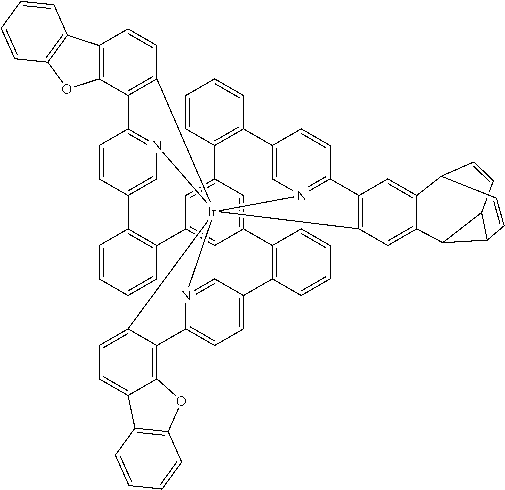 | 34% |

### 7) Synthesis of the Platinum Complexes

### Example PtL8

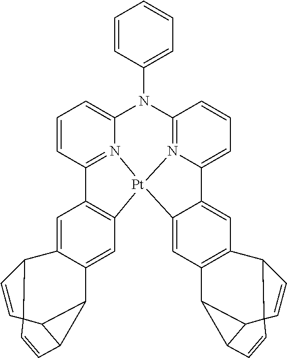

A mixture of 1.86 g (3.0 mmol) of L5 and 1.14 g (3.5 mmol) of dimethyl-bis-DMSO-platinum(II) [70423-98-2] in 30 ml of DMSO is heated to 70° C. for 24 h. The reaction mixture is allowed to cool, the DMSO is largely removed under reduced pressure, and the black residue is taken up in 50 ml of DCM and chromatographed with DCM on silica gel. The red core fraction is extracted, 50 ml of methanol is added, and DCM is distilled off at 50° C. The crystallized product is filtered off with suction and washed twice with 20 ml each time of MeOH. Further purification is effected by hot extraction, once with DCM/methanol (1:1, vv) and three times with DCM/acetonitrile (2:1, w), and heating at T ˜ 250° C. and p ˜ 10⁻⁶ mbar. Yield: 782 mg (0.96), 32%; purity: >99.9% by HPLC.

The following compounds can be prepared analogously:

| Ex. | Ligand | Product | Yield |
|---|---|---|---|
| PtL9 | L9 | 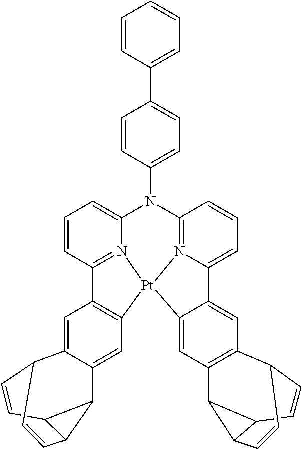 | 27% |
| PtL10 | L10 | 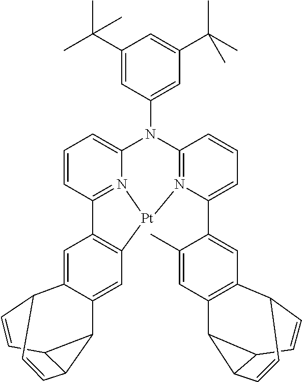 | 25% |
| PtL11 | L11 | 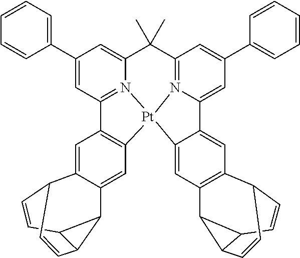 | 20% |
| PtL12 | L12 | 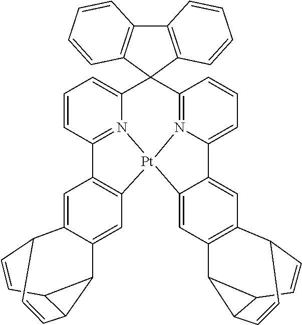 | 26% |

### Solution-Processed Devices:

### From Soluble Functional Materials of Low Molecular Weight

The compounds of the invention may also be processed from solution and lead therein to OLEDs which are much simpler in terms of process technology compared to the vacuum-processed OLEDs, but nevertheless have good properties. The production of such components is based on the production of polymeric light-emitting diodes (PLEDs), which has already been described many times in the literature (for example in WO 2004/037887). The structure is composed of substrate/ITO/hole injection layer (60 nm)/interlayer (20 nm)/emission layer (60 nm)/hole blocker layer (10 nm)/electron transport layer (40 nm)/cathode. For this purpose, substrates from Technoprint (soda-lime glass) are used, to which the ITO structure (indium tin oxide, a transparent conductive anode) is applied. The substrates are cleaned in a cleanroom with D1 water and a detergent (Deconex 15 PF) and then activated by a UV/ozone plasma treatment. Thereafter, likewise in a cleanroom, a 20 nm hole injection layer (PEDOT:PSS from Clevios™) is applied by spin-coating. The required spin rate depends on the degree of dilution and the specific spin-coater geometry. In order to remove residual water from the layer, the substrates are baked on a hotplate at 200° C. for 30 minutes. The interlayer used serves for hole transport; in this case, HL-X from Merck is used. The interlayer may alternatively also be replaced by one or more layers which merely have to fulfill the condition of not being leached off again by the subsequent processing step of EML deposition from solution. For production of the emission layer, the triplet emitters of the invention are dissolved together with the matrix materials in toluene or chlorobenzene. The typical solids content of such solutions is between 16 and 25 g/l when, as here, the layer thickness of 60 nm which is typical of a device is to be achieved by means of spin-coating. The solution-processed devices contain an emission layer composed of Matrix1:Matrix2:Ir(L) with the percentages specified. The emission layer is spun on in an inert gas atmosphere, argon in the present case, and baked at 160° C. for 10 min. Vapor-deposited atop the latter are the hole blocker layer (10 nm RETM1) and the electron transport layer (40 nm RETM1 (50%)/RETM2 (50%)) (vapor deposition systems from Lesker or the like, typical vapor deposition pressure 5×10⁻⁶ mbar). Finally, a cathode of aluminum (100 nm) (high-purity metal from Aldrich) is applied by vapor deposition. In order to protect the device from air and air humidity, the device is finally encapsulated and then characterized. The OLED examples cited are yet to be optimized; table 1 summarizes the data obtained. The lifetime LT50 is defined as the time after which the luminance in operation drops to 50% of the starting luminance with a starting brightness of 1000 cd/m². Table 2 shows the materials used.

TABLE 1
Results with materials processed from solution

| | | | Voltage | | |
|---|---|---|---|---|---|
| | Matrix1 | EQE (%) | (V) | | LT50 (h) |
| | Matrix2 | 1000 | 1000 | | 1000 |
| Ex. | Emitter | cd/m² | cd/m² | CIE x/y | cd/m² |
| Sol-D1 | TMM1 (25%) | 21.8 | 4.6 | 0.35/0.62 | 190000 |
| | TMM2 (55%) | | | | |
| | Ir(L1)₃ (20%) | | | | |
| Sol-D2 | TMM1 (25%) | 21.0 | 4.2 | 0.43/0.55 | 240000 |
| | TMM2 (55%) | | | | |
| | Ir(L2)₃ (20%) | | | | |
| Sol-D3 | TMM1 (25%) | 18.3 | 4.4 | 0.68/0.32 | 190000 |
| | TMM2 (55%) | | | | |
| | Ir(L3)₃ (20%) | | | | |
| Sol-D4 | TMM1 (25%) | 20.9 | 4.2 | 0.350./61 | 260000 |
| | TMM2 (55%) | | | | |
| | Ir(L5)₃ (20%) | | | | |
| Sol-D5 | TMM1 (30%) | 17.5 | 4.6 | 0.69/0.30 | 200000 |
| | TMM2 (55%) | | | | |
| | Pt(L10)₃ (15%) | | | | |
| Sol-D6 | T1 (30%) | 20.4 | 4.1 | 0.43/0.55 | 250000 |
| | C1 (10%) | | | | |
| | TMM2 (40%) | | | | |
| | Ir(L2)₃ (20%) | | | | |
| Sol-D7 | T3 (30%) | 20.8 | 4.1 | 0.41/0.56 | 210000 |
| | C2 (10%) | | | | |
| | TMM2 (40%) | | | | |
| | Ir(L7)₃ (20%) | | | | |
| Sol-D8 | T3 (40%) | 21.2 | 4.6 | 0.43/0.52 | 170000 |
| | A1 (10%) | | | | |
| | TMM2 (40%) | | | | |
| | Pt(L11)₃ (10%) | | | | |

TABLE 2
Structural formulae of the materials used

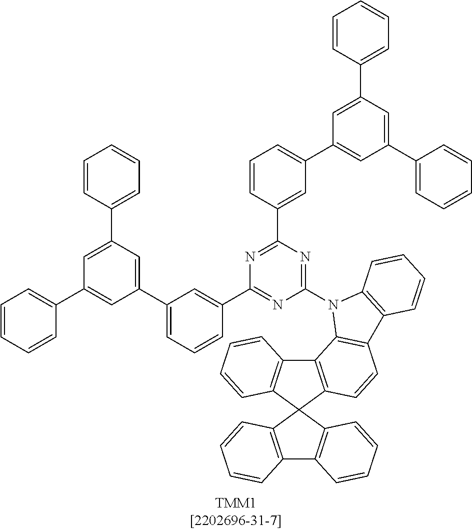 [SMILES: c1ccc(-c2cc(-c3ccccc3)cc(-c3cccc(-c4nc(-c5cccc(-c6cc(-c7ccccc7)cc(-c7ccccc7)c6)c5)nc(-n5c6ccccc6c6ccc7c(c65)-c5ccccc5C75c6ccccc6-c6ccccc65)n4)c3)c2)cc1]

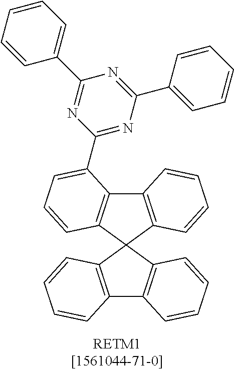 [SMILES: c1ccc(-c2nc(-c3ccccc3)nc(-c3cccc4c3-c3ccccc3C43c4ccccc4-c4ccccc43)n2)cc1]

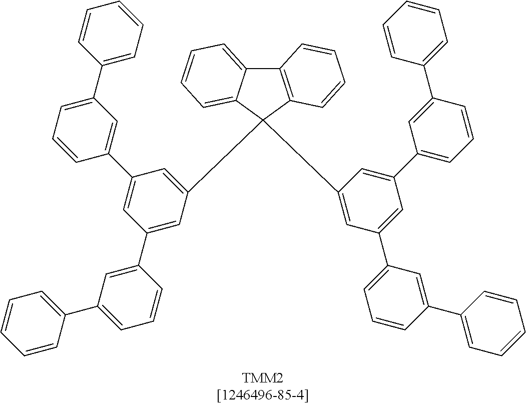 [SMILES: c1ccc(-c2cccc(-c3cc(-c4cccc(-c5ccccc5)c4)cc(C4(c5cc(-c6cccc(-c7ccccc7)c6)cc(-c6cccc(-c7ccccc7)c6)c5)c5ccccc5-c5ccccc54)c3)c2)cc1]

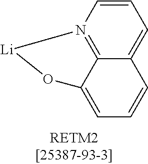

1. An organofunctional compound usable for production of functional layers of electronic devices, wherein the compound comprises at least one structural element of the formula (I) and/or (Ia) 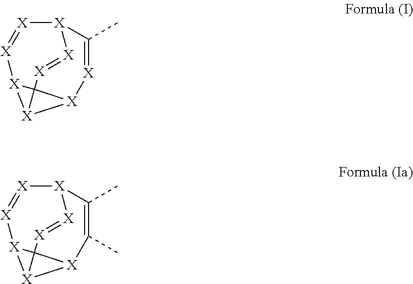
where the dotted bond represents the linkage of this group to another part of the organofunctional compound and in addition:
X is the same or different at each instance and is CR or N, with the proviso that not more than three, symbols X are N;
R is the same or different at each instance and is H, D, OH, F, Cl, Br, I, CN, NO₂, N(Ar)₂, N(R¹)₂, C(═O)N(Ar)₂, C(═O)N(R¹)₂, Si(Ar)₃, Si(R¹)₃, Ge(Ar)₃, Ge(R¹)₃, B(Ar)₂, B(R¹)₂, C(═O)Ar, C(═O)R¹, P(═O)(Ar)₂, P(═O)(R¹)₂, P(Ar¹)₂, P(R¹)₂, S(═O)Ar, S(═O)R¹, S(═O)₂Ar, S(═O)₂R¹, OSO₂Ar, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C≡C, Si(R¹)₂, Ge(R¹)₂, Sn(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; at the same time, two R radicals together may also form a ring system;
Ar is the same or different at each instance and is an aromatic or heteroaromatic ring system which has 5 to 60 aromatic ring atoms and may be substituted by one or more R¹ radicals; at the same time, it is possible for two Ar radicals bonded to the same silicon atom, nitrogen atom, phosphorus atom or boron atom also to be joined together via a bridge by a single bond or a bridge selected from B(R¹), C(R¹)₂, Si(R¹)₂, Ge(R¹)₂, C═O, C═NR¹, C═C(R¹)₂, O, S, S═O, SO₂, R¹), P(R¹) and P(═O)R¹;
R¹ is the same or different at each instance and is H, D, F, Cl, Br, I, CN, NO₂, N(Ar¹)₂, N(R²)₂, C(═O)Ar¹, C(═O)R², P(═O)(Ar¹)₂, P(Ar¹)₂, B(Ar¹)₂, B(OR²)₂, Si(Ar¹)₃, Si(R²)₃, Ge(Ar¹)₃, Ge(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, Ge(R²)₂, Sn(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 40 aromatic ring atoms, each of which may be substituted by one or more R² radicals, or an aryloxy or heteroaryloxy group which has 5 to 40 aromatic ring atoms and may be substituted by one or more R² radicals, or an aralkyl or heteroaralkyl group which has 5 to 40 aromatic ring atoms and may be substituted by one or more R² radicals, or a combination of these systems; at the same time, two or more, R¹ radicals together may form a ring system; at the same time, one or more R¹ radicals with a further part of the organofunctional compound may form a ring system;
Ar¹ is the same or different at each instance and is an aromatic or heteroaromatic ring system which has 5 to 30 aromatic ring atoms and may be substituted by one or more, preferably nonaromatic R² radicals; at the same time, it is possible for two Ar¹ radicals bonded to the same silicon atom, nitrogen atom, phosphorus atom or boron atom also to be joined to one another via a bridge by a single bond or a bridge selected from B(R²), C(R²)₂, Si(R²)₂, C═O, C═NR², C═C(R²)₂, O, S, S═O, SO₂, N(R²), P(R²) and P(═O)R²;
R² is the same or different at each instance and is H, D, F, Cl, Br, I, CN, B(OR³)₂, NO₂, C(═O)R³, CR³═C(R³)₂, C(═O)OR³, C(═O)N(R³)₂, Si(R³)₃, Ge(R³)₃, P(R³)₂, B(R³)₂, N(R³)₂, NO₂, P(═O)(R³)₂, OSO₂R³, OR³, S(═O)R³, S(═O)₂R³, a straight-chain alkyl, alkoxy or thioalkoxy group having 1 to 40 carbon atoms or a branched or cyclic alkyl, alkoxy or thioalkoxy group having 3 to 40 carbon atoms, each of which may be substituted by one or more R³ radicals, where one or more nonadjacent CH₂ groups may be replaced by —R³C═CR³—, —C≡C—, Si(R³)₂, Ge(R³)₂, Sn(R³)₂, C═O, C═S, C═NR³, —C(═O)O—, —C(═O)NR³—, NR³, P(═O)(R³), —O—, —S—, SO or SO₂ and where one or more hydrogen atoms may be replaced by D, F, Cl, Br, I, CN or NO₂, or an aromatic or heteroaromatic ring system which has 5 to 40 aromatic ring atoms and may be substituted in each case by one or more R³ radicals, or an aryloxy or heteroaryloxy group which has 5 to 40 aromatic ring atoms and may be substituted by one or more R³ radicals, or a combination of these systems; at the same time, two or more, preferably adjacent substituents R² together may form a ring system;
R³ is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, Cl, Br, I or CN and which may be substituted by one or more alkyl groups each having 1 to 4 carbon atoms; at the same time, two or more, substituents R³ together may form a ring system.

2. The compound as claimed in claim 1, wherein the compound comprises at least one structural element of the formula (II) 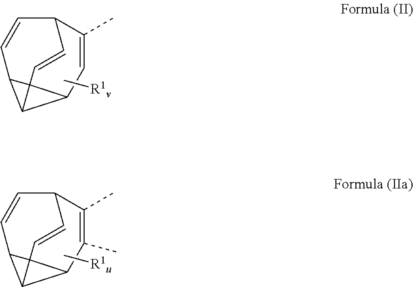
where the dotted bond represents the linkage of this group to another part of the organofunctional compound, the R¹ radicals have the definition given in claim 1, the index v is 0, 1, 2, 3, 4, 5, 6, 7, 8 or 9, and the index u is 0, 1, 2, 3, 4, 5, 6, 7 or 8.

3. The compound as claimed in claim 1 wherein the organofunctional compound usable for production of functional layers of electronic devices is selected from the group consisting of fluorescent emitters, phosphorescent emitters, emitters that exhibit TADF (thermally activated delayed fluorescence), host materials, electron transport materials, exciton blocker materials, electron injection materials, hole conductor materials, hole injection materials, n-dopants, p-dopants, wide band gap materials, electron blocker materials and hole blocker materials.

4. The compound of claim 1, wherein the organofunctional compound is selected from the group of the fluorenes, indenofluorenes, spirobifluorenes, carbazoles, indenocarbazoles, indolocarbazoles, spirocarbazoles, pyrimidines, triazines, lactams, triarylamines, dibenzofurans, dibenzothiophenes, dibenzothienes, imidazoles, benzimidazoles, benzoxazoles, benzothiazoles, 5-arylphenanthridin-6-ones, 9,10-dehydrophenanthrenes, fluoranthenes, anthracenes, benzanthracenes, and fluoradenes.

5. The compound of claim 1, wherein the organofunctional compound comprises at least one group conforming to at least one of the formulae (IIIa), (IIIb), (IIIc), (IIId), (IIIe), (IIIf), (IIIg) and/or (IIIh) 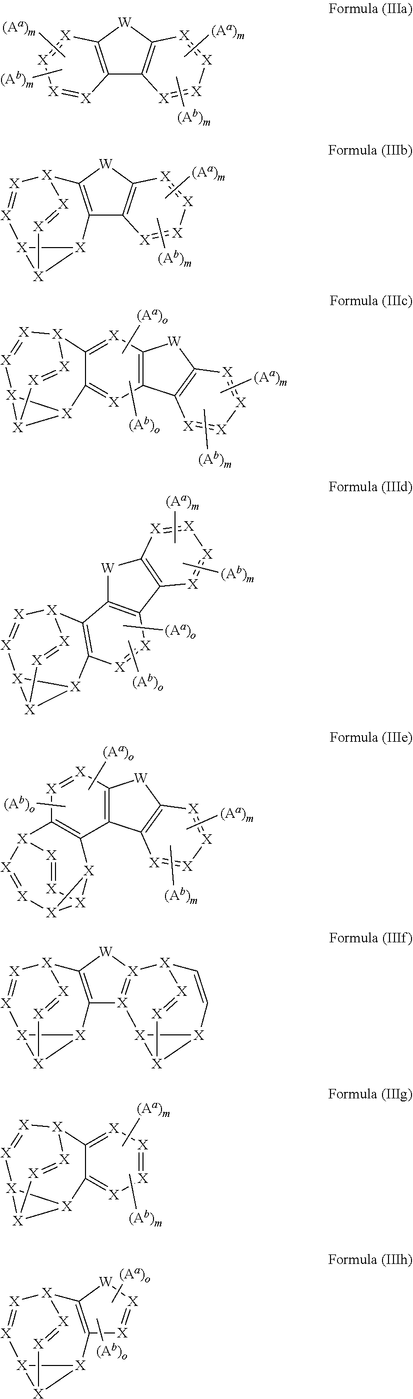
where the symbols used are as follows:
X is the same or different at each instance and is N or CR, preferably CR, or C if an A^{a} or A^{b} group is bonded to this atom, with the proviso that not more than two of the X groups in one cycle are N;
W is O, S, NR, NA^{a}, NA^{b}, BR, BA^{a}, BA^{b}, C(R)₂, CRA^{a}, C(A^{a})₂, CRA^{b}, C(A^{b})₂, CA^{a}A^{b}, —RC═CR—, —RC═CA^{a}-, -A^{a}C═CA^{a}-, —RC═CA^{b}-, -A^{b}C═CA^{b}-, -A^{b}C═CA^{a}-, SO, SO₂, Ge(R)₂, Ge(A^{a})₂, Ge(A^{b})₂, GeA^{a}A^{b}, Si(R)₂, Si(A^{a})₂, Si(A^{b})₂, SiA^{a}A^{b} or C═O;
m at each instance is independently 0, 1, 2, 3 or 4, preferably 0, 1 or 2, with the proviso that the sum total of the indices m per ring is not more than 4;
o at each instance is independently 0, 1 or 2, preferably 0 or 1, with the proviso that the sum total of the indices o per ring is not more than 2;
A^{a} is a functional structural element, preferably an aromatic or heteroaromatic ring system which has 5 to 40 ring atoms in each case and may be substituted by one or more substituents R;
A^{b} comprises, a structure of formula (I) and/or (Ia) according to claim 1 or a structure of formula (II) and/or (IIa) according to claim 2,
where the symbol R has the definition given in claim 1, with the proviso that the structure of formula (IIIa) has at least one A^{b} group.

6. The compound of claim 1, wherein the organofunctional compound comprises at least one hole transport group.

7. The compound of claim 1, wherein the organofunctional compound comprises at least one electron transport group.

8. The compound of claim 1, wherein the organofunctional compound comprises at least one group that leads to wide bandgap materials.

9. The compound of claim 1, wherein the organofunctional compound comprises at least one group that leads to materials that are used as host material.

10. The compound of claim 1, wherein the organofunctional compound comprises a group selected from the group consisting of phenyl, ortho-, meta- or para-biphenyl, terphenyl, especially branched terphenyl, quaterphenyl, especially branched quaterphenyl, 1-, 2-, 3- or 4-fluorenyl, 9,9′-diarylfluorenyl 1-, 2-, 3- or 4-spirobifluorenyl, pyridyl, pyrimidinyl, 1-, 2-, 3- or 4-dibenzofuranyl, 1-, 2-, 3- or 4-dibenzothienyl, pyrenyl, triazinyl, imidazolyl, benzimidazolyl, benzoxazolyl, benzothiazolyl, 1-, 2-, 3- or 4-carbazolyl, 1- or 2-naphthyl, anthracenyl, preferably 9-anthracenyl, trans- and cis-indenofluorenyl, indenocarbazolyl, indolocarbazolyl, spirocarbazolyl, 5-aryl-phenanthridin-6-on-yl, 9,10-dehydrophenanthrenyl, fluoranthenyl, tolyl, mesityl, phenoxytolyl, anisolyl, triarylaminyl, bis(triarylaminyl), tris(triarylaminyl), hexamethylindanyl, tetralinyl, monocycloalkyl, biscycloalkyl, tricycloalkyl, alkyl, for example tert-butyl, methyl, propyl, alkoxyl, alkylsulfanyl, alkylaryl, triarylsilyl, trialkylsilyl, xanthenyl, 10-arylphenoxazinyl, phenanthrenyl and triphenylenyl, each of which may be substituted by one or more radicals, where the functional structural element A^{a} preferably comprises a corresponding group or can be represented by a corresponding group.

11. The compound of claim 1, wherein the compound contains at least one solubilizing structural element or solubilizing group and at least one functional structural element or functional group, the functional structural element or the functional group being selected from hole transport groups, electron transport groups, structural elements or groups which lead to host materials, or structural elements or groups having wide bandgap properties.

12. The compound of claim 1, wherein the compound is a ligand in a metal complex.

13. A metal complex comprising at least one structure of the general Formula (1)
M(L)ₙ(L′)ₘ Formula (1)
where the symbols and indices used are as follows:
M is a transition metal;
L is the same or different at each instance and is a bidentate ligand;
L′ is the same or different at each instance and is a ligand;
n is 1, 2 or 3;
m is 0, 1, 2, 3 or 4;
at the same time, two or more ligands L may be joined to one another or for L to be joined to L′ by a single bond or a bivalent or trivalent bridge, thus forming a tridentate, tetradentate, pentadentate or hexadentate ligand system, wherein the metal complex contains at least one substructure of the formula (2) and/or (2a): 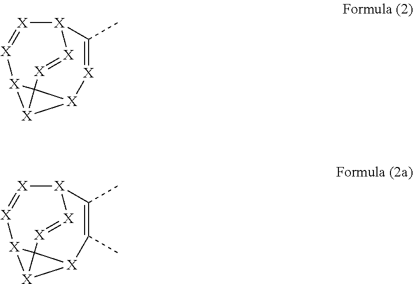
where the dotted bond represents the linkage of this group to a further part of the metal complex of the formula (1), and the symbol X has the definition set out in claim 1.

14. An oligomer, polymer or dendrimer containing one or more compounds as claimed in claim 1, wherein, rather than a hydrogen atom or a substituent, there are one or more bonds of the compounds to the polymer, oligomer or dendrimer.

15. A composition comprising at least one compound as claimed in claim 1 and at least one further compound selected from the group consisting of fluorescent emitters, phosphorescent emitters, emitters that exhibit TADF (thermally activated delayed fluorescence), host materials, electron transport materials, electron injection materials, hole conductor materials, hole injection materials, electron blocker materials and hole blocker materials.

16. A formulation comprising at least one compound as claimed in claim 1 and at least one solvent.

17. A method comprising utilizing the compound as claimed in claim 1 in an electronic device as emitter, host material, electron transport material, electron injection material, hole conductor material, hole injection material, electron blocker material, hole blocker material and/or wide bandgap material.

18. A process for preparing the compound as claimed in claim 1, wherein, in a coupling reaction, a compound comprising a structure of formula (I) and/or (Ia) is joined to a compound comprising at least one aromatic or heteroaromatic group.

19. An electronic device comprising at least one compound as claimed in claim 1, wherein the electronic device is preferably selected from the group consisting of organic electroluminescent devices, organic integrated circuits, organic field-effect transistors, organic thin-film transistors, organic light-emitting transistors, organic solar cells, organic optical detectors, organic photoreceptors, organic field-quench devices, light-emitting electrochemical cells and organic laser diodes.
